SEQUENCE LISTING (4 sequences)

SEQ ID NO: 1 - DNA, 55 bp, Artificial
  Probe for Mus musculus S100a8 gene
aaaagtgggtgtggcatctcacaaagacagccacaaggagtagcagagcttctgg

SEQ ID NO: 2 - DNA, 52 bp, Artificial
  Probe for Mus musculus S100a9 gene
gccatgtgacagctgcccaaccaagtctaaagggaatggcttactcaatggc

SEQ ID NO: 3 - DNA, 60 bp, Artificial
  Probe for Mus musculus Tubb3 gene
actgggttgtgtttatattcggggggaggggtatacttaataaagttactgctgtctgtc

SEQ ID NO: 4 - DNA, 57 bp, Artificial
  Probe for Mus musculus Tubb2c gene
gctgtcctgtgtcctgacatcacttgtacagataccaccattaaagcaattcatagt

(19) United States
(12) Patent Application Publication
(10) Pub. No.: US 2018/0284095 A1
(43) Pub. Date: Oct. 4, 2018

(54) BIOMARKERS ASSOCIATED WITH LSD1 INHIBITORS AND USES THEREOF

(71) Applicant: Oryzon Genomics, S.A., Madrid (ES)

(72) Inventors: Tamara MAES, Cornellà De Llobregat (ES); Cristina MASCARÓ CRUSAT, Cornellà De Llobregat (ES); David ROTLLANT POZO, Cornellà De Llobregat (ES)

(21) Appl. No.: 15/735,377

(22) PCT Filed: Jun. 10, 2016
(86) PCT No.: PCT/EP2016/063368

§ 371 Date: Dec. 11, 2017

(30) Foreign Application Priority Data
Jun. 12, 2015 (EP) 15382310.9
Jul. 17, 2015 (EP) 15382369.5

(51) Int. Cl. G01N 33/15; A61K 31/4245; A61K 31/165; A61K 31/495; A61K 31/40; A61P 25/28

(52) U.S. Cl. CPC G01N 33/15; A61K 31/4245; A61K 31/165; G01N 2800/52; A61K 31/40; A61P 25/28; G01N 2500/00; A61K 31/495

(57) ABSTRACT

The invention relates to biomarkers associated with LSD1 inhibitors and uses thereof to assess target engagement and to follow patient response to treatment. The invention further relates to novel therapeutic uses for LSD1 inhibitors based on said biomarkers.

### FIELD OF THE INVENTION

The invention relates to biomarkers associated with LSD1 inhibitors and uses thereof. In particular, the invention relates to the use of the biomarkers as disclosed herein to assess target engagement and to follow patient response to treatment. The invention further relates to novel therapeutic uses for LSD1 inhibitors based on said biomarkers.

### BACKGROUND

Aberrant gene expression in affected tissue as compared to normal tissue is a common characteristic of many human diseases. This is true for cancer and many neurological diseases which are characterized by changes in gene expression patterns. Gene expression patterns are controlled at multiple levels in the cell. Control of gene expression can occur through modifications of DNA: DNA promoter methylation is associated with suppression of gene expression. Another class of modifications involve histones, which are proteins, present in the nucleus of eukaryotic cells, that organize DNA strands into nucleosomes by forming molecular complexes around which the DNA winds. Histones play a critical role in modulating chromatin structure and DNA accessibility for replication, repair, and transcription. The covalent modification of histones is closely associated with regulation of gene transcription. Chromatin modifications have been suggested to represent an epigenetic code that is dynamically ‘written’ and ‘erased’ by specialized proteins, and ‘read’ or interpreted by proteins that translate the code into gene expression changes. A number of histone modifications have been discovered including histone acetylation, histone lysine methylation, histone arginine methylation, histone ubiquinylation, and histone sumoylation.

A group of enzymes known as histone lysine methyl transferases and histone lysine demethylases are involved in histone lysine modifications. One particular human histone lysine demethylase enzyme called Lysine Specific Demethylase-1 (LSD1) (Shi et al. (2004) Cell 119:941) has been reported to be involved in this crucial histone modification. LSD1 has a fair degree of structural similarity, and amino acid identity/homology to polyamine oxidases and monoamine oxidases, all of which (i.e., MAO-A, MAO-B and LSD1) are flavin dependent amine oxidases which catalyze the oxidation of nitrogen-hydrogen bonds and/or nitrogen carbon bonds.

LSD1 has been recognized as an interesting target for the development of new drugs to treat cancer, neurological diseases and other conditions, and a number of LSD1 inhibitors are currently under preclinical or clinical development for use in human therapy.

Finding pharmacodynamic (PD) biomarkers which indicate that a drug is active can be very valuable for use during clinical trials or in clinical practice. PD biomarkers can be used to monitor target engagement, i.e. to see if the drug is inhibiting the target against which the drug is designed to act in a subject receiving such drug. They can also be used to monitor the response of those patients receiving the drug. If the biomarker indicates that the patient is not responding appropriately to the drug treatment, then the dosage administered can be increased, reduced or treatment can be discontinued. Biomarkers can also be used to identify particular groups of patients that would benefit, or that would benefit the most, from receiving the drug treatment.

There are no well established PD markers currently available for use in combination with LSD1 inhibitors. There is thus a need to develop biomarkers associated with LSD1 inhibitors.

### SUMMARY OF THE INVENTION

The invention relates to the identification of biomarkers associated with LSD1 inhibitors and their use. The present invention is based, in part, on the discovery that a set of genes, as described in more detail below, act as PD markers for the activity of LSD1 inhibitors (henceforth “LSD1i”) and are thus useful to monitor the responsiveness of human subjects to LSD1 inhibition.

### BRIEF DESCRIPTION OF THE DRAWINGS

FIG. 1 shows the restoration of the discrimination index (DI) after 2 h retention test in female SAMP8 mice when treated for 2 (FIG. 1A) and 4 (FIG. 1B) months with compound 1 (Comp1) as described in Example 3.

FIG. 2 shows the restoration of the discrimination index (DI) after 2 h retention test in male SAMP8 mice when treated for 2 (FIG. 2A) and 4 (FIG. 2B) months with compound 1 (Comp1) as described in Example 3.

FIG. 3 shows the restoration of the discrimination index (DI) after 24 h retention test in male SAMP8 mice when treated for 2 (FIG. 3A) and 4 (FIG. 3B) months with compound 1 (Comp1) as described in Example 3.

FIG. 4 shows no changes in the platelet blood count of SAMP8 mice treated for 4 months with vehicle or compound 1 (Comp1) as described in Example 3.

FIG. 5 shows the reduction of S100A9 expression (Δ Cp) in female (FIG. 5A) and male (FIG. 5B) SAMP8 mice when treated with compound 1 (Comp1) as described in Example 5.

FIG. 6 shows S100A9 mRNA levels (Δ Cp S100A9-GADPH) in human cerebrospinal fluid samples from Alzheimer's disease donors determined as described in Example 8.

FIG. 7 shows the results obtained with compound 1 in the murine experimental autoimmune encephalomyelitis model as described in Example 9. Data represent the progression of the disease for each group measured as the mean clinical score (±SEM).

### DETAILED DESCRIPTION OF THE INVENTION

In one aspect, the disclosure relates to the analysis of genes that can act as PD markers for LSD1i and the identification of two closely related genes, S100A9 and S100A8, that can be used as such PD markers for monitoring LSD1 inhibition. As disclosed in more detail in the Examples below, S100A9 and S100A8 have been found to be downregulated by treatment with LSD1i in vivo in various tissues, including brain. Importantly, these genes are modulated by LSD1 inhibitors irrespective of gender, i.e. they are modulated in the same direction in both males and females. Moreover, downregulation of S100A9 and S100A8 by LSD1i has been confirmed by several techniques, including microarray and quantitative reverse transcriptase polymerase chain reaction (qRT-PCT).

Accordingly, the invention provides a method for monitoring LSD1 inhibition in a subject receiving treatment with an LSD1 inhibitor, comprising determining the level of a biomarker which is S100A9 and/or S100A8 in a sample obtained from the subject, wherein a decrease in the level of the biomarker in the sample as compared to the level of the biomarker in a control is indicative that LSD1 is being inhibited in the subject. Preferably, the method is performed in vitro.

In another aspect, the invention provides a method for monitoring the degree of LSD1 inhibition in a subject receiving treatment with an LSD1 inhibitor, comprising determining the level of a biomarker which is S100A9 and/or S100A8 in a sample obtained from the subject, wherein the degree of decrease in the level of the biomarker in the sample as compared to the level of the biomarker in a control is indicative of the degree of LSD1 inhibition in the subject. Preferably, the method is performed in vitro.

In yet another aspect, the invention provides a method for monitoring the response of a subject to treatment with an LSD1 inhibitor, comprising determining the level of a biomarker which is S100A9 and/or S100A8 in a sample obtained from the subject, wherein a decrease in the level of the biomarker in the sample as compared to the level of the biomarker in a control indicates response to the treatment with the LSD1 inhibitor. Preferably, the method is performed in vitro.

In another aspect, the invention provides a method for monitoring LSD1 inhibition in a subject receiving treatment with an LSD1 inhibitor, comprising (i) administering an LSD1 inhibitor to the subject, (ii) obtaining a sample from the subject, (iii) determining the level of a biomarker which is S100A9 and/or S100A8 in the sample obtained from the subject, and (iv) comparing the level of the biomarker in the sample with the level of the biomarker in a control, wherein a decrease in the level of the biomarker in the sample as compared to the level of the biomarker in a control is indicative that LSD1 is being inhibited in the subject. Preferably, the method is performed in vitro.

In another aspect, the invention provides a method for monitoring the degree of LSD1 inhibition in a subject receiving treatment with an LSD1 inhibitor, comprising (i) administering an LSD1 inhibitor to the subject, (ii) obtaining a sample from the subject, (iii) determining the level of a biomarker which is S100A9 and/or S100A8 in a sample obtained from the subject, and (iv) comparing the level of the biomarker in the sample with the level of the biomarker in a control, wherein the degree of decrease in the level of the biomarker in the sample as compared to the level of the biomarker in a control is indicative of the degree of LSD1 inhibition in the subject. Preferably, the method is performed in vitro.

In yet another aspect, the invention provides a method for monitoring the response of a subject to treatment with an LSD1 inhibitor, comprising (i) administering an LSD1 inhibitor to the subject, (ii) obtaining a sample from the subject, (iii) determining the level of a biomarker which is S100A9 and/or S100A8 in a sample obtained from the subject, and (iv) comparing the level of the biomarker in the sample with the level of the biomarker in a control, wherein a decrease in the level of the biomarker in the sample as compared to the level of the biomarker in a control indicates response to the treatment with the LSD1 inhibitor. Preferably, the method is performed in vitro.

S100A8 and S100A9 are mammalian calcium- and zinc-binding proteins which play a prominent role in the regulation of inflammatory processes and immune response, among others, as disclosed in more detail below. S100A8, also known as S100 Calcium Binding Protein A8, has the following aliases according to GeneCards:

| | |
|---|---|
| S100 Calcium Binding Protein A8 | CGLA |
| CAGA | CP-10 |
| CFAG | L1Ag |
| S100 Calcium-Binding Protein A8 (Calgranulin A) | MA387 |
| Calprotectin L1L Subunit | MIF |
| Cystic Fibrosis Antigen | NIF |
| Leukocyte L1 Complex Light Chain | P8 |
| Migration Inhibitory Factor-Related Protein 8 | Calgranulin A |
| Urinary Stone Protein Band A | calgranulin-A |
| MRP-8 | Protein S100-A8 |
| MRP8 | Calgranulin-A |
| S100 Calcium Binding Protein A8 (Calgranulin A) | p8 |
| 60B8AG | S100 Calcium- |
| | Binding Protein A8 |

S100A9, also known as S100 Calcium Binding Protein A9, has the following aliases according to GeneCards:

| | |
|---|---|
| S100 Calcium Binding Protein A9 | L1AG |
| CAGB | LIAG |
| CFAG | MAC387 |
| S100 Calcium-Binding Protein A9 (Calgranulin B) | MIF |
| Calprotectin L1H Subunit | NIF |
| Leukocyte L1 Complex Heavy Chain | P14 |
| Migration Inhibitory Factor-Related Protein 14 | Calgranulin B |
| MRP-14 | calgranulin-B |
| MRP14 | Protein S100-A9 |
| S100 Calcium Binding Protein A9 (Calgranulin B) | Calgranulin-B |
| 60B8AG | p14 |
| CGLB | S100 Calcium- |
| | Binding Protein A9 |

DNA and protein sequences of human and murine S100A8 and human and murine S100A9 have been previously reported, see GenBank Numbers (NCBI-GenBank Flat File Release 207.0, Apr. 15, 2015) and UniProtKB/Swiss-Prot Numbers (Knowledgebase Release 2015_06) listed below, each of which is incorporated herein by reference in its entirety for all purposes. Such sequences can be used to design procedures for detection of and analysis of the level of S100A8 and/or S100A9 by ways known to one skilled in the art.

| | NCBI Reference Sequence | | UniProtKB/Swiss-Prot | |
|---|---|---|---|---|
| Name | Human | Mice | Human | Mice |
| S100a9 | NM_002965.3 | NM_001281852.1 | S10A9_HUMAN, | P31725- |
| | | | P06702 | S10A9_MOUSE |
| S100a8 | NM_002964.4 | NM_013650.2 | S10A8_HUMAN, | P27005- |
| | | | P05109 | S10A8_MOUSE |

Exemplary amino acid sequences and nucleotide sequences of human and murine S100A9 and S100A8, respectively, are shown in the present application in SEQ ID NO: 1 to 8.

S100A8 and S100A9 are preferentially found in humans as a S100A8/S100A9 heterodimer (i.e. a dimer formed by the protein monomers S100A8 and S100A9), also known as Calprotectin. Calprotectin S100A8/S100A9 heterodimers can non-covalently pair with one another to form heterotetramers.

As used herein, the term “biomarker which is S100A9 and/or S100A8” encompasses any of S100A9 and/or S100A8 in any of the forms in which they can be found, including without limitation all monomeric forms and all heterodimeric or heterotetrameric forms thereof, such as Calprotectin. Preferably, the biomarkers of the invention relate to the human forms of S100A9 and S100A8.

As used herein, the term “determining the level of a biomarker which is S100A9 and/or S100A8” encompasses determining the level of any of S100A9 and/or S100A8 (in any of the forms in which each of them can be found) using any method known in the art to measure gene expression product levels, including mRNA and protein levels.

In the methods for monitoring described herein, the level of the biomarker can be determined as mRNA.

In the methods for monitoring described herein, the level of the biomarker can be determined as protein.

In the methods for monitoring described herein, the biomarker is preferably S100A9. In the methods for monitoring described herein, the level of S100A9 can be determined as mRNA. In the methods for monitoring described herein, the level of S100A9 can be determined as protein. In the methods for monitoring described herein, the level of the biomarker can be determined as S100A9 monomer. In the methods for monitoring described herein, the level of the biomarker can be determined as a S100A8/S100A9 heterodimer.

In another aspect, the invention provides a method for monitoring LSD1 inhibition in a subject receiving treatment with an LSD1 inhibitor, comprising determining the level of S100A9 in a sample obtained from the subject, wherein a decrease in the level of S100A9 in the sample as compared to the level of S100A9 in a control is indicative that LSD1 is being inhibited in the subject. Preferably, the method is performed in vitro.

In another aspect, the invention provides a method for monitoring the degree of LSD1 inhibition in a subject receiving treatment with an LSD1 inhibitor, comprising determining the level of S100A9 in a sample obtained from the subject, wherein the degree of decrease in the level of S100A9 in the sample as compared to the level of S100A9 in a control is indicative of the degree of LSD1 inhibition in the subject. Preferably, the method is performed in vitro.

In yet another aspect, the invention provides a method for monitoring the response of a subject to treatment with an LSD1 inhibitor, comprising determining the level of S100A9 in a sample obtained from the subject, wherein a decrease in the level of S100A9 in the sample as compared to the level of S100A9 in a control indicates response to the treatment with the LSD1 inhibitor. Preferably, the method is performed in vitro.

In yet another aspect, the invention provides a method for monitoring LSD1 inhibition in a subject receiving treatment with an LSD1 inhibitor, comprising determining the level of S100A8 in a sample obtained from the subject, wherein a decrease in the level of S100A8 in the sample as compared to the level of S100A8 in a control is indicative that LSD1 is being inhibited in the subject. Preferably, the method is performed in vitro.

In another aspect, the invention provides a method for monitoring the degree of LSD1 inhibition in a subject receiving treatment with an LSD1 inhibitor, comprising determining the level of S100A8 in a sample obtained from the subject, wherein the degree of decrease in the level of S100A8 in the sample as compared to the level of S100A8 in a control is indicative of the degree of LSD1 inhibition in the subject. Preferably, the method is performed in vitro.

In yet another aspect, the invention provides a method for monitoring the response of a subject to treatment with an LSD1 inhibitor, comprising determining the level of S100A8 in a sample obtained from the subject, wherein a decrease in the level of S100A8 in the sample as compared to the level of S100A8 in a control indicates response to the treatment with the LSD1 inhibitor. Preferably, the method is performed in vitro.

In yet another aspect, the invention provides a method for monitoring LSD1 inhibition in a subject receiving treatment with an LSD1 inhibitor, comprising determining the level of a S100A8/S100A9 heterodimer in a sample obtained from the subject, wherein a decrease in the level of the S100A8/S100A9 heterodimer in the sample as compared to the level of the S100A8/S100A9 heterodimer in a control is indicative that LSD1 is being inhibited in the subject. Preferably, the method is performed in vitro.

In another aspect, the invention provides a method for monitoring the degree of LSD1 inhibition in a subject receiving treatment with an LSD1 inhibitor, comprising determining the level of a S100A8/S100A9 heterodimer in a sample obtained from the subject, wherein the degree of decrease in the level of the S100A8/S100A9 heterodimer in the sample as compared to the level of the S100A8/S100A9 heterodimer in a control is indicative of the degree of LSD1 inhibition in the subject. Preferably, the method is performed in vitro.

In yet another aspect, the invention provides a method for monitoring the response of a subject to treatment with an LSD1 inhibitor, comprising determining the level of a S100A8/S100A9 heterodimer in a sample obtained from the subject, wherein a decrease in the level of the S100A8/S100A9 heterodimer in the sample as compared to the level of the S100A8/S100A9 heterodimer in a control indicates response to the treatment with the LSD1 inhibitor. Preferably, the method is performed in vitro.

In the methods for monitoring according to the present invention, the sample obtained from the subject to be compared to a control can be obtained at different time points, i.e. after the subject has been treated or has received a first, second, third etc dosage of the LSD1 inhibitor. The “subject receiving treatment with an LSD1 inhibitor”, i.e. the subject being monitored using the methods for monitoring according to the invention, can be either a subject under active treatment with the LSD1 inhibitor or a subject within a treatment break when the treatment with an LSD1 inhibitor may consist of multiple cycles of drug administration separated by break periods during which the subject may also be monitored.

As used in context of the methods for monitoring according to the present invention, a non-limiting example of a “control” is preferably a sample obtained from the to be monitored subject before the start of the treatment or at an earlier time point.

In the methods for monitoring described herein the sample is preferably a peripheral sample. The peripheral sample can be e.g. cerebrospinal fluid (CSF), blood, plasma, serum, stool, saliva, sputum, gingival crevicular fluid, hair follicle or skin biopsy.

In the methods for monitoring described herein the LSD1 inhibitor can be an irreversible LSD1 inhibitor or a reversible LSD1 inhibitor. Preferably, the LSD1 inhibitor is an irreversible LSD1 inhibitor.

In the methods for monitoring described herein the LSD1 inhibitor is preferably a 2-(hetero)arylcyclopropylamino compound.

In the methods for monitoring described herein the LSD1 inhibitor is preferably a compound disclosed in WO2010/043721, WO2010/084160, WO2011/035941, WO2011/042217, WO2011/131697, WO2012/013727, WO2012/013728, WO2012/045883, WO2013/057320, WO2013/057322, WO2012/135113, WO2013/022047, WO2014/058071, WO2010/143582, US2010-0324147, WO2011/131576, WO2014/084298, WO2014/086790, WO2014/164867, or WO2015/021128.

In the methods for monitoring described herein the LSD1 inhibitor is preferably a compound of formula (I), (II), (III), (IV), (V), (VI), (VII), (VIII), (IX), (X), (XI), (XII) or (XIII), as described in more detail below. More preferably, the LSD1 inhibitor is a compound of formula (III), (VI), (VIII), (IX), (X), (XI), (XII) or (XIII). Still more preferably, the LSD1 inhibitor is a compound from the lists of examples provided below for compounds of formulae (III), (VI), (VIII), (IX), (X) or (XI).

Preferably, in the methods for monitoring described herein the LSD1 inhibitor is (−) 5-((((trans)-2-(4-(benzyloxy)phenyl)cyclopropyl)amino)methyl)-1,3,4-oxadiazol-2-amine or a pharmaceutically acceptable salt or solvate thereof.

In the methods for monitoring described herein, the subject is preferably a human.

In the methods for monitoring described herein, the subject can be a patient or a healthy individual.

In the methods for monitoring described herein the subject can be a subject that has a CNS disease.

In the methods for monitoring described herein the subject can be a subject that has a neurodegenerative disease, for example Alzheimer's disease, Mild Cognitive Impairment, Parkinson's disease, difuse Lewy body disease, synucleinopathies, Huntington's disease, Down syndrome, or Amyotrophic lateral sclerosis, preferably Alzheimer's disease or Mild Cognitive Impairment.

In the methods for monitoring described herein the subject can be a subject that has a cognitive function related disease, for example dementia such as vascular dementia, Lewy body dementia, senile dementia, frontotemporal dementia and mixed dementia, delirium, amnesia, Rett disease, schizophrenia, attention-deficit/hyperactivity disorder, or postoperative cognitive dysfunction.

In the methods for monitoring described herein the subject can be a subject that has an autoimmune disease. For example, the autoimmune disease can be an acute or chronic autoimmune neuropathy such as multiple sclerosis. Multiple sclerosis can be for example chronic progressive multiple sclerosis.

In the methods for monitoring described herein the subject can be a subject that has an infection or a disease caused by an infection, preferably a bacterial infection, a fungal infection, a protozoan infection, an influenza infection, or a disease caused by any of said infections.

In the methods for monitoring described herein the subject can be a subject that has cancer.

In the methods for monitoring described herein the subject can be a subject that has a cardiovascular disease.

In another aspect, the invention provides a method for monitoring LSD1 inhibition in a subject receiving treatment with (−) 5-((((trans)-2-(4-(benzyloxy)phenyl)cyclopropyl)amino)methyl)-1,3,4-oxadiazol-2-amine or a pharmaceutically acceptable salt or solvate thereof, comprising determining the level of a biomarker which is S100A9 and/or S100A8 in a sample obtained from the subject, wherein a decrease in the level of the biomarker in the sample as compared to the level of the biomarker in a control is indicative that LSD1 is being inhibited in the subject. Preferably, the method is performed in vitro.

In another aspect, the invention provides a method for monitoring the degree of LSD1 inhibition in a subject receiving treatment with (−) 5-((((trans)-2-(4-(benzyloxy)phenyl)cyclopropyl)amino)methyl)-1,3,4-oxadiazol-2-amine or a pharmaceutically acceptable salt or solvate thereof, comprising determining the level of a biomarker which is S100A9 and/or S100A8 in a sample obtained from the subject, wherein the degree of decrease in the level of the biomarker in the sample as compared to the level of the biomarker in a control is indicative of the degree of LSD1 inhibition in the subject. Preferably, the method is performed in vitro.

In yet another aspect, the invention provides a method for monitoring the response of a subject to treatment with (−) 5-((((trans)-2-(4-(benzyloxy)phenyl)cyclopropyl)amino)methyl)-1,3,4-oxadiazol-2-amine or a pharmaceutically acceptable salt or solvate thereof, comprising determining the level of a biomarker which is S100A9 and/or S100A8 in a sample obtained from the subject, wherein a decrease in the level of the biomarker in the sample as compared to the level of the biomarker in a control indicates response to the treatment with the LSD1 inhibitor. Preferably, the method is performed in vitro.

In another aspect, the invention provides a method for determining whether a patient is likely to respond to treatment with an LSD1 inhibitor, comprising determining the level of a biomarker which is S100A9 and/or S100A8 in a sample obtained from the patient prior to treatment with the LSD1 inhibitor, where if the level of the biomarker in the sample is elevated as compared to a control, it is more likely that the LSD1 inhibitor would have a therapeutic effect on the patient. Preferably, the method is performed in vitro.

In another aspect, the invention provides a method for determining if a patient is a candidate to receive treatment with an LSD1 inhibitor, comprising determining the level of a biomarker which is S100A9 and/or S100A8 in a sample obtained from the patient prior to treatment with the LSD1 inhibitor, where if the level of the biomarker in the sample is elevated as compared to a control, the patient is regarded as a candidate to receive treatment with the LSD1 inhibitor. Preferably, the method is performed in vitro.

In another aspect, the invention provides a method for assessing whether a diseased cell is likely responsive to an LSD1 inhibitor, the method comprising

(i) determining the level of a biomarker which is S100A9 and/or S100A8 in a sample obtained from a patient prior to treatment with the LSD1 inhibitor,
(ii) assessing that the cell is likely responsive to the LSD1 inhibitor, when the level of the biomarker in the sample is elevated compared to a control. Preferably, the method is performed in vitro.

In another aspect, the invention provides a method for assessing whether a patient is likely responsive to an LSD1 inhibitor, the method comprising

(i) determining the level of a biomarker which is S100A9 and/or S100A8 in a sample obtained from the patient prior to treatment with the LSD1 inhibitor,
(ii) assessing that the patient is likely responsive to the LSD1 inhibitor, when the level of the biomarker in the sample is elevated compared to a control. Preferably, the method is performed in vitro.

In the methods described herein, the level of the biomarker can be determined as mRNA.

In the methods described herein, the level of the biomarker can be determined as protein.

In the methods described herein the biomarker is preferably S100A9. In the methods described herein, the level of S100A9 can be determined as mRNA. In the methods described herein, the level of S100A9 can be determined as protein. In the methods described herein, the level of the biomarker can be determined as S100A9 monomer. In the methods described herein, the level of the biomarker can be determined as a S100A8/S100A9 heterodimer.

In another aspect, the invention provides a method for determining whether a patient is likely to respond to treatment with an LSD1 inhibitor, comprising determining the level of S100A9 in a sample obtained from the patient prior to treatment with the LSD1 inhibitor, where if the level of S100A9 in the sample is elevated as compared to a control, it is more likely that the LSD1 inhibitor would have a therapeutic effect on the patient. Preferably, the method is performed in vitro.

In another aspect, the invention provides a method for determining if a patient is a candidate to receive treatment with an LSD1 inhibitor, comprising determining the level of S100A9 in a sample obtained from the patient prior to treatment with the LSD1 inhibitor, where if the level of S100A9 in the sample is elevated as compared to a control, the patient is regarded as a candidate to receive treatment with the LSD1 inhibitor. Preferably, the method is performed in vitro.

In another aspect, the invention provides a method for assessing whether a diseased cell is likely responsive to an LSD1 inhibitor, the method comprising

(i) determining the level of S100A9 in a sample obtained from a patient prior to treatment with the LSD1 inhibitor,
(ii) assessing that the cell is likely responsive to the LSD1 inhibitor, when the level of S100A9 in the sample is elevated compared to a control. Preferably, the method is performed in vitro.

In another aspect, the invention provides a method for assessing whether a patient is likely responsive to an LSD1 inhibitor, the method comprising

(i) determining the level of S100A9 in a sample obtained from the patient prior to treatment with the LSD1 inhibitor,
(ii) assessing that the patient is likely responsive to the LSD1 inhibitor, when the level of S100A9 in the sample is elevated compared to a control. Preferably, the method is performed in vitro.

In another aspect, the invention provides a method for determining whether a patient is likely to respond to treatment with an LSD1 inhibitor, comprising determining the level of S100A8 in a sample obtained from the patient prior to treatment with the LSD1 inhibitor, where if the level of S100A8 in the sample is elevated as compared to a control, it is more likely that the LSD1 inhibitor would have a therapeutic effect on the patient. Preferably, the method is performed in vitro.

In another aspect, the invention provides a method for determining if a patient is a candidate to receive treatment with an LSD1 inhibitor, comprising determining the level of S100A8 in a sample obtained from the patient prior to treatment with the LSD1 inhibitor, where if the level of S100A8 in the sample is elevated as compared to a control, the patient is regarded as a candidate to receive treatment with the LSD1 inhibitor. Preferably, the method is performed in vitro.

In another aspect, the invention provides a method for assessing whether a diseased cell is likely responsive to an LSD1 inhibitor, the method comprising

(i) determining the level of S100A8 in a sample obtained from a patient prior to treatment with the LSD1 inhibitor,
(ii) assessing that the cell is likely responsive to the LSD1 inhibitor, when the level of S100A8 in the sample is elevated compared to a control. Preferably, the method is performed in vitro.

In another aspect, the invention provides a method for assessing whether a patient is likely responsive to an LSD1 inhibitor, the method comprising

(i) determining the level of S100A8 in a sample obtained from the patient prior to treatment with the LSD1 inhibitor,
(ii) assessing that the patient is likely responsive to the LSD1 inhibitor, when the level of S100A8 in the sample is elevated compared to a control. Preferably, the method is performed in vitro.

In another aspect, the invention provides a method for determining whether a patient is likely to respond to treatment with an LSD1 inhibitor, comprising determining the level of a S100A8/S100A9 heterodimer in a sample obtained from the patient prior to treatment with the LSD1 inhibitor, where if the level of the S100A8/S100A9 heterodimer in the sample is elevated as compared to a control, it is more likely that the LSD1 inhibitor would have a therapeutic effect on the patient. Preferably, the method is performed in vitro.

In another aspect, the invention provides a method for determining if a patient is a candidate to receive treatment with an LSD1 inhibitor, comprising determining the level of a S100A8/S100A9 heterodimer in a sample obtained from the patient prior to treatment with the LSD1 inhibitor, where if the level of the S100A8/S100A9 heterodimer in the sample is elevated as compared to a control, the patient is regarded as a candidate to receive treatment with the LSD1 inhibitor. Preferably, the method is performed in vitro.

In another aspect, the invention provides a method for assessing whether a diseased cell is likely responsive to an LSD1 inhibitor, the method comprising

(i) determining the level of a S100A8/S100A9 heterodimer in a sample obtained from a patient prior to treatment with the LSD1 inhibitor,
(ii) assessing that the cell is likely responsive to the LSD1 inhibitor, when the level of the S100A8/S100A9 heterodimer in the sample is elevated compared to a control. Preferably, the method is performed in vitro.

In another aspect, the invention provides a method for assessing whether a patient is likely responsive to an LSD1 inhibitor, the method comprising

(i) determining the level of a S100A8/S100A9 heterodimer in a sample obtained from the patient prior to treatment with the LSD1 inhibitor,
(ii) assessing that the patient is likely responsive to the LSD1 inhibitor, when the level of the S100A8/S100A9 heterodimer in the sample is elevated compared to a control. Preferably, the method is performed in vitro.

As used in context of the methods of the present invention for assessing/determining/predicting if a patient will be likely responsive to an LSD1 inhibitor or to treatment with an LSD1 inhibitor and/or for selecting patients for receiving treatment with an LSD1 inhibitor, a non-limiting example of a “control” is preferably a healthy control.

In the methods described herein the sample is preferably a peripheral sample. The peripheral sample can be e.g. cerebrospinal fluid (CSF), blood, plasma, serum, stool, saliva, sputum, gingival crevicular fluid, hair follicle or skin biopsy.

In the methods described herein the LSD1 inhibitor can be an irreversible LSD1 inhibitor or a reversible LSD1 inhibitor. Preferably, the LSD1 inhibitor is an irreversible LSD1 inhibitor.

In the methods described herein the LSD1 inhibitor is preferably a 2-(hetero)arylcyclopropylamino compound.

In the methods described herein the LSD1 inhibitor is preferably a compound disclosed in WO2010/043721, WO2010/084160, WO2011/035941, WO2011/042217, WO2011/131697, WO2012/013727, WO2012/013728, WO2012/045883, WO2013/057320, WO2013/057322, WO2012/135113, WO2013/022047, WO2014/058071 WO2010/143582, US2010-0324147, WO2011/131576, WO2014/084298, WO2014/086790, WO2014/164867, or WO2015/021128.

In the methods described herein the LSD1 inhibitor is preferably a compound of formula (I), (II), (III), (IV), (V), (VI), (VII), (VIII), (IX), (X), (XI), (XII) or (XIII). More preferably, the LSD1 inhibitor is a compound of formula (III), (VI), (VIII), (IX), (X), (XI), (XII) or (XIII). Still more preferably, the LSD1 inhibitor is a compound from the lists of examples provided below for compounds of formulae (III), (VI), (VIII), (IX), (X) or (XI) Preferably, in the methods described herein the LSD1 inhibitor is (−) 5-((((trans)-2-(4-(benzyloxy)phenyl)cyclopropyl)amino)methyl)-1,3,4-oxadiazol-2-amine or a pharmaceutically acceptable salt or solvate thereof.

In the methods described herein, the patient is preferably a human.

In the methods described herein the patient can be a patient that has a CNS disease.

In the methods described herein the patient can be a patient that has a neurodegenerative disease, for example Alzheimer's disease, Mild Cognitive Impairment, Parkinson's disease, difuse Lewy body disease, synucleinopathies, Huntington's disease, Down syndrome, or Amyotrophic lateral sclerosis, preferably Alzheimer's disease or Mild Cognitive Impairment.

In the methods described herein the patient can be a patient that has a cognitive function related disease, for example dementia such as vascular dementia, Lewy body dementia, senile dementia, frontotemporal dementia and mixed dementia, delirium, amnesia, Rett disease, schizophrenia, attention-deficit/hyperactivity disorder, or postoperative cognitive dysfunction.

In the methods described herein the patient can be a patient that has an autoimmune disease. For example, the autoimmune disease can be an acute or chronic autoimmune neuropathy such as multiple sclerosis. Multiple sclerosis can be for example chronic progressive multiple sclerosis.

In the methods described herein the patient can be a patient that has an infection or a disease caused by an infection, preferably a bacterial infection, a fungal infection, a protozoan infection, an influenza infection, or a disease caused by any of said infections.

In the methods described herein the patient can be a patient that has cancer.

In the methods described herein the patient can be a patient that has a cardiovascular disease.

In another aspect, the invention provides a method for determining whether a patient is likely to respond to treatment with an LSD1 inhibitor which is (−) 5-((((trans)-2-(4-(benzyloxy)phenyl)cyclopropyl)amino)methyl)-1,3,4-oxadiazol-2-amine, comprising determining the level of a biomarker which is S100A9 and/or S100A8 in a sample obtained from the patient prior to treatment with the LSD1 inhibitor, where if the level of the biomarker in the sample is elevated as compared to a control, it is more likely that the LSD1 inhibitor would have a therapeutic effect on the patient. Preferably, the method is performed in vitro.

In another aspect, the invention provides a method for determining if a patient is a candidate to receive treatment with an LSD1 inhibitor which is (−) 5-((((trans)-2-(4-(benzyloxy)phenyl)cyclopropyl)amino)methyl)-1,3,4-oxadiazol-2-amine, comprising determining the level of a biomarker which is S100A9 and/or S100A8 in a sample obtained from the patient prior to treatment with the LSD1 inhibitor, where if the level of the biomarker in the sample is elevated as compared to a control, the patient is regarded as a candidate to receive treatment with the LSD1 inhibitor. Preferably, the method is performed in vitro.

In another aspect, the invention provides a method for assessing whether a diseased cell is likely responsive to an LSD1 inhibitor which is (−) 5-((((trans)-2-(4-(benzyloxy)phenyl)cyclopropyl)amino)methyl)-1,3,4-oxadiazol-2-amine, the method comprising

(i) determining the level of a biomarker which is S100A9 and/or S100A8 in a sample obtained from a patient prior to treatment with the LSD1 inhibitor,
(ii) assessing that the cell is likely responsive to the LSD1 inhibitor, when the level of the biomarker in the sample is elevated compared to a control. Preferably, the method is performed in vitro.

In another aspect, the invention provides a method for assessing whether a patient is likely responsive to an LSD1 inhibitor which is (−) 5-((((trans)-2-(4-(benzyloxy)phenyl)cyclopropyl)amino)methyl)-1,3,4-oxadiazol-2-amine, the method comprising

(i) determining the level of a biomarker which is S100A9 and/or S100A8 in a sample obtained from the patient prior to treatment with the LSD1 inhibitor,
(ii) assessing that the patient is likely responsive to the LSD1 inhibitor, when the level of the biomarker in the sample is elevated compared to a control. Preferably, the method is performed in vitro.

In the methods described herein, the method can comprise an extra step of obtaining a sample from the patient prior to determining the level of the biomarker.

In yet another aspect, the invention provides for a use of a biomarker which is S100A9 and/or S100A8 as a selection tool to identify patients with increased likelihood to benefit from treatment with an LSD1 inhibitor. Preferably, the use is an in vitro use.

In the uses described herein the biomarker is preferably S100A9.

In the uses described herein the biomarker can be S100A9 mRNA.

In the uses described herein the biomarker can be S100A9 protein.

In the uses described herein the biomarker can be a S100A8/S100A9 heterodimer.

In the uses described herein the LSD1 inhibitor can be an irreversible LSD1 inhibitor or a reversible LSD1 inhibitor. Preferably, the LSD1 inhibitor is an irreversible LSD1 inhibitor.

In the uses described herein the LSD1 inhibitor is preferably a 2-(hetero)arylcyclopropylamino compound.

In the uses described herein the LSD1 inhibitor is preferably a compound disclosed in WO2010/043721, WO2010/084160, WO2011/035941, WO2011/042217, WO2011/131697, WO2012/013727, WO2012/013728, WO2012/045883, WO2013/057320, WO2013/057322, WO2012/135113, WO2013/022047, WO2014/058071, WO2010/143582, US2010-0324147, WO2011/131576, WO2014/084298, WO2014/086790, WO2014/164867, or WO2015/021128.

In the uses described herein the LSD1 inhibitor is preferably a compound of formula (I), (II), (III), (IV), (V), (VI), (VII), (VIII), (IX), (X), (XI), (XII) or (XIII). More preferably, the LSD1 inhibitor is a compound of formula (III), (VI), (VIII), (IX), (X), (XI), (XII) or (XIII). Still more preferably, the LSD1 inhibitor is a compound from the lists of examples provided below for compounds of formulae (III), (VI), (VIII), (IX), (X) or (XI).).

Preferably, in the uses described herein the LSD1 inhibitor is (−) 5-((((trans)-2-(4-(benzyloxy)phenyl)cyclopropyl)amino)methyl)-1,3,4-oxadiazol-2-amine or a pharmaceutically acceptable salt or solvate thereof.

In the uses described herein, the patient is preferably a human.

In the uses described herein the patient can be a patient that has a CNS disease.

In the uses described herein the patient can be a patient that has a neurodegenerative disease, for example Alzheimer's disease, Mild Cognitive Impairment, Parkinson's disease, difuse Lewy body disease, synucleinopathies, Huntington's disease, Down syndrome, or Amyotrophic lateral sclerosis, preferably Alzheimer's disease or Mild Cognitive Impairment.

In the uses described herein the patient can be a patient that has cognitive function related disease, for example dementia (such as vascular dementia, Lewy body dementia, senile dementia, frontotemporal dementia and mixed dementia), delirium, amnesia, Rett disease, schizophrenia, attention-deficit/hyperactivity disorder, or postoperative cognitive dysfunction.

In the uses described herein the patient can be a patient that has an autoimmune disease. For example, the autoimmune disease can be an acute or chronic autoimmune neuropathy such as multiple sclerosis. Multiple sclerosis can be for example chronic progressive multiple sclerosis.

In the uses described herein the patient can be a patient that has an infection or a disease caused by an infection, preferably a bacterial infection, a fungal infection, a protozoan infection, an influenza infection, or a disease caused by any of said infections.

In the uses described herein the patient can be a patient that has cancer.

In the uses described herein the patient can be a patient that has a cardiovascular disease.

In another aspect, the invention provides a method for determining whether a beneficial effect in cognitive function is likely to be produced by treatment with an LSD1 inhibitor in a patient suffering from a neurodegenerative disease, comprising determining the level of a biomarker which is S100A9 and/or S100A8 in a sample obtained from the patient prior to treatment with the LSD1 inhibitor, where if the level of the biomarker in the sample is elevated compared to a control, it is more likely that the LSD1 inhibitor would produce a beneficial effect in cognitive function in the patient. Preferably, the method is performed in vitro.

In another aspect, the invention provides a method for determining whether a beneficial effect in cognitive function is likely to be produced by treatment with an LSD1 inhibitor in a patient suffering from a cognitive function related disease, comprising determining the level of a biomarker which is S100A9 and/or S100A8 in a sample obtained from the patient prior to treatment with the LSD1 inhibitor, where if the level of the biomarker in the sample is elevated compared to a control, it is more likely that the LSD1 inhibitor would produce a beneficial effect in cognitive function in the patient. Preferably, the method is performed in vitro.

In the methods described above the biomarker is preferably S100A9. The level of S100A9 can be determined as mRNA. The level of S100A9 can be determined as protein. The level of the biomarker can be determined as S100A9 monomer. The level of the biomarker can be determined as a S100A8/S100A9 heterodimer.

In the methods described above the sample is preferably a peripheral sample. The peripheral sample is preferably cerebrospinal fluid (CSF), blood, plasma, or serum.

In the methods described above the LSD1 inhibitor can be an irreversible LSD1 inhibitor or a reversible LSD1 inhibitor. Preferably, the LSD1 inhibitor is an irreversible LSD1 inhibitor.

In the methods described above the LSD1 inhibitor is preferably a 2-(hetero)arylcyclopropylamino compound.

In the methods described above the LSD1 inhibitor is preferably a compound disclosed in WO2010/043721, WO2010/084160, WO2011/035941, WO2011/042217, WO2011/131697, WO2012/013727, WO2012/013728, WO2012/045883, WO2013/057320, WO2013/057322, WO2012/135113, WO2013/022047 and WO2014/058071 WO2010/143582, US2010-0324147, WO2011/131576, WO2014/084298, WO2014/086790, WO2014/164867, or WO2015/021128.

In the methods described above the LSD1 inhibitor is preferably a compound of formula (I), (II), (III), (IV), (V), (VI), (VII), (VIII), (IX), (X), (XI), (XII) or (XIII). More preferably, the LSD1 inhibitor is a compound of formula (III), (VI), (VIII), (IX), (X), (XI), (XII) or (XIII). Still more preferably, the LSD1 inhibitor is a compound from the lists of examples provided below for compounds of formulae (III), (VI), (VIII), (IX), (X) or (XI).

Preferably, in the methods described above the LSD1 inhibitor is (−) 5-((((trans)-2-(4-(benzyloxy)phenyl)cyclopropyl)amino)methyl)-1,3,4-oxadiazol-2-amine or a pharmaceutically acceptable salt or solvate thereof. Accordingly, the invention provides a method for determining whether a beneficial effect in cognitive function is likely to be produced by treatment with an LSD1 inhibitor which is (−) 5-((((trans)-2-(4-(benzyloxy)phenyl)cyclopropyl)amino)methyl)-1,3,4-oxadiazol-2-amine or a pharmaceutically acceptable salt or solvate thereof in a patient suffering from a neurodegenerative disease, comprising determining the level of a biomarker which is S100A9 and/or S100A8 in a sample obtained from the patient prior to treatment with the LSD1 inhibitor, where if the level of the biomarker in the sample is elevated compared to a control, it is more likely that the LSD1 inhibitor would produce a beneficial effect in cognitive function in the patient. The invention further provides a method for determining whether a beneficial effect in cognitive function is likely to be produced by treatment with an LSD1 inhibitor which is (−) 5-((((trans)-2-(4-(benzyloxy)phenyl)cyclopropyl)amino)methyl)-1,3,4-oxadiazol-2-amine or a pharmaceutically acceptable salt or solvate thereof in a patient suffering from a cognitive function related disease, comprising determining the level of a biomarker which is S100A9 and/or S100A8 in a sample obtained from the patient prior to treatment with the LSD1 inhibitor, where if the level of the biomarker in the sample is elevated compared to a control, it is more likely that the LSD1 inhibitor would produce a beneficial effect in cognitive function in the patient.

Preferably, the methods are performed in vitro.

In the methods described above the neurodegenerative disease can be e.g. Alzheimer's disease or Mild Cognitive Impairment.

In another aspect, the invention provides a method for selecting a patient having mild cognitive impairment for receiving treatment with an LSD1 inhibitor, comprising determining the level of a biomarker which is S100A9 and/or S100A8 in a sample obtained from the patient prior to treatment with the LSD1 inhibitor, and selecting the patient to receive treatment with the LSD1 inhibitor if the level of the biomarker in the sample is elevated compared to a control. Preferably, the method is performed in vitro.

In another aspect, the invention provides a method for selecting a patient having mild cognitive impairment for receiving treatment with an LSD1 inhibitor, comprising determining the level of S100A9 in a sample obtained from the patient prior to treatment with the LSD1 inhibitor, and selecting the patient to receive treatment with the LSD1 inhibitor if the level of S100A9 in the sample is elevated compared to a control. Preferably, the method is performed in vitro.

In another aspect, the invention provides a method for selecting a patient having mild cognitive impairment for receiving treatment with an LSD1 inhibitor, comprising determining the level of S100A8 in a sample obtained from the patient prior to treatment with the LSD1 inhibitor, and selecting the patient to receive treatment with the LSD1 inhibitor if the level of S100A8 in the sample is elevated compared to a control. Preferably, the method is performed in vitro.

In another aspect, the invention provides a method for selecting a patient having mild cognitive impairment for receiving treatment with an LSD1 inhibitor, comprising determining the level of a S100A8/S100A9 heterodimer in a sample obtained from the patient prior to treatment with the LSD1 inhibitor, and selecting the patient to receive treatment with the LSD1 inhibitor if the level of the S100A8/S100A9 heterodimer in the sample is elevated compared to a control. Preferably, the method is performed in vitro.

In the methods described above the sample is preferably a peripheral sample. The peripheral sample is preferably cerebrospinal fluid (CSF), blood, plasma, or serum.

In the methods described above the LSD1 inhibitor can be an irreversible LSD1 inhibitor or a reversible LSD1 inhibitor. Preferably, the LSD1 inhibitor is an irreversible LSD1 inhibitor.

In the methods described above the LSD1 inhibitor is preferably a 2-(hetero)arylcyclopropylamino compound.

In the methods described above the LSD1 inhibitor is preferably a compound disclosed in WO2010/043721, WO2010/084160, WO2011/035941, WO2011/042217, WO2011/131697, WO2012/013727, WO2012/013728, WO2012/045883, WO2013/057320, WO2013/057322, WO2012/135113, WO2013/022047, WO2014/058071, WO2010/143582, US2010-0324147, WO2011/131576, WO2014/084298, WO2014/086790, WO2014/164867, or WO2015/021128.

In the method described above the LSD1 inhibitor is preferably a compound of formula (I), (II), (III), (IV), (V), (VI), (VII), (VIII), (IX), (X), (XI), (XII) or (XIII). More preferably, the LSD1 inhibitor is a compound of formula (III), (VI), (VIII), (IX), (X), (XI), (XII) or (XIII). Still more preferably, the LSD1 inhibitor is a compound from the lists of examples provided below for compounds of formulae (III), (VI), (VIII), (IX), (X) or (XI).

Preferably, in the methods described above the LSD1 inhibitor is (−) 5-((((trans)-2-(4-(benzyloxy)phenyl)cyclopropyl)amino)methyl)-1,3,4-oxadiazol-2-amine or a pharmaceutically acceptable salt or solvate thereof. Accordingly, the invention provides a method for selecting a patient having mild cognitive impairment for receiving treatment with an LSD1 inhibitor which is (−) 5-((((trans)-2-(4-(benzyloxy)phenyl)cyclopropyl)amino)methyl)-1,3,4-oxadiazol-2-amine or a pharmaceutically acceptable salt or solvate thereof, comprising determining the level of a biomarker which is S100A9 and/or S100A8 in a sample obtained from the patient prior to treatment with the LSD1 inhibitor, and selecting the patient to receive treatment with the LSD1 inhibitor if the level of the biomarker in the sample is elevated compared to a control.

Preferably, the method is performed in vitro.

In the above methods the method can comprise an extra step of obtaining a sample from the patient prior to determining the level of the biomarker.

In a certain aspect, the present invention relates to the use of a primer/a primer pair in the in vitro methods of the present invention. In a certain aspect, the present invention relates to a primer/a primer pair for use in the in vitro methods of the present invention. The primer/primer pair can be used for determining the level of a biomarker which is S100A9 and/or S100A8. For example, the primer/primer pair can specifically bind to the nucleotide sequence of a biomarker which is S100A9 and/or S100A8. In a certain aspect, the present invention relates to the use of a primer/a primer pair for a biomarker which is S100A9 and/or S100A8 in the in vitro methods of the present invention. The primer/primer pair can, for example, be used in amplifying the nucleotide sequence of a biomarker which is S100A9 and/or S100A8, or in amplifying a part of the sequence. Thus, the primer/a primer pair can, for example, be useful to determine the mRNA level of a biomarker which is S100A9 and/or S100A8. The term “primer pair” as used herein refers normally to a forward primer and a reverse primer that are used to amplify a nucleotide sequence of a biomarker which is S100A9 and/or S100A8, or a part of that sequence. It is understood that the forward primer normally binds to the strand that is complementary to the strand that the reverse primer binds to.

In a further aspect, the present invention relates to an in vitro use of a primer/a primer pair for monitoring the response of a subject to treatment with an LSD1 inhibitor, wherein the primer/primer pair is for determining the level of a biomarker which is S100A9 and/or S100A8. For example, the primer/primer pair can specifically bind to the nucleotide sequence of a biomarker which is S100A9 and/or S100A8.

In a further aspect, the present invention relates to an in vitro use of a primer/a primer pair for identifying patients with increased likelihood to benefit from treatment with an LSD1 inhibitor, wherein the primer/primer pair is for determining the level of a biomarker which is S100A9 and/or S100A8. For example, the primer/primer pair can specifically bind to the nucleotide sequence of a biomarker which is S100A9 and/or S100A8.

In a further aspect, the present invention relates to a primer/a primer pair for use in monitoring the response of a subject to treatment with an LSD1 inhibitor, wherein the primer/primer pair is for determining the level of a biomarker which is S100A9 and/or S100A8. For example, the primer/primer pair can specifically bind to the nucleotide sequence of a biomarker which is S100A9 and/or S100A8.

In a further aspect, the present invention relates to a primer/a primer pair for use in identifying patients with increased likelihood to benefit from treatment with an LSD1 inhibitor, wherein the primer/primer pair is for determining the level of a biomarker which is S100A9 and/or S100A8. For example, the primer/primer pair can specifically bind to the nucleotide sequence of a biomarker which is S100A9 and/or S100A8.

In a certain aspect, the present invention relates to the use of a binding molecule in the in vitro methods of the present invention. In a certain aspect, the present invention relates to a binding molecule for use in the in vitro methods of the present invention. The binding molecule specifically binds to a biomarker which is S100A9 and/or S100A8, wherein the biomarker is a protein. The binding molecule can be an antibody. In a certain aspect, the present invention relates to the use of an antibody in the in vitro methods of the present invention.

In a further aspect, the present invention relates to an in vitro use of a binding molecule specifically binding to a biomarker which is S100A9 protein and/or S100A8 protein for monitoring the response of a subject to treatment with an LSD1 inhibitor. The binding molecule can be for example an antibody.

In a further aspect, the present invention relates to an in vitro use of a binding molecule specifically binding to a biomarker which is S100A9 protein and/or S100A8 protein for identifying patients with increased likelihood to benefit from treatment with an LSD1 inhibitor. The binding molecule can be for example an antibody.

In a further aspect, the present invention relates to a binding molecule specifically binding to a biomarker which is S100A9 protein and/or S100A8 protein for use in monitoring the response of a subject to treatment with an LSD1 inhibitor. The binding molecule can be for example an antibody.

In a further aspect, the present invention relates to a binding molecule specifically binding to a biomarker which is S100A9 protein and/or S100A8 protein for use in identifying patients with increased likelihood to benefit from treatment with an LSD1 inhibitor. The binding molecule can be for example an antibody.

In a certain aspect, the present invention relates to the use of a kit in the in vitro methods of the present invention, wherein the kit comprises means and methods for determining the level of a biomarker which is S100A9 and/or S100A8 in accordance with the present invention. In a certain aspect, the present invention relates to a kit for use in the in vitro methods of the present invention, wherein the kit comprises means and methods for determining the level of a biomarker which is S100A9 and/or S100A8 in accordance with the present invention. The kit can, for example, comprise a primer/a primer pair for determining the level of a biomarker which is S100A9 and/or S100A8. The kit can, for example, comprise a binding molecule, such as an antibody, specifically binding to a biomarker which is S100A9 and/or S100A8, wherein the biomarker is a protein.

In a further aspect, the invention provides an in vitro use of a kit comprising a primer/a primer pair for determining the level of a biomarker which is S100A9 and/or S100A8 for monitoring the response of a subject to treatment with an LSD1 inhibitor. For example, the primer/primer pair can specifically bind to the nucleotide sequence of a biomarker which is S100A9 and/or S100A8.

In a further aspect, the invention provides an in vitro use of a kit comprising a primer/a primer pair for determining the level of a biomarker which is S100A9 and/or S100A8 for identifying patients with increased likelihood to benefit from treatment with an LSD1 inhibitor. For example, the primer/primer pair can specifically bind to the nucleotide sequence of a biomarker which is S100A9 and/or S100A8.

In a further aspect, the invention provides an in vitro use of a kit comprising a binding molecule specifically binding to a biomarker which is S100A9 protein and/or S100A8 protein for monitoring the response of a subject to treatment with an LSD1 inhibitor. The binding molecule can be for example an antibody.

In a further aspect, the invention provides an in vitro use of a kit comprising a binding molecule specifically binding to a biomarker which is S100A9 protein and/or S100A8 protein for identifying patients with increased likelihood to benefit from treatment with an LSD1 inhibitor. The binding molecule can be for example an antibody.

In a further aspect, the invention provides a use of a primer/a primer pair for the preparation of a kit for monitoring the response of a subject to treatment with an LSD1 inhibitor, wherein the primer/primer pair is for determining the level of a biomarker which is S100A9 and/or S100A8. For example, the primer/primer pair can specifically bind to the nucleotide sequence of a biomarker which is S100A9 and/or S100A8.

In a further aspect, the invention provides a use of a primer/a primer pair for the preparation of a kit for identifying patients with increased likelihood to benefit from treatment with an LSD1 inhibitor, wherein the primer/primer pair is for determining the level of a biomarker which is S100A9 and/or S100A8. For example, the primer/primer pair can specifically bind to the nucleotide sequence of a biomarker which is S100A9 and/or S100A8.

In a further aspect, the invention provides a use of a binding molecule for the preparation of a kit for monitoring the response of a subject to treatment with an LSD1 inhibitor, wherein the binding molecule is specifically binding to a biomarker which is S100A9 protein and/or S100A8 protein. The binding molecule can be for example an antibody.

In a further aspect, the invention provides a use of a binding molecule for the preparation of a kit for identifying patients with increased likelihood to benefit from treatment with an LSD1 inhibitor, wherein the binding molecule is specifically binding to a biomarker which is S100A9 protein and/or S100A8 protein. The binding molecule can be for example an antibody.

The present invention also provides methods of using the biomarkers and active agents of the invention in the field of therapy, particularly human therapy.

As demonstrated in the appended Examples, LSD1 inhibitors, including selective LSD1 inhibitors and dual LSD1/MAO-B inhibitors, have been found to down-regulate S100A9 and S100A8. Since S100A9 and S100A8 have been reported in the literature to have a relevant role in a number of diseases, as explained in more detail below, LSD1 inhibitors can be useful to treat any disease that is characterized by induction of S100A9 and/or S100A8, including the diseases discussed below. The term “induction of S100A9 and/or S100A8” includes, but it not limited to, overexpression of S100A9 and/or S100A8, i.e. an increased expression of S100A9 and/or S100A8 compared to a control (e.g. a healthy control, like (pooled) sample(s) from healthy individuals). “Overexpression of S100A9 and/or S100A8” as used herein can refer to an increased amount or concentration of a gene product of S100A9 and/or S100A8. The gene product can be mRNA or protein.

### Central Nervous System (CNS) Diseases:

An increase in S100A9 expression has been reported in several CNS diseases and models, including lesion-related insults like focal cerebral isquemia (Ziegler et al., 2009, Biochim Biophys Acta 1792:1198-1204) and diseases with cognitive function impairment such as Alzheimer's disease (AD) (Chang et al., 2012, Neurodegener Dis 10:27-29), schizophrenia (Foster et al., 2006, Eur J Neurosci 24:3561-3566) and depression (Webster et al., 1999, Mol Psychiatry 4:46-52); cerebral amyloid angiopathy (Kametani F, 2014, J Neurol Stroke 1(2): 00006), post operative cognitive dysfunction (Lu et al., Brain Behav Immun. 2015 February; 44:221-34); traumatic brain injury (Engel et al., Acta Neuropathol. 2000, 100(3):313-22), autoimmune encephalomyelitis (Björk et al. PLoS Biol. 2009, Apr. 28; 7(4):e97), Rett disease (Urdinguio RG1 et al., PLoS One. 2008; 3(11):e3669) and in human cerebral malaria (Schluesener et al., Acta Neuropathol. 1998 December; 96(6):575-80). In particular, mean S100A9 levels were higher in Mild Cognitive Impairment (MCI) than in control CSF and were elevated in AD brain protein extracts and cerebrospinal fluid (CSF). Moreover, in APP/PS1 mice, a mouse model for AD, S100A9 and S100A8 were upregulated in microglial cells surrounding amyloid plaques (Kummer et al., 2012, J Neurosci 32:17824-17829).

After 1 h focal cerebral ischemia, S100A9-deficient mice had significantly smaller lesion volumes when compared to wild-type results, supporting that upregulation and signaling of S100A8/9 contributes to neuroinflammation and the progression of ischemic damage (Ziegler et al., 2009, Biochim Biophys Acta 1792:1198-1204).

The functional implication of S100A9 was also proven in mice models of familiar Alzheimer's disease, where the S100A9 knockout decreases the memory impairment and neuropathology (Kummer et al., 2012, J Neurosci 32:17824-17829; Kim et al., 2014, Plos One, 9:e88924). As reported by Kummer et al, loss of S100A9 in APP/PS1 led to increased phagocytosis of fibrillar amyloid 3 (AP) in microglia cells in vitro and in vivo. APP/PS1+S100A9−/−mice have lower levels of key cytokines involved in APP processing, BACE1 and A3 deposition. S100A9 promotes APP processing and A3 accumulation under neuroinflammatory conditions.

Based on the above findings and additional evidence reported by others, it is expected that downregulation of S100A9 and/or S100A8 by LSD1i may be beneficial in the treatment of CNS diseases, particularly neurodegenerative diseases.

### Autoimmune Diseases:

S100A8 and S100A9 expression has been shown to be increased in human patients of autoimmune diseases like rheumatoid arthritis, inflammatory bowel disease (IBD), systemic lupus erythematosus or systemic sclerosis (Foell and Roth, 2004, Arthritis Rheum 50:3762-3771). S100A9 has been reported in autoimmunee diseases, such as multiple sclerosis (Björk et al. PLoS Biol. 2009, Apr. 28; 7(4):e97). The Experimental Autoimmune Encephalomyelitis (EAE) mouse model used in Björk et al. (loc. cit.) shows pathologic and clinical similarities to human multiple sclerosis (MS) and is widely used as a model for MS. Elevated concentrations of fecal S100A8/S100A9 have been demonstrated in numerous studies of patients with IBD. Fecal calprotectin correlates well with histological inflammation as detected by colonoscopy with biopsies and has been shown successfully to predict relapses in patients with IBD (Konikoff and Denson, 2006, Inflamm Bowel Dis 12:524-534).

Moreover, using a model of antigen-induced arthritis in S100A9 knockout mice, it was shown that S100A8/S100A9 regulates joint inflammation and cartilage destruction (van Lent et al., 2007, Ann Rheum Dis 67:1750-1758). In this experiment S100A9-KO mice showed less cartilage damage than the wild type animals. Based on the above findings and additional evidence reported by others, it is expected that downregulation of S100A9 and/or S100A8 by LSD1i may be beneficial in the treatment of autoimmune diseases.

Infections:

Both S100A8 and S100A9 are upregulated in local bacterial infection (Mares et al., 2008, Infec Immun 76:3001-3010) as well as in infection-derived complications like sepsis (Payen et al., 2008, Intensive Care Med 34:1371-1376; Fontaine et al., 2011, Crit Care Med 39:2684-2690) or cardiovascular pathologies (Hokamura and Umemura, 2010, J Pharmacol Sci 113:110-114). Induction of S100A9 has also been observed in fungal (Yano et al., 2012, Cytokine 58:118-128), protozoan (Jaramillo et al., 2004, J Immunol 172:3101-3110) and viral (Teran et al., 2012, Arch Med Res 43:464-469) infections.

The functional implication of S100A9 has been proven using knockout mice for this gene in local infection (Wache et al., 2015, J Infect Dis, pii: jiv028) and sepsis (Vogl et al., 2007, Nat Med 13:1042-1049) models. In both cases, animals lacking S100A9 gene were less severally affected or survived longer than wild type animals.

Based on the above findings and additional evidence reported by others, it is expected that downregulation of S100A9 and/or S100A8 by LSD1i may be beneficial in the treatment of infections, particularly bacterial, fungal, protozoan and viral infections and diseases associated with said infections.

### Cancer:

S100A8 and S100A9 proteins have been reported to participate in tumor progression (Srikrishna, 2012 J Innate Immun 4:31-40). Tumor-derived factors promote sustained up-regulation of S100A9 (both in tumor cells and infiltrating immune cells) which bind to Receptor for Advanced Glycation End products (RAGE) or Toll-Like Receptor 4 (TLR4) on tumor cells, promoting activation of cancer relevant intracellular signaling pathways (i.e. MAPK, NF-κB). Intracellular activation of these signaling pathways enhances expression of pro-tumorigenic genes and promotes tumor proliferation and migration.

Functional evidence of S100A9 has been proven in knockout mice models of prostate cancer (Kallberg et al., 2012 Plos One 7: e34207), lung cancer (Ortiz et al., 2014, Cancer Immunol Res 2:50-58).

Based on the above findings and additional evidence reported by others, it is expected that downregulation of S100A9 and/or S100A8 by LSD1i may be beneficial in the treatment of cancer.

### Cardiovascular Diseases:

High circulating levels of S100A8/A9 have been reported in patients suffering from acute and chronic inflammatory disorders, including conditions increasing cardiovascular risk (Averill et al., 2012, Arterioscler Thromb Vasc Biol 32:223-229). Elevated plasma levels of S100A8/A9 are associated with increased risk of future coronary events in healthy individuals and in myocardial infarction survivors (Schiopu and Cotoi, 2013, Mediators Inflamm 2013: Article ID 828354). Thus, S100A8/A9 might represent a useful biomarker and therapeutic target in cardiovascular disease.

The functional implication of S100A9 in atherosclerotic lesions was proven crossing S100A9^{-/-} with Apoe^{-/-} mice (Croce et al., 2009, Circulation 120:427-436). These double knockout mice had an approximate 30% reduction in en face aortic lesion area in response to a high-fat diet, as compared with Apoe^{-/-} controls.

S100A9^{-/-} mice also showed significant reductions in neutrophil accumulation, lesion severity, and hemorrhagic area in a model of vascular injury (Croce et al., 2009 Circulation 120: 427-436).

Based on the above findings and additional evidence reported by others, it is expected that downregulation of S100A9 and/or S100A8 by LSD1i may be beneficial in the treatment of cardiovascular disorders.

Accordingly, it is expected that LSD1 inhibitors can be useful to treat diseases characterized by S100A9 and/or S100A8 as discussed above, and can be especially useful in those patients in the disease population that have S100A9 and/or S100A8 levels elevated above control levels. “Control levels” as used herein means a healthy control (i.e. the levels of the biomarker in a healthy control).

Non-limiting examples of diseases characterized by S100A9 and/or S100A8 induction that may be treated with an LSD1 inhibitor include:

1) CNS diseases: including neurodegenerative diseases (including Alzheimer's disease, Mild Cognitive Impairment, Parkinson's disease, difuse Lewy body disease, synucleinopathies, Huntington's disease, Down syndrome, and Amyotrophic lateral sclerosis); autism spectrum disease (including autism, Asperger syndrome, pervasive developmental disorder not otherwise specified (PDD-NOS), and childhood disintegrative disorder); cognitive function related disease (including dementia such as vascular dementia, Lewy body dementia, senile dementia, frontotemporal dementia and mixed dementia, delirium, amnesia, Rett disease, schizophrenia, attention-deficit/hyperactivity disorder, and postoperative cognitive dysfunction); mood disorders (including anxiety, stress disorder, post-traumatic stress disorder, panic disorder, phobia, mania, depressive disorders such as major depression, recurrent depression and postpartum disorder, bipolar disorders, and obsessive-compulsive disorder); and stroke and lesion-related diseases (including Traumatic Brain Injury, brain ischemia, intracranial hemorrhage, intracranial aneurysm, and Cerebral Amyloid Angiopathy);
2) autoimmune diseases: including arthritis (including rheumatoid arthritis, psoriatic arthritis, reactive arthritis and juvenile idiopathic arthritis); inflammatory bowel disease (including Crohn's disease and ulcerative colitis); sclerosis (including systemic sclerosis); acute and chronic autoimmune neuropathies (including autoimmune encephalomyelitis and multiple sclerosis); lupus (including lupus erythematosus, glomerulonephritis, and vasculitis); autoimmune pancreas disease (including autoimmune pancreatitis and diabetes mellitus type 1); autoimmune skin diseases (including psoriasis); autoimmune muscle disease (including dermatomyositis, polymyositis, and inclusion body myositis); and Kawasaki disease;
3) infections: particularly bacterial, fungal, protozoan and viral infections and diseases caused by said infections: including bacterial infections (for example caused by E. coli, Pneumococcus, Helicobacter pylori, Salmonella, Staphylococcus aureus, Pseudomonas aeruginosa, Ureaplasma parvum, Francisella tularensis, and Porphyromonas gingivalis), and diseases caused by said bacterial infections such as acute bacterial infections (including acute appendicitis, meningitis, caries, gastritis, gastric ulceration, and acne) and sepsis (including Severe sepsis, septic shock, perinatal and neonatal sepsis); fungal infections (for example Candidiasis or Aspergillosis) and diseases caused by said fungal infections, protozoan infections (for example caused by Plasmodium or Trypanomoma cruzi) and diseases caused by said protozoan infections (for example malaria or Chagas' disease); and viral infections (influenza virus) and diseases caused by said viral infections (for example Influenza).
4) cancer: including carcinomas such as colorectal cancer, bladder cancer, prostate cancer, anaplastic thyroid carcinoma, cutaneous squamous cell carcinoma, gastric cancer, lung cancer and breast cancer (including metastatic breast cancer to brain); and sarcomas such as glioma (for example astrocytoma); and
5) cardiovascular diseases: including arteriosclerotic vascular disease (including atherosclerosis and atherogenesis), acute coronary syndromes (like myocardial infarction) and vascular injury (including thrombosis, embolism, vasculitis, venous ulcers, and aortic aneurysms).

This list of diseases, recited here in the context of therapy with LSD1 inhibitors, applies likewise in the context of the diagnostic methods of the invention, i.e. in relation to the methods for monitoring and the methods for predicting/determining likeliness of response of patients to LSD1 inhibitors described above.

Accordingly, in another aspect, the invention provides a method for treating a patient, comprising: (i) determining the level of a biomarker which is S100A9 and/or S100A8 in a sample obtained from the patient prior to treatment with an LSD1 inhibitor, and (ii) administering to the patient a therapeutically effective amount of the LSD1 inhibitor if the level of the biomarker in the sample is elevated as compared to a control.

In another aspect, the invention provides a method for treating a patient, comprising: (i) determining the level of a biomarker which is S100A9 and/or S100A8 in a sample obtained from the patient prior to treatment with an LSD1 inhibitor, (ii) determining whether the patient is likely responsive to treatment with the LSD1 inhibitor, wherein an elevated level of the biomarker in the sample as compared to a control is indicative of the patient being likely responsive to the treatment with the LSD1 inhibitor, and (iii) administering to the patient a therapeutically effective amount of the LSD1 inhibitor if the patient has been identified as being likely responsive to the treatment with the LSD1 inhibitor.

In another aspect, the invention provides a method for treating a patient, comprising: (i) determining likeliness of responsiveness of the patient to an LSD1 inhibitor by any of the methods as described herein; and (ii) administering to the patient a therapeutically effective amount of the LSD1 inhibitor if the patient is identified to be likely responsive thereto.

In the above methods the patient can be a patient that has a CNS disease.

In the above methods the patient can be a patient that has a neurodegenerative disease, for example Alzheimer's disease, Mild Cognitive Impairment, Parkinson's disease, difuse Lewy body disease, synucleinopathies, Huntington's disease, Down syndrome, or Amyotrophic lateral sclerosis, preferably Alzheimer's disease or Mild Cognitive Impairment.

In the above methods the patient can be a patient that has cognitive function related disease, for example dementia (such as vascular dementia, Lewy body dementia, senile dementia, frontotemporal dementia and mixed dementia), delirium, amnesia, Rett disease, schizophrenia, attention-deficit/hyperactivity disorder, or postoperative cognitive dysfunction.

In the above methods the patient can be a patient that has an autoimmune disease. For example, the autoimmune disease can be an acute or chronic autoimmune neuropathy such as multiple sclerosis. Multiple sclerosis can be for example chronic progressive multiple sclerosis.

In the above methods the patient can be a patient that has an infection or a disease caused by an infection, preferably a bacterial infection, a fungal infection, a protozoan infection, an influenza infection, or a disease caused by any of said infections.

In the above methods the patient can be a patient that has cancer.

In the above methods the patient can be a patient that has a cardiovascular disease.

In another aspect, the invention further provides a method for treating a disease characterized by induction of S100A9 and/or S100A8 in a patient, comprising: (i) determining the level of a biomarker which is S100A9 and/or S100A8 in a sample obtained from the patient prior to treatment with an LSD1 inhibitor, and (ii) administering to the patient a therapeutically effective amount of the LSD1 inhibitor if the level of the biomarker in the sample is elevated compared to a control.

In another aspect, the invention further provides a method for treating a patient having a disease characterized by induction of S100A9 and/or S100A8, comprising: (i) determining the level of a biomarker which is S100A9 and/or S100A8 in a sample obtained from the patient prior to treatment with an LSD1 inhibitor, and (ii) administering to the patient a therapeutically effective amount of the LSD1 inhibitor if the level of the biomarker in the sample is elevated compared to a control.

In a certain aspect, the invention relates to a method for treating a patient having a disease characterized by S100A9 and/or S100A8 induction, the method comprising obtaining a sample of a patient for whom LSD1 inhibitor therapy is contemplated, and testing the sample to determine an elevated level of a biomarker which is S100A9 and/or S100A8 compared to a control therein and administering an effective amount of the LSD1 inhibitor to the patient having a disease characterized by S100A9 and/or S100A8 induction.

In another aspect, the invention provides a method for treating a CNS disease in a patient, comprising: (i) determining the level of a biomarker which is S100A9 and/or S100A8 in a sample obtained from the patient prior to treatment with an LSD1 inhibitor, and (ii) administering to the patient a therapeutically effective amount of the LSD1 inhibitor if the level of the biomarker in the sample is elevated compared to a control.

In another aspect, the invention provides a method for treating a neurodegenerative disease in a patient, comprising: (i) determining the level of a biomarker which is S100A9 and/or S100A8 in a sample obtained from the patient prior to treatment with an LSD1 inhibitor, and (ii) administering to the patient a therapeutically effective amount of the LSD1 inhibitor if the level of the biomarker in the sample is elevated compared to a control. The neurodegenerative disease can be for example Alzheimer's disease, Mild Cognitive Impairment, Parkinson's disease, difuse Lewy body disease, synucleinopathies, Huntington's disease, Down syndrome, or Amyotrophic lateral sclerosis.

In another aspect, the invention provides a method for treating Alzheimer's disease in a patient, comprising: (i) determining the level of a biomarker which is S100A9 and/or S100A8 in a sample obtained from the patient prior to treatment with an LSD1 inhibitor, and (ii) administering to the patient a therapeutically effective amount of the LSD1 inhibitor if the level of the biomarker in the sample is elevated compared to a control.

In another aspect, the invention provides a method for treating mild cognitive impairment in a patient, comprising: (i) determining the level of a biomarker which is S100A9 and/or S100A8 in a sample obtained from the patient prior to treatment with an LSD1 inhibitor, and (ii) administering to the patient a therapeutically effective amount of the LSD1 inhibitor if the level of the biomarker in the sample is elevated compared to a control.

In another aspect, the invention provides a method for treating a cognitive function related disease in a patient, comprising: (i) determining the level of a biomarker which is S100A9 and/or S100A8 in a sample obtained from the patient prior to treatment with an LSD1 inhibitor, and (ii) administering to the patient a therapeutically effective amount of the LSD1 inhibitor if the level of the biomarker in the sample is elevated compared to a control. The cognitive function related disease can be for example dementia (such as vascular dementia, Lewy body dementia, senile dementia, frontotemporal dementia and mixed dementia), delirium, amnesia, Rett disease, schizophrenia, attention-deficit/hyperactivity disorder, or postoperative cognitive dysfunction.

In another aspect, the invention provides a method for treating an autoimmune disease in a patient, comprising: (i) determining the level of a biomarker which is S100A9 and/or S100A8 in a sample obtained from the patient prior to treatment with an LSD1 inhibitor, and (ii) administering to the patient a therapeutically effective amount of the LSD1 inhibitor if the level of the biomarker in the sample is elevated compared to a control. The autoimmune disease can be for example an acute or chronic autoimmune neuropathy such as multiple sclerosis. Multiple sclerosis can be for example chronic progressive multiple sclerosis.

In another aspect, the invention provides a method for treating an autoimmune disease in a patient, comprising: (i) obtaining a sample from the patient prior to treatment with an LSD1 inhibitor, (ii) determining the level of a biomarker which is S100A9 and/or S100A8 in the sample, and (iii) administering to the patient an amount of the LSD1 inhibitor sufficient to decrease the biomarker levels while not causing a clinically relevant reduction in platelet levels if the level of the biomarker in the sample is elevated compared to a control. The autoimmune disease can be for example an acute or chronic autoimmune neuropathy such as multiple sclerosis. Multiple sclerosis can be for example chronic progressive multiple sclerosis.

In another aspect, the invention provides a method for treating an infection or a disease caused by an infection, preferably a bacterial infection, a fungal infection, a protozoan infection, an influenza infection, or a disease caused by any of said infections in a patient, comprising: (i) determining the level of a biomarker which is S100A9 and/or S100A8 in a sample obtained from the patient prior to treatment with an LSD1 inhibitor, and (ii) administering to the patient a therapeutically effective amount of the LSD1 inhibitor if the level of the biomarker in the sample is elevated compared to a control.

In another aspect, the invention provides a method for treating cancer in a patient, comprising: (i) determining the level of a biomarker which is S100A9 and/or S100A8 in a sample obtained from the patient prior to treatment with an LSD1 inhibitor, and (ii) administering to the patient a therapeutically effective amount of the LSD1 inhibitor if the level of the biomarker in the sample is elevated compared to a control.

In another aspect, the invention provides a method for treating a cardiovascular disease in a patient, comprising: (i) determining the level of a biomarker which is S100A9 and/or S100A8 in a sample obtained from the patient prior to treatment with an LSD1 inhibitor, and (ii) administering to the patient a therapeutically effective amount of the LSD1 inhibitor if the level of the biomarker in the sample is elevated compared to a control.

In another aspect, the invention provides a method for treating a cardiovascular disease in a patient, comprising: (i) obtaining a sample from the patient prior to treatment with an LSD1 inhibitor, (ii) determining the level of a biomarker which is S100A9 and/or S100A8 in the sample, and (iii) administering to the patient an amount of the LSD1 inhibitor sufficient to decrease the biomarker levels while not causing a clinically relevant reduction in platelet levels if the level of the biomarker in the sample is elevated compared to a control.

In the above methods, the method can comprise an extra step of obtaining a sample from the patient prior to determining the level of the biomarker.

In the methods described above, the level of the biomarker can be determined as mRNA.

In the methods described above, the level of the biomarker can be determined as protein.

In the methods described above the biomarker is preferably S100A9. In the methods described above, the level of S100A9 can be determined as mRNA. In the methods described above the level of S100A9 can be determined as protein. In the methods described above, the level of the biomarker can be determined as S100A9 monomer. In the methods described above, the level of the biomarker can be determined as a S100A8/S100A9 heterodimer.

In in the methods described above the sample is preferably a peripheral sample. The peripheral sample can be e.g. cerebrospinal fluid (CSF), blood, plasma, serum, stool, saliva, sputum, gingival crevicular fluid, hair follicle or skin biopsy.

In the methods described above the LSD1 inhibitor can be an irreversible LSD1 inhibitor or a reversible LSD1 inhibitor. Preferably, the LSD1 inhibitor is an irreversible LSD1 inhibitor.

In the methods described above the LSD1 inhibitor is preferably a 2-(hetero)arylcyclopropylamino compound.

In the methods described above the LSD1 inhibitor is preferably a compound disclosed in WO2010/043721, WO2010/084160, WO2011/035941, WO2011/042217, WO2011/131697, WO2012/013727, WO2012/013728, WO2012/045883, WO2013/057320, WO2013/057322, WO2012/135113, WO2013/022047, WO2014/058071, WO2010/143582, US2010-0324147, WO2011/131576, WO2014/084298, WO2014/086790, WO2014/164867, or WO2015/021128.

In the methods described above the LSD1 inhibitor is preferably a compound of formula (I), (II), (III), (IV), (V), (VI), (VII), (VIII), (IX), (X), (XI), (XII) or (XIII). More preferably, the LSD1 inhibitor is a compound of formula (III), (VI), (VIII), (IX), (X), (XI), (XII) or (XIII). Still more preferably, the LSD1 inhibitor is a compound from the lists of examples provided below for compounds of formulae (III), (VI), (VIII), (IX), (X) or (XI).

Preferably, in the methods described above the LSD1 inhibitor is (−) 5-((((trans)-2-(4-(benzyloxy)phenyl)cyclopropyl)amino)methyl)-1,3,4-oxadiazol-2-amine or a pharmaceutically acceptable salt or solvate thereof.

In the methods described above, the patient is preferably a human.

In another aspect, the invention provides a method for treating Mild Cognitive Impairment in a patient, comprising administering to the patient a therapeutically effective amount of an LSD1 inhibitor.

In another aspect, the invention provides a method for treating an infection or a disease caused by an infection, preferably a bacterial infection, a fungal infection, a protozoan infection, an influenza infection, or a disease caused by any of said infections in a patient, comprising administering to the patient a therapeutically effective amount of an LSD1 inhibitor.

In another aspect, the invention provides a method for treating an autoimmune disease in a patient, comprising administering to the patient an amount of an LSD1 inhibitor that decreases the level of a biomarker which is S100A9 and/or S100A8 while not causing a clinically relevant reduction in platelet levels. The autoimmune disease can be for example an acute or chronic autoimmune neuropathy such as multiple sclerosis. Multiple sclerosis can be for example chronic progressive multiple sclerosis.

In another aspect, the invention provides a method for treating a cardiovascular disease in a patient, comprising administering to the patient an amount of an LSD1 inhibitor that decreases the level of a biomarker which is S100A9 and/or S100A8 while not causing a clinically relevant reduction in platelet levels.

In the methods described above the LSD1 inhibitor can be an irreversible LSD1 inhibitor or a reversible LSD1 inhibitor. Preferably, the LSD1 inhibitor is an irreversible LSD1 inhibitor.

In the methods described above the LSD1 inhibitor is preferably a 2-(hetero)arylcyclopropylamino compound.

In in the methods described above the LSD1 inhibitor is preferably a compound disclosed in WO2010/043721, WO2010/084160, WO2011/035941, WO2011/042217, WO2011/131697, WO2012/013727, WO2012/013728, WO2012/045883, WO2013/057320, WO2013/057322, WO2012/135113, WO2013/022047, WO2014/058071, WO2010/143582, US2010-0324147, WO2011/131576, WO2014/084298, WO2014/086790, WO2014/164867, or WO2015/021128.

In the methods described above the LSD1 inhibitor is preferably a compound of formula (I), (II), (III), (IV), (V), (VI), (VII), (VIII), (IX), (X), (XI), (XII) or (XIII). More preferably, the LSD1 inhibitor is a compound of formula (III), (VI), (VIII), (IX), (X), (XI), (XII) or (XIII). Still more preferably, the LSD1 inhibitor is a compound from the lists of examples provided below for compounds of formulae (Ill), (VI), (VIII), (IX), (X) or (XI).

Preferably, in the methods described above the LSD1 inhibitor is (−) 5-((((trans)-2-(4-(benzyloxy)phenyl)cyclopropyl)amino)methyl)-1,3,4-oxadiazol-2-amine or a pharmaceutically acceptable salt or solvate thereof.

In the methods described above, the patient is preferably a human.

In another aspect, the invention provides an LSD1 inhibitor for use in therapy, wherein said therapy comprises: (i) determining the level of a biomarker which is S100A9 and/or S100A8 in a sample obtained from a patient prior to treatment with an LSD1 inhibitor, and (ii) administering the LSD1 inhibitor to the patient if the level of the biomarker in the sample is elevated as compared to a control.

In another aspect, the invention provides an LSD1 inhibitor for use in a method of treating a disease selected from the group consisting of a CNS disease, an autoimmune disease, an infection or a disease caused by an infection (preferably a bacterial infection, a fungal infection, a protozoan infection, an influenza infection, or a disease caused by any of said infections), cancer and a cardiovascular disease in a patient, the method comprising: (i) determining the level of a biomarker which is S100A9 and/or S100A8 in a sample obtained from the patient prior to treatment with an LSD1 inhibitor, and (ii) administering the LSD1 inhibitor to the patient if the level of the biomarker in the sample is elevated as compared to a control.

In another aspect, the invention provides an LSD1 inhibitor for use in a method of treating a disease selected from the group consisting of a CNS disease, an autoimmune disease, an infection or a disease caused by an infection (preferably a bacterial infection, a fungal infection, a protozoan infection, an influenza infection, or a disease caused by any of said infections), cancer and a cardiovascular disease in a patient, the method comprising: (i) determining the level of a biomarker which is S100A9 and/or S100A8 in a sample obtained from the patient prior to treatment with an LSD1 inhibitor, (ii) determining whether the patient is likely responsive to the treatment with the LSD1 inhibitor, wherein an elevated level of the biomarker in the sample as compared to a control is indicative of the patient being likely responsive to the treatment with the LSD1 inhibitor, and (iii) administering the LSD1 inhibitor to the patient if the patient has been identified as being likely responsive to the treatment with the LSD1 inhibitor.

In another aspect, the invention provides an LSD1 inhibitor for use in treating a disease selected from the group consisting of a CNS disease, an autoimmune disease, a an infection or a disease caused by an infection (preferably a bacterial infection, a fungal infection, a protozoan infection, an influenza infection, or a disease caused by any of said infections), cancer and a cardiovascular disease in a patient, wherein the patient has been predicted to be likely responsive to treatment with an LSD1 inhibitor by any of the methods described herein.

In another aspect, the invention provides an LSD1 inhibitor for use in treating a disease characterized by induction of S100A9 and/or S100A8 in a patient, wherein the level of a biomarker which is S100A9 and/or S100A8 is determined in a sample from the patient prior to treatment with an LSD1 inhibitor, and the LSD1 inhibitor is administered to the patient if the level of the biomarker in the sample is elevated compared to a control.

In a certain aspect, the invention relates to an LSD1 inhibitor for use in a method of treating a disease characterized by S100A9 and/or S100A8 induction in a patient, wherein the patient has an elevated level of a biomarker which is S100A9 and/or S100A8 compared to a control.

In a certain aspect, the invention relates to an LSD1 inhibitor for use in a method of treating a disease characterized by S100A9 and/or S100A8 induction in a patient that was assessed positive for an elevated level of a biomarker which is S100A9 and/or S100A8 compared to a control.

In a certain aspect, the invention relates to an LSD1 inhibitor for use in a method of treating a disease characterized by S100A9 and/or S100A8 induction in a patient that has been tested positive for an elevated level of a biomarker which is S100A9 and/or S100A8 compared to a control.

In a certain aspect, the invention relates to an LSD1 inhibitor for use in the treatment of a disease characterized by S100A9 and/or S100A8 induction wherein the patient has an elevated level of a biomarker which is S100A9 and/or S100A8 compared to a control and the method of treatment comprises the step of determining whether or not the patient has an elevated level of a biomarker which is S100A9 and/or S100A8 compared to a control.

In a certain aspect, the invention relates to an LSD1 inhibitor for use in a method of treating a disease characterized by S100A9 and/or S100A8 induction in a patient identified as having an elevated level of a biomarker which is S100A9 and/or S100A8 compared to a control using the herein provided methods.

In a certain aspect, the invention relates to an LSD1 inhibitor for use in a method of treating a disease characterized by S100A9 and/or S100A8 induction, wherein said method comprises testing a patient using the herein provided methods of determining an elevated level of a biomarker which is S100A9 and/or S100A8, in order to determine whether the patient has an elevated level of a biomarker which is S100A9 and/or S100A8 compared to a control, and providing treatment with an LSD1 inhibitor if the patient is identified as having an elevated level of a biomarker which is S100A9 and/or S100A8 compared to a control.

In a certain aspect, the invention relates to an LSD1 inhibitor for use in a method of treating a disease characterized by S100A9 and/or S100A8 induction in a patient assessed positive for an elevated level of a biomarker which is S100A9 and/or S100A8 compared to a control.

In another aspect, the invention provides an LSD1 inhibitor for use in treating a CNS disease in a patient, wherein the level of a biomarker which is S100A9 and/or S100A8 is determined in a sample from the patient prior to treatment with an LSD1 inhibitor, and the LSD1 inhibitor is administered to the patient if the level of the biomarker in the sample is elevated compared to a control.

In another aspect, the invention provides an LSD1 inhibitor for the treatment of a CNS disease in a subgroup of patients with elevated levels of S100A9 and/or S100A8.

In another aspect, the invention provides an LSD1 inhibitor for use in treating a neurodegenerative disease in a patient, wherein the level of a biomarker which is S100A9 and/or S100A8 is determined in a sample from the patient prior to treatment with an LSD1 inhibitor, and the LSD1 inhibitor is administered to the patient if the level of the biomarker in the sample is elevated compared to a control. The neurodegenerative disease can be for example Alzheimer's disease, Mild Cognitive Impairment, Parkinson's disease, difuse Lewy body disease, synucleinopathies, Huntington's disease, Down syndrome, or Amyotrophic lateral sclerosis.

In another aspect, the invention provides an LSD1 inhibitor for the treatment of a neurodegenerative disease in a subgroup of patients with elevated levels of S100A9 and/or S100A8.

In another aspect, the invention provides an LSD1 inhibitor for use in treating Alzheimer's disease in a patient, wherein the level of a biomarker which is S100A9 and/or S100A8 is determined in a sample from the patient prior to treatment with an LSD1 inhibitor, and the LSD1 inhibitor is administered to the patient if the level of the biomarker in the sample is elevated compared to a control.

In another aspect, the invention provides an LSD1 inhibitor for use in treating mild cognitive impairment in a patient, wherein the level of a biomarker which is S100A9 and/or S100A8 is determined in a sample from the patient prior to treatment with an LSD1 inhibitor, and the LSD1 inhibitor is administered to the patient if the level of the biomarker in the sample is elevated compared to a control.

In another aspect, the invention provides an LSD1 inhibitor for use in treating a cognitive function related disease in a patient, wherein the level of a biomarker which is S100A9 and/or S100A8 is determined in a sample from the patient prior to treatment with an LSD1 inhibitor, and the LSD1 inhibitor is administered to the patient if the level of the biomarker in the sample is elevated compared to a control. The cognitive function related disease can be for example dementia (such as vascular dementia, Lewy body dementia, senile dementia, frontotemporal dementia and mixed dementia), delirium, amnesia, Rett disease, schizophrenia, attention-deficit/hyperactivity disorder, or postoperative cognitive dysfunction.

In another aspect, the invention provides an LSD1 inhibitor for use in treating an autoimmune disease in a patient, wherein the level of a biomarker which is S100A9 and/or S100A8 is determined in a sample from the patient prior to treatment with an LSD1 inhibitor, and the LSD1 inhibitor is administered to the patient if the level of the biomarker in the sample is elevated compared to a control. The autoimmune disease can be for example an acute or chronic autoimmune neuropathy such as multiple sclerosis. Multiple sclerosis can be for example chronic progressive multiple sclerosis.

In another aspect, the invention provides an LSD1 inhibitor for use in treating an autoimmune disease in a patient, wherein the level of a biomarker which is S100A9 and/or S100A8 is determined in a sample from the patient prior to treatment with an LSD1 inhibitor, and an amount of the LSD1 inhibitor sufficient to decrease the biomarker levels while not causing a clinically relevant reduction in platelet levels is administered to the patient if the level of the biomarker in the sample is elevated compared to a control. The autoimmune disease can be for example an acute or chronic autoimmune neuropathy such as multiple sclerosis. Multiple sclerosis can be for example chronic progressive multiple sclerosis.

In another aspect, the invention provides an LSD1 inhibitor for the treatment of an autoimmune disease in a subgroup of patients with elevated levels of S100A9 and/or S100A8. The autoimmune disease can be for example an acute or chronic autoimmune neuropathy such as multiple sclerosis. Multiple sclerosis can be for example chronic progressive multiple sclerosis. In another aspect, the invention provides an LSD1 inhibitor for use in treating an infection or a disease caused by an infection, preferably a bacterial infection, a fungal infection, a protozoan infection, an influenza infection, or a disease caused by any of said infections in a patient, wherein the level of a biomarker which is S100A9 and/or S100A8 is determined in a sample from the patient prior to treatment with an LSD1 inhibitor, and the LSD1 inhibitor is administered to the patient if the level of the biomarker in the sample is elevated compared to a control.

In another aspect, the invention provides an LSD1 inhibitor for use in treating cancer in a patient, wherein the level of a biomarker which is S100A9 and/or S100A8 is determined in a sample from the patient prior to treatment with an LSD1 inhibitor, and the LSD1 inhibitor is administered to the patient if the level of the biomarker in the sample is elevated compared to a control.

In another aspect, the invention provides an LSD1 inhibitor for use in treating a cardiovascular disease in a patient, wherein the level of a biomarker which is S100A9 and/or S100A8 is determined in a sample from the patient prior to treatment with an LSD1 inhibitor, and the LSD1 inhibitor is administered to the patient if the level of the biomarker in the sample is elevated compared to a control.

In another aspect, the invention provides an LSD1 inhibitor for use in treating a cardiovascular disease in a patient, wherein the level of a biomarker which is S100A9 and/or S100A8 is determined in a sample from the patient prior to treatment with an LSD1 inhibitor, and an amount of the LSD1 inhibitor sufficient to decrease the biomarker levels while not causing a clinically relevant reduction in platelet levels is administered to the patient if the level of the biomarker in the sample is elevated compared to a control.

In another aspect, the invention provides an LSD1 inhibitor for use in treating Mild Cognitive Impairment.

In another aspect, the invention provides an LSD1 inhibitor for use in treating a bacterial infection, a fungal infection, a protozoan infection, an influenza infection, or a disease caused by any of said infections.

In another aspect, the invention provides an LSD1 inhibitor for use in treating an autoimmune disease in a patient, wherein the LSD1 inhibitor is to be administered to the patient in an amount sufficient to decrease the level of a biomarker which is S100A9 and/or S100A8 while not causing a clinically relevant reduction in platelet levels. The autoimmune disease can be for example an acute or chronic autoimmune neuropathy such as multiple sclerosis. Multiple sclerosis can be for example chronic progressive multiple sclerosis.

In another aspect, the invention provides an LSD1 inhibitor for use in treating a cardiovascular disease in a patient, wherein the LSD1 inhibitor is to be administered to the patient in an amount sufficient to decrease the level of a biomarker which is S100A9 and/or S100A8 while not causing a clinically relevant reduction in platelet levels.

In the uses described above, the level of the biomarker can be determined as mRNA.

In the uses described above, the level of the biomarker can be determined as protein.

In the uses described above the biomarker is preferably S100A9. In the uses described above the level of S100A9 can be determined as mRNA. In the uses described above, the level of S100A9 can be determined as protein. In the uses described above, the level of the biomarker can be determined as S100A9 monomer. In the uses described above, the level of the biomarker can be determined as a S100A8/S100A9 heterodimer.

In the uses described above the sample is preferably a peripheral sample. The peripheral sample can be e.g. cerebrospinal fluid (CSF), blood, plasma, serum, stool, saliva, sputum, gingival crevicular fluid, hair follicle or skin biopsy.

In the uses described above the LSD1 inhibitor can be an irreversible LSD1 inhibitor or a reversible LSD1 inhibitor. Preferably, the LSD1 inhibitor is an irreversible LSD1 inhibitor.

In the uses described above the LSD1 inhibitor is preferably a 2-(hetero)arylcyclopropylamino compound.

In the uses described above the LSD1 inhibitor is preferably a compound disclosed in WO2010/043721, WO2010/084160, WO2011/035941, WO2011/042217, WO2011/131697, WO2012/013727, WO2012/013728, WO2012/045883, WO2013/057320, WO2013/057322, WO2012/135113, WO2013/022047, WO2014/058071, WO2010/143582, US2010-0324147, WO2011/131576, WO2014/084298, WO2014/086790, WO2014/164867, or WO2015/021128.

In the uses described above the LSD1 inhibitor is preferably a compound of formula (I), (II), (III), (IV), (V), (VI), (VII), (VIII), (IX), (X), (XI), (XII) or (XIII). More preferably, the LSD1 inhibitor is a compound of formula (III), (VI), (VIII), (IX), (X), (XI), (XII) or (XIII). Still more preferably, the LSD1 inhibitor is a compound from the lists of examples provided below for compounds of formulae (III), (VI), (VIII), (IX), (X) or (XI).

Preferably, in the uses described above the LSD1 inhibitor is (−) 5-((((trans)-2-(4-(benzyloxy)phenyl)cyclopropyl)amino)methyl)-1,3,4-oxadiazol-2-amine or a pharmaceutically acceptable salt or solvate thereof.

In the uses described above, the patient is preferably a human.

Analysis of S100A9 and/or S100A8 in human subjects, for example patients enrolled in a clinical trial, can be performed following the methods described in the present specification.

When used in methods for monitoring the response of a subject to treatment with an LSD1 inhibitor, typically samples (for example peripheral samples) are collected from each subject following standard procedures at different time points, starting with a sample obtained prior to the start of the treatment with the LSD1 inhibitor. Samples are then processed to prepare them for biomarker analysis following standard procedures, and the level of the biomarker of interest, i.e. S100A9 and/or S100A8, is determined in each sample by measuring mRNA levels thereof (for example by qRT-PCR) or protein levels thereof (for example by ELISA).

Typically when measuring mRNA levels, expression levels are normalized relative to the expression level of an endogenous reference gene. Said reference gene is selected following standard criteria, typically among housekeeping genes whose expression is unchanged over a wide range of conditions. An example of a suitable endogenous reference gene is GADPH (glyceraldehyde phosphate dehydrogenase, also known as GAPDH), as disclosed in the Examples.

Typically, when measuring protein levels of a protein of interest, for example by ELISA, a standard curve (obtained using samples with known concentrations of the target protein) can be used to quantify the concentration of target protein in the test sample.

An example of a peripheral sample, for example for use in patients having a CNS disease, is CSF.CSF samples are collected by lumbar puncture using standard procedures in participating healthcare facilities. Typically, a CSF volume ranging from 1 to 10 mL is obtained from each subject.

Fresh CSF samples are processed by centrifugation in order to obtain cell pellets and supernatant, which can either be analyzed then or be frozen and maintained at −80° C. until further analysis.

Cell pellets can be used to obtain RNA to analyze S100A9 and/or S100A8 expression levels using methods as described herein, for example by qRT-PCR. Liquid supernatant can be used to analyze S100A9 and/or S100A8 protein levels using methods as described herein, for example by ELISA. S100A9 protein levels can be analyzed as S100A9 monomer and/or S100A8/S100A9 heterodimer protein concentration, for example by ELISA.

The same procedure can be followed in methods for determining whether a patient or subject is likely to respond to treatment with an LSD1 inhibitor (i.e. predicting responsiveness to an LSD1 inhibitor) with the exception that the samples (for example peripheral samples) are then typically collected from each subject/patient solely prior to the start of the treatment with the LSD1 inhibitor. The thus selected patients/patient group can then be subjected to treatment with the LSD1 inhibitor in accordance with the invention.

In another aspect, the present invention provides (−) 5-((((trans)-2-(4-(benzyloxy)phenyl)cyclopropyl)amino)methyl)-1,3,4-oxadiazol-2-amine or a pharmaceutically acceptable salt or solvate thereof for use in the treatment of multiple sclerosis.

In another aspect, the present invention provides (−) 5-((((trans)-2-(4-(benzyloxy)phenyl)cyclopropyl)amino)methyl)-1,3,4-oxadiazol-2-amine or a pharmaceutically acceptable salt or solvate thereof for use in the treatment of chronic progressive multiple sclerosis.

In another aspect, the present invention provides a method for treating multiple sclerosis in a patient (preferably a human), comprising administering to the patient a therapeutically effective amount of (−) 5-((((trans)-2-(4-(benzyloxy)phenyl)cyclopropyl)amino)methyl)-1,3,4-oxadiazol-2-amine or a pharmaceutically acceptable salt or solvate thereof.

In another aspect, the present invention provides a method for treating chronic progressive multiple sclerosis in a patient (preferably a human), comprising administering to the patient a therapeutically effective amount of (−) 5-((((trans)-2-(4-(benzyloxy)phenyl)cyclopropyl)amino)methyl)-1,3,4-oxadiazol-2-amine or a pharmaceutically acceptable salt or solvate thereof.

In another aspect, the present invention provides the use of (−) 5-((((trans)-2-(4-(benzyloxy)phenyl)cyclopropyl)amino)methyl)-1,3,4-oxadiazol-2-amine or a pharmaceutically acceptable salt or solvate thereof for the manufacture of a medicament for the treatment of multiple sclerosis.

In another aspect, the present invention provides the use of (−) 5-((((trans)-2-(4-(benzyloxy)phenyl)cyclopropyl)amino)methyl)-1,3,4-oxadiazol-2-amine or a pharmaceutically acceptable salt or solvate thereof for the manufacture of a medicament for the treatment of chronic progressive multiple sclerosis.

In addition, the invention relates to the following items:

1. A method for monitoring LSD1 inhibition in a subject receiving treatment with an LSD1 inhibitor, comprising determining the level of a biomarker which is S100A9 and/or S100A8 in a sample obtained from the subject, wherein a decrease in the level of the biomarker in the sample as compared to the level of the biomarker in a control is indicative that LSD1 is being inhibited in the subject.
2. A method for monitoring the degree of LSD1 inhibition in a subject receiving treatment with an LSD1 inhibitor, comprising determining the level of a biomarker which is S100A9 and/or S100A8 in a sample obtained from the subject, wherein the degree of decrease in the level of the biomarker in the sample as compared to the level of the biomarker in a control is indicative of the degree of LSD1 inhibition in the subject.
3. A method for monitoring the response of a subject to treatment with an LSD1 inhibitor, comprising determining the level of a biomarker which is S100A9 and/or S100A8 in a sample obtained from the subject, wherein a decrease in the level of the biomarker in the sample as compared to the level of the biomarker in a control indicates response to the treatment with the LSD1 inhibitor.
4. The method of any of items 1 to 3, wherein the biomarker is S100A9.
5. The method of item 4, wherein the level of the biomarker is determined as mRNA.
6. The method of item 4, wherein the level of the biomarker is determined as protein.
7. The method of item 6, wherein the level of the biomarker is determined as S100A9 monomer.
8. The method of item 6, wherein the level of the biomarker is determined as a S100A8/S100A9 heterodimer.
9. The method of any of items 1 to 8, wherein the sample is a peripheral sample.
10. The method of item 9, wherein the peripheral sample is cerebrospinal fluid (CSF), blood, plasma, serum, urine, stool, saliva, sputum, gingival crevicular fluid, hair follicle or skin biopsy.
11. The method of any of items 1 to 10, wherein the LSD1 inhibitor is an irreversible LSD1 inhibitor.
12. The method of any of items 1 to 11, wherein the LSD1 inhibitor is a 2-(hetero)arylcyclopropylamino compound.
13. The method of any of items 1 to 12, wherein the LSD1 inhibitor is a compound disclosed in WO2010/043721, WO2010/084160, WO2011/035941, WO2011/042217, WO2011/131697, WO2012/013727, WO2012/013728, WO2012/045883, WO2013/057320, WO2013/057322, WO2012/135113, WO2013/022047, WO2014/058071, WO2010/143582, US2010-0324147, WO2011/131576, WO2014/084298, WO2014/086790, WO2014/164867, or WO2015/021128.
14. The method of any of items 1 to 12, wherein the LSD1 inhibitor is a compound of formula (I), (II), (III), (IV), (V), (VI), (VII), (VIII), (IX), (X), (XI), (XII) or (XIII).
15. The method of any of items 1 to 12, wherein the LSD1 inhibitor is a compound of formula (III).
16. The method of any of items 1 to 12, wherein the LSD1 inhibitor is a compound of formula (VI).
17. The method of any of items 1 to 12, wherein the LSD1 inhibitor is a compound of formula (VIII).
18. The method of any of items 1 to 12, wherein the LSD1 inhibitor is a compound of formula (IX).
19. The method of any of items 1 to 12, wherein the LSD1 inhibitor is a compound of formula (X).
20. The method of any of items 1 to 12, wherein the LSD1 inhibitor is a compound of formula (XI).
21. The method of any of items 1 to 10, wherein the LSD1 inhibitor is (−) 5-((((trans)-2-(4-(benzyloxy)phenyl)cyclopropyl)amino)methyl)-1,3,4-oxadiazol-2-amine or a pharmaceutically acceptable salt or solvate thereof.
22. The method of any of items 1 to 10, wherein the LSD1 inhibitor is (trans)-N1-((1R,2S)-2-phenylcyclopropyl)cyclohexane-1,4-diamine or a pharmaceutically acceptable salt or solvate thereof.
23. The method of any of items 1 to 10, wherein the LSD1 inhibitor is 4-((4-((((1R,2S)-2-phenylcyclopropyl)amino)methyl)piperidin-1-yl)methyl)benzoic acid or a pharmaceutically acceptable salt or solvate thereof.
24. The method of any of items 1 to 23, wherein the subject is a human.
25. The method of any of items 1 to 24, wherein the subject has a CNS disease
26. The method of item 25, wherein the CNS disease is a neurodegenerative disease (e.g. Alzheimer's disease, Mild Cognitive Impairment, Parkinson's disease, difuse Lewy body disease, synucleinopathies, Huntington's disease, Down syndrome, and Amyotrophic lateral sclerosis); an autism spectrum disease (e.g. autism, Asperger syndrome, pervasive developmental disorder not otherwise specified (PDD-NOS), and childhood disintegrative disorder); a cognitive function related disease (e.g. dementia such as vascular dementia, Lewy body dementia, senile dementia, frontotemporal dementia and mixed dementia, delirium, amnesia, Rett disease, schizophrenia, attention-deficit/hyperactivity disorder, and postoperative cognitive dysfunction); a mood disorder (e.g. anxiety, stress disorder, post-traumatic stress disorder, panic disorder, phobia, mania, depressive disorders such as major depression, recurrent depression and postpartum disorder, bipolar disorders, and obsessive-compulsive disorder); stroke or a lesion-related disease (e.g. Traumatic Brain Injury, brain ischemia, intracranial hemorrhage, intracranial aneurysm, and Cerebral Amyloid Angiopathy).
27. The method of any of items 1 to 24, wherein the subject has a neurodegenerative disease, preferably Alzheimer's disease, Mild Cognitive Impairment, Parkinson's disease, difuse Lewy body disease, synucleinopathies, Huntington's disease, Down syndrome, or Amyotrophic lateral sclerosis, more preferably Alzheimer's disease or Mild Cognitive Impairment.
28. The method of any of items 1 to 24, wherein the subject has a cognitive function related disease, preferably a dementia (e.g. vascular dementia, Lewy body dementia, senile dementia, frontotemporal dementia and mixed dementia), delirium, amnesia, Rett disease, schizophrenia, attention-deficit/hyperactivity disorder, or postoperative cognitive dysfunction.
29. The method of any of items 1 to 24, wherein the subject has an autoimmune disease.
30. The method of item 29, wherein the autoimmune disease is arthritis (e.g. rheumatoid arthritis, psoriatic arthritis, reactive arthritis or juvenile idiopathic arthritis); inflammatory bowel disease (e.g. Crohn's disease and ulcerative colitis); sclerosis (e.g. systemic sclerosis); an acute or chronic autoimmune neuropathy (e.g. autoimmune encephalomyelitis or multiple sclerosis); lupus (e.g. lupus erythematosus, glomerulonephritis, or vasculitis); an autoimmune pancreas disease (e.g. autoimmune pancreatitis or diabetes mellitus type 1); an autoimmune skin disease (e.g. psoriasis); an autoimmune muscle disease (e.g. dermatomyositis, polymyositis, or inclusion body myositis); or Kawasaki disease.
31. The method of any of items 1 to 24, wherein the subject has an infection or a disease caused by an infection, preferably a bacterial infection, a fungal infection, a protozoan infection, an influenza infection, or a disease caused by any of said infections.
32. The method of item 31, wherein the infection is a bacterial infection (e.g. caused by E. coli, Pneumococcus, Helicobacter pylori, Salmonella, Staphylococcus aureus, Pseudomonas aeruginosa, Ureaplasma parvum, Francisella tularensis, and Porphyromonas gingivalis) or a disease caused by a bacterial infection such as an acute bacterial infection (e.g. acute appendicitis, meningitis, caries, gastritis, gastric ulceration, and acne) or sepsis (e.g. Severe sepsis, septic shock, perinatal or neonatal sepsis); a fungal infection (e.g. Candidiasis or Aspergillosis) or a disease caused by a fungal infection, a protozoan infection (e.g. caused by Plasmodium or Trypanomoma cruzi) or a disease caused by a protozoan infection (e.g. malaria or Chagas' disease); a viral infection (e.g. influenza virus) or a disease caused by a viral infection (e.g. Influenza).
33. The method of any of items 1 to 24, wherein the subject has cancer.
34. The method of item 33, wherein the cancer is a carcinoma, preferably colorectal cancer, bladder cancer, prostate cancer, anaplastic thyroid carcinoma, cutaneous squamous cell carcinoma, gastric cancer, lung cancer or breast cancer (including metastatic breast cancer to brain); or a sarcoma, preferably glioma (e.g. astrocytoma).
35. The method of any of items 1 to 24, wherein the subject has a cardiovascular disease.
36. The method of item 35, wherein the cardiovascular disease is arteriosclerotic vascular disease (e.g. atherosclerosis and atherogenesis), acute coronary syndromes (e.g. myocardial infarction) or vascular injury (e.g. thrombosis, embolism, vasculitis, venous ulcer, or aortic aneurysm).
37. A method for determining whether a patient is likely to respond to treatment with an LSD1 inhibitor, comprising determining the level of a biomarker which is S100A9 and/or S100A8 in a sample obtained from the patient prior to treatment with the LSD1 inhibitor, where if the level of the biomarker in the sample is elevated as compared to a control, it is more likely that the LSD1 inhibitor would have a therapeutic effect on the patient.
38. A method for determining if a patient is a candidate to receive treatment with an LSD1 inhibitor, comprising determining the level of a biomarker which is S100A9 and/or S100A8 in a sample obtained from the patient prior to treatment with the LSD1 inhibitor, where if the level of the biomarker in the sample is elevated as compared to a control, the patient is regarded as a candidate to receive treatment with the LSD1 inhibitor.
39. A method for assessing whether a diseased cell is likely responsive to an LSD1 inhibitor, the method comprising
(i) determining the level of a biomarker which is S100A9 and/or S100A8 in a sample obtained from a patient prior to treatment with the LSD1 inhibitor,
(ii) assessing that the cell is likely responsive to the LSD1 inhibitor, when the level of the biomarker in the sample is elevated compared to a control.
   40. A method for assessing whether a patient is likely responsive to an LSD1 inhibitor, the method comprising
(i) determining the level of a biomarker which is S100A9 and/or S100A8 in a sample obtained from the patient prior to treatment with the LSD1 inhibitor,
(ii) assessing that the patient is likely responsive to the LSD1 inhibitor, when the level of the biomarker in the sample is elevated compared to a control.
   41. Use of a biomarker which is S100A9 and/or S100A8 as a selection tool to identify patients with increased likelihood to benefit from treatment with an LSD1 inhibitor.
   42. The method of any of items 37 to 40 or the use of item 41, wherein the patient has a CNS disease.
   43. The method of item 42 or the use of item 42, wherein the CNS disease is a neurodegenerative disease (e.g. Alzheimer's disease, Mild Cognitive Impairment, Parkinson's disease, difuse Lewy body disease, synucleinopathies, Huntington's disease, Down syndrome, and Amyotrophic lateral sclerosis); an autism spectrum disease (e.g. autism, Asperger syndrome, pervasive developmental disorder not otherwise specified (PDD-NOS), and childhood disintegrative disorder); a cognitive function related disease (e.g. dementia such as vascular dementia, Lewy body dementia, senile dementia, frontotemporal dementia and mixed dementia, delirium, amnesia, Rett disease, schizophrenia, attention-deficit/hyperactivity disorder, and postoperative cognitive dysfunction); a mood disorder (e.g. anxiety, stress disorder, post-traumatic stress disorder, panic disorder, phobia, mania, depressive disorders such as major depression, recurrent depression and postpartum disorder, bipolar disorders, and obsessive-compulsive disorder); stroke or a lesion-related disease (e.g. Traumatic Brain Injury, brain ischemia, intracranial hemorrhage, intracranial aneurysm, and Cerebral Amyloid Angiopathy).
   44. The method of any of items 37 to 40 or the use of item 41, wherein the patient has a neurodegenerative disease, preferably Alzheimer's disease, Mild Cognitive Impairment, Parkinson's disease, difuse Lewy body disease, synucleinopathies, Huntington's disease, Down syndrome, or Amyotrophic lateral sclerosis, more preferably Alzheimer's disease or Mild Cognitive Impairment.
   45. The method of any of items 37 to 40 or the use of item 41, wherein the patient has a cognitive function related disease, preferably dementia (e.g. vascular dementia, Lewy body dementia, senile dementia, frontotemporal dementia and mixed dementia), delirium, amnesia, Rett disease, schizophrenia, attention-deficit/hyperactivity disorder, or postoperative cognitive dysfunction.
   46. The method of any of items 37 to 40 or the use of item 41, wherein the patient has an autoimmune disease.
   47. The method of item 46 or the use of item 46, wherein the autoimmune disease is arthritis (e.g. rheumatoid arthritis, psoriatic arthritis, reactive arthritis or juvenile idiopathic arthritis); inflammatory bowel disease (e.g. Crohn's disease and ulcerative colitis); sclerosis (e.g. systemic sclerosis); an acute or chronic autoimmune neuropathy (e.g. autoimmune encephalomyelitis or multiple sclerosis); lupus (e.g. lupus erythematosus, glomerulonephritis, or vasculitis); an autoimmune pancreas disease (e.g. autoimmune pancreatitis or diabetes mellitus type 1); an autoimmune skin disease (e.g. psoriasis); an autoimmune muscle disease (e.g. dermatomyositis, polymyositis, or inclusion body myositis); or Kawasaki disease.
   48. The method of any of items 37 to 40 or the use of item 41, wherein the patient has an infection or a disease caused by an infection, preferably a bacterial infection, a fungal infection, a protozoan infection, an influenza infection, or a disease caused by any of said infections.
   49. The method of item 48 or the use of item 48, wherein the infection is a bacterial infection (e.g. caused by E. coli, Pneumococcus, Helicobacter pylori, Salmonella, Staphylococcus aureus, Pseudomonas aeruginosa, Ureaplasma parvum, Francisella tularensis, and Porphyromonas gingivalis) or a disease caused by a bacterial infection such as an acute bacterial infection (e.g. acute appendicitis, meningitis, caries, gastritis, gastric ulceration, and acne) or sepsis (e.g. Severe sepsis, septic shock, perinatal or neonatal sepsis); a fungal infection (e.g. Candidiasis or Aspergillosis) or a disease caused by a fungal infection, a protozoan infection (e.g. caused by Plasmodium or Trypanomoma cruzi) or a disease caused by a protozoan infection (e.g. malaria or Chagas' disease); a viral infection (e.g. influenza virus) or a disease caused by a viral infection (e.g. Influenza).
   50. The method of any of items 37 to 40 or the use of item 41, wherein the patient has cancer.
   51. The method of item 50 or the use of item 50, wherein the cancer is a carcinoma, preferably colorectal cancer, bladder cancer, prostate cancer, anaplastic thyroid carcinoma, cutaneous squamous cell carcinoma, gastric cancer, lung cancer or breast cancer (including metastatic breast cancer to brain); or a sarcoma, preferably glioma (e.g. astrocytoma).
   52. The method of any of items 37 to 40 or the use of item 41, wherein the patient has a cardiovascular disease.
   53. The method of item 52 or the use of item 52, wherein the cardiovascular disease is arteriosclerotic vascular disease (e.g. atherosclerosis and atherogenesis), acute coronary syndromes (e.g. myocardial infarction) or vascular injury (e.g. thrombosis, embolism, vasculitis, venous ulcer, or aortic aneurysm).
   54. A method for determining whether a beneficial effect in cognitive function is likely to be produced by treatment with an LSD1 inhibitor in a patient suffering from a neurodegenerative disease, comprising determining the level of a biomarker which is S100A9 and/or S100A8 in a sample obtained from the patient prior to treatment with the LSD1 inhibitor, where if the level of the biomarker in the sample is elevated compared to a control, it is more likely that the LSD1 inhibitor would produce a beneficial effect in cognitive function in the patient.
   55. A method for determining whether a beneficial effect in cognitive function is likely to be produced by treatment with an LSD1 inhibitor in a patient suffering from a cognitive function related disease, comprising determining the level of a biomarker which is S100A9 and/or S100A8 in a sample obtained from the patient prior to treatment with the LSD1 inhibitor, where if the level of the biomarker in the sample is elevated compared to a control, it is more likely that the LSD1 inhibitor would produce a beneficial effect in cognitive function in the patient.
   56. A method for selecting a patient having mild cognitive impairment for receiving treatment with an LSD1 inhibitor, comprising determining the level of a biomarker which is S100A9 and/or S100A8 in a sample obtained from the patient prior to treatment with the LSD1 inhibitor, and selecting the patient to receive treatment with the LSD1 inhibitor if the level of the biomarker in the sample is elevated compared to a control.
   57. The method of any of items 37 to 40 or 42 to 56 or the use of any of items 41 to 53, wherein the biomarker is S100A9.
   58. The method of item 57 or the use of item 57, wherein the level of the biomarker is determined as mRNA.
   59. The method of item 57 or the use of item 57, wherein the level of the biomarker is determined as protein.
   60. The method of item 59 or the use of item 59, wherein the level of the biomarker is determined as S100A9 monomer.
   61. The method of item 59 or the use of item 59, wherein the level of the biomarker is determined as a S100A8/S100A9 heterodimer.
   62. The method of any of items 37 to 40 or 42 to 61, wherein the sample is a peripheral sample.
   63. The method of item 62, wherein the peripheral sample is cerebrospinal fluid (CSF), blood, plasma, serum, urine, stool, saliva, sputum, gingival crevicular fluid, hair follicle or skin biopsy.
   64. The method of any of items 42 to 45, 54 to 56 or 62, wherein the peripheral sample is cerebrospinal fluid (CSF), blood, plasma, or serum.
   65. The method of any of items 37 to 40 or 42 to 64 or the use of any of items 41 to 53 or 57 to 61, wherein the LSD1 inhibitor is an irreversible LSD1 inhibitor.
   66. The method of any of items 37 to 40 or 42 to 65 or the use of any of items 41 to 53, 57 to 61 or 65, wherein the LSD1 inhibitor is a 2-(hetero)arylcyclopropylamino compound.
   67. The method of any of items 37 to 40 or 42 to 66 or the use of any of items 41 to 53, 57 to 61, 65 or 66, wherein the LSD1 inhibitor is a compound disclosed in WO2010/043721, WO2010/084160, WO2011/035941, WO2011/042217, WO2011/131697, WO2012/013727, WO2012/013728, WO2012/045883, WO2013/057320, WO2013/057322, WO2012/135113, WO2013/022047, WO2014/058071, WO2010/143582, US2010-0324147, WO2011/131576, WO2014/084298, WO2014/086790, WO2014/164867, or WO2015/021128.
   68. The method of any of items 37 to 40 or 42 to 65 or the use of any of items 41 to 53, 57 to 61 or 65, wherein the LSD1 inhibitor is a compound of formula (I), (II), (III), (IV), (V), (VI), (VII), (VIII), (IX), (X), (XI), (XII) or (XIII).
   69. The method of any of items 37 to 40 or 42 to 65 or the use of any of items 41 to 53, 57 to 61 or 65, wherein the LSD1 inhibitor is a compound of formula (III).
   70. The method of any of items 37 to 40 or 42 to 65 or the use of any of items 41 to 53, 57 to 61 or 65, wherein the LSD1 inhibitor is a compound of formula (VI).
   71. The method of any of items 37 to 40 or 42 to 65 or the use of any of items 41 to 53, 57 to 61 or 65, wherein the LSD1 inhibitor is a compound of formula (VIII).
   72. The method of any of items 37 to 40 or 42 to 65 or the use of any of items 41 to 53, 57 to 61 or 65, wherein the LSD1 inhibitor is a compound of formula (IX).
   73. The method of any of items 37 to 40 or 42 to 65 or the use of any of items 41 to 53, 57 to 61 or 65, wherein the LSD1 inhibitor is a compound of formula (X).
   74. The method of any of items 37 to 40 or 42 to 65 or the use of any of items 41 to 53, 57 to 61 or 65, wherein the LSD1 inhibitor is a compound of formula (XI).
   75. The method of any of items 37 to 40 or 42 to 65 or the use of any of items 41 to 53, 57 to 61 or 65, wherein the LSD1 inhibitor is (−) 5-((((trans)-2-(4-(benzyloxy)phenyl)cyclopropyl)amino)methyl)-1,3,4-oxadiazol-2-amine or a pharmaceutically acceptable salt or solvate thereof.
   76. The method of any of items 37 to 40 or 42 to 65 or the use of any of items 41 to 53, 57 to 61 or 65, wherein the LSD1 inhibitor is (trans)-N1-((1R,2S)-2-phenylcyclopropyl)cyclohexane-1,4-diamine or a pharmaceutically acceptable salt or solvate thereof.
   77. The method of any of items 37 to 40 or 42 to 65 or the use of any of items 41 to 53, 57 to 61 or 65, wherein the LSD1 inhibitor is 4-((4-((((1R,2S)-2-phenylcyclopropyl)amino)methyl)piperidin-1-yl)methyl)benzoic acid or a pharmaceutically acceptable salt or solvate thereof.
   78. A method for treating a patient, comprising: (i) determining the level of a biomarker which is S100A9 and/or S100A8 in a sample obtained from the patient prior to treatment with an LSD1 inhibitor, and (ii) administering to the patient a therapeutically effective amount of the LSD1 inhibitor if the level of the biomarker in the sample is elevated as compared to a control.
   79. A method for treating a patient, comprising: (i) determining the level of a biomarker which is S100A9 and/or S100A8 in a sample obtained from the patient prior to treatment with an LSD1 inhibitor, (ii) determining whether the patient is likely responsive to treatment with the LSD1 inhibitor, wherein an elevated level of the biomarker in the sample as compared to a control is indicative of the patient being likely responsive to the treatment with the LSD1 inhibitor, and (iii) administering to the patient a therapeutically effective amount of the LSD1 inhibitor if the patient has been identified as being likely responsive to the treatment with the LSD1 inhibitor.
   80. A method for treating a patient, comprising: (i) determining likeliness of responsiveness of the patient to an LSD1 inhibitor by the method as defined in any of items 37 to 40; and (ii) administering to the patient a therapeutically effective amount of the LSD1 inhibitor if the patient is identified to be likely responsive thereto.
   81. The method of any of items 78 to 80, wherein the method is for treating a patient having a CNS disease.
   82. The method of any of items 78 to 80, wherein the method is for treating a patient having a neurodegenerative disease, preferably Alzheimer's disease, Mild Cognitive Impairment, Parkinson's disease, difuse Lewy body disease, synucleinopathies, Huntington's disease, Down syndrome, or Amyotrophic lateral sclerosis, more preferably Alzheimer's disease or Mild Cognitive Impairment.
   83. The method of any of items 78 to 80, wherein the method is for treating a patient having a cognitive function related disease, preferably dementia (e.g. vascular dementia, Lewy body dementia, senile dementia, frontotemporal dementia and mixed dementia), delirium, amnesia, Rett disease, schizophrenia, attention-deficit/hyperactivity disorder, or postoperative cognitive dysfunction.
   84. The method of any of items 78 to 80, wherein the method is for treating a patient having an autoimmune disease.
   85. The method of any of items 78 to 80, wherein the method is for treating a patient having an infection or a disease caused by an infection, preferably a bacterial infection, a fungal infection, a protozoan infection, an influenza infection, or a disease caused by any of said infections.
   86. The method of any of items 78 to 80, wherein the method is for treating a patient having cancer.
   87. The method of any of items 78 to 80, wherein the method is for treating a patient having a cardiovascular disease.
   88. A method for treating a disease characterized by induction of S100A9 and/or S100A8 in a patient, comprising: (i) determining the level of a biomarker which is S100A9 and/or S100A8 in a sample obtained from the patient prior to treatment with an LSD1 inhibitor, and (ii) administering to the patient a therapeutically effective amount of the LSD1 inhibitor if the level of the biomarker in the sample is elevated compared to a control.
   89. A method for treating a patient having a disease characterized by induction of S100A9 and/or S100A8, comprising: (i) determining the level of a biomarker which is S100A9 and/or S100A8 in a sample obtained from the patient prior to treatment with an LSD1 inhibitor, and (ii) administering to the patient a therapeutically effective amount of the LSD1 inhibitor if the level of the biomarker in the sample is elevated compared to a control.
   90. A method for treating a patient having a disease characterized by S100A9 and/or S100A8 induction, the method comprising obtaining a sample of a patient for whom LSD1 inhibitor therapy is contemplated, and testing the sample to determine an elevated level of a biomarker which is S100A9 and/or S100A8 compared to a control therein and administering an effective amount of the LSD1 inhibitor to the patient having a disease characterized by S100A9 and/or S100A8 induction.
   91. A method for treating a CNS disease in a patient, comprising: (i) determining the level of a biomarker which is S100A9 and/or S100A8 in a sample obtained from the patient prior to treatment with an LSD1 inhibitor, and (ii) administering to the patient a therapeutically effective amount of the LSD1 inhibitor if the level of the biomarker in the sample is elevated compared to a control.
   92. A method for treating a neurodegenerative disease in a patient, comprising: (i) determining the level of a biomarker which is S100A9 and/or S100A8 in a sample obtained from the patient prior to treatment with an LSD1 inhibitor, and (ii) administering to the patient a therapeutically effective amount of the LSD1 inhibitor if the level of the biomarker in the sample is elevated compared to a control.
   93. A method for treating Alzheimer's disease in a patient, comprising: (i) determining the level of a biomarker which is S100A9 and/or S100A8 in a sample obtained from the patient prior to treatment with an LSD1 inhibitor, and (ii) administering to the patient a therapeutically effective amount of the LSD1 inhibitor if the level of the biomarker in the sample is elevated compared to a control
   94. A method for treating mild cognitive impairment in a patient, comprising: (i) determining the level of a biomarker which is S100A9 and/or S100A8 in a sample obtained from the patient prior to treatment with an LSD1 inhibitor, and (ii) administering to the patient a therapeutically effective amount of the LSD1 inhibitor if the level of the biomarker in the sample is elevated compared to a control.
   95. A method for treating a cognitive function related disease in a patient, comprising: (i) determining the level of a biomarker which is S100A9 and/or S100A8 in a sample obtained from the patient prior to treatment with an LSD1 inhibitor, and (ii) administering to the patient a therapeutically effective amount of the LSD1 inhibitor if the level of the biomarker in the sample is elevated compared to a control.
   96. A method for treating an autoimmune disease in a patient, comprising: (i) determining the level of a biomarker which is S100A9 and/or S100A8 in a sample obtained from the patient prior to treatment with an LSD1 inhibitor, and (ii) administering to the patient a therapeutically effective amount of the LSD1 inhibitor if the level of the biomarker in the sample is elevated compared to a control.
   97. A method for treating an autoimmune disease in a patient, comprising: (i) obtaining a sample from the patient prior to treatment with an LSD1 inhibitor, (ii) determining the level of a biomarker which is S100A9 and/or S100A8 in the sample, and (iii) administering to the patient an amount of the LSD1 inhibitor sufficient to decrease the biomarker levels while not causing a clinically relevant reduction in platelet levels if the level of the biomarker in the sample is elevated compared to a control.
   98. A method for treating an infection or a disease caused by an infection, preferably a bacterial infection, a fungal infection, a protozoan infection, an influenza infection, or a disease caused by any of said infections, in a patient, comprising: (i) determining the level of a biomarker which is S100A9 and/or S100A8 in a sample obtained from the patient prior to treatment with an LSD1 inhibitor, and (ii) administering to the patient a therapeutically effective amount of the LSD1 inhibitor if the level of the biomarker in the sample is elevated compared to a control.
   99. A method for treating cancer in a patient, comprising: (i) determining the level of a biomarker which is S100A9 and/or S100A8 in a sample obtained from the patient prior to treatment with an LSD1 inhibitor, and (ii) administering to the patient a therapeutically effective amount of the LSD1 inhibitor if the level of the biomarker in the sample is elevated compared to a control.
   100. A method for treating a cardiovascular disease in a patient, comprising: (i) determining the level of a biomarker which is S100A9 and/or S100A8 in a sample obtained from the patient prior to treatment with an LSD1 inhibitor, and (ii) administering to the patient a therapeutically effective amount of the LSD1 inhibitor if the level of the biomarker in the sample is elevated compared to a control.
   101. A method for treating a cardiovascular disease in a patient, comprising: (i) obtaining a sample from the patient prior to treatment with an LSD1 inhibitor, (ii) determining the level of a biomarker which is S100A9 and/or S100A8 in the sample, and (iii) administering to the patient an amount of the LSD1 inhibitor sufficient to decrease the biomarker levels while not causing a clinically relevant reduction in platelet levels if the level of the biomarker in the sample is elevated compared to a control.
   102. A method for treating Mild Cognitive Impairment in a patient, comprising administering to the patient a therapeutically effective amount of an LSD1 inhibitor.
   103. A method for treating an infection or a disease caused by an infection, preferably a bacterial infection, a fungal infection, a protozoan infection, an influenza infection, or a disease caused by any of said infections, in a patient, comprising administering to the patient a therapeutically effective amount of an LSD1 inhibitor.
   104. A method for treating an autoimmune disease in a patient, comprising administering to the patient an amount of an LSD1 inhibitor that decreases the level of a biomarker which is S100A9 and/or S100A8 while not causing a clinically relevant reduction in platelet levels.
   105. A method for treating a cardiovascular disease in a patient, comprising administering to the patient an amount of an LSD1 inhibitor that decreases the level of a biomarker which is S100A9 and/or S100A8 while not causing a clinically relevant reduction in platelet levels.
   106. An LSD1 inhibitor for use in therapy, wherein said therapy comprises: (i) determining the level of a biomarker which is S100A9 and/or S100A8 in a sample obtained from a patient prior to treatment with an LSD1 inhibitor, and (ii) administering the LSD1 inhibitor to the patient if the level of the biomarker in the sample is elevated as compared to a control.
   107. An LSD1 inhibitor for use in a method of treating a disease selected from the group consisting of a CNS disease, an autoimmune disease, an infection or a disease caused by an infection (preferably a bacterial infection, a fungal infection, a protozoan infection, an influenza infection, or a disease caused by any of said infections), cancer and a cardiovascular disease in a patient, the method comprising: (i) determining the level of a biomarker which is S100A9 and/or S100A8 in a sample obtained from the patient prior to treatment with an LSD1 inhibitor, and (ii) administering the LSD1 inhibitor to the patient if the level of the biomarker in the sample is elevated as compared to a control.
   108. An LSD1 inhibitor for use in a method of treating a disease selected from the group consisting of a CNS disease, an autoimmune disease, an infection or a disease caused by an infection (preferably a bacterial infection, a fungal infection, a protozoan infection, an influenza infection, or a disease caused by any of said infections), cancer and a cardiovascular disease in a patient, the method comprising: (i) determining the level of a biomarker which is S100A9 and/or S100A8 in a sample obtained from the patient prior to treatment with an LSD1 inhibitor, (ii) determining whether the patient is likely responsive to the treatment with the LSD1 inhibitor, wherein an elevated level of the biomarker in the sample as compared to a control is indicative of the patient being likely responsive to the treatment with the LSD1 inhibitor, and (iii) administering the LSD1 inhibitor to the patient if the patient has been identified as being likely responsive to the treatment with the LSD1 inhibitor.
   109. An LSD1 inhibitor for use in treating a disease selected from the group consisting of a CNS disease, an autoimmune disease, an infection or a disease caused by an infection (preferably a bacterial infection, a fungal infection, a protozoan infection, an influenza infection, or a disease caused by any of said infections), cancer and a cardiovascular disease in a patient, wherein the patient has been predicted to be likely responsive to treatment with an LSD1 inhibitor by the method defined in any of items 37 to 40.
   110. An LSD1 inhibitor for use in treating a disease characterized by induction of S100A9 and/or S100A8 in a patient, wherein the level of a biomarker which is S100A9 and/or S100A8 is determined in a sample from the patient prior to treatment with an LSD1 inhibitor, and the LSD1 inhibitor is administered to the patient if the level of the biomarker in the sample is elevated compared to a control.
   111. An LSD1 inhibitor for use in a method of treating a disease characterized by S100A9 and/or S100A8 induction in a patient, wherein the patient has an elevated level of a biomarker which is S100A9 and/or S100A8 compared to a control.
   112. An LSD1 inhibitor for use in a method of treating a disease characterized by S100A9 and/or S100A8 induction in a patient that was assessed positive for an elevated level of a biomarker which is S100A9 and/or S100A8 compared to a control.
   113. An LSD1 inhibitor for use in a method of treating a disease characterized by S100A9 and/or S100A8 induction in a patient that has been tested positive for an elevated level of a biomarker which is S100A9 and/or S100A8 compared to a control.
   114. An LSD1 inhibitor for use in the treatment of a disease characterized by S100A9 and/or S100A8 induction wherein the patient has an elevated level of a biomarker which is S100A9 and/or S100A8 compared to a control and the method of treatment comprises the step of determining whether or not the patient has an elevated level of a biomarker which is S100A9 and/or S100A8 compared to a control.
   115. An LSD1 inhibitor for use in a method of treating a disease characterized by S100A9 and/or S100A8 induction in a patient identified as having an elevated level of a biomarker which is S100A9 and/or S100A8 compared to a control using the method of any of items 37 to 40.
   116. An LSD1 inhibitor for use in a method of treating a disease characterized by S100A9 and/or S100A8 induction, wherein said method comprises testing a patient using the method of determining an elevated level of a biomarker which is S100A9 and/or S100A8 according to any of items 37 to 40, in order to determine whether the patient has an elevated level of a biomarker which is S100A9 and/or S100A8 compared to a control, and providing treatment with an LSD1 inhibitor if the patient is identified as having an elevated level of a biomarker which is S100A9 and/or S100A8 compared to a control.
   117. An LSD1 inhibitor for use in a method of treating a disease characterized by S100A9 and/or S100A8 induction in a patient assessed positive for an elevated level of a biomarker which is S100A9 and/or S100A8 compared to a control.
   118. An LSD1 inhibitor for use in treating a CNS disease in a patient, wherein the level of a biomarker which is S100A9 and/or S100A8 is determined in a sample from the patient prior to treatment with an LSD1 inhibitor, and the LSD1 inhibitor is administered to the patient if the level of the biomarker in the sample is elevated compared to a control.
   119. An LSD1 inhibitor for use in treating a neurodegenerative disease in a patient, wherein the level of a biomarker which is S100A9 and/or S100A8 is determined in a sample from the patient prior to treatment with an LSD1 inhibitor, and the LSD1 inhibitor is administered to the patient if the level of the biomarker in the sample is elevated compared to a control.
   120. An LSD1 inhibitor for use in treating Alzheimer's disease in a patient, wherein the level of a biomarker which is S100A9 and/or S100A8 is determined in a sample from the patient prior to treatment with an LSD1 inhibitor, and the LSD1 inhibitor is administered to the patient if the level of the biomarker in the sample is elevated compared to a control.
   121. An LSD1 inhibitor for use in treating mild cognitive impairment in a patient, wherein the level of a biomarker which is S100A9 and/or S100A8 is determined in a sample from the patient prior to treatment with an LSD1 inhibitor, and the LSD1 inhibitor is administered to the patient if the level of the biomarker in the sample is elevated compared to a control.
   122. An LSD1 inhibitor for use in treating a cognitive function related disease in a patient, wherein the level of a biomarker which is S100A9 and/or S100A8 is determined in a sample from the patient prior to treatment with an LSD1 inhibitor, and the LSD1 inhibitor is administered to the patient if the level of the biomarker in the sample is elevated compared to a control.
   123. An LSD1 inhibitor for use in treating an autoimmune disease in a patient, wherein the level of a biomarker which is S100A9 and/or S100A8 is determined in a sample from the patient prior to treatment with an LSD1 inhibitor, and the LSD1 inhibitor is administered to the patient if the level of the biomarker in the sample is elevated compared to a control.
   124. An LSD1 inhibitor for use in treating an autoimmune disease in a patient, wherein the level of a biomarker which is S100A9 and/or S100A8 is determined in a sample from the patient prior to treatment with an LSD1 inhibitor, and an amount of the LSD1 inhibitor sufficient to decrease the biomarker levels while not causing a clinically relevant reduction in platelet levels is administered to the patient if the level of the biomarker in the sample is elevated compared to a control.
   125. An LSD1 inhibitor for use in treating an infection or a disease caused by an infection, preferably a bacterial infection, a fungal infection, a protozoan infection, an influenza infection, or a disease caused by any of said infections in a patient, wherein the level of a biomarker which is S100A9 and/or S100A8 is determined in a sample from the patient prior to treatment with an LSD1 inhibitor, and the LSD1 inhibitor is administered to the patient if the level of the biomarker in the sample is elevated compared to a control.
   126. An LSD1 inhibitor for use in treating cancer in a patient, wherein the level of a biomarker which is S100A9 and/or S100A8 is determined in a sample from the patient prior to treatment with an LSD1 inhibitor, and the LSD1 inhibitor is administered to the patient if the level of the biomarker in the sample is elevated compared to a control.
   127. An LSD1 inhibitor for use in treating a cardiovascular disease in a patient, wherein the level of a biomarker which is S100A9 and/or S100A8 is determined in a sample from the patient prior to treatment with an LSD1 inhibitor, and the LSD1 inhibitor is administered to the patient if the level of the biomarker in the sample is elevated compared to a control.
   128. An LSD1 inhibitor for use in treating a cardiovascular disease in a patient, wherein the level of a biomarker which is S100A9 and/or S100A8 is determined in a sample from the patient prior to treatment with an LSD1 inhibitor, and an amount of the LSD1 inhibitor sufficient to decrease the biomarker levels while not causing a clinically relevant reduction in platelet levels is administered to the patient if the level of the biomarker in the sample is elevated compared to a control.
   129. An LSD1 inhibitor for use in treating Mild Cognitive Impairment.
   130. An LSD1 inhibitor for use in treating a bacterial infection, a fungal infection, a protozoan infection, an influenza infection, or a disease caused by any of said infections.
   131. An LSD1 inhibitor for use in treating an autoimmune disease in a patient, wherein the LSD1 inhibitor is to be administered to the patient in an amount sufficient to decrease the level of a biomarker which is S100A9 and/or S100A8 while not causing a clinically relevant reduction in platelet levels.
   132. An LSD1 inhibitor for use in treating a cardiovascular disease in a patient, wherein the LSD1 inhibitor is to be administered to the patient in an amount sufficient to decrease the level of a biomarker which is S100A9 and/or S100A8 while not causing a clinically relevant reduction in platelet levels.
   133. The method of any of items 88 to 90 or the LSD1 inhibitor of any of items 110 to 117, wherein the disease characterized by S100A9 and/or S100A8 induction is a disease selected from the group consisting of a CNS disease, an autoimmune disease, an infection or a disease caused by an infection (preferably a bacterial infection, a fungal infection, a protozoan infection, an influenza infection, or a disease caused by any of said infections), cancer and a cardiovascular disease.
   134. The method of any of items 81, 91 or 133 or the LSD1 inhibitor of any of items 107 to 109, 118 or 133, wherein the CNS disease is a neurodegenerative disease (e.g. Alzheimer's disease, Mild Cognitive Impairment, Parkinson's disease, difuse Lewy body disease, synucleinopathies, Huntington's disease, Down syndrome, and Amyotrophic lateral sclerosis); an autism spectrum disease (e.g. autism, Asperger syndrome, pervasive developmental disorder not otherwise specified (PDD-NOS), and childhood disintegrative disorder); a cognitive function related disease (e.g. dementia such as vascular dementia, Lewy body dementia, senile dementia, frontotemporal dementia and mixed dementia, delirium, amnesia, Rett disease, schizophrenia, attention-deficit/hyperactivity disorder, and postoperative cognitive dysfunction); a mood disorder (e.g. anxiety, stress disorder, post-traumatic stress disorder, panic disorder, phobia, mania, depressive disorders such as major depression, recurrent depression and postpartum disorder, bipolar disorders, and obsessive-compulsive disorder); stroke or a lesion-related disease (e.g. Traumatic Brain Injury, brain ischemia, intracranial hemorrhage, intracranial aneurysm, and Cerebral Amyloid Angiopathy).
   135. The method of any of items 84, 96, 97, 104 or 133 or the LSD1 inhibitor of any of items 107 to 109, 123, 124, 131 or 133, wherein the autoimmune disease is arthritis (e.g. rheumatoid arthritis, psoriatic arthritis, reactive arthritis or juvenile idiopathic arthritis); inflammatory bowel disease (e.g. Crohn's disease and ulcerative colitis); sclerosis (e.g. systemic sclerosis); an acute or chronic autoimmune neuropathy (e.g. autoimmune encephalomyelitis or multiple sclerosis); lupus (e.g. lupus erythematosus, glomerulonephritis, or vasculitis); an autoimmune pancreas disease (e.g. autoimmune pancreatitis or diabetes mellitus type 1); an autoimmune skin disease (e.g. psoriasis); an autoimmune muscle disease (e.g. dermatomyositis, polymyositis, or inclusion body myositis); or Kawasaki disease.
   136. The method of any of items 85, 98, 103 or 133 or the LSD1 inhibitor of any of items 107 to 109, 125 or 133, wherein the infection is a bacterial infection (e.g. caused by E. coli, Pneumococcus, Helicobacter pylori, Salmonella, Staphylococcus aureus, Pseudomonas aeruginosa, Ureaplasma parvum, Francisella tularensis, and Porphyromonas gingivalis) or a disease caused by a bacterial infection such as an acute bacterial infection (e.g. acute appendicitis, meningitis, caries, gastritis, gastric ulceration, and acne) or sepsis (e.g. Severe sepsis, septic shock, perinatal or neonatal sepsis); a fungal infection (e.g. Candidiasis or Aspergillosis) or a disease caused by a fungal infection, a protozoan infection (e.g. caused by Plasmodium or Trypanomoma cruzi) or a disease caused by a protozoan infection (e.g. malaria or Chagas' disease); a viral infection (e.g. influenza virus) or a disease caused by a viral infection (e.g. Influenza).
   137. The method of any of items 86, 99 or 133 or the LSD1 inhibitor of any of items 107 to 109, 126 or 133, wherein the cancer is a carcinoma, preferably colorectal cancer, bladder cancer, prostate cancer, anaplastic thyroid carcinoma, cutaneous squamous cell carcinoma, gastric cancer, lung cancer or breast cancer (including metastatic breast cancer to brain); or a sarcoma, preferably glioma (e.g. astrocytoma).
   138. The method of any of items 87, 100, 101, 105 or 133 or the LSD1 inhibitor of any of items 107 to 109, 127, 128, 132 or 133, wherein the cardiovascular disease is arteriosclerotic vascular disease (e.g. atherosclerosis and atherogenesis), acute coronary syndromes (e.g. myocardial infarction) or vascular injury (e.g. thrombosis, embolism, vasculitis, venous ulcer, or aortic aneurysm).
   139. The method of any of items 78 to 105 or 133 to 138 or the LSD1 inhibitor of any of items 106 to 138, wherein the biomarker is S100A9.
   140. The method of item 139 or the LSD1 inhibitor of item 139, wherein the level of biomarker is determined as mRNA.
   141. The method of item 139 or the LSD1 inhibitor of item 139, wherein the level of biomarker is determined as protein.
   142. The method of item 141 or the LSD1 inhibitor of item 141, wherein the level of the biomarker is determined as S100A9 monomer.
   143. The method of item 141 or the LSD1 inhibitor of item 141, wherein the level of the biomarker is determined as a S100A8/S100A9 heterodimer.
   144. The method of any of items 78 to 105 or 133 to 143 or the LSD1 inhibitor of any of items 106 to 108, 110, 118 to 128 or 133 to 143, wherein the sample is a peripheral sample.
   145. The method of item 144 or the LSD1 inhibitor of item 144, wherein the peripheral sample is cerebrospinal fluid (CSF), blood, plasma, serum, stool, saliva, sputum, gingival crevicular fluid, hair follicle or skin biopsy.
   146. The method of any of items 81 to 83, 91 to 95 or 134 or the LSD1 inhibitor of any of items 118 to 122 or 134, wherein the peripheral sample is cerebrospinal fluid (CSF), blood, plasma, or serum.
   147. The method of any of items 78 to 105 or 133 to 146 or the LSD1 inhibitor of any of items 106 to 146, wherein the LSD1 inhibitor is an irreversible LSD1 inhibitor.
   148. The method of any of items 78 to 105 or 133 to 146 or the LSD1 inhibitor of any of items 106 to 146, wherein the LSD1 inhibitor is a 2-(hetero)arylcyclopropylamino compound.
   149. The method of any of items 78 to 105 or 133 to 146 or the LSD1 inhibitor of any of items 106 to 146, wherein the LSD1 inhibitor is a compound disclosed in WO2010/043721, WO2010/084160, WO2011/035941, WO2011/042217, WO2011/131697, WO2012/013727, WO2012/013728, WO2012/045883, WO2013/057320, WO2013/057322, WO2012/135113, WO2013/022047, WO2014/058071, WO2010/143582, US2010-0324147, WO2011/131576, WO2014/084298, WO2014/086790, WO2014/164867, or WO2015/021128.
   150. The method of any of items 78 to 105 or 133 to 146 or the LSD1 inhibitor of any of items 106 to 146, wherein the LSD1 inhibitor is a compound of formula (I), (II), (III), (IV), (V), (VI), (VII), (VIII), (IX), (X), (XI), (XII) or (XIII).
   151. The method of any of items 78 to 105 or 133 to 146 or the LSD1 inhibitor of any of items 106 to 146, wherein the LSD1 inhibitor is a compound of formula (III).
   152. The method of any of items 78 to 105 or 133 to 146 or the LSD1 inhibitor of any of items 106 to 146, wherein the LSD1 inhibitor is a compound of formula (VI).
   153. The method of any of items 78 to 105 or 133 to 146 or the LSD1 inhibitor of any of items 106 to 146, wherein the LSD1 inhibitor is a compound of formula (VIII).
   154. The method of any of items 78 to 105 or 133 to 146 or the LSD1 inhibitor of any of items 106 to 146, wherein the LSD1 inhibitor is a compound of formula (IX).
   155. The method of any of items 78 to 105 or 133 to 146 or the LSD1 inhibitor of any of items 106 to 146, wherein the LSD1 inhibitor is a compound of formula (X).
   156. The method of any of items 78 to 105 or 133 to 146 or the LSD1 inhibitor of any of items 106 to 146, wherein the LSD1 inhibitor is a compound of formula (XI).
   157. The method of any of items 78 to 105 or 133 to 146 or the LSD1 inhibitor of any of items 106 to 146, wherein the LSD1 inhibitor is (−) 5-((((trans)-2-(4-(benzyloxy)phenyl)cyclopropyl)amino)methyl)-1,3,4-oxadiazol-2-amine or a pharmaceutically acceptable salt or solvate thereof.
   158. The method of any of items 78 to 105 or 133 to 146 or the LSD1 inhibitor of any of items 106 to 146, wherein the LSD1 inhibitor is (trans)-N1-((1R,2S)-2-phenylcyclopropyl)cyclohexane-1,4-diamine or a pharmaceutically acceptable salt or solvate thereof.
   159. The method of any of items 78 to 105 or 133 to 146 or the LSD1 inhibitor of any of items 106 to 146, wherein the LSD1 inhibitor is 4-((4-((((1R,2S)-2-phenylcyclopropyl)amino)methyl)piperidin-1-yl)methyl)benzoic acid or a pharmaceutically acceptable salt or solvate thereof.
   160. The method of any of items 37 to 40, 42 to 105 or 133 to 159, the use of any of items 41 to 53, 57 to 61 or 65 to 77 or the LSD1 inhibitor of any of items 106 to 128 or 131 to 159, wherein the patient is a human.
   161. A combination comprising a LSD1 inhibitor and a S100A9 and/or S100A8 inhibitor.
   162. A combination comprising a LSD1 inhibitor and a S100A9 and/or S100A8 inhibitor for use in the treatment of a disease characterized by S100A9 and/or S100A8 induction.
   163. The combination of item 161 or 162, wherein the S100A9 and/or S100A8 inhibitor is a corticosteroid.
   164. The combination of item 161 or 162, wherein the S100A9 and/or S100A8 inhibitor is an agent that inhibits the interaction between S100A9 and/or S100A8 and TLR4 or RAGE.
   165. The combination of item 164, wherein the agent that inhibits the interaction between S100A9 and/or S100A8 and TLR4 or RAGE is a quinoline-3-carboxamide.
   166. The combination of item 164 or 165, wherein the agent that inhibits the interaction between S100A9 and/or S100A8 and TLR4 or RAGE is paquinimod, tasquinimod, or laquinimod.
   167. A combination comprising an LSD1 inhibitor and an antibacterial agent.
   168. A combination comprising an LSD1 inhibitor and an antibacterial agent for use in the treatment of a bacterial infection or a disease caused by a bacterial infection.
   169. The combination of any of items 161 to 168, wherein the LSD1 inhibitor is an irreversible LSD1 inhibitor.
   170. The combination of any of items 161 to 168, wherein the LSD1 inhibitor is a 2-(hetero)arylcyclopropylamino compound.
   171. The combination of any of items 161 to 168, wherein the LSD1 inhibitor is a compound disclosed in WO2010/043721, WO2010/084160, WO2011/035941, WO2011/042217, WO2011/131697, WO2012/013727, WO2012/013728, WO2012/045883, WO2013/057320, WO2013/057322, WO2012/135113, WO2013/022047, WO2014/058071, WO2010/143582, US2010-0324147, WO2011/131576, WO2014/084298, WO2014/086790, WO2014/164867, or WO2015/021128.
   172. The combination of any of items 161 to 168, wherein the LSD1 inhibitor is compound of formula (I), (II), (III), (IV), (V), (VI), (VII), (VIII), (IX), (X), (XI), (XII) or (XIII).
   173. The combination of any of items 161 to 168, wherein the LSD1 inhibitor is a compound of formula (III).
   174. The combination of any of items 161 to 168, wherein the LSD1 inhibitor is a compound of formula (VI).
   175. The combination of any of items 161 to 168, wherein the LSD1 inhibitor is a compound of formula (VIII).
   176. The combination of any of items 161 to 168, wherein the LSD1 inhibitor is a compound of formula (IX).
   177. The combination of any of items 161 to 168, wherein the LSD1 inhibitor is a compound of formula (X).
   178. The combination of any of items 161 to 168, wherein the LSD1 inhibitor is a compound of formula (XI).
   179. The combination of any of items 161 to 168, wherein the LSD1 inhibitor is (−) 5-((((trans)-2-(4-(benzyloxy)phenyl)cyclopropyl)amino)methyl)-1,3,4-oxadiazol-2-amine or a pharmaceutically acceptable salt or solvate thereof.
   180. The combination of any of items 161 to 168, wherein the LSD1 inhibitor is (trans)-N1-((1R,2S)-2-phenylcyclopropyl)cyclohexane-1,4-diamine or a pharmaceutically acceptable salt or solvate thereof.
   181. The combination of any of items 161 to 168, wherein the LSD1 inhibitor is 4-((4-((((1R,2S)-2-phenylcyclopropyl)amino)methyl)piperidin-1-yl)methyl)benzoic acid or a pharmaceutically acceptable salt or solvate thereof.
   182. The method of any of items 1 to 24 wherein the subject has Alzheimer's disease.
   183. The method of any of items 1 to 24 wherein the subject has Mild Cognitive Impairment.
   184. The method of any of items 1 to 24 wherein the subject has Huntington's disease.
   185. The method of any of items 1 to 24 wherein the subject has Parkinson's disease.
   186. The method of any of items 1 to 24 wherein the subject has dementia.
   187. The method of any of items 1 to 24 wherein the subject has an acute or chronic autoimmune neuropathy.
   188. The method of any of items 1 to 24 wherein the subject has multiple sclerosis.
   189. The method of any of items 1 to 24 wherein the subject has chronic progressive multiple sclerosis.
   190. The method of any of items 37 to 40 or 57 to 77 or the use of any of items 41 or 57 to 77 wherein the subject has Alzheimer's disease.
   191. The method of any of items 37 to 40 or 57 to 77 or the use of any of items 41 or 57 to 77 wherein the subject has Mild Cognitive Impairment.
   192. The method of any of items 37 to 40 or 57 to 77 or the use of any of items 41 or 57 to 77 wherein the subject has Huntington's disease.
   193. The method of any of items 37 to 40 or 57 to 77 or the use of any of items 41 or 57 to 77 wherein the subject has Parkinson's disease.
   194. The method of any of items 37 to 40 or 57 to 77 or the use of any of items 41 or 57 to 77 wherein the subject has dementia.
   195. The method of any of items 37 to 40 or 57 to 77 or the use of any of items 41 or 57 to 77 wherein the subject has an acute or chronic autoimmune neuropathy.
   196. The method of any of items 37 to 40 or 57 to 77 or the use of any of items 41 or 57 to 77 wherein the subject has multiple sclerosis.
   197. The method of any of items 37 to 40 or 57 to 77 or the use of any of items 41 or 57 to 77 wherein the subject has chronic progressive multiple sclerosis.
   198. The method of any of items 78 to 80 or 139 to 160, wherein the patient has Alzheimer's disease.
   199. The method of any of items 78 to 80 or 139 to 160, wherein the patient has Mild Cognitive Impairment.
   200. The method of any of items 78 to 80 or 139 to 160, wherein the patient has Huntington's disease.
   201. The method of any of items 78 to 80 or 139 to 160, wherein the patient has Parkinson's disease.
   202. The method of any of items 78 to 80 or 139 to 160, wherein the patient has dementia.
   203. The method of any of items 78 to 80 or 139 to 160, wherein the patient has an acute or chronic autoimmune neuropathy.
   204. The method of any of items 78 to 80 or 139 to 160, wherein the patient has multiple sclerosis.
   205. The method of any of items 78 to 80 or 139 to 160, wherein the patient has chronic progressive multiple sclerosis.
   206. The method of any of items 88 to 90 or 139 to 160, wherein the disease is a CNS disease.
   207. The method of any of items 88 to 90 or 139 to 160, wherein the disease is a neurodegenerative disease.
   208. The method of any of items 88 to 90 or 139 to 160, wherein the disease is Alzheimer's disease.
   209. The method of any of items 88 to 90 or 139 to 160, wherein the disease is Mild Cognitive Impairment.
   210. The method of any of items 88 to 90 or 139 to 160, wherein the disease is Huntington's disease.
   211. The method of any of items 88 to 90 or 139 to 160, wherein the disease is Parkinson's disease.
   212. The method of any of items 88 to 90 or 139 to 160, wherein the disease is dementia.
   213. The method of any of items 88 to 90 or 139 to 160, wherein the disease is an autoimmune disease.
   214. The method of any of items 88 to 90 or 139 to 160, wherein the disease is an acute or chronic autoimmune neuropathy.
   215. The method of any of items 88 to 90 or 139 to 160, wherein the disease is multiple sclerosis.
   216. The method of any of items 88 to 90 or 139 to 160, wherein the disease is chronic progressive multiple sclerosis.
   217. The LSD1 inhibitor for use of any of items 110 to 117 or 139 to 160, wherein the disease is a CNS disease.
   218. The LSD1 inhibitor for use of any of items 110 to 117 or 139 to 160, wherein the disease is a neurodegenerative disease.
   219. The LSD1 inhibitor for use of any of items 110 to 117 or 139 to 160, wherein the disease is Alzheimer's disease.
   220. The LSD1 inhibitor for use of any of items 110 to 117 or 139 to 160, wherein the disease is Mild Cognitive Impairment.
   221. The LSD1 inhibitor for use of any of items 110 to 117 or 139 to 160, wherein the disease is Huntington's disease.
   222. The LSD1 inhibitor for use of any of items 110 to 117 or 139 to 160, wherein the disease is Parkinson's disease.
   223. The LSD1 inhibitor for use of any of items 110 to 117 or 139 to 160, wherein the disease is dementia.
   224. The LSD1 inhibitor for use of any of items 110 to 117 or 139 to 160, wherein the disease is an autoimmune disease.
   225. The LSD1 inhibitor for use of any of items 110 to 117 or 139 to 160, wherein the disease is an acute or chronic autoimmune neuropathy.
   226. The LSD1 inhibitor for use of any of items 110 to 117 or 139 to 160, wherein the disease is multiple sclerosis.
   227. The LSD1 inhibitor for use of any of items 110 to 117 or 139 to 160, wherein the disease is chronic progressive multiple sclerosis.

As used herein, determining the level of a biomarker in a sample is used interchangeably with determining or measuring the level of gene expression of the biomarker in the sample. The level of a biomarker in a sample can be determined by any suitable method known in the art to measure gene products, including mRNA and protein. Non-limiting examples of such methods include detecting the quantity of mRNA transcribed from the gene, the quantity of cDNA produced from the reverse transcription of the mRNA transcribed from the gene, or the quantity of protein encoded by the gene.

In the methods according to the invention, mRNA from a sample can be directly used in determining the level of the biomarker. In the methods according to the present invention, the level can be determined by hybridization.

In the methods according to the present invention, the RNA can be transformed into cDNA (complementary DNA) copy using methods known in the art. Methods for detecting can include but are not limited to quantitative reverse transcriptase polymerase chain reaction (qRT-PCR), gene expression analyses, microarray analyses, gene expression chip analyses, hybridization techniques and chromatography as well as any other techniques known in the art, e.g. those described in Ralph Rapley, “The Nucleic Acid Protocols Handbook”, published 2000, ISBN: 978-0-89603-459-4. Methods for detecting DNA can include but are not limited to PCR, real-time PCR, digital PCR, hybridization, microarray analyses, as well as any other techniques known in the art, e.g. those described in Leland et al, “Handbook of Molecular and cellular Methods in Biology and Medicine”, published 2011, ISBN 9781420069389.

In the methods according to the invention, the method can comprise detecting the protein expression level of a biomarker. Any suitable methods of protein detection, quantization and comparison can be used, such as those described in John M. Walker, “The Protein Protocols Handbook”, published 2009, ISBN 978-1-59745-198-7. The protein expression level of a biomarker can be detected by immune assays which include the recognition of the protein or protein complex by anti antibody or antibody fragment, comprising but not limited to enzyme linked immunosorbent assays (ELISA), “sandwich” immunoassays, immunoradiometric assays, in situ immunoassays, alphaLISA immunoassays, protein proximity assays, proximity ligation assay technology (e.g. protein qPCR), western blot analysis, immunoprecipitation assays, immunofluorescent assays, flow cytometry, immunohistochemistry (IHC), immuneeletrophoresis, protein immunestaining, confocal microscopy; or by similar methods in which the antibody or antibody fragment is substituted by a chemical probe, aptamer, receptor, interacting protein or other by another biomolecule recognizing the biomarker protein in a specific manner; or by Förster/fluorescence resonance energy transfer (FRET), differential scanning fluorimetry (DSF), microfluidics, spectrophotometry, mass spectrometry, enzymatic assays, surface plasmon resonance, or combinations thereof. Immunoassays may be homogeneous assays or heterogeneous assays. In a homogeneous assay the immunological reaction usually involves the specific antibody, a labeled analyte, and the sample of interest. The signal arising from the label is modified, directly or indirectly, upon the binding of the antibody to the labeled analyte. Both the immunological reaction and detection of the extent thereof can be carried out in a homogeneous solution. Immunochemical labels which may be employed include free radicals, radioisotopes, fluorescent dyes, enzymes, bacteriophages, or coenzymes. In a heterogeneous assay approach, the reagents are usually the sample, the antibody, and means for producing a detectable signal. The antibody can be immobilized on a support, such as a bead, plate or slide, and contacted with the specimen suspected of containing the antigen in a liquid phase. The support is then separated from the liquid phase and either the support phase or the liquid phase is examined for a detectable signal employing means for producing such signal. The signal is related to the presence of the analyte in the sample. Means for producing a detectable signal include the use of radioactive labels, fluorescent labels, or enzyme labels.

In the methods according to the invention, an antibody to the biomarker of interest can be used. In the methods according to the present invention, a kit for detection can be used. Such antibodies and kits are available from commercial sources such as EMD Millipore, R&D Systems for biochemical assays, Thermo Scientific Pierce Antibodies, Novus Biologicals, Aviva Systems Biology, Abnova Corporation, AbD Serotec or others. Alternatively, antibodies can also be synthesized by any known method. The term “antibody” as used herein is intended to include monoclonal antibodies, polyclonal antibodies, and chimeric antibodies. Antibodies can be conjugated to a suitable solid support (e.g., beads such as protein A or protein G agarose, microspheres, plates, slides or wells formed from materials such as latex or polystyrene) in accordance with known techniques, such as passive binding. Antibodies as described herein may likewise be conjugated to detectable labels or groups such as radiolabels (e.g., ³⁵S), enzyme labels (e.g., horseradish peroxidase, alkaline phosphatase), fluorescent labels (e.g., fluorescein, Alexa, green fluorescent protein, rhodamine), can generated by release of singlet oxygen by phthalocyanine containing beads after irradiation at 680 nM and subsequent absorption and emission of light by acceptor beads containing Europium or Therbium, and oligonucleotide labels. Labels can generate signal directly or indirectly. Signal generated can include fluorescence, radioactivity, luminescence, in accordance with known techniques.

Preferably, in the methods according to the invention the level of the biomarker is measured either as mRNA using qRT-PCT or as protein using an ELISA assay or a proximity ligation assay technology such as a protein qPCR.

As used herein, an LSD1 inhibitor (LSD1i) is a compound which inhibits LSD1. Any LSD1 inhibitor known in the art can be used in the methods and therapeutic uses of the invention. Both irreversible and reversible LSD1i have been reported. Most LSD1i reported to date are irreversible LSD1i, which exert their inhibitory activity by becoming covalently bound to the FAD cofactor within the LSD1 active site and are generally based on a 2-(hetero)arylcyclopropylamino moiety. Some reversible inhibitors of LSD1 have also been reported in the literature (see e.g. DP Mould et al, Med. Res. Rev., 2015, 35:586-618. doi:10.1002/med.21334, epub 24 Nov. 2014).

Non-limiting examples of LSD1i are disclosed e.g. in: WO2010/043721, WO2010/084160, WO2011/035941, WO2011/042217, WO2011/131697, WO2012/013727, WO2012/013728, WO2012/045883, WO2013/057320, WO2013/057322, WO2010/143582, US2010-0324147, WO2011/022489, WO2011/131576, WO2012/034116, WO2012/135113, WO2013/022047, WO2013/025805, WO2014/058071, WO2014/084298, WO2014/086790, WO2014/164867, WO2014/205213, WO2015/021128, WO2015/031564, US2015-0065434, WO2007/021839, WO2008/127734, WO2015/089192, CN104119280, CN103961340, CN103893163, CN103319466, CN103054869, K Taeko et al, Bioorg Med Chem Lett. 2015, 25(9):1925-8. doi: 10.1016/j.bmcl.2015.03.030. Epub 2015 Mar. 20, PMID: 25827526; S Valente et al, Eur J Med Chem. 2015, 94:163-74. doi: 10.1016/j.ejmech.2015.02.060. Epub 2015 Mar. 3, PMID:25768700; MN Ahmed Khan et al Med. Chem. Commun., 2015, 6, 407-412, DOI: 10.1039/C4MD00330F epub 29 Sep. 2014; M Pieroni et al, Eur J Med Chem. 2015; 92:377-386. doi: 10.1016/j.ejmech.2014.12.032. Epub 2015 Jan. 7. PMID:25585008; V Rodriguez et al, Med. Chem. Commun., 2015, 6, 665-670 DOI: 10.1039/C4MD00507D, Epub 23 Dec. 2014; P Vianello et al, Eur J Med Chem. 2014, 86:352-63. doi: 10.1016/j.ejmech.2014.08.068. Epub 2014 Aug. 27; DP Mould et al, Med. Res. Rev., 2015, 35:586-618. doi:10.1002/med.21334, epub 24 Nov. 2014; LY Ma et al, 2015, 58(4):1705-16. doi: 10.1021/acs.jmedchem.5b00037. Epub 2015 Feb. 6; S L Nowotarski et al, 2015, 23(7):1601-12. doi: 10.1016/j.bmc.2015.01.049. Epub 2015 Feb. 7. PMID:25725609; C J Kutz et al Medchemcomm. 2014, 5(12):1863-1870 PMID: 25580204; C Zhou et al, Chemical Biology & Drug Design, 2015, 85(6):659-671. doi:10.1111/cbdd.12461, epub 22 Dec. 2014; P Prusevich et al, ACS Chem Biol. 2014, 9(6):1284-93. doi: 10.1021/cb500018s. Epub 2014 Apr. 7; B Dulla et al, Org Biomol Chem 2013, 11, 3103-3107, doi: 10.1039/c3ob40217g; J R Hitchin et al, MedChemCommun, 2013, 4, 1513-1522 DOI: 10.1039/c3md00226h; and Y Zhou et al,” Synthesis and biological evaluation of novel (E)-N′-(2,3-dihydro-1H-inden-1-ylidene)benzohydrazides as potent LSD1 inhibitors”, Biorg Med Chem Lett, 2015, online publication 20 Jun. 2015, doi:10.1016/j.bmcl.2015.06.054, WO2014/194280,WO2015/120281, WO2015/123465, WO2015/123437, WO2015/123424, WO2015/123408, WO2015/134973, WO2015/156417, WO2015/168466, WO2015/181380, WO2015/200843, WO2016/003917, WO2016/004105, WO2016/007722, WO2016/007727, WO2016/007731, WO2016/007736,WO2016/034946, WO2016/037005.

Among the references listed above, the following disclose irreversible LSD1i: WO2010/043721, WO2010/084160, WO2011/035941, WO2011/042217, WO2011/131697, WO2012/013727, WO2012/013728, WO2012/045883, WO2013/057320, WO2013/057322, WO2010/143582, US2010-0324147, WO2011/131576, WO2012/135113, WO2013/022047, WO2014/058071, WO2014/084298, WO2014/086790, WO2014/164867, WO2015/021128; K Taeko et al, Bioorg Med Chem Lett. 2015, 25(9):1925-8. doi: 10.1016/j.bmcl.2015.03.030. Epub 2015 Mar. 20, PMID: 25827526; S Valente et al, Eur J Med Chem. 2015, 94:163-74. doi: 10.1016/j.ejmech.2015.02.060. Epub 2015 Mar. 3, PMID:25768700; MN Ahmed Khan et al Med. Chem. Commun., 2015, 6, 407-412, DOI: 10.1039/C4MD00330F epub 29 Sep. 2014; M Pieroni et al, Eur J Med Chem. 2015; 92:377-386. doi: 10.1016/j.ejmech.2014.12.032. Epub 2015 Jan. 7. PMID:25585008; V Rodriguez et al, Med. Chem. Commun., 2015, 6, 665-670 DOI: 10.1039/C4MD00507D, Epub 23 Dec. 2014; P Vianello et al, Eur J Med Chem. 2014, 86:352-63. doi: 10.1016/j.ejmech.2014.08.068. Epub 2014 Aug. 27, WO2014/194280, WO2015/123465, WO2015/123437, WO2015/123424, WO2015/123408, WO2015/156417, WO2015/181380.

In the methods and therapeutic uses of the invention the LSD1i is preferably an irreversible LSD1i. In the methods and uses according to the invention, the LSD1 inhibitor is preferably a 2-(hetero)arylcyclopropylamino LSD1i. As used herein, a “2-(hetero)arylcyclopropylamino LSD i” or a “2-(hetero)arylcyclopropylamino compound” means a LSD1i whose chemical structure comprises a cyclopropyl ring substituted at position 1 with an amino group, which can be optionally substituted, and substituted at position 2 with an aryl or heteroaryl group (wherein the aryl or heteroaryl group can be optionally substituted). The ability of a compound to inhibit LSD1 can be tested in vitro using any method known in the art to determine LSD1 inhibition, for example the method disclosed in Example 1.

In the methods and uses according to the invention, the LSD1 inhibitor is preferably a 2-(hetero)arylcyclopropylamino LSD1i as disclosed in any of WO2010/043721, WO2010/084160, WO2011/035941, WO2011/042217, WO2011/131697, WO2012/013727, WO2012/013728, WO2012/045883, WO2013/057320, WO2013/057322, WO2012/135113, WO2013/022047, WO2014/058071, WO2010/143582, US2010-0324147, WO2011/131576, WO2014/084298, WO2014/086790, WO2014/164867, WO2015/021128, WO2014/194280, WO2015/123465, WO2015/123437, WO2015/123424, WO2015/123408, WO2015/156417, or WO2015/181380, the disclosure of each of which is incorporated by reference herein in their entirety, and preferably as disclosed in WO2010/043721, WO2010/084160, WO2011/035941, WO2011/042217, WO2011/131697, WO2012/013727, WO2012/013728, WO2012/045883, WO2013/057320, WO2013/057322, WO2012/135113, WO2013/022047 or WO2014/058071.

In the methods and uses according to the invention, the LSD1 inhibitor can be a compound of formula (I) or an enantiomer, a diastereomer or a mixture of stereoisomers (such as a racemic mixture or a diastereomer mixture) thereof, or a pharmaceutically acceptable salt or solvate thereof:

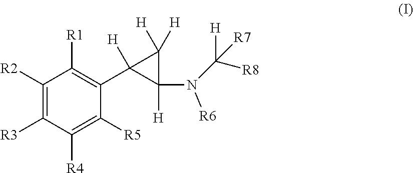

In formula (I), each of R1-R5 is optionally substituted and independently chosen from —H, halo, alkyl, alkoxy, cycloalkoxy, haloalkyl, haloalkoxy, -L-aryl, -L-heteroaryl, -L-heterocyclyl, -L-carbocycle, acylamino, acyloxy, alkylthio, cycloalkylthio, alkynyl, amino, aryl, arylalkyl, arylalkenyl, arylalkynyl, arylalkoxy, aryloxy, arylthio, heteroarylthio, cyano, cyanato, haloaryl, hydroxyl, heteroaryloxy, heteroarylalkoxy, isocyanato, isothiocyanato, nitro, sulfinyl, sulfonyl, sulfonamide, thiocarbonyl, thiocyanato, trihalomethanesulfonamido, O-carbamyl, N-carbamyl, O-thiocarbamyl, N-thiocarbamyl, and C-amido;

R6 is chosen from —H and alkyl;
R7 is chosen from —H, alkyl, and cycloalkyl;
R8 is chosen from —C(═O)NRₓR_{y} and —C(═O)R_{z};
Rₓ when present is chosen from —H, alkyl, alkynyl, alkenyl, -L-carbocycle, -L-aryl, -L-heterocyclyl, all of which are optionally substituted;
R_{y} when present is chosen from —H, alkyl, alkynyl, alkenyl, -L-carbocycle, -L-aryl, -L-heterocyclyl, all of which are optionally substituted;
R_{z} when present is chosen from —H, alkoxy, -L-carbocyclic, -L-heterocyclic, -L-aryl, wherein the aryl, heterocyclyl, or carbocycle is optionally substituted;
each L can be saturated, partially saturated, or unsaturated, and is independently chosen from —(CH₂)ₙ—(CH₂)ₙ—, —(CH₂)ₙC(═O)(CH₂)ₙ—, —(CH₂)ₙC(═O)NH(CH₂)ₙ—, —(CH₂)ₙNHC(═O)O(CH₂)ₙ—, —(CH₂)ₙNHC(═O)NH(CH₂)ₙ—, —(CH₂)ₙNHC(═S)S(CH₂)ₙ—, —(CH₂)ₙOC(═O)S(CH₂)ₙ—, —(CH₂)ₙNH(CH₂)ₙ—, —(CH₂)ₙO(CH₂)ₙ—, —(CH₂)ₙS(CH₂)ₙ—, and —(CH₂)ₙNHC(═S)NH(CH₂)ₙ—, where each n is independently chosen from 0, 1, 2, 3, 4, 5, 6, 7, and 8, wherein optionally substituted refers to zero or 1 to 4 optional substituents independently chosen from acylamino, acyloxy, alkenyl, alkoxy, cycloalkoxy, alkyl, alkylthio, cycloalkylthio, alkynyl, amino, aryl, arylalkyl, arylalkenyl, arylalkynyl, arylalkoxy, aryloxy, arylthio, heteroarylthio, carbocyclyl, cyano, cyanato, halo, haloalkyl, haloaryl, hydroxyl, heteroaryl, heteroaryloxy, heterocyclyl, heteroarylalkoxy, isocyanato, isothiocyanato, nitro, sulfinyl, sulfonyl, sulfonamide, thiocarbonyl, thiocyanato, trihalomethanesulfonamido, O-carbamyl, N-carbamyl, O-thiocarbamyl, N-thiocarbamyl, and C-amido.

Compounds of formula (I) having a (trans) disposition on the substituents on the cyclopropyl ring are preferred.

Preferably, the compound of formula (I) is a compound from the list below:
N-cyclopropyl-2-{[(trans)-2-phenylcyclopropyl]amino}acetamide;
2-{[(trans)-2-phenylcyclopropyl]amino}acetamide;
N-cyclopropyl-2-{[(trans)-2-phenylcyclopropyl]amino}propanamide;
2-{[(trans)-2-phenylcyclopropyl]amino}-N-prop-2-ynylacetamide;
N-isopropyl-2-{[(trans)-2-phenylcyclopropyl]amino}acetamide;
N-(tert-butyl)-2-{[(trans)-2-phenylcyclopropyl]amino}acetamide;
N-(2-morpholin-4-yl-2-oxoethyl)-N-[(trans)-2-phenylcyclopropyl]amine;
2-{[(trans)-2-phenylcyclopropyl]amino}propanamide;
Methyl 2-{[(trans)-2-phenylcyclopropyl]amino}propanoate;
N-cyclopropyl-2-{methyl[(trans)-2-phenylcyclopropyl]amino}acetamide;
2-{methyl[(trans)-2-phenylcyclopropyl]amino}acetamide;
N-methyl-trans-2-(Phenylcyclopropylamino)propanamide;
1-(4-methylpiperazin-1-yl)-2-((trans)-2-phenylcyclopropylamino)ethanone;
1-(4-ethylpiperazin-1-yl)-2-((trans)-2-phenylcyclopropylamino)ethanone;
1-(4-benzyl piperazin-1-yl)-2-((trans)-2-phenylcyclopropylamino)ethanone;
2-((trans)-2-phenylcyclopropylamino)-1-(4-phenylpiperazin-1-yl)ethanone;
2-((trans)-2-(4-(benzyloxy)phenyl)cyclopropylamino)-1-(4-methylpiperazin-1-yl)ethanone;
2-((trans)-2-(4-(benzyloxy)phenyl)cyclopropylamino)-N-cyclopropylacetamide;
2-((trans)-2-(4-(3-fluorobenzyloxy)phenyl)cyclopropylamino)-1-(4-methylpiperazin-1-yl)ethanone;
2-((trans)-2-(4-(3-chlorobenzyloxy)phenyl)cyclopropylamino)-1-(4-methylpiperazin-1-yl)ethanone;
2-((trans)-2-(biphenyl-4-yl)cyclopropylamino)-1-(4-methylpiperazin-1-yl)ethanone;
1-(4-methylpiperazin-1-yl)-2-((trans)-2-(4-phenethoxyphenyl)cyclopropylamino)ethanone;
2-((trans)-2-(4-(4-fluorobenzyloxy)phenyl)cyclopropylamino)-1-(4-methylpiperazin-1-yl)ethanone;
2-((trans)-2-(4-(biphenyl-4-ylmethoxy)phenyl)cyclopropylamino)-1-(4-methylpiperazin-1-yl)ethanone;
2-({(trans)-2-[4-(benzyloxy)phenyl]cyclopropyl}amino)-N-cyclopropylacetamide,
N-[(trans)-2-(4-benzyloxyphenyl)cyclopropyl]}-N-[2-(4-methylpiperazin-1-yl)-2-oxoethyl]amine,
N-[2-oxo-2-(4-phenyl piperazin-1-yl)ethyl]-N-[(trans)-2-phenylcyclopropyl]amine,
N-[2-(4-benzyl piperazin-1-yl)-2-oxoethyl]-N-[(trans)-2-phenylcyclopropyl]amine,
N-[2-(4-ethyl piperazin-1-yl)-2-oxoethyl]-N-[(trans)-2-phenylcyclopropyl]amine,
N-[2-(4-methylpiperazin-1-yl)-2-oxoethyl]-N-[(trans)-2-phenylcyclopropyl]amine,
2-((trans)-2-(4-pyridin-3-ylphenyl)cyclopropylamino)-1-(4-methylpiperazin-1-yl)ethanone, and
2-((trans)-2-(3′-methoxy-1,1′-biphenyl-4-yl)cyclopropylamino)-1-(4-methylpiperazin-1-yl)ethanone, and pharmaceutically acceptable salts thereof.

Compounds of formula (I) can be prepared by the methods disclosed in WO2010/043721, the disclosure of which is incorporated by reference herein in its entirety.

In the methods and uses according to the invention, the LSD1 inhibitor can be a compound of formula (II) or an enantiomer, a diastereomer or a mixture of stereoisomers (such as a racemic mixture or a diastereomer mixture) thereof, or a pharmaceutically acceptable salt or solvate thereof:

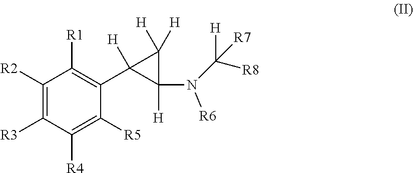

In formula (II), each of R1-R5 is independently chosen from —H, halo, alkyl, alkoxy, cycloalkoxy, haloalkyl, haloalkoxy, -L-aryl, -L-heterocyclyl, -L-carbocyclyl, acylamino, acyloxy, alkylthio, cycloalkylthio, alkynyl, amino, alkylamino, aryl, arylalkyl, arylalkenyl, arylalkynyl, arylalkoxy, aryloxy, arylthio, heteroarylthio, cyano, cyanato, haloaryl, hydroxyl, heteroaryloxy, heteroarylalkoxy, isocyanato, isothiocyanato, nitro, sulfinyl, sulfonyl, sulfonamido, thiocarbonyl, thiocyanato, trihalomethanesulfonamido, O-carbamyl, N-carbamyl, O-thiocarbamyl, N-thiocarbamyl, and C-amido;

R6 is chosen from —H and alkyl;
R7 is chosen from —H, alkyl, and cycloalkyl;
R8 is a -L-heterocyclyl wherein the ring or ring system of said -L-heterocyclyl has from 0-3 substituents chosen from halo, alkyl, alkoxy, cycloalkoxy, haloalkyl, haloalkoxy, -L-aryl, -L-heterocyclyl, -L-carbocyclyl, acylamino, acyloxy, alkylthio, cycloalkylthio, alkynyl, amino, alkylamino, aryl, arylalkyl, arylalkenyl, arylalkynyl, arylalkoxy, aryloxy, arylthio, heteroarylthio, cyano, cyanato, haloaryl, hydroxyl, heteroaryloxy, heteroarylalkoxy, isocyanato, isothiocyanato, nitro, sulfinyl, sulfonyl, sulfonamido, thiocarbonyl, thiocyanato, trihalomethanesulfonamido, O-carbamyl, N-carbamyl, O-thiocarbamyl, N-thiocarbamyl, and C-amido; or
R8 is -L-aryl wherein the ring or ring system of said -L-aryl has from 1-3 substituents chosen from halo, alkyl, alkoxy, cycloalkoxy, haloalkyl, haloalkoxy, -L-aryl, -L-heterocyclyl, -L-carbocyclyl, acylamino, acyloxy, alkylthio, cycloalkylthio, alkynyl, amino, alkylamino, aryl, arylalkyl, arylalkenyl, arylalkynyl, arylalkoxy, aryloxy, arylthio, heteroarylthio, cyano, cyanato, haloaryl, hydroxyl, heteroaryloxy, heteroarylalkoxy, isocyanato, isothiocyanato, nitro, sulfinyl, sulfonyl, sulfonamido, thiocarbonyl, thiocyanato, trihalomethanesulfonamido, O-carbamyl, N-carbamyl, O-thiocarbamyl, N-thiocarbamyl, and C-amido;
each L is independently chosen from —(CH₂)ₙ—(CH₂)ₙ—, —(CH₂)ₙNH(CH₂)ₙ—, —(CH₂)ₙO(CH₂)ₙ—, and —(CH₂)ₙS(CH₂)ₙ—, and where each n is independently chosen from 0, 1, 2, and 3.

Compounds of formula (II) having a (trans) disposition on the substituents on the cyclopropyl ring are preferred.

Preferably the compound of formula (II) is a compound from the list below:
(trans)-N-(4-fluorobenzyl)-2-phenylcyclopropanamine;
(trans)-N-(4-fluorobenzyl)-2-phenylcyclopropanaminium;
4-(((trans)-2-phenylcyclopropylamino)methyl)benzonitrile;
(trans)-N-(4-cyanobenzyl)-2-phenylcyclopropanaminium;
(trans)-2-phenyl-N-(4-(trifluoromethyl)benzyl)cyclopropanamine;
(trans)-2-phenyl-N-(4-(trifluoromethyl)benzyl)cyclopropanaminium;
(trans)-2-phenyl-N-(pyridin-2-ylmethyl)cyclopropanamine;
(trans)-2-phenyl-N-(pyridin-3-ylmethyl)cyclopropanamine;
(trans)-2-phenyl-N-(pyridin-4-ylmethyl)cyclopropanamine;
(trans)-N-((6-methylpyridin-2-yl)methyl)-2-phenylcyclopropanamine;
(trans)-2-phenyl-N-(thiazol-2-ylmethyl)cyclopropanamine;
(trans)-2-phenyl-N-(thiophen-2-ylmethyl)cyclopropanamine;
(trans)-N-((3-bromothiophen-2-yl)methyl)-2-phenylcyclopropanamine;
(trans)-N-((4-bromothiophen-2-yl)methyl)-2-phenylcyclopropanamine;
(trans)-N-(3,4-dichlorobenzyl)-2-phenylcyclopropanamine;
(trans)-N-(3-fluorobenzyl)-2-phenylcyclopropanaminium;
(trans)-N-(2-fluorobenzyl)-2-phenylcyclopropanamine;
(trans)-2-phenyl-N-(quinolin-4-ylmethyl)cyclopropanamine;
(trans)-N-(3-methoxybenzyl)-2-phenylcyclopropanamine;
(trans)-2-phenyl-N-((6-(trifluoromethyl)pyridin-3-yl)methyl)cyclopropanamine;
(trans)-N-((6-chloropyridin-3-yl)methyl)-2-phenylcyclopropanamine;
(trans)-N-((4-methylpyridin-2-yl)methyl)-2-phenylcyclopropanamine;
(trans)-N-((6-methoxypyridin-2-yl)methyl)-2-phenylcyclopropanamine;
2-(((trans)-2-phenylcyclopropylamino)methyl)pyridin-3-ol;
(trans)-N-((6-bromopyridin-2-yl)methyl)-2-phenylcyclopropanamine;
4-(((trans)-2-(4-(benzyloxy)phenyl)cyclopropylamino)methyl)benzonitrile;
(trans)-N-(4-(benzyloxy)benzyl)-2-phenylcyclopropanamine;
(trans)-N-benzyl-2-(4-(benzyloxy)phenyl)cyclopropanamine;
(trans)-2-(4-(benzyloxy)phenyl)-N-(4-methoxybenzyl)cyclopropanamine;
(trans)-2-(4-(benzyloxy)phenyl)-N-(4-fluorobenzyl)cyclopropanamine;
(trans)-2-phenyl-N-(quinolin-2-ylmethyl)cyclopropanamine;
(trans)-2-phenyl-N-((5-(trifluoromethyl)pyridin-2-yl)methyl)cyclopropanamine;
(trans)-N-((3-fluoropyridin-2-yl)methyl)-2-phenylcyclopropanamine;
(trans)-2-phenyl-N-(quinolin-3-ylmethyl)cyclopropanamine;
(trans)-N-((6-methoxypyridin-3-yl)methyl)-2-phenylcyclopropanamine;
(trans)-N-((5-methoxypyridin-3-yl)methyl)-2-phenylcyclopropanamine;
(trans)-N-((2-methoxypyridin-3-yl)methyl)-2-phenylcyclopropanamine;
(trans)-N-((3H-indol-3-yl)methyl)-2-phenylcyclopropanamine;
3-(((trans)-2-phenylcyclopropylamino)methyl)benzonitrile;
(trans)-N-(2-methoxybenzyl)-2-phenylcyclopropanamine;
3-(((trans)-2-phenylcyclopropylamino)methyl)pyridin-2-amine;
(trans)-N-((2-chloropyridin-3-yl)methyl)-2-phenylcyclopropanamine;
(trans)-N-(3,4-dimethoxybenzyl)-2-phenylcyclopropanamine;
(trans)-N-((2,3-dihydrobenzofuran-5-yl)methyl)-2-phenylcyclopropanamine;
(trans)-N-(benzo[d][1,3]dioxol-5-ylmethyl)-2-phenylcyclopropanamine;
(trans)-N-((2,3-dihydrobenzo[b][1,4]dioxin-6-yl)methyl)-2-phenylcyclopropanamine;
(trans)-N-(2,6-difluoro-4-methoxybenzyl)-2-phenylcyclopropanamine;
(trans)-2-phenyl-N-(4-(trifluoromethoxy)benzyl)cyclopropanamine;
(trans)-N-(5-fluoro-2-methoxybenzyl)-2-phenylcyclopropanamine;
(trans)-N-(2-fluoro-4-methoxybenzyl)-2-phenylcyclopropanamine;
(trans)-N-((4-methoxynaphthalen-1-yl)methyl)-2-phenylcyclopropanamine;
(trans)-N-(2-fluoro-6-methoxybenzyl)-2-phenylcyclopropanamine;
(trans)-N-((2-methoxynaphthalen-1-yl)methyl)-2-phenylcyclopropanamine;
(trans)-N-((4,7-dimethoxynaphthalen-1-yl)methyl)-2-phenylcyclopropanamine;
(trans)-N-(4-methoxy-3-methylbenzyl)-2-phenylcyclopropanamine;
(trans)-N-(3-chloro-4-methoxybenzyl)-2-phenylcyclopropanamine;
(trans)-N-(3-fluoro-4-methoxybenzyl)-2-phenylcyclopropanamine;
(trans)-N-(4-methoxy-2-methylbenzyl)-2-phenylcyclopropanamine;
(trans)-N-((3,4-dihydro-2H-benzo[b][1,4]dioxepin-6-yl)methyl)-2-phenylcyclopropanamine;
(trans)-N-((3,4-dihydro-2H-benzo[b][1,4]dioxepin-7-yl)methyl)-2-phenylcyclopropanamine;
(trans)-N-((2,2-dimethylchroman-6-yl)methyl)-2-phenylcyclopropanamine;
(trans)-N-(4-methoxy-2,3-dimethylbenzyl)-2-phenylcyclopropanamine;
(trans)-N-(4-methoxy-2,5-dimethylbenzyl)-2-phenylcyclopropanamine;
(trans)-N-(2-fluoro-4,5-dimethoxybenzyl)-2-phenylcyclopropanamine;
(trans)-N-(3-chloro-4,5-dimethoxybenzyl)-2-phenylcyclopropanamine;
(trans)-N-(2-chloro-3,4-dimethoxybenzyl)-2-phenylcyclopropanamine;
(trans)-N-(2,4-dimethoxy-6-methylbenzyl)-2-phenylcyclopropanamine;
(trans)-N-(2,5-dimethoxybenzyl)-2-phenylcyclopropanamine;
(trans)-N-(2,3-dimethoxybenzyl)-2-phenylcyclopropanamine;
(trans)-N-(2-chloro-3-methoxybenzyl)-2-phenylcyclopropanamine;
(trans)-N-((1H-indol-5-yl)methyl)-2-phenylcyclopropanamine;
(trans)-2-(4-(benzyloxy)phenyl)-N-(pyridin-2-ylmethyl)cyclopropanamine;
(trans)-2-(4-(benzyloxy)phenyl)-N-(2-methoxybenzyl)cyclopropanamine;
(trans)-N-(1-(4-methoxyphenyl)ethyl)-2-phenylcyclopropanamine;
(trans)-N-(1-(3,4-dimethoxyphenyl)ethyl)-2-phenylcyclopropanamine;
(trans)-N-(1-(2,3-dihydrobenzo[b][1,4]dioxin-6-yl)ethyl)-2-phenylcyclopropanamine;
(trans)-N-(1-(5-fluoro-2-methoxyphenyl)ethyl)-2-phenylcyclopropanamine;
(trans)-N-(1-(3,4-dimethoxyphenyl)propan-2-yl)-2-phenylcyclopropanamine;
(trans)-N-((3-methyl-1,2,4-oxadiazol-5-yl)methyl)-2-phenylcyclopropanamine; and pharmaceutically acceptable salts thereof.

Compounds of formula (II) can be prepared by the methods disclosed in WO2010/084160, the disclosure of which is incorporated by reference herein in its entirety.

In the methods and uses according to the invention, the LSD1 inhibitor can be a compound of formula (III) or an enantiomer, a diastereomer or a mixture of stereoisomers (such as a racemic mixture or a diastereomer mixture) thereof, or a pharmaceutically acceptable salt or solvate thereof:

(A′)_{X}-(A)-(B)-(Z)-(L)-(D) (III)

In formula (III), (A) is heteroaryl or aryl;

each (A′), if present, is independently chosen from aryl, arylalkoxy, arylalkyl, heterocyclyl, aryloxy, halo, alkoxy, haloalkyl, cycloalkyl, haloalkoxy, and cyano, wherein each (A′) is substituted with 0, 1, 2, or 3 substituents independently chosen from halo, haloalkyl, aryl, arylalkoxy, alkyl, alkoxy, cyano, sulfonyl, amido, and sulfinyl;

### X is 0, 1, 2, or 3;

(B) is a cyclopropyl ring, wherein (A) and (Z) are covalently bonded to different carbon atoms of (B);

### (Z) is —NH—;

(L) is chosen from —CH₂CH₂—, —CH₂CH₂CH₂—, and —CH₂CH₂CH₂CH₂—; and
(D) is chosen from —N(—R1)-R2, —O—R3, and —S—R3, wherein:
R1 and R2 are mutually linked to form a heterocyclic ring together with the nitrogen atom that R1 and R2 are attached to, wherein said heterocyclic ring has 0, 1, 2, or 3 substituents independently chosen from —NH₂, —NH(C₁-C₆ alkyl), —N(C₁-C₆ alkyl)(C₁-C₆ alkyl), alkyl, halo, cyano, alkoxy, haloalkyl, and haloalkoxy, or
R1 and R2 are independently chosen from —H, alkyl, cycloalkyl, haloalkyl, and heterocyclyl, wherein the sum of substituents on R1 and R2 together is 0, 1, 2, or 3, and the substituents are independently chosen from —NH₂, —NH(C₁-C₆ alkyl), —N(C₁-C₆ alkyl)(C₁-C₆ alkyl), and fluoro; and
R3 is chosen from —H, alkyl, cycloalkyl, haloalkyl, and heterocyclyl, wherein R3 has 0, 1, 2, or 3 substituents independently chosen from —NH₂, —NH(C₁-C₆ alkyl), —N(C₁-C₆ alkyl)(C₁-C₆ alkyl), and fluoro.

Compounds of formula (III) having a (trans) disposition on the substituents on the cyclopropyl ring are preferred.

Preferably the compound of formula (III) is a compound from the list below:
N-[2-(4-methylpiperazin-1-yl)ethyl]-N-[(trans)-2-phenylcyclopropyl] amine;
N-cyclopropyl-N′-[(trans)-2-phenylcyclopropyl]ethane-1,2-diamine;
N,N-dimethyl-N′-(2-{[(trans)-2-phenylcyclopropyl]amino}ethyl)ethane-1,2-diamine;
(3R)-1-(2-{[(trans)-2-phenylcyclopropyl]amino}ethyl)pyrrolidin-3-amine;
(3S)—N,N-dimethyl-1-(2-{[(trans)-2-phenylcyclopropyl]amino}ethyl) pyrrolidin-3-amine;
(3R)-N,N-dimethyl-1-(2-{[(trans)-2-phenylcyclopropyl]amino}ethyl)pyrrolidin-3-amine;
N-[(trans)-2-phenylcyclopropyl]-N-(2-piperazin-1-ylethyl)amine;
N1,N1-diethyl-N2-((trans)-2-phenylcyclopropyl)ethane-1,2-diamine;
N-[(trans)-2-phenylcyclopropyl]-N-(2-piperidin-1-ylethyl)amine;
(trans)-2-(4-(benzyloxy)phenyl)-N-(2-(4-methylpiperazin-1-yl)ethyl)cyclopropanamine;
(trans)-N-(2-(4-methylpiperazin-1-yl)ethyl)-2-(3′-(trifluoromethyl) biphenyl-4-yl)cyclopropanamine;
(trans)-2-(3′-chlorobiphenyl-4-yl)-N-(2-(4-methylpiperazin-1-yl)ethyl)cyclopropanamine;
(R)-1-(2-((trans)-2-(3′-(trifluoromethyl)biphenyl-4-yl)cyclopropylamino)ethyl)pyrrolidin-3-amine; and
N¹-cyclopropyl-N²-((trans)-2-(3′-(trifluoromethyl)biphenyl-4-yl)cyclopropyl)ethane-1,2-diamine;
N1-((trans)-2-(4-(3-bromobenzyloxy)phenyl)cyclopropyl)-N2-cyclopropylethane-2-diamine;
N1-((trans)-2-(3′-chlorobiphenyl-4-yl)cyclopropyl)-N2-cyclopropylethane-1,2-diamine;
N1-cyclopropyl-N2-((trans)-2-(4-phenethoxyphenyl)cyclopropyl)ethane-1,2-diamine;
N1,N1-diethyl-N2-((trans)-2-(4-(3-fluorobenzyloxy)phenyl)cyclopropyl)ethane-1,2-diamine;
(trans)-2-(4-bromophenyl)-N-(2-(4-methylpiperazin-1-yl)ethyl)cyclopropanamine;
N1-((trans)-2-(terphenyl-4-yl)cyclopropyl)-N2-cyclopropylethane-1,2-diamine;
(trans)-N-(2-(piperidin-1-yl)ethyl)-2-(3′-(trifluoromethyl)biphenyl-4-yl)cyclopropanamine;
N1,N1-diethyl-N2-((trans)-2-(3′-(trifluoromethyl)biphenyl-4-yl)cyclopropyl)ethane-1,2-diamine;
(trans)-N-(2-(piperazin-1-yl)ethyl)-2-(3′-(trifluoromethyl)biphenyl-4-yl)cyclopropanamine;
(S)-1-(2-((trans)-2-(3′-(trifluoromethyl)biphenyl-4-yl)cyclopropylamino)ethyl)pyrrolidin-3-amine;
(R)-1-(2-((trans)-2-(3′-chlorobiphenyl-4-yl)cyclopropylamino)ethyl)pyrrolidin-3-amine;
(R)-1-(2-((trans)-2-(4′-chlorobiphenyl-4-yl)cyclopropylamino)ethyl)pyrrolidin-3-amine;
(R)-1-(2-((trans)-2-(3′-methoxybiphenyl-4-yl)cyclopropylamino)ethyl)pyrrolidin-3-amine;
(R)-1-(2-((trans)-2-(4-(3-bromobenzyloxy)phenyl)cyclopropylamino)ethyl)pyrrolidin-3-amine; and
(R)-1-(2-((trans)-2-(6-(3-(trifluoromethyl)phenyl)pyridin-3-yl)cyclopropylamino)ethyl)pyrrolidin-3-amine;
N-(trans)-2-(isobutylthio)-ethyl-2-phenylcyclopropanamine,
N-trans-(2-ethoxyethyl)-2-phenylcyclopropanamine, and
N-trans-(2-methoxyethyl)-2-phenylcyclopropanamine,
(R)-1-(2-((trans)-2-(4-(4-bromobenzyloxy)phenyl)cyclopropylamino)ethyl)pyrrolidin-3-amine;
(R)-1-(2-((trans)-2-(4-(4-chlorobenzyloxy)phenyl)cyclopropylamino)ethyl)pyrrolidin-3-amine;
(R)-1-(2-((trans)-2-(4-(biphenyl-4-ylmethoxy)phenyl)cyclopropylamino)ethyl)pyrrolidin-3-amine;
(R)-1-(2-((trans)-2-(3′,5-dichlorobiphenyl-4-yl)cyclopropylamino)ethyl)pyrrolidin-3-amine;
N1-((trans)-2-(2-[1,1′;4′,1″]terphenyl-4″-yl-cyclopropyl)-N2-cyclopropylethane-1,2-diamine;
(R)-1-(2-((trans)-2-(6-(benzyloxy)-4-(trifluoromethyl)biphenyl-3-yl)cyclopropylamino)ethyl)pyrrolidin-3-amine; and
(R)-1-(2-((trans)-2-(6-(benzyloxy)biphenyl-3-yl)cyclopropylamino)ethyl)pyrrolidin-3-amine;
(R)-1-(2-((trans)-2-(4-phenethoxyphenyl)cyclopropylamino)ethyl)pyrrolidin-3-amine;
(R)-1-(2-((trans)-2-(6-(3-methoxyphenyl)pyridin-3-yl)cyclopropylamino)ethyl)pyrrolidin-3-amine;
(R)-1-(2-((trans)-2-(6-(4-chlorophenyl)pyridin-3-yl)cyclopropylamino)ethyl)pyrrolidin-3-amine; and
4-((4-((trans)-2-(2-((R)-3-aminopyrrolidin-1-yl)ethylamino)cyclopropyl)phenoxy)methyl)benzonitrile;
   and pharmaceutically acceptable salts thereof.

Compounds of formula (III) can be prepared by the methods disclosed in WO2011/035941, the disclosure of which is incorporated by reference herein in its entirety.

In the methods and uses according to the invention, the LSD1 inhibitor can be a compound of formula (IV) or an enantiomer, a diastereomer or a mixture of stereoisomers (such as a racemic mixture or a diastereomer mixture) thereof, or a pharmaceutically acceptable salt or solvate thereof:

(A′)_{X}-(A)-(B)-(Z)-(L)-C(═O)NH₂ (IV)

In formula (IV), (A) is heteroaryl or aryl;

each (A′), if present, is independently chosen from aryl, arylalkoxy, arylalkyl, heterocyclyl, aryloxy, halo, alkoxy, haloalkyl, cycloalkyl, haloalkoxy, and cyano, wherein each (A′) is substituted with 0, 1, 2 or 3 substituents independently chosen from halo, haloalkyl, aryl, arylalkoxy, alkyl, alkoxy, cyano, sulfonyl, sulfinyl, and carboxamide;

### X is 0, 1, 2, or 3;

(B) is a cyclopropyl ring, wherein (A) and (Z) are covalently bonded to different carbon atoms of (B);

### (Z) is —NH—; and

(L) is —(CH₂)ₘCR₁R₂—, wherein m is 0, 1, 2, 3, 4, 5, or 6, and wherein R₁ and R₂ are each independently hydrogen or C₁-C₆ alkyl;
provided that, if (L) is —CH₂— or —CH(CH₃)—, then X is not 0.

Compounds of formula (IV having a (trans) disposition on the substituents on the cyclopropyl ring are preferred.

Preferably, the compound of formula (IV) is a compound from the list below:
2-((trans)-2-(4-(4-cyanobenzyloxy)phenyl)cyclopropylamino)acetamide,
2-((trans)-2-(4-(3-cyanobenzyloxy)phenyl)cyclopropylamino)acetamide,
2-((trans)-2-(4-(benzyloxy)phenyl)cyclopropylamino)acetamide,
2-((trans)-2-(4-(4-fluorobenzyloxy)phenyl)cyclopropylamino)acetamide,
2-((trans)-2-(4-(3-fluorobenzyloxy)phenyl)cyclopropylamino)acetamide,
2-((trans)-2-(4-(3-chlorobenzyloxy)phenyl)cyclopropylamino)acetamide,
2-((trans)-2-(4-(4-chlorobenzyloxy)phenyl)cyclopropylamino)acetamide,
2-((trans)-2-(4-(3-bromobenzyloxy)phenyl)cyclopropylamino)acetamide,
2-((trans)-2-(4-(3,5-difluorobenzyloxy)phenyl)cyclopropylamino)acetamide,
2-((trans)-2-(4-phenethoxyphenyl)cyclopropylamino)acetamide,
2-((trans)-2-(3′-(trifluoromethyl)biphenyl-4-yl)cyclopropylamino)acetamide,
2-((trans)-2-(3′-chlorobiphenyl-4-yl)cyclopropylamino)acetamide,
2-((trans)-2-(6-(4-chlorophenyl)pyridin-3-yl)cyclopropylamino)acetamide,
(R)-2-((trans)-2-(4-(3-fluorobenzyloxy)phenyl)cyclopropylamino)propanamide,
(S)-2-((trans)-2-(4-(4-fluorobenzyloxy)phenyl)cyclopropylamino)propanamide,
(R)-2-((trans)-2-(4-(4-fluorobenzyloxy)phenyl)cyclopropylamino)propanamide,
(S)-2-((trans)-2-(4-(4-fluorobenzyloxy)phenyl)cyclopropylamino)propanamide,
(R)-2-((trans)-2-(4-(benzyloxy)phenyl)cyclopropylamino)propanamide,
(S)-2-((trans)-2-(4-(benzyloxy)phenyl)cyclopropylamino)propanamide,
2-(2-[1,1′;4′,1″]Terphenyl-4″-yl-cyclopropylamino)acetamide,
5′-((trans)-2-(2-amino-2-oxoethylamino)cyclopropyl)-2′-(benzyloxy)biphenyl-3-carboxamide,
5-((trans)-2-(4′-chlorobiphenyl-4-yl)cyclopropylamino)pentanamide,
3-((trans)-2-(4-(3-bromobenzyloxy)phenyl)cyclopropylamino)propanamide,
4-((trans)-2-phenylcyclopropylamino)butanamide,
5-((trans)-2-phenylcyclopropylamino)pentanamide,
5-((trans)-2-(4′-chlorobiphenyl-4-yl)cyclopropylamino)-2-methylpentanamide,
4-((trans)-2-(4′-chlorobiphenyl-4-yl)cyclopropylamino)-2-methylbutanamide,
3-((trans)-2-(4-(3-fluorobenzyloxy)phenyl)cyclopropylamino)-2,2-dimethylpropanamide,
3-((trans)-2-(4′-chlorobiphenyl-4-yl)cyclopropylamino)propanamide,
4-((trans)-2-(4′-chlorobiphenyl-4-yl)cyclopropylamino)butanamide,
4-((trans)-2-(4-(3-bromobenzyloxy)phenyl)cyclopropylamino)butanamide,
5-((trans)-2-(4-(3-bromobenzyloxy)phenyl)cyclopropylamino)pentanamide,
5-((trans)-2-(6-(benzyloxy)biphenyl-3-yl)cyclopropylamino)pentanamide, and
4-((trans)-2-(6-(benzyloxy)biphenyl-3-yl)cyclopropylamino)butanamide,
   and pharmaceutically acceptable salts thereof.

Compounds of formula (IV) can be prepared by the methods disclosed in WO2011/042217, the disclosure of which is incorporated by reference herein in its entirety.

In the methods and uses according to the invention, the LSD1 inhibitor can be a compound of formula (V) or an enantiomer, a diastereomer or a mixture of stereoisomers (such as a racemic mixture or a diastereomer mixture) thereof, or a pharmaceutically acceptable salt or solvate thereof:

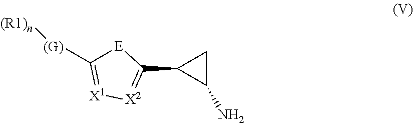

In formula (V), E is —N(R3)-, —O—, or —S—, or is —X³═X⁴—;

X¹ and X² are independently C(R2) or N;
X³ and X⁴, when present, are independently C(R2) or N;
(G) is a cyclyl group;
each (R1) is independently chosen from alkyl, alkenyl, alkynyl, cyclyl, -L1-cyclyl, -L1-amino, -L1-hydroxyl, amino, amido, nitro, halo, haloalkyl, haloalkoxy, cyano, sulfinyl, sulfonyl, sulfonamide, hydroxyl, alkoxy, urea, carbamate, acyl, or carboxyl;
each (R2) is independently chosen from —H, alkyl, alkenyl, alkynyl, cyclyl, -L1-cyclyl, -L1-amino, -L1-hydroxyl, amino, amido, nitro, halo, haloalkyl, haloalkoxy, cyano, sulfinyl, sulfonyl, sulfonamide, hydroxyl, alkoxy, urea, carbamate, acyl, or carboxyl, wherein each (R2) group has 1, 2, or 3 independently chosen optional substituents or two (R2) groups can be taken together to form a heterocyclyl or aryl group having 1, 2, or 3 independently chosen optional substituents, wherein said optional substituents are independently chosen from alkyl, alkanoyl, heteroalkyl, heterocyclyl, haloalkyl, cycloalkyl, carbocyclyl, arylalkoxy, heterocyclylalkoxy, aryl, aryloxy, heterocyclyloxy, alkoxy, haloalkoxy, oxo, acyloxy, carbonyl, carboxyl, carboxamido, cyano, halogen, hydroxyl, amino, aminoalkyl, amidoalkyl, amido, nitro, thiol, alkylthio, arylthio, sulfonamide, sulfinyl, sulfonyl, urea, or carbamate;
R3 is —H or a (C₁-C₆)alkyl group;
each L1 is independently alkylene or heteroalkylene; and
n is 0, 1, 2, 3, 4 or 5.

Compounds of formula (V) having a (trans) disposition on the substituents on the cyclopropyl ring are preferred.

Preferably, the compound of formula (V) is a compound from the list below:
(trans)-2-(3′-(trifluoromethyl)biphenyl-4-yl)cyclopropanamine;
(trans)-2-(terphenyl-4-yl)cyclopropanamine;
4′-((trans)-2-aminocyclopropyl)biphenyl-4-ol;
4′-((trans)-2-aminocyclopropyl)biphenyl-3-ol;
(trans)-2-(6-(3-(trifluoromethyl)phenyl)pyridin-3-yl)cyclopropanamine;
(Trans)-2-(6-(3,5-dichlorophenyl)pyridin-3-yl)cyclopropanamine;
(trans)-2-(6-(4-chlorophenyl)pyridin-3-yl)cyclopropanamine;
(trans)-2-(6-(3-chlorophenyl)pyridin-3-yl)cyclopropanamine;
(trans)-2-(6-(4-(trifluoromethyl)phenyl)pyridin-3-yl)cyclopropanamine;
(trans)-2-(6-(4-methoxyphenyl)pyridin-3-yl)cyclopropanamine;
(trans)-2-(6-(3-methoxyphenyl)pyridin-3-yl)cyclopropanamine;
4-(5-((trans)-2-aminocyclopropyl)pyridin-2-yl)benzonitrile;
3-(5-((trans)-2-aminocyclopropyl)pyridin-2-yl)benzonitrile;
(Trans)-2-(6-p-tolylpyridin-3-yl)cyclopropanamine;
(Trans)-2-(6-m-tolylpyridin-3-yl)cyclopropanamine;
4-(5-((trans)-2-aminocyclopropyl)pyridin-2-yl)phenol;
3-(5-((trans)-2-aminocyclopropyl)pyridin-2-yl)phenol;
4-(5-((trans)-2-aminocyclopropyl)pyridin-2-yl)benzamide;
3-(5-((trans)-2-aminocyclopropyl)pyridin-2-yl)benzamide;
2-(5-((trans)-2-aminocyclopropyl)pyridin-2-yl)phenol;
3-(5-((trans)-2-aminocyclopropyl)pyridin-2-yl)phenol;
(Trans)-2-(6-(3-methoxy-4-methylphenyl)pyridin-3-yl)cyclopropanamine;
5-(5-((trans)-2-aminocyclopropyl)pyridin-2-yl)-2-fluorophenol;
3-(5-((trans)-2-aminocyclopropyl)pyridin-2-yl)-5-fluorophenol;
3-(5-((trans)-2-aminocyclopropyl)pyridin-2-yl)-4-fluorophenol;
3-(5-((trans)-2-aminocyclopropyl)pyridin-2-yl)-2-fluorophenol;
3-(5-((trans)-2-aminocyclopropyl)pyridin-2-yl)-2,4-difluorophenol;
3-(5-((trans)-2-aminocyclopropyl)pyridin-2-yl)-2,4,6-trifluorophenol;
3-(5-((trans)-2-aminocyclopropyl)pyridin-2-yl)-5-chlorophenol;
(Trans)-2-(6-(2-fluoro-3-(trifluoromethyl)phenyl)pyridin-3-yl)cyclopropanamine;
(Trans)-2-(6-(5-chlorothiophen-2-yl)pyridin-3-yl)cyclopropanamine;
(Trans)-2-(6-(5-methyl thiophen-2-yl)pyridin-3-yl)cyclopropanamine;
(Trans)-2-(6-(1H-indol-6-yl)pyridin-3-yl)cyclopropanamine;
(Trans)-2-(6-(benzo[b]thiophen-5-yl)pyridin-3-yl)cyclopropanamine;
3-(5-((trans)-2-aminocyclopropyl)-3-methylpyridin-2-yl)phenol;
(trans)-2-(6-(3-chlorophenyl)-5-methylpyridin-3-yl)cyclopropanamine;
(trans)-2-(5-methyl-6-(3-(trifluoromethyl)phenyl)pyridin-3-yl)cyclopropanamine;
(trans)-2-(6-(4-fluoro-3-methoxyphenyl)pyridin-3-yl)cyclopropanamine;
(trans)-2-(6-(3-fluoro-5-methoxyphenyl)pyridin-3-yl)cyclopropanamine;
(trans)-2-(6-(2-fluoro-5-methoxyphenyl)pyridin-3-yl)cyclopropanamine;
(trans)-2-(6-(2-fluoro-3-methoxyphenyl)pyridin-3-yl)cyclopropanamine;
(trans)-2-(6-(3-chloro-5-methoxyphenyl)pyridin-3-yl)cyclopropanamine;
(trans)-2-(6-(2-chloro-5-methoxyphenyl)pyridin-3-yl)cyclopropanamine;
(trans)-2-(6-(3-methoxy-5-(trifluoromethyl)phenyl)pyridin-3-yl)cyclopropanamine;
3-(5-((trans)-2-aminocyclopropyl)pyridin-2-yl)-5-methoxybenzonitrile;
5-(5-((trans)-2-aminocyclopropyl)pyridin-2-yl)-2-methylphenol;
3-(5-((trans)-2-aminocyclopropyl)pyridin-2-yl)-4-chlorophenol;
3-(5-((trans)-2-aminocyclopropyl)pyridin-2-yl)-5-(trifluoromethyl)phenol;
(trans)-2-(6-(2-fluoro-5-(trifluoromethyl)phenyl)pyridin-3-yl)cyclopropanamine;
(trans)-2-(6-(2-chloro-5-(trifluoromethyl)phenyl)pyridin-3-yl)cyclopropanamine;
(trans)-2-(6-(3,5-bis(trifluoromethyl)phenyl)pyridin-3-yl)cyclopropanamine;
N-(3-(5-((trans)-2-aminocyclopropyl)pyridin-2-yl)phenyl)acetamide;
N-(3-(5-((trans)-2-aminocyclopropyl)pyridin-2-yl)phenyl)methanesulfonamide;
(trans)-2-(6-(benzo[b]thiophen-2-yl)pyridin-3-yl)cyclopropanamine;
(trans)-2-(6-(benzo[b]thiophen-3-yl)pyridin-3-yl)cyclopropanamine;
5-(5-((trans)-2-aminocyclopropyl)pyridin-2-yl)thiophene-2-carbonitrile;
(trans)-2-(6-(4-methylthiophen-3-yl)pyridin-3-yl)cyclopropanamine;
(trans)-2-(2-chloro-6-(3-(trifluoromethyl)phenyl)pyridin-3-yl)cyclopropanamine;
(trans)-2-(2-(4-chlorophenyl)-6-(3-(trifluoromethyl)phenyl)pyridin-3-yl)cyclopropanamine;
4-(3-((trans)-2-aminocyclopropyl)-6-(3-(trifluoromethyl)phenyl)pyridin-2-yl)phenol;
4-(3-((trans)-2-aminocyclopropyl)-6-(3-(trifluoromethyl)phenyl)pyridin-2-yl)benzamide;
(trans)-2-(2-methyl-6-(3-(trifluoromethyl)phenyl)pyridin-3-yl)cyclopropanamine;
3-(5-((trans)-2-aminocyclopropyl)pyridin-2-yl)-5-hydroxybenzonitrile;
(trans)-2-(6-(3,4-difluoro-5-methoxyphenyl)pyridin-3-yl)cyclopropanamine;
5-(5-((trans)-2-aminocyclopropyl)pyridin-2-yl)-2,3-difluorophenol;
(trans)-2-(6-(3-chloro-4-fluoro-5-methoxyphenyl)pyridin-3-yl)cyclopropanamine;
5-(5-((trans)-2-aminocyclopropyl)pyridin-2-yl)-3-chloro-2-fluorophenol;
(trans)-2-(6-(1H-indazol-6-yl)pyridin-3-yl)cyclopropanamine;
(trans)-2-(6-(9H-carbazol-2-yl)pyridin-3-yl)cyclopropanamine;
6-(5-((trans)-2-aminocyclopropyl)pyridin-2-yl)indolin-2-one;
6-(5-((trans)-2-aminocyclopropyl)pyridin-2-yl)benzofuran-2(3H)-one;
4-(5-((trans)-2-aminocyclopropyl)pyridin-2-yl)pyridin-2(1H)-one;
N-(3-(5-((trans)-2-aminocyclopropyl)pyridin-2-yl)phenyl)benzenesulfonamide;
N-(3-(5-((trans)-2-aminocyclopropyl)pyridin-2-yl)phenyl)propane-2-sulfonamide;
4′-((trans)-2-aminocyclopropyl)-4-fluorobiphenyl-3-ol;
4′-((trans)-2-aminocyclopropyl)-5-chlorobiphenyl-3-ol;
4′-((trans)-2-aminocyclopropyl)-5-chloro-4-fluorobiphenyl-3-ol;
N-(4′-((trans)-2-aminocyclopropyl)biphenyl-3-yl)benzenesulfonamide;
N-(4′-((trans)-2-aminocyclopropyl)biphenyl-3-yl)propane-2-sulfonamide;
N-(4′-((trans)-2-aminocyclopropyl)biphenyl-3-yl)methanesulfonamide;
N-(2-(5-((trans)-2-aminocyclopropyl)pyridin-2-yl)phenyl)methanesulfonamide;
3-(5-((trans)-2-aminocyclopropyl)pyridin-2-yl)-4-methoxybenzonitrile;
N-(4′-((trans)-2-aminocyclopropyl)biphenyl-2-yl)methanesulfonamide;
4′-((trans)-2-aminocyclopropyl)-6-methoxybiphenyl-3-carbonitrile;
N-(4′-((trans)-2-aminocyclopropyl)-6-methoxybiphenyl-3-yl)methanesulfonamide;
4′-((trans)-2-aminocyclopropyl)-6-hydroxybiphenyl-3-carbonitrile;
N-(4′-((trans)-2-aminocyclopropyl)-6-hydroxybiphenyl-3-yl)methanesulfonamide;
3-(5-((trans)-2-aminocyclopropyl)pyridin-2-yl)-4-hydroxybenzonitrile;
N-(3-(5-((trans)-2-aminocyclopropyl)pyridin-2-yl)-4-hydroxyphenyl)methanesulfonamide;
N-(3-(5-((trans)-2-aminocyclopropyl)pyridin-2-yl)-5-(trifluoromethyl)phenyl)ethanesulfonamide;
N-(3-(5-((trans)-2-aminocyclopropyl)pyridin-2-yl)-5-(trifluoromethyl)phenyl)methanesulfonamide;
3-(6-((trans)-2-aminocyclopropyl)pyridin-3-yl)phenol;
(Trans)-2-(5-(3-methoxyphenyl)pyridin-2-yl)cyclopropanamine;
4-(6-((trans)-2-aminocyclopropyl)pyridin-3-yl)phenol;
2-(6-((trans)-2-aminocyclopropyl)pyridin-3-yl)phenol;
2-(5-((trans)-2-aminocyclopropyl)thiophen-2-yl)phenol;
3-(5-((trans)-2-aminocyclopropyl)thiophen-2-yl)phenol;
4-(5-((trans)-2-aminocyclopropyl)thiophen-2-yl)phenol;
2-(5-((trans)-2-aminocyclopropyl)thiazol-2-yl)phenol;
3-(5-((trans)-2-aminocyclopropyl)thiazol-2-yl)phenol;
4-(5-((trans)-2-aminocyclopropyl)thiazol-2-yl)phenol;
2-(2-((trans)-2-aminocyclopropyl)thiazol-5-yl)phenol;
3-(2-((trans)-2-aminocyclopropyl)thiazol-5-yl)phenol;
2-(2-((trans)-2-aminocyclopropyl)thiazol-5-yl)phenol;
3-(2-((trans)-2-aminocyclopropyl)thiazol-5-yl)phenol;
3-(5-((trans)-2-aminocyclopropyl)pyrimidin-2-yl)phenol;
4-(5-((trans)-2-aminocyclopropyl)pyrimidin-2-yl)phenol;
N-(3-(5-((trans)-2-aminocyclopropyl)pyridin-2-yl)-4-methoxyphenyl)methanesulfonamide;
N-(4′-((trans)-2-aminocyclopropyl)-5-chloro-[1,1′-biphenyl]-3-yl)methanesulfonamide;
N-(3-(5-((trans)-2-aminocyclopropyl)pyridin-2-yl)-5-chlorophenyl)methanesulfonamide;
N-(4′-((trans)-2-aminocyclopropyl)-4-fluoro-[1,1′-biphenyl]-3-yl)methanesulfonamide;
N-(5-(5-((trans)-2-aminocyclopropyl)pyridin-2-yl)-2-fluorophenyl)methanesulfonamide;
N-(3-(5-((trans)-2-aminocyclopropyl)pyridin-2-yl)phenyl)ethanesulfonamide;
N-(3-(5-((trans)-2-aminocyclopropyl)pyridin-2-yl)phenyl)-4-cyanobenzenesulfonamide;
N-(3-(5-((trans)-2-aminocyclopropyl)pyridin-2-yl)phenyl)-3-cyanobenzenesulfonamide;
N-(3-(5-((trans)-2-aminocyclopropyl)pyridin-2-yl)phenyl)-2-cyanobenzenesulfonamide;
N-(3-(5-((trans)-2-aminocyclopropyl)pyridin-2-yl)-5-(trifluoromethyl)phenyl)-4-cyanobenzenesulfonamide;
N-(4′-((trans)-2-aminocyclopropyl)-[1,1′-biphenyl]-3-yl)-1,1,1-trifluoromethanesulfonamide;
4′-((trans)-2-aminocyclopropyl)-6-hydroxy-[1,1′-biphenyl]-3-carbonitrile;
4′-((trans)-2-aminocyclopropyl)-[1,1′-biphenyl]-2-ol;
4′-((trans)-2-aminocyclopropyl)-3′-methoxy-[1,1′-biphenyl]-3-ol;
N-(3-(5-((trans)-2-aminocyclopropyl)thiazol-2-yl)phenyl)-2-cyanobenzenesulfonamide;
   and pharmaceutically acceptable salts thereof.

Compounds of formula (V) can be prepared by the methods disclosed in WO2012/013727, the disclosure of which is incorporated by reference herein in its entirety.

In the methods and uses according to the invention, the LSD1 inhibitor can be a compound of formula (VI) or an enantiomer, a diastereomer or a mixture of stereoisomers (such as a racemic mixture or a diastereomer mixture) thereof, or a pharmaceutically acceptable salt or solvate thereof:

(A′)_{X}-(A)-(B)-(Z)-(L)-(D) (VI)

In formula (VI), (A) is heteroaryl or aryl;

each (A′), if present, is independently chosen from aryl, arylalkoxy, arylalkyl, heterocyclyl, aryloxy, halo, alkoxy, haloalkyl, cycloalkyl, haloalkoxy, and cyano, wherein each (A′) is substituted with 0, 1, 2, or 3 substituents independently chosen from halo, haloalkyl, haloalkoxy, aryl, arylalkoxy, alkyl, alkoxy, amido, —CH₂C(═O)NH₂, heteroaryl, cyano, sulfonyl, and sulfinyl;

### X is 0, 1, 2, or 3;

(B) is a cyclopropyl ring, wherein (A) and (Z) are covalently bonded to different carbon atoms of (B);

### (Z) is —NH—;

(L) is chosen from a single bond, —CH₂—, —CH₂CH₂—, —CH₂CH₂CH₂—, and —CH₂CH₂CH₂CH₂—; and
(D) is an aliphatic carbocyclic group or benzocycloalkyl, wherein said aliphatic carbocyclic group or said benzocycloalkyl has 0, 1, 2, or 3 substituents independently chosen from —NH₂, —NH(C₁-C₆ alkyl), —N(C₁-C₆ alkyl)(C₁-C₆ alkyl), alkyl, halo, amido, cyano, alkoxy, haloalkyl, and haloalkoxy.

Preferably in formula (VI),

(A) is aryl or heteroaryl. Said aryl is preferably phenyl. Said heteroaryl is preferably pyridinyl, pyrimidinyl, or thiophenyl; and/or
(A′), if present, is aryl or arylalkoxy. Said aryl is preferably phenyl. Said arylalkoxy is preferably benzyloxy, all of which can be optionally substituted as provided above; and/or
(L) is a single bond.

Compounds of formula (VI) having a (trans) disposition on the substituents on the cyclopropyl ring are preferred.

Preferably, the compound of formula (VI) is a compound from the list below:
N-((trans)-2-(4-(benzyloxy)phenyl)cyclopropyl)-6-methoxy-2,3-dihydro-1H-inden-1-amine;
N-((trans)-2-(4-(benzyloxy)phenyl)cyclopropyl)-5,6-dimethoxy-2,3-dihydro-1H-inden-1-amine;
N-((trans)-2-(4-(benzyloxy)phenyl)cyclopropyl)-4,5-dimethoxy-2,3-dihydro-1H-inden-1-amine;
N-((trans)-2-phenylcyclopropyl)-2,3-dihydro-1H-inden-1-amine;
6-methoxy-N-((trans)-2-phenylcyclopropyl)-2,3-dihydro-1H-inden-1-amine;
6-chloro-N-((trans)-2-phenylcyclopropyl)-2,3-dihydro-1H-inden-1-amine;
N-((trans)-2-phenylcyclopropyl)-6-(trifluoromethyl)-2,3-dihydro-1H-inden-1-amine;
7-methoxy-N-((trans)-2-phenylcyclopropyl)-1,2,3,4-tetrahydronaphthalen-1-amine;
N-((trans)-2-(3′-chlorobiphenyl-4-yl)cyclopropyl)-6-methoxy-2,3-dihydro-1H-inden-1-amine;
N-((trans)-2-(4′-chlorobiphenyl-4-yl)cyclopropyl)-6-methoxy-2,3-dihydro-1H-inden-1-amine;
6-methoxy-N-((trans)-2-(3′-methoxybiphenyl-4-yl)cyclopropyl)-2,3-dihydro-1H-inden-1-amine;
N-trans-(2-cyclohexylethyl)-2-phenylcyclopropanamine;
(Trans)-N-(3-cyclohexylpropyl)-2-phenylcyclopropanamine;
(Trans)-N-(2-cycloheptylethyl)-2-phenylcyclopropanamine;
(Trans)-2-(4-(3-bromobenzyloxy)phenyl)-N-(2-cyclohexylethyl)cyclopropanamine;
N-((trans)-2-(4-(3-bromobenzyloxy)phenyl)cyclopropyl)-6-methoxy-2,3-dihydro-1H-inden-1-amine;
(Trans)-2-(3′-chlorobiphenyl-4-yl)-N-(2-cyclohexylethyl)cyclopropanamine;
(Trans)-2-(4′-chlorobiphenyl-4-yl)-N-(2-cyclohexylethyl)cyclopropanamine;
(Trans)-N-(2-cyclohexylethyl)-2-(3′-methoxybiphenyl-4-yl)cyclopropanamine;
N-((trans)-2-(4-(benzyloxy)phenyl)cyclopropyl)-7-methoxy-1,2,3,4-tetrahydronaphthalen-1-amine; and
1-((trans)-2-(4-(benzyloxy)phenyl)cyclopropylamino)cyclopropanecarboxamide;
   and pharmaceutically acceptable salts thereof.

Compounds of formula (VI) can be prepared by the methods disclosed in WO2011/131697, the disclosure of which is incorporated by reference herein in its entirety.

In the methods and uses according to the invention, the LSD1 inhibitor can be a compound of formula (VII) or an enantiomer, a diastereomer or a mixture of stereoisomers (such as a racemic mixture or a diastereomer mixture) thereof, or a pharmaceutically acceptable salt or solvate thereof:

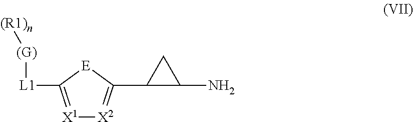

In formula (VII), E is —X³═X⁴—, —N(R3)-, —S—, or —O—;

X¹ and X² are each independently C(R2) or N;
X³ and X⁴, when present, are each independently C(R2) or N;

### L1 is —NH— or —NH—CH₂—;

G is a cyclyl group;
each R1 is independently chosen from alkyl, alkenyl, alkynyl, cyclyl, -L2-cyclyl, -L2-amino, -L2-hydroxyl, amino, amido, nitro, halo, haloalkyl, haloalkoxy, cyano, sulfinyl, sulfonyl, sulfonamide, hydroxyl, alkoxy, urea, carbamate, acyl, or carboxyl;
each R2 is independently chosen from —H, alkyl, alkenyl, alkynyl, cyclyl, -L2-cyclyl, -L2-amino, -L2-hydroxyl, amino, amido, nitro, halo, haloalkyl, haloalkoxy, cyano, sulfinyl, sulfonyl, sulfonamide, hydroxyl, alkoxy, urea, carbamate, acyl, or carboxyl, wherein each R2 group has 1, 2, or 3 independently chosen optional substituents, and further wherein two R2 groups bound to adjacent carbon atoms can be taken together to form a heterocyclyl or aryl group having 1, 2, or 3 independently chosen optional substituents; wherein said optional substituents are each independently chosen from alkyl, alkanoyl, heteroalkyl, heterocyclyl, haloalkyl, cycloalkyl, carbocyclyl, arylalkoxy, heterocyclylalkoxy, aryl, aryloxy, heterocyclyloxy, alkoxy, haloalkoxy, oxo, acyloxy, carbonyl, carboxyl, carboxamido, cyano, halogen, hydroxyl, amino, aminoalkyl, amidoalkyl, amido, nitro, thiol, alkylthio, arylthio, sulfinyl, sulfonyl, sulfonamide, urea or carbamate;
R3 is —H or an (C1-C6)alkyl group;
each L2 is independently chosen from alkylene or heteroalkylene; and
n is 0, 1, 2, 3, 4 or 5.

Compounds of formula (VII) having a (trans) disposition on the substituents on the cyclopropyl ring are preferred.

Preferably, the compound of formula (VII) is a compound from the list below:
5-((trans)-2-aminocyclopropyl)-N-(3-chlorophenyl)pyridin-2-amine;
5-((trans)-2-aminocyclopropyl)-N-(4-chlorophenyl)pyridin-2-amine;
5-((trans)-2-aminocyclopropyl)-N-(4-(trifluoromethyl)phenyl)pyridin-2-amine;
5-((trans)-2-aminocyclopropyl)-N-(3-methoxyphenyl)pyridin-2-amine;
5-((trans)-2-aminocyclopropyl)-N-(4-methoxyphenyl)pyridin-2-amine;
5-((trans)-2-aminocyclopropyl)-N-p-tolyl pyridin-2-amine;
5-((trans)-2-aminocyclopropyl)-N-m-tolyl pyridin-2-amine;
4-(5-((trans)-2-aminocyclopropyl)pyridin-2-ylamino)benzonitrile;
3-(5-((trans)-2-aminocyclopropyl)pyridin-2-ylamino)benzonitrile;
3-(5-((trans)-2-aminocyclopropyl)pyridin-2-ylamino)benzamide;
4-(5-((trans)-2-aminocyclopropyl)pyridin-2-ylamino)benzamide;
5-((trans)-2-aminocyclopropyl)-N-(3-chlorobenzyl)pyridin-2-amine;
5-((trans)-2-aminocyclopropyl)-N-(4-chlorobenzyl)pyridin-2-amine;
5-((trans)-2-aminocyclopropyl)-N-(3-(trifluoromethyl)benzyl)pyridin-2-amine;
5-((trans)-2-aminocyclopropyl)-N-(4-(trifluoromethyl)benzyl)pyridin-2-amine;
5-((trans)-2-aminocyclopropyl)-N-(3-methylbenzyl)pyridin-2-amine;
5-((trans)-2-aminocyclopropyl)-N-(4-methylbenzyl)pyridin-2-amine;
3-((5-((trans)-2-aminocyclopropyl)pyridin-2-ylamino)methyl)benzonitrile;
4-((5-((trans)-2-aminocyclopropyl)pyridin-2-ylamino)methyl)benzonitrile;
5-((trans)-2-aminocyclopropyl)-N-(3-methoxybenzyl)pyridin-2-amine;
5-((trans)-2-aminocyclopropyl)-N-(4-methoxybenzyl)pyridin-2-amine;
4-(5-((trans)-2-aminocyclopropyl)pyridin-2-ylamino)phenol;
3-((5-((trans)-2-aminocyclopropyl)pyridin-2-ylamino)methyl)benzamide;
4-((5-((trans)-2-aminocyclopropyl)pyridin-2-ylamino)methyl)benzamide;
4-((5-((trans)-2-aminocyclopropyl)pyridin-2-ylamino)methyl)phenol;
5-((trans)-2-aminocyclopropyl)-N-(3-ethynylphenyl)pyridin-2-amine;
N-(5-((trans)-2-aminocyclopropyl)pyridin-2-yl)-1H-indol-7-amine;
N-(5-((trans)-2-aminocyclopropyl)pyridin-2-yl)-1H-indazol-7-amine;
3-(5-((trans)-2-aminocyclopropyl)pyridin-2-ylamino)phenol;
4-((trans)-2-aminocyclopropyl)-N-(4-methylbenzyl)aniline;
4-((trans)-2-aminocyclopropyl)-N-(4-(trifluoromethyl)benzyl)aniline;
4-((trans)-2-aminocyclopropyl)-N-(3-chlorobenzyl)aniline;
3-(((4-((trans)-2-aminocyclopropyl)phenyl)amino)methyl)benzonitrile;
4-((trans)-2-aminocyclopropyl)-N-(p-tolyl)aniline;
4-((trans)-2-aminocyclopropyl)-N-(4-chlorophenyl)aniline;
3-((4-((trans)-2-aminocyclopropyl)phenyl)amino)benzonitrile;
N-(4-((trans)-2-aminocyclopropyl)phenyl)-3-methoxyaniline;
3-((4-((trans)-2-aminocyclopropyl)phenyl)amino)benzamide;
   and pharmaceutically acceptable salts thereof.

Compounds of formula (VII) can be prepared by the methods disclosed in WO2012/045883, the disclosure of which is incorporated by reference herein in its entirety.

In the methods and uses according to the invention, the LSD1 inhibitor can be a compound of formula (VIII) or an enantiomer, a diastereomer or a mixture of stereoisomers (such as a racemic mixture or a diastereomer mixture) thereof, or a pharmaceutically acceptable salt or solvate thereof:

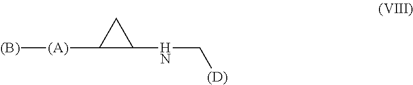

In formula (VIII), (A) is a cyclyl group having n substituents (R3);

(B) is a cyclyl group or an -(L1)-cyclyl group, wherein said cyclyl group or the cyclyl moiety comprised in said -(L1)-cyclyl group has n substituents (R2);
(L1) is —O—, —NH—, —N(alkyl)-, alkylene or heteroalkylene;
(D) is a heteroaryl group or an -(L2)-heteroaryl group, wherein said heteroaryl group or the heteroaryl moiety comprised in said -(L2)-heteroaryl group has one substituent (R1), and further wherein said heteroaryl group is covalently bonded to the remainder of the molecule through a ring carbon atom or the heteroaryl moiety comprised in said -(L2)-heteroaryl group is covalently bonded to the (L2) moiety through a ring carbon atom;
(L2) is —O—, —NH—, —N(alkyl)-, alkylene or heteroalkylene;
(R1) is a hydrogen bonding group, including but not limited to —OH, —NH₂, amido, —S(O)₂NH₂, —C(═O)NH₂, —CH₂—C(═O)NH₂, —NH—C(═O)CH₃, —NHCH₃, —N(CH₃)₂ or —CH₂—NH₂;
each (R2) is independently selected from alkyl, alkenyl, alkynyl, cyclyl, amino, amido, C-amido, alkylamino, hydroxyl, nitro, halo, haloalkyl, haloalkoxy, cyano, sulfinyl, sulfonyl, sulfonamide, alkoxy, acyl, carboxyl, carbamate or urea;
each (R3) is independently selected from alkyl, alkenyl, alkynyl, cyclyl, amino, amido, C-amido, alkylamino, hydroxyl, nitro, halo, haloalkyl, haloalkoxy, cyano, sulfinyl, sulfonyl, sulfonamide, alkoxy, acyl, carboxyl, carbamate, or urea; and
n is independently 0, 1, 2, 3 or 4.

Preferably in formula (VIII),

(A) is aryl or heteroaryl. Said aryl is preferably phenyl. Said heteroaryl is preferably pyridinyl, and/or;
(B) is —O—CH₂-phenyl or phenyl, each of which can be optionally substituted with n substituents R2, and/or;
(D) is a monocyclic heteroaryl, preferably thiazolyl, oxadiazolyl or pyrimidinyl, and more preferably oxadiazolyl; and/or;
(R1) is —NH₂ or —NHCH₃ and more preferably —NH₂.

Compounds of formula (VIII) having a (trans) disposition on the substituents on the cyclopropyl ring are preferred.

Preferably, the compound of formula (VIII) is a compound from the list below:
5-(((trans)-2-(4-(benzyloxy)phenyl)cyclopropylamino)methyl)pyrimidin-2-amine;
5-(((trans)-2-(4-(benzyloxy)phenyl)cyclopropylamino)methyl)thiazol-2-amine;
5-(((trans)-2-(6-(3-(trifluoromethyl)phenyl)pyridin-3-yl)cyclopropylamino)methyl)pyrimidin-2-amine;
5-(((trans)-2-(6-(3-(trifluoromethyl)phenyl)pyridin-3-yl)cyclopropylamino)methyl)thiazol-2-amine;
3-(5-((trans)-2-((2-aminopyrimidin-5-yl)methylamino)cyclopropyl)pyridin-2-yl)phenol;
3-(5-((trans)-2-((2-aminothiazol-5-yl)methylamino)cyclopropyl)pyridin-2-yl)phenol;
4′-((trans)-2-((2-aminopyrimidin-5-yl)methylamino)cyclopropyl)biphenyl-3-ol;
4′-((trans)-2-((2-aminothiazol-5-yl)methylamino)cyclopropyl)biphenyl-3-ol;
5-(((trans)-2-(4-(benzyloxy)phenyl)cyclopropylamino)methyl)-1,2,4-oxadiazol-3-amine;
5-(((trans)-2-(4-(benzyloxy)phenyl)cyclopropylamino)methyl)-1,3,4-oxadiazol-2-amine;
5-((((trans)-2-(4-((4-fluorobenzyl)oxy)phenyl)cyclopropyl)amino)methyl)-1,3,4-oxadiazol-2-amine;
5-((((trans)-2-(4-((3-fluorobenzyl)oxy)phenyl)cyclopropyl)amino)methyl)-1,3,4-oxadiazol-2-amine;
5-((((trans)-2-(4-((3,5-difluorobenzyl)oxy)phenyl)cyclopropyl)amino)methyl)-1,3,4-oxadiazol-2-amine;
5-((((trans)-2-(4-((4-chlorobenzyl)oxy)phenyl)cyclopropyl)amino)methyl)-1,3,4-oxadiazol-2-amine;
5-((((trans)-2-(4-((3-chlorobenzyl)oxy)phenyl)cyclopropyl)amino)methyl)-1,3,4-oxadiazol-2-amine;
5-((((trans)-2-(4-((2-fluorobenzyl)oxy)phenyl)cyclopropyl)amino)methyl)-1,3,4-oxadiazol-2-amine;
5-((((trans)-2-(4-(benzyloxy)phenyl)cyclopropyl)amino)methyl)-N-methyl-1,3,4-oxadiazol-2-amine;
N-(5-((((trans)-2-(4-(benzyloxy)phenyl)cyclopropyl)amino)methyl)-1,3,4-oxadiazol-2-yl)acetamide;
4′-((trans)-2-(((5-amino-1,3,4-oxadiazol-2-yl)methyl)amino)cyclopropyl)-[1,1′-biphenyl]-3-ol;
5-((((trans)-2-(6-(3-(trifluoromethyl)phenyl)pyridin-3-yl)cyclopropyl)amino)methyl)-1,3,4-oxadiazol-2-amine;
5-((((trans)-2-(4-(benzyloxy)phenyl)cyclopropyl)amino)methyl)-1,3,4-thiadiazol-2-amine;
2-((((trans)-2-(4-(benzyloxy)phenyl)cyclopropyl)amino)methyl)thiazol-5-amine;
4-((((trans)-2-(3-(trifluoromethyl)-[1,1′-biphenyl]-4-yl)cyclopropyl)amino)methyl)thiazol-2-amine;
2-((((trans)-2-(4-(benzyloxy)phenyl)cyclopropyl)amino)methyl)oxazol-5-amine;
3-((((trans)-2-(4-(benzyloxy)phenyl)cyclopropyl)amino)methyl) isoxazol-5-amine;
5-((((trans)-2-(4-(benzyloxy)phenyl)cyclopropyl)amino)methyl)-N,N-dimethyl -1,3,4-oxadiazol-3-amine;
3-((((trans)-2-(4-(benzyloxy)phenyl)cyclopropyl)amino)methyl)-1,2,4-oxadiazol-5-amine;
5-((((trans)-2-(4-(benzyloxy)phenyl)cyclopropyl)amino)methyl)-1,2,4-thiadiazol-3-amine;
5-((((trans)-2-(4-(benzyloxy)phenyl)cyclopropyl)amino)methyl)pyridin-2-amine;
6-((((trans)-2-(4-(benzyloxy)phenyl)cyclopropyl)amino)methyl)pyridazin-3-amine;
5-((((trans)-2-(4-(benzyloxy)phenyl)cyclopropyl)amino)methyl)pyrazin-2-amine;
2-((((trans)-2-(4-(benzyloxy)phenyl)cyclopropyl)amino)methyl)pyrimidin-5-amine;
6-((((trans)-2-(4-(benzyloxy)phenyl)cyclopropyl)amino)methyl)-1,2,4-triazin-3-amine;
3-((((trans)-2-(4-(benzyloxy)phenyl)cyclopropyl)amino)methyl)-1,2,4-triazin-6-amine;
4′-((trans)-2-((2-aminothiazol-5-yl)methylamino)cyclopropyl)biphenyl-3-ol;
5-(((trans)-2-(4-(benzyloxy)phenyl)cyclopropylamino)methyl)-1,2,4-oxadiazol-3-amine;
5-(((trans)-2-(4-(benzyloxy)phenyl)cyclopropylamino)methyl)-1,3,4-oxadiazol-2-amine;
5-((((trans)-2-(4-((4-fluorobenzyl)oxy)phenyl)cyclopropyl)amino)methyl)-1,3,4-oxadiazol-2-amine;
5-((((trans)-2-(4-((3-fluorobenzyl)oxy)phenyl)cyclopropyl)amino)methyl)-1,3,4-oxadiazol-2-amine;
(−) 5-((((trans)-2-(4-(benzyloxy)phenyl)cyclopropylamino)methyl)-1,3,4-oxadiazol-2-amine;
(−) 5-((((trans)-2-(4-((3-fluorobenzyl)oxy)phenyl)cyclopropyl)amino)methyl)-1,3,4-oxadiazol-2-amine;
(−) 5-((((trans)-2-(4-(benzyloxy)phenyl)cyclopropyl)amino)methyl)-N-methyl-1,3,4-oxadiazol-2-amine;
(−) N-(5-((((trans)-2-(4-(benzyloxy)phenyl)cyclopropyl)amino)methyl)-1,3,4-oxadiazol-2-yl)acetamide;
(−) 5-((((trans)-2-(4-(benzyloxy)phenyl)cyclopropyl)amino)methyl)pyrimidin-2-amine;
(−) 5-((((trans)-2-(4-(benzyloxy)phenyl)cyclopropyl)amino)methyl)-1,3,4-thiadiazol-2-amine;
(−) 5-((((trans)-2-(4-((2-fluorobenzyl)oxy)phenyl)cyclopropyl)amino)methyl)-1,3,4-oxadiazol-2-amine;
4′-((trans)-2-((2-aminothiazol-5-yl)methylamino)cyclopropyl)biphenyl-3-ol;
5-(((trans)-2-(4-(benzyloxy)phenyl)cyclopropylamino)methyl)-1,2,4-oxadiazol-3-amine;
5-(((trans)-2-(4-(benzyloxy)phenyl)cyclopropylamino)methyl)-1,3,4-oxadiazol-2-amine;
5-((((trans)-2-(4-((4-fluorobenzyl)oxy)phenyl)cyclopropyl)amino)methyl)-1,3,4-oxadiazol-2-amine;
5-((((trans)-2-(4-((3-fluorobenzyl)oxy)phenyl)cyclopropyl)amino)methyl)-1,3,4-oxadiazol-2-amine;
(−) 5-((((trans)-2-(4-(benzyloxy)phenyl)cyclopropylamino)methyl)-1,3,4-oxadiazol-2-amine;
(−) 5-((((trans)-2-(4-((3-fluorobenzyl)oxy)phenyl)cyclopropyl)amino)methyl)-1,3,4-oxadiazol-2-amine;
(−) 5-((((trans)-2-(4-(benzyloxy)phenyl)cyclopropyl)amino)methyl)-N-methyl-1,3,4-oxadiazol-2-amine;
(−)N-(5-((((trans)-2-(4-(benzyloxy)phenyl)cyclopropyl)amino)methyl)-1,3,4-oxadiazol-2-yl)acetamide;
(−) 5-((((trans)-2-(4-(benzyloxy)phenyl)cyclopropyl)amino)methyl)pyrimidin-2-amine;
(−) 5-((((trans)-2-(4-(benzyloxy)phenyl)cyclopropyl)amino)methyl)-1,3,4-thiadiazol-2-amine;
(−) 5-((((trans)-2-(4-((2-fluorobenzyl)oxy)phenyl)cyclopropyl)amino)methyl)-1,3,4-oxadiazol-2-amine;
   and pharmaceutically acceptable salts thereof.

Still more preferably, the compound of formula (VIII) is (−) 5-((((trans)-2-(4-(benzyloxy)phenyl)cyclopropyl)amino)methyl)-1,3,4-oxadiazol-2-amine, or a pharmaceutically acceptable salt thereof.

Compounds of formula (VIII) can be prepared by the methods disclosed in WO2012/013728, the disclosure of which is incorporated by reference herein in its entirety.

In the methods and uses according to the invention, the LSD1 inhibitor can be a compound of formula (IX) or an enantiomer, a diastereomer or a mixture of stereoisomers (such as a racemic mixture or a diastereomer mixture) thereof, or a pharmaceutically acceptable salt or solvate thereof:

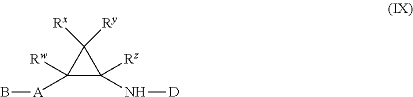

wherein:
A is aryl or heteroaryl, wherein said aryl or said heteroaryl is optionally substituted with one or more R¹;
B is hydrogen, R¹ or -L-E;
E is aryl or heteroaryl, wherein said aryl or said heteroaryl is optionally substituted with one or more R²;
L is a bond, —O—, —NH—, —N(C₁₋₄ alkyl)-, C₁₋₄ alkylene or heteroC₁₋₄ alkylene;
D is a cycloalkyl group having from 4 to 7 C atoms, wherein said cycloalkyl group has one or two substituents
R³ and is further optionally substituted with one or more R⁴, and wherein the cycloalkyl group optionally:
(a) is fused to a phenyl or a 5- or 6-membered aromatic heterocyclic ring containing from 1 to 3 heteroatoms independently selected from N, O and S, wherein said fused phenyl or said fused aromatic heterocyclic ring is optionally substituted with one or more R⁵; or
(b) is bonded to a linker group —(C(R^{a})₂)ₚ— linking together any two non-adjacent ring carbon atoms of the cycloalkyl group, wherein p is 1 or 2 and each R^{a} independently is hydrogen or C₁₋₄ alkyl; or
(c) is linked to a second ring that is either a 3- to 7-membered saturated carbocyclic ring or a 3- to 7-membered saturated heterocyclic ring containing from 1 to 3 heteroatoms independently selected from N, O and S, wherein said second ring is linked together with the cycloalkyl group via a single carbon atom common to both rings, and wherein said second ring is optionally substituted with one or more R⁶;
   each R¹ is independently selected from C₁₋₈ alkyl, C₂₋₈ alkenyl, C₂₋₈ alkynyl, cyclyl, amino, amido, hydroxyl, nitro, halo, haloC₁₋₈ alkyl, haloC₁₋₈ alkoxy, cyano, sulfinyl, sulfonyl, sulfonamide, C₁₋₈ alkoxy, acyl, carboxyl, O-carboxy, C-carboxy, carbamate and urea;
   each R² is independently selected from C₁₋₈ alkyl, C₂₋₈ alkenyl, C₂₋₈ alkynyl, cyclyl, amino, amido, hydroxyl, nitro, halo, haloC₁₋₈ alkyl, haloC₁₋₈ alkoxy, cyano, sulfinyl, sulfonyl, sulfonamide, C₁₋₈ alkoxy, acyl, carboxyl, O-carboxy, C-carboxy, carbamate and urea;
   each R³ is independently selected from —NR⁷R⁸, —NHOH, —NR⁹COR¹⁰, —NR⁹SO₂R¹⁰, —NR⁹COOR¹⁰, —NR⁹CONR⁷R⁸, —NR⁹SO₂NR⁷R⁸, —OH, —CONR⁷R⁸, oxo, —C₁₋₄ alkylene-NR⁷R⁸, —C₁₋₄ alkylene-NHOH, —C₁₋₄ alkyene-NR⁹COR¹⁰, —C₁₋₄ alkylene-NR⁹SO₂R¹⁰, —C₁₋₄ alkylene-NR⁹COOR¹⁰, —C₁₋₄ alkylene-NR⁹CONR⁷R⁸, —C₁₋₄ alkylene-NR⁹SO₂NR⁷R⁸, —C₁₋₄ alkylene-OH and —C₁₋₄ alkylene-CONR⁷R⁸;
   each R⁴ and each R⁶ is independently selected from C₁₋₈ alkyl, halo, haloC₁₋₈ alkyl, haloC₁₋₈ alkoxy and C₁₋₈ alkoxy;
   each R5 is independently selected from C₁₋₈ alkyl, C₂₋₈ alkenyl, C₂₋₈ alkynyl, cyclyl, amino, amido, hydroxyl, nitro, halo, haloC₁₋₈ alkyl, haloC₁₋₈ alkoxy, cyano, sulfinyl, sulfonyl, sulfonamide, C₁₋₈ alkoxy, acyl, carboxyl, O-carboxy, C-carboxy, carbamate and urea;
   each R⁷ and each R⁸ is independently selected from hydrogen, C₁₋₈ alkyl, R¹²R¹³N—C₁₋₈ alkyl and hydroxyC₁₋₈ alkyl, or R⁷ and R⁸ are linked together to form, along with the N atom to which they are bound, a saturated 3- to 7-membered heterocyclic ring which optionally contains one further heteroatom selected from N, O and S, wherein one or more C atoms in said heterocyclic ring are optionally oxidized to form CO groups, wherein one or more S atoms in said heterocyclic ring, if present, are optionally oxidized to form independently SO groups or SO₂ groups, and wherein said heterocyclic ring is optionally substituted with one or more R¹¹;
   each R⁹ is independently selected from hydrogen and C₁₋₄ alkyl;
   each R¹⁰ is independently selected from C₁₋₈ alkyl, haloC₁₋₈ alkyl, cyclyl and cyclylC₁₋₈ alkyl, wherein said cyclyl or the cyclyl moiety comprised in said cyclylC₁₋₈ alkyl is optionally substituted with one or more R¹⁴;
   each R¹¹ is independently selected from C₁₋₈ alkyl, halo, C₁₋₈alkoxy, hydroxyl and —NR¹²R¹³;
   each R¹² and each R¹³ is independently selected from hydrogen and C₁₋₈alkyl;
   each R¹⁴ is independently selected from C₁₋₈ alkyl, C₂₋₈ alkenyl, C₂₋₈ alkynyl, amino, amido, hydroxyl, nitro, halo, haloC₁₋₈ alkyl, haloC₁₋₈ alkoxy, cyano, sulfinyl, sulfonyl, sulfonamide, C₁₋₈ alkoxy, acyl, carboxyl, O-carboxy, C-carboxy, carbamate and urea; and
   each R^{w}, R^{x}, R^{y} and R^{z} is independently selected from hydrogen, halo and C₁₋₄ alkyl.

Preferably in formula (IX),

(A) is phenyl, thiazolyl or pyridyl, preferably phenyl, which rings can be optionally substituted with one or more R¹, and/or
(B) is H, and/or
(R1) is C₁₋₈ alkyl, amino, amido, hydroxyl, halo, haloC₁₋₈ alkyl, haloC₁₋₈ alkoxy, cyano, sulfonamide, C₁₋₈ alkoxy, acyl, carboxyl, carbamate, and urea, and more preferably halo, C₁₋₄ alkyl, haloC₁₋₄ alkyl, C₁₋₄ alkoxy and C₃₋₆ cycloalkyl; and/or
(D) is selected from D1, D2, D3 and D4:

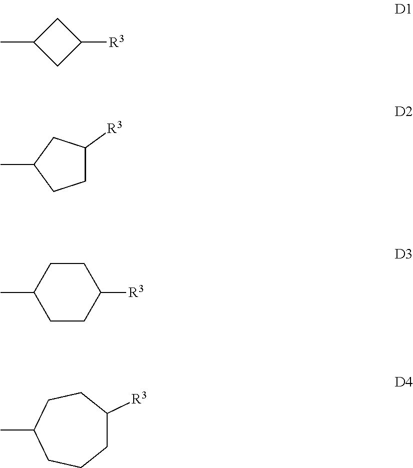

and more preferably D3; and/or
(R3) is selected from —NR⁷R⁸, —NHOH, —NR⁹COR¹⁰, —NR⁹SO₂R¹⁰, —NR⁹COOR¹⁰, —NR⁹CONR⁷R⁸, —NR⁹SO₂NR⁷R⁸, —OH, —CONR⁷R⁸, oxo, —C₁₋₄ alkylene-NR⁷R⁸, —C₁₋₄ alkylene-OH and —C₁₋₄ alkylene-CONR⁷R⁸, more preferably from —NR⁷R⁸, —OH, —C₁₋₄ alkylene-NR⁷R⁸, and —C₁₋₄ alkylene-OH, still more preferably —NR⁷R⁸ (such as —NH2); and/or
each R^{w}, R^{x}, R^{y} and R^{z} is hydrogen.

Compounds of formula (IX) having a (trans) disposition on the substituents on the cyclopropyl ring are preferred.

Preferably, the compound of formula (IX) is a compound from the list below:
N1-((trans)-2-phenylcyclopropyl)cyclohexane-1,4-diamine;
(cis)-N1-((1S,2R)-2-phenylcyclopropyl)cyclohexane-1,4-diamine;
(trans)-N1-((1S,2R)-2-phenylcyclopropyl)cyclohexane-1,4-diamine;
(cis)-N1-((1R,2S)-2-phenylcyclopropyl)cyclohexane-1,4-diamine;
(trans)-N1-((1R,2S)-2-phenylcyclopropyl)cyclohexane-1,4-diamine;
N1-((trans)-2-(thiazol-5-yl)cyclopropyl)cyclohexane-1,4-diamine;
N1-((trans)-2-(pyridin-3-yl)cyclopropyl)cyclohexane-1,4-diamine;
N1-((trans)-2-(6-(3-(trifluoromethyl)phenyl)pyridin-3-yl)cyclopropyl)cyclohexane-1,4-diamine;
N1-((trans)-2-(3′-(trifluoromethyl)-[1,1′-biphenyl]-4-yl)cyclopropyl)cyclohexane-1,4-diamine;
N1-((trans)-2-(4-(benzyloxy)phenyl)cyclopropyl)cyclohexane-1,4-diamine;
4-(((trans)-2-(6-(3-(trifluoromethyl)phenyl)pyridin-3-yl)cyclopropyl)amino)cyclohexanol;
4-(((trans)-2-(6-(3-(trifluoromethyl)phenyl)pyridin-3-yl)cyclopropyl)amino)cyclohexanecarboxamide;
N-(4-(((trans)-2-(6-(3-(trifluoromethyl)phenyl)pyridin-3-yl)cyclopropyl)amino)cyclohexyl)acetamide;
N-(4-(((trans)-2-(6-(3-(trifluoromethyl)phenyl)pyridin-3-yl)cyclopropyl)amino)cyclohexyl)methanesulfonamide;
(R)-1-(4-(((trans)-2-phenylcyclopropyl)amino)cyclohexyl)pyrrolidin-3-amine;
N1-((trans)-2-(4′-chloro-[1,1′-biphenyl]-4-yl)cyclopropyl)cyclohexane-1,4-diamine;
N1-((trans)-2-(3′-chloro-[1,1′-biphenyl]-4-yl)cyclopropyl)cyclohexane-1,4-diamine;
4′-((trans)-2-((4-aminocyclohexyl)amino)cyclopropyl)-[1,1′-biphenyl]-3-ol;
N-(4′-((trans)-2-((4-aminocyclohexyl)amino)cyclopropyl)-[1,1′-biphenyl]-3-yl)methanesulfonamide;
N1-((trans)-2-(4-((2-fluorobenzyl)oxy)phenyl)cyclopropyl)cyclohexane-1,4-diamine;
N1-((trans)-2-(4-((3-fluorobenzyl)oxy)phenyl)cyclopropyl)cyclohexane-1,4-diamine;
N1-((trans)-2-(4-((4-fluorobenzyl)oxy)phenyl)cyclopropyl)cyclohexane-1,4-diamine;
N1-methyl-N4-((trans)-2-phenylcyclopropyl)cyclohexane-1,4-diamine;
N1-methyl-N4-((trans)-2-(3′-(trifluoromethyl)-[1,1′-biphenyl]-4-yl)cyclopropyl)cyclohexane-1,4-diamine;
N1-((trans)-2-(4-(benzyloxy)phenyl)cyclopropyl)-N4-methylcyclohexane-1,4-diamine;
N1-((trans)-2-phenylcyclopropyl)cyclobutane-1,3-diamine;
N1-((trans)-2-(3′-(trifluoromethyl)-[1,1′-biphenyl]-4-yl)cyclopropyl)cyclobutane-1,3-diamine;
N1-((trans)-2-(4-(benzyloxy)phenyl)cyclopropyl)cyclobutane-1,3-diamine;
N1-((trans)-2-phenylcyclopropyl)-2,3-dihydro-1H-indene-1,3-diamine;
N1-((trans)-2-(3′-(trifluoromethyl)-[1,1′-biphenyl]-4-yl)cyclopropyl)-2,3-dihydro-1H-indene-1,3-diamine;
N1-((trans)-2-(4-(benzyloxy)phenyl)cyclopropyl)-2,3-dihydro-1H-indene-1,3-diamine;
N1-((trans)-2-fluoro-2-phenylcyclopropyl)cyclohexane-1,4-diamine;
N1-((1S,2S)-2-fluoro-2-phenylcyclopropyl)cyclohexane-1,4-diamine;
N1-((1R,2R)-2-fluoro-2-phenylcyclopropyl)cyclohexane-1,4-diamine;
1-methyl-N4-((trans)-2-phenylcyclopropyl)cyclohexane-1,4-diamine;
4-(aminomethyl)-N-((trans)-2-phenylcyclopropyl)cyclohexanamine;
N1-((trans)-2-phenylcyclopropyl)cyclohexane-1,3-diamine;
N1-((cis)-2-phenylcyclopropyl)cyclohexane-1,4-diamine;
Tert-butyl (4-(((trans)-2-phenylcyclopropyl)amino)cyclohexyl)carbamate;
1-ethyl-3-(4-(((trans)-2-phenylcyclopropyl)amino)cyclohexyl)urea;
4-morpholino-N-((trans)-2-phenylcyclopropyl)cyclohexanamine;
N1-((trans)-2-(4-bromophenyl)cyclopropyl)cyclohexane-1,4-diamine;
N1-(2-(o-tolyl)cyclopropyl)cyclohexane-1,4-diamine;
N1-(2-(4-(trifluoromethyl)phenyl)cyclopropyl)cyclohexane-1,4-diamine;
N1-(2-(4-methoxyphenyl)cyclopropyl)cyclohexane-1,4-diamine;
4-(2-((4-aminocyclohexyl)amino)cyclopropyl)phenol;
N1-(2-(2-fluorophenyl)cyclopropyl)cyclohexane-1,4-diamine;
N1-(2-(3,4-difluorophenyl)cyclopropyl)cyclohexane-1,4-diamine;
N1-(2-(naphthalen-2-yl)cyclopropyl)cyclohexane-1,4-diamine;
N1-(2-methyl-2-phenylcyclopropyl)cyclohexane-1,4-diamine;
(R)-1-(4-(((trans)-2-(3′-(trifluoromethyl)-[1,1′-biphenyl]-4-yl)cyclopropyl)amino)cyclohexyl)pyrrolidin-3-amine;
(Cis)-N1-((1S,2R)-2-(3′-(trifluoromethyl)-[1,1′-biphenyl]-4-yl)cyclopropyl)cyclohexane-1,4-diamine;
(Trans)-N1-((1S,2R)-2-(3′-(trifluoromethyl)-[1,1′-biphenyl]-4-yl)cyclo-propyl)cyclohexane-1,4-diamine;
(Cis)-N1-((1R,2S)-2-(3′-(trifluoromethyl)-[1,1′-biphenyl]-4-yl)cyclo-propyl)cyclohexane-1,4-diamine;
(Trans)-N1-((1R,2S)-2-(3′-(trifluoromethyl)-[1,1′-biphenyl]-4-yl)cyclo-propyl)cyclohexane-1,4-diamine;
N1-((trans)-2-(4-cyclopropylphenyl)cyclopropyl)cyclohexane-1,4-diamine;
N1-((trans)-2-(4-(pyridin-3-yl)phenyl)cyclopropyl)cyclohexane-1,4-diamine;
N1-((trans)-2-(4-(1H-indazol-6-yl)phenyl)cyclopropyl)cyclohexane-1,4-diamine;
N1-((trans)-2-(4-(1H-pyrazol-5-yl)phenyl)cyclopropyl)cyclohexane-1,4-diamine;
3-(5-((trans)-2-((4-aminocyclohexyl)amino)cyclopropyl)thiophen-2-yl)phenol;
3-(5-((trans)-2-((4-aminocyclohexyl)amino)cyclopropyl)thiazol-2-yl)phenol;
3-(5-((trans)-2-((4-aminocyclohexyl)amino)cyclopropyl)pyridin-2-yl)-5-methoxybenzonitrile;
5-(5-((trans)-2-((4-aminocyclohexyl)amino)cyclopropyl)pyridin-2-yl)-2-methylphenol;
N-(4′-((trans)-2-((4-aminocyclohexyl)amino)cyclopropyl)-6-methoxy-[1,1′-biphenyl]-3-yl)methanesulfonamide;
N-(3-(5-((trans)-2-((4-aminocyclohexyl)amino)cyclopropyl)thiazol-2-yl)phenyl)-2-cyanobenzenesulfonamide
N-(4′-((trans)-2-((4-aminocyclohexyl)amino)cyclopropyl)-[1,1′-biphenyl]-3-yl)-2-cyanobenzenesulfonamide;
6-amino-N-(4′-((trans)-2-((4-aminocyclohexyl)amino)cyclopropyl)-[1,1′-biphenyl]-3-yl)pyridine-3-sulfonamide;
N-(4′-((trans)-2-((4-aminocyclohexyl)amino)cyclopropyl)-[1,1′-biphenyl]-3-yl)piperazine-1-sulfonamide;
N1-((cis)-2-fluoro-2-phenylcyclopropyl)cyclohexane-1,4-diamine;
N1-((trans)-2-(4-((3-(piperazin-1-yl)benzyl)oxy)phenyl)cyclopropyl)cyclohexane-1,4-diamine;
N1-((trans)-2-(4-(pyridin-3-ylmethoxy)phenyl)cyclopropyl)cyclohexane-1,4-diamine;
N1-((trans)-2-(6-((3-methylbenzyl)amino)pyridin-3-yl)cyclopropyl)cyclohexane-1,4-diamine;
3-((5-((trans)-2-((4-aminocyclohexyl)amino)cyclopropyl)pyridin-2-yl)amino)benzonitrile;
N1-((trans)-2-(naphthalen-2-yl)cyclopropyl)cyclohexane-1,4-diamine;
N1-((trans)-2-(o-tolyl)cyclopropyl)cyclohexane-1,4-diamine;
N1-((trans)-2-(4-(trifluoromethyl)phenyl)cyclopropyl)cyclohexane-1,4-diamine;
N1-((trans)-2-(4-methoxyphenyl)cyclopropyl)cyclohexane-1,4-diamine;
N1-((trans)-2-(2-fluorophenyl)cyclopropyl)cyclohexane-1,4-diamine;
N1-((trans)-2-(3,4-difluorophenyl)cyclopropyl)cyclohexane-1,4-diamine;
N1-((trans)-2-methyl-2-phenylcyclopropyl)cyclohexane-1,4-diamine;
(cis)-N1-((1S,2R)-2-(pyridin-3-yl)cyclopropyl)cyclohexane-1,4-diamine;
(trans)-N1-((1R,2S)-2-(pyridin-3-yl)cyclopropyl)cyclohexane-1,4-diamine;
(cis)-N1-((1R,2S)-2-(pyridin-3-yl)cyclopropyl)cyclohexane-1,4-diamine;
(trans)-N1-((1S,2R)-2-(pyridin-3-yl)cyclopropyl)cyclohexane-1,4-diamine;
(cis)-N1-((1S,2R)-2-phenylcyclopropyl)cyclobutane-1,3-diamine;
(trans)-N1-((1R,2S)-2-phenylcyclopropyl)cyclobutane-1,3-diamine;
(cis)-N1-((1R,2S)-2-phenylcyclopropyl)cyclobutane-1,3-diamine;
(trans)-N1-((1S,2R)-2-phenylcyclopropyl)cyclobutane-1,3-diamine;
(cis)-N1-((1S,2R)-2-(3,4-difluorophenyl)cyclopropyl)cyclohexane-1,4-diamine;
(trans)-N1-((1R,2S)-2-(3,4-difluorophenyl)cyclopropyl)cyclohexane-1,4-diamine;
(cis)-N1-((1R,2S)-2-(3,4-difluorophenyl)cyclopropyl)cyclohexane-1,4-diamine;
(trans)-N1-((1S,2R)-2-(3,4-difluorophenyl)cyclopropyl)cyclohexane-1,4-diamine;
(cis)-N1-((1S,2R)-2-(naphthalen-2-yl)cyclopropyl)cyclohexane-1,4-diamine;
(trans)-N1-((1R,2S)-2-(naphthalen-2-yl)cyclopropyl)cyclohexane-1,4-diamine;
(cis)-N1-((1R,2S)-2-(naphthalen-2-yl)cyclopropyl)cyclohexane-1,4-diamine;
(trans)-N1-((1S,2R)-2-(naphthalen-2-yl)cyclopropyl)cyclohexane-1,4-diamine;
(cis)-N1-((1S,2R)-2-(4-(1H-pyrazol-5-yl)phenyl)cyclopropyl)cyclohexane-1,4-diamine;
(trans)-N1-((1R,2S)-2-(4-(1H-pyrazol-5-yl)phenyl)cyclopropyl)cyclohexane-1,4-diamine;
(cis)-N1-((1R,2S)-2-(4-(1H-pyrazol-5-yl)phenyl)cyclopropyl)cyclohexane-1,4-diamine;
(trans)-N1-((1S,2R)-2-(4-(1H-pyrazol-5-yl)phenyl)cyclopropyl)cyclohexane-1,4-diamine;
N-(4′-((1R,2S)-2-(((cis)-4-aminocyclohexyl)amino)cyclopropyl)-[1,1′-biphenyl]-3-yl)piperazine-1-sulfonamide;
N-(4′-((1S,2R)-2-(((trans)-4-aminocyclohexyl)amino)cyclopropyl)-[1,1′-biphenyl]-3-yl)piperazine-1-sulfonamide;
N-(4′-((1S,2R)-2-(((cis)-4-aminocyclohexyl)amino)cyclopropyl)-[1,1′-biphenyl]-3-yl)piperazine-1-sulfonamide;
N-(4′-((1R,2S)-2-(((trans)-4-aminocyclohexyl)amino)cyclopropyl)-[1,1′-biphenyl]-3-yl)piperazine-1-sulfonamide;
(cis)-N1-((1S,2R)-2-(4-((2-fluorobenzyl)oxy)phenyl)cyclopropyl)cyclohexane-1,4-diamine;
(trans)-N1-((1R,2S)-2-(4-((2-fluorobenzyl)oxy)phenyl)cyclopropyl)cyclohexaane-1,4-diamine;
(cis)-N1-((1R,2S)-2-(4-((2-fluorobenzyl)oxy)phenyl)cyclopropyl)cyclohexane-1,4-diamine;
(trans)-N1-((1S,2R)-2-(4-((2-fluorobenzyl)oxy)phenyl)cyclopropyl)cyclohexane-1,4-diamine;
   and pharmaceutically acceptable salts thereof.

Still more preferably, the compound of formula (IX) is (trans)-N1-((1R,2S)-2-phenylcyclopropyl)cyclohexane-1,4-diamine; or a pharmaceutically acceptable salt thereof.

Compounds of formula (IX) can be prepared by the methods disclosed in WO2013/057322, the disclosure of which is incorporated by reference herein in its entirety.

In the methods and uses according to the invention, the LSD1 inhibitor can be a compound of formula (X) or an enantiomer, a diastereomer or a mixture of stereoisomers (such as a racemic mixture or a diastereomer mixture) thereof, or a pharmaceutically acceptable salt or solvate thereof:

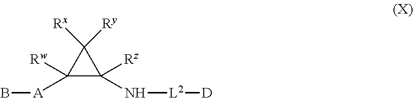

wherein:
A is aryl or heteroaryl, wherein said aryl or heteroaryl is optionally substituted with one or more R¹;
B is H, R¹ or -L¹-E;
E is aryl or heteroaryl, wherein said aryl or said heteroaryl is optionally substituted with one or more R²;
L′ is a bond, —O—, —NH—, —N(C₁₋₄ alkyl)-, C₁₋₄ alkylene or heteroC₁₋₄alkylene;
L² is a bond and D is a cyclic group selected from:
(i) a 3- to 7-membered monocyclic saturated heterocyclic ring containing 1 or 2 heteroatoms independently selected from N, O and S, and
(ii) a 7- to 15-membered polycyclic ring system which comprises at least one saturated heterocyclic ring, wherein the polycyclic ring system contains from 1 to 4 heteroatoms independently selected from N, O and S,
   wherein the cyclic group (i) or (ii) is linked to the remainder of the compound of Formula I through a ring C atom,
   wherein one or more ring C atoms in the cyclic group (i) or (ii) are optionally oxidized to form CO groups,
   wherein one or more S atoms in the cyclic group (i) or (ii), if present, are optionally oxidized to form independently SO groups or SO₂ groups, and
   wherein the cyclic group (i) or (ii) is optionally substituted with one or more R³;
   or L² is C₁₋₄ alkylene and D is a cyclic group selected from:
(iii) a 3- to 7-membered monocyclic saturated heterocyclic ring containing 1 or 2 heteroatoms independently selected from N, O and S, and
(iv) a 7- to 15-membered polycyclic saturated ring system which comprises at least one heterocyclic ring, wherein the polycyclic saturated ring system contains from 1 to 4 heteroatoms independently selected from N, O and S,
   wherein the cyclic group (iii) or (iv) is linked to the remainder of the compound of Formula I through a ring C atom,
   wherein one or more ring C atoms in the cyclic group (iii) or (iv) are optionally oxidized to form CO groups,
   wherein one or more S atoms in the cyclic group (iii) or (iv), if present, are optionally oxidized to form independently SO groups or SO₂ groups, and
   wherein the cyclic group (iii) or (iv) is optionally substituted with one or more R³;
   each R¹ is independently selected from C₁₋₈ alkyl, C₂₋₈ alkenyl, C₂₋₈ alkynyl, cyclyl, amino, amido, hydroxyl, nitro, halo, haloC₁₋₈ alkyl, haloC₁₋₈ alkoxy, cyano, sulfinyl, sulfonyl, sulfonamide, C₁₋₈ alkoxy, acyl, carboxyl, O-carboxy, C-carboxy, carbamate and urea;
   each R² is independently selected from C₁₋₈ alkyl, C₂₋₈ alkenyl, C₂₋₈ alkynyl, cyclyl, amino, amido, hydroxyl, nitro, halo, haloC₁₋₈ alkyl, haloC₁₋₈ alkoxy, cyano, sulfinyl, sulfonyl, sulfonamide, C₁₋₈ alkoxy, acyl, carboxyl, O-carboxy, C-carboxy, carbamate and urea;
   each R³ is independently selected from C₁₋₈ alkyl, C₂₋₈ alkenyl, C₂₋₈ alkynyl, cyclyl, amino, amido, hydroxyl, nitro, halo, haloC₁₋₈ alkyl, haloC₁₋₈ alkoxy, cyano, sulfinyl, sulfonyl, sulfonamide, C₁₋₈ alkoxy, acyl, carboxyl, O-carboxy, C-carboxy, carbamate and urea; and
   each R^{w}, R^{x}, R^{y} and R^{z} is independently selected from hydrogen, halo and C₁₋₄ alkyl.

Preferably in formula (X),

(A) is phenyl, thiazolyl or pyridyl, preferably phenyl, which rings can be optionally substituted with one or more R¹, and/or
(B) is H, and/or
(R¹) is C₁₋₈ alkyl, amino, amido, hydroxyl, halo, haloC₁₋₈ alkyl, haloC₁₋₈ alkoxy, cyano, sulfonamide, C₁₋₈ alkoxy, acyl, carboxyl, carbamate, and urea and more preferably halo, C₁₋₄ alkyl, haloC₁₋₄ alkyl, C₁₋₄ alkoxy and C₃₋₆ cycloalkyl; and/or
L2 is a bond and (D) is a 3- to 7-membered monocyclic saturated heterocyclic ring containing 1 heteroatom selected from N, O and S wherein D is linked to the remainder of the compound of formula (X) through a C, more preferably a 3- to 7-membered monocyclic saturated heterocyclic ring containing 1 N atom wherein D is linked to the remainder of the compound of formula (X) through a C, and even more preferably D is 4-piperidinyl, or L2 is a bond and (D) is a ring system selected from (a), (b), (c) and (d)

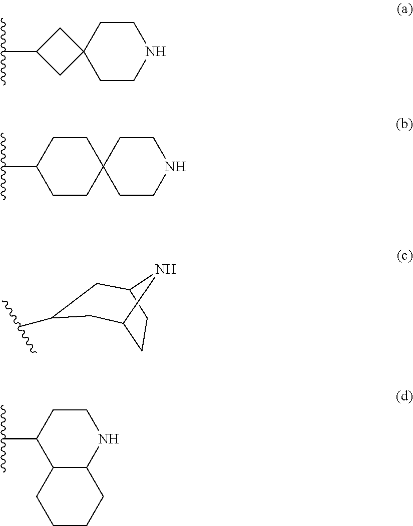 [SMILES: *C1CC2(CCNCC2)C1 ; *C1CCC2(CCNCC2)CC1 ; *C1CC2CCC(C1)N2 ; *C1CCNC2CCCCC12]

wherein any D is optionally substituted with one or more R³; and/or
each R^{w}, R^{x}, R^{y} and R^{z} is hydrogen.

Compounds of formula (X) having a (trans) disposition on the substituents on the cyclopropyl ring are preferred.

Preferably, the compound of formula (X) is a compound from the list below:
N-((trans)-2-phenylcyclopropyl)piperidin-4-amine;
N-((1S,2R)-2-phenylcyclopropyl)piperidin-4-amine;
N-((1R,2S)-2-phenylcyclopropyl)piperidin-4-amine;
N-((trans)-2-(4-(benzyloxy)phenyl)cyclopropyl)piperidin-4-amine;
N-((trans)-2-(6-(3-(trifluoromethyl)phenyl)pyridin-3-yl)cyclopropyl)tetrahydro-2H-pyran-4-amine;
N-((trans)-2-(pyridin-3-yl)cyclopropyl)piperidin-4-amine;
N-((trans)-2-(thiazol-5-yl)cyclopropyl)piperidin-4-amine;
N-((trans)-2-(3′-(trifluoromethyl)-[1,1′-biphenyl]-4-yl)cyclopropyl)piperidin-4-amine;
N-((trans)-2-phenylcyclopropyl)piperidin-3-amine;
N-((trans)-2-(3′-(trifluoromethyl)-[1,1′-biphenyl]-4-yl)cyclopropyl)piperidin-3-amine;
N-((trans)-2-(4-(benzyloxy)phenyl)cyclopropyl)piperidin-3-amine;
N-((trans)-2-phenylcyclopropyl)pyrrolidin-3-amine;
N-((trans)-2-(3′-(trifluoromethyl)-[1,1′-biphenyl]-4-yl)cyclopropyl)pyrrolidin-3-amine;
N-((trans)-2-(4-(benzyloxy)phenyl)cyclopropyl)pyrrolidin-3-amine;
N-((trans)-2-phenylcyclopropyl)azetidin-3-amine;
N-((trans)-2-(3′-(trifluoromethyl)-[1,1′-biphenyl]-4-yl)cyclopropyl)azetidin-3-amine;
N-((trans)-2-(4-(benzyloxy)phenyl)cyclopropyl)azetidin-3-amine;
N-((trans)-2-phenylcyclopropyl)azepan-3-amine;
N-((trans)-2-phenylcyclopropyl)-8-azabicyclo[3.2.1]octan-3-amine;
N-((trans)-2-phenylcyclopropyl)-3-azabicyclo[3.2.1]octan-8-amine;
N-((trans)-2-phenylcyclopropyl)decahydroquinolin-4-amine;
N-((trans)-2-phenylcyclopropyl)-1,2,3,4-tetrahydroquinolin-4-amine;
N-((trans)-2-phenylcyclopropyl)-3-azaspiro[5.5]undecan-9-amine;
N-((trans)-2-phenylcyclopropyl)-2-azaspiro[4.5]decan-8-amine;
N-((trans)-2-phenylcyclopropyl)-2,3-dihydrospiro[indene-1,4′-piperidin]-3-amine;
N-((1S,2R)-2-(4-(benzyloxy)phenyl)cyclopropyl)piperidin-4-amine;
N-((1R,2S)-2-(4-(benzyloxy)phenyl)cyclopropyl)piperidin-4-amine;
N-((1S,2R)-2-(pyridin-3-yl)cyclopropyl)piperidin-4-amine;
N-((1R,2S)-2-(pyridin-3-yl)cyclopropyl)piperidin-4-amine;
N-((1S,2S)-2-(thiazol-5-yl)cyclopropyl)piperidin-4-amine;
N-((1R,2R)-2-(thiazol-5-yl)cyclopropyl)piperidin-4-amine;
N-((1S,2R)-2-(3-(trifluoromethyl)-[1,1′-biphenyl]-4-yl)cyclopropyl)piperidin-4-amine;
N-((1R,2S)-2-(3-(trifluoromethyl)-[1,1′-biphenyl]-4-yl)cyclopropyl)piperidin-4-amine;
N-((trans)-2-phenylcyclopropyl)-7-azaspiro[3.5]nonan-2-amine;
N-(2-(o-tolyl)cyclopropyl)piperidin-4-amine;
N-(2-(2-fluorophenyl)cyclopropyl)piperidin-4-amine;
N-(2-(3,4-difluorophenyl)cyclopropyl)piperidin-4-amine;
N-(2-(4-methoxyphenyl)cyclopropyl)piperidin-4-amine;
N-(2-(naphthalen-2-yl)cyclopropyl)piperidin-4-amine;
N-(2-methyl-2-phenylcyclopropyl)piperidin-4-amine;
N-(6-methoxy-4′-((trans)-2-(piperidin-4-ylamino)cyclopropyl)-[1,1′-biphenyl]-3-yl)methanesulfonamide;
N-(4′-((trans)-2-(piperidin-4-ylamino)cyclopropyl)-[1,1′-biphenyl]-3-yl)propane-2-sulfonamide;
1-(methylsulfonyl)-N-((trans)-2-phenylcyclopropyl)piperidin-4-amine;
1-(4-(((trans)-2-(4-bromophenyl)cyclopropyl)amino)piperidin-1-yl)ethanone;
4-(((trans)-2-(4-bromophenyl)cyclopropyl)amino)piperidine-1-carboxamide;
N-((trans)-2-(4-bromophenyl)cyclopropyl)tetrahydro-2H-pyran-4-amine;
2,2,6,6-tetramethyl-N-((trans)-2-phenylcyclopropyl)piperidin-4-amine;
1-methyl-N-((trans)-2-phenylcyclopropyl)piperidin-4-amine;
1-isopropyl-N-((trans)-2-phenylcyclopropyl)piperidin-4-amine;
N-((trans)-2-phenylcyclopropyl)-1-(2,2,2-trifluoroethyl)piperidin-4-amine;
N-((trans)-2-phenylcyclopropyl)-1-(pyridin-4-yl)piperidin-4-amine;
4-(((trans)-2-(4-bromophenyl)cyclopropyl)amino)tetrahydro-2H-thiopyran 1,1-dioxide;
N-((trans)-2-fluoro-2-phenylcyclopropyl)piperidin-4-amine;
N-((1S,2S)-2-fluoro-2-phenylcyclopropyl)piperidin-4-amine;
N-((1R,2R)-2-fluoro-2-phenylcyclopropyl)piperidin-4-amine;
N-((trans)-2-(naphthalen-2-yl)cyclopropyl)piperidin-4-amine;
N-((trans)-2-methyl-2-phenylcyclopropyl)piperidin-4-amine;
N-((trans)-2-(o-tolyl)cyclopropyl)piperidin-4-amine;
N-((trans)-2-(2-fluorophenyl)cyclopropyl)piperidin-4-amine;
N-((trans)-2-(3,4-difluorophenyl)cyclopropyl)piperidin-4-amine;
N-((trans)-2-(4-methoxyphenyl)cyclopropyl)piperidin-4-amine;
(Trans)-2-phenyl-N-(piperidin-4-ylmethyl)cyclopropanamine;
(Trans)-2-phenyl-N-(2-(piperidin-4-yl)ethyl)cyclopropanamine;
(Trans)-2-phenyl-N-(2-(tetrahydro-2H-pyran-4-yl)ethyl)cyclopropanamine;
(Trans)-2-(4′-chloro-[, 1′-biphenyl]-4-yl)-N-(2-(tetrahydro-2H-pyran-4-yl)ethyl)cyclopropanamine;
(Trans)-N-(piperidin-4-ylmethyl)-2-(pyridin-3-yl)cyclopropanamine;
(Trans)-N-(piperidin-4-ylmethyl)-2-(thiazol-5-yl)cyclopropanamine;
(Trans)-N-(piperidin-4-ylmethyl)-2-(3-(trifluoromethyl)-[1,1′-biphenyl]-4-yl)cyclopropanamine;
(Trans)-2-(4-(benzyloxy)phenyl)-N-(piperidin-4-ylmethyl)cyclopropanamine;
(Trans)-N-(2-(piperidin-4-yl)ethyl)-2-(pyridin-3-yl)cyclopropanamine;
(Trans)-N-(2-(piperidin-4-yl)ethyl)-2-(thiazol-5-yl)cyclopropanamine;
(Trans)-N-(2-(piperidin-4-yl)ethyl)-2-(3-(trifluoromethyl)-[1,1′-biphenyl]-4-yl)cyclopropanamine;
(Trans)-2-(4-(benzyloxy)phenyl)-N-(2-(piperidin-4-yl)ethyl)cyclopropanamine;
(1S,2R)-2-phenyl-N-(piperidin-4-ylmethyl)cyclopropanamine;
(1R,2S)-2-phenyl-N-(piperidin-4-ylmethyl)cyclopropanamine;
(1S,2R)-2-phenyl-N-(2-(piperidin-4-yl)ethyl)cyclopropanamine;
(1R,2S)-2-phenyl-N-(2-(piperidin-4-yl)ethyl)cyclopropanamine;
(1S,2R)—N-(piperidin-4-ylmethyl)-2-(pyridin-3-yl)cyclopropanamine;
(1R,2S)—N-(piperidin-4-ylmethyl)-2-(pyridin-3-yl)cyclopropanamine;
(1S,2S)—N-(piperidin-4-ylmethyl)-2-(thiazol-5-yl)cyclopropanamine;
(1R,2R)—N-(piperidin-4-ylmethyl)-2-(thiazol-5-yl)cyclopropanamine;
(1S,2R)—N-(piperidin-4-ylmethyl)-2-(3′-(trifluoromethyl)-[1,1′-biphenyl]-4-yl)cyclopropanamine;
(1R,2S)—N-(piperidin-4-ylmethyl)-2-(3-(trifluoromethyl)-[1,1′-biphenyl]-4-yl)cyclopropanamine;
(1S,2R)-2-(4-(benzyloxy)phenyl)-N-(piperidin-4-ylmethyl)cyclopropanamine;
(1R,2S)-2-(4-(benzyloxy)phenyl)-N-(piperidin-4-ylmethyl)cyclopropanamine;
(1S,2R)—N-(2-(piperidin-4-yl)ethyl)-2-(pyridin-3-yl)cyclopropanamine;
(1R,2S)—N-(2-(piperidin-4-yl)ethyl)-2-(pyridin-3-yl)cyclopropanamine;
(1S,2S)—N-(2-(piperidin-4-yl)ethyl)-2-(thiazol-5-yl)cyclopropanamine;
(1R,2R)—N-(2-(piperidin-4-yl)ethyl)-2-(thiazol-5-yl)cyclopropanamine;
(1S,2R)—N-(2-(piperidin-4-yl)ethyl)-2-(3′-(trifluoromethyl)-[1,1′-biphenyl]-4-yl)cyclopropanamine;
(1R,2S)—N-(2-(piperidin-4-yl)ethyl)-2-(3′-(trifluoromethyl)-[1,1′-biphenyl]-4-yl)cyclopropanamine;
(1S,2R)-2-(4-(benzyloxy)phenyl)-N-(2-(piperidin-4-yl)ethyl)cyclopropanamine;
(1R,2S)-2-(4-(benzyloxy)phenyl)-N-(2-(piperidin-4-yl)ethyl)cyclopropanamine;
(Trans)-2-phenyl-N-(pyrrolidin-3-ylmethyl)cyclopropanamine;
(Trans)-2-(4-((2-fluorobenzyl)oxy)phenyl)-N-(piperidin-4-yl methyl)cyclopropanamine;
(Trans)-N-(azetidin-3-ylmethyl)-2-phenylcyclopropanamine;
(Trans)-2-(4-cyclopropylphenyl)-N-(piperidin-4-ylmethyl)cyclopropanamine;
(Trans)-N-(piperidin-4-yl methyl)-2-(4-(pyridin-3-yl)phenyl)cyclopropanamine;
(Trans)-2-(4-(1H-pyrazol-5-yl)phenyl)-N-(piperidin-4-ylmethyl)cyclopropanamine;
(Trans)-2-(naphthalen-2-yl)-N-(piperidin-4-ylmethyl)cyclopropanamine;
2-methyl-2-phenyl-N-(piperidin-4-ylmethyl)cyclopropanamine;
(trans)-2-methyl-2-phenyl-N-(piperidin-4-ylmethyl)cyclopropanamine;
(trans)-2-(4-(benzyloxy)phenyl)-N-((1-methylpiperidin-4-yl)methyl)cyclopropanamine;
   and pharmaceutically acceptable salts thereof.

Still more preferably, the compound of formula (X) is a compound from the list below:
N-((trans)-2-phenylcyclopropyl)piperidin-4-amine;
N-((1S,2R)-2-phenylcyclopropyl)piperidin-4-amine;
N-((1R,2S)-2-phenylcyclopropyl)piperidin-4-amine;
N-((trans)-2-(4-(benzyloxy)phenyl)cyclopropyl)piperidin-4-amine;
N-((trans)-2-(6-(3-(trifluoromethyl)phenyl)pyridin-3-yl)cyclopropyl)tetrahydro-2H-pyran-4-amine;
N-((trans)-2-(pyridin-3-yl)cyclopropyl)piperidin-4-amine;
N-((trans)-2-(thiazol-5-yl)cyclopropyl)piperidin-4-amine;
N-((trans)-2-(3′-(trifluoromethyl)-[1,1′-biphenyl]-4-yl)cyclopropyl)piperidin-4-amine;
N-((trans)-2-phenylcyclopropyl)piperidin-3-amine;
N-((trans)-2-(3′-(trifluoromethyl)-[1,1′-biphenyl]-4-yl)cyclopropyl)piperidin-3-amine;
N-((trans)-2-(4-(benzyloxy)phenyl)cyclopropyl)piperidin-3-amine;
N-((trans)-2-phenylcyclopropyl)pyrrolidin-3-amine;
N-((trans)-2-(3′-(trifluoromethyl)-[1,1′-biphenyl]-4-yl)cyclopropyl)pyrrolidin-3-amine;
N-((trans)-2-(4-(benzyloxy)phenyl)cyclopropyl)pyrrolidin-3-amine;
N-((trans)-2-phenylcyclopropyl)azetidin-3-amine;
N-((trans)-2-(3′-(trifluoromethyl)-[1,1′-biphenyl]-4-yl)cyclopropyl)azetidin-3-amine;
N-((trans)-2-(4-(benzyloxy)phenyl)cyclopropyl)azetidin-3-amine;
N-((trans)-2-phenylcyclopropyl)azepan-3-amine;
N-((trans)-2-phenylcyclopropyl)-8-azabicyclo[3.2.1]octan-3-amine;
N-((trans)-2-phenylcyclopropyl)-3-azabicyclo[3.2.1]octan-8-amine;
N-((trans)-2-phenylcyclopropyl)decahydroquinolin-4-amine;
N-((trans)-2-phenylcyclopropyl)-1,2,3,4-tetrahydroquinolin-4-amine;
N-((trans)-2-phenylcyclopropyl)-3-azaspiro[5.5]undecan-9-amine;
N-((trans)-2-phenylcyclopropyl)-2-azaspiro[4.5]decan-8-amine;
N-((trans)-2-phenylcyclopropyl)-2,3-dihydrospiro[indene-1,4′-piperidin]-3-amine;
N-((1S,2R)-2-(4-(benzyloxy)phenyl)cyclopropyl)piperidin-4-amine;
N-((1R,2S)-2-(4-(benzyloxy)phenyl)cyclopropyl)piperidin-4-amine;
N-((1S,2R)-2-(pyridin-3-yl)cyclopropyl)piperidin-4-amine;
N-((1R,2S)-2-(pyridin-3-yl)cyclopropyl)piperidin-4-amine;
N-((1S,2S)-2-(thiazol-5-yl)cyclopropyl)piperidin-4-amine;
N-((1R,2R)-2-(thiazol-5-yl)cyclopropyl)piperidin-4-amine;
N-((1S,2R)-2-(3′-(trifluoromethyl)-[1,1′-biphenyl]-4-yl)cyclopropyl)piperidin-4-amine;
N-((1R,2S)-2-(3′-(trifluoromethyl)-[1,1′-biphenyl]-4-yl)cyclopropyl)piperidin-4-amine;
N-((trans)-2-phenylcyclopropyl)-7-azaspiro[3.5]nonan-2-amine;
N-(2-(o-tolyl)cyclopropyl)piperidin-4-amine;
N-(2-(2-fluorophenyl)cyclopropyl)piperidin-4-amine;
N-(2-(3,4-difluorophenyl)cyclopropyl)piperidin-4-amine;
N-(2-(4-methoxyphenyl)cyclopropyl)piperidin-4-amine;
N-(2-(naphthalen-2-yl)cyclopropyl)piperidin-4-amine;
N-(2-methyl-2-phenylcyclopropyl)piperidin-4-amine;
N-(6-methoxy-4′-((trans)-2-(piperidin-4-ylamino)cyclopropyl)-[1,1′-biphenyl]-3-yl)methanesulfonamide;
N-(4′-((trans)-2-(piperidin-4-ylamino)cyclopropyl)-[1,1′-biphenyl]-3-yl)propane-2-sulfonamide;
1-(methylsulfonyl)-N-((trans)-2-phenylcyclopropyl)piperidin-4-amine;
1-(4-(((trans)-2-(4-bromophenyl)cyclopropyl)amino)piperidin-1-yl)ethanone;
4-(((trans)-2-(4-bromophenyl)cyclopropyl)amino)piperidine-1-carboxamide;
N-((trans)-2-(4-bromophenyl)cyclopropyl)tetrahydro-2H-pyran-4-amine;
2,2,6,6-tetramethyl-N-((trans)-2-phenylcyclopropyl)piperidin-4-amine;
1-methyl-N-((trans)-2-phenylcyclopropyl)piperidin-4-amine;
1-isopropyl-N-((trans)-2-phenylcyclopropyl)piperidin-4-amine;
N-((trans)-2-phenylcyclopropyl)-1-(2,2,2-trifluoroethyl)piperidin-4-amine;
N-((trans)-2-phenylcyclopropyl)-1-(pyridin-4-yl)piperidin-4-amine;
4-(((trans)-2-(4-bromophenyl)cyclopropyl)amino)tetrahydro-2H-thiopyran 1,1-dioxide;
N-((trans)-2-fluoro-2-phenylcyclopropyl)piperidin-4-amine;
N-((1S,2S)-2-fluoro-2-phenylcyclopropyl)piperidin-4-amine;
N-((1R,2R)-2-fluoro-2-phenylcyclopropyl)piperidin-4-amine;
N-((trans)-2-(naphthalen-2-yl)cyclopropyl)piperidin-4-amine;
N-((trans)-2-methyl-2-phenylcyclopropyl)piperidin-4-amine;
N-((trans)-2-(o-tolyl)cyclopropyl)piperidin-4-amine;
N-((trans)-2-(2-fluorophenyl)cyclopropyl)piperidin-4-amine;
N-((trans)-2-(3,4-difluorophenyl)cyclopropyl)piperidin-4-amine;
N-((trans)-2-(4-methoxyphenyl)cyclopropyl)piperidin-4-amine;
   or a pharmaceutically acceptable salt thereof.

Compounds of formula (X) can be prepared by the methods disclosed in WO2013/057320, the disclosure of which is incorporated by reference herein in its entirety.

In the methods and uses according to the invention, the LSD1 inhibitor can be a compound of formula (XI) or an enantiomer, a diastereomer or a mixture of stereoisomers (such as a racemic mixture or a diastereomer mixture) thereof, or a pharmaceutically acceptable salt or solvate thereof:

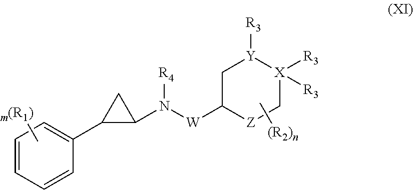

wherein
R₁ is selected from the group consisting of: C₁-C₆alkyl, —NSO₂Me, —NSO₂Ph, arylalkoxy, C₃-C₇cycloalkyl, —NC(O)Rₐ, 1-methyl-1H-pyrazol-4-yl, hydroxyl, C₁-C₄alkoxy, halogen, amide, amino, substituted amino, and —C(O)ORₐ;
R₂ is hydrogen or COOH;
each R₃ is independently selected from the group consisting of: aryl, heteoaryl, hydrogen, C₁-C₆alkyl, —SO₂Rₐ, —NC(O)Rₐ, —CH₂C(O)ORₐ, —C(O)ORₐ, —C(O)Rₐ, —C(O)NRₐR_{b}, substituted amino, amino, urea, amide, sulfonamide, arylalkyl, and heteroarylalkyl;
each Rₐ is independently hydrogen, phenyl, phenylmethyl, 3,5-dimethylisoxazol-4-yl, 1,2-dimethyl-1H-imidazol-4-yl, C₃-C₇cycloalkyl, C₁-C₆alkyl, C₁-C₄alkoxy, C₁-C₃alkylamino, or —NHPh;
R_{b} is hydrogen or C₁-C₃alkyl, or when attached to the same atom; or
Rₐ and R_{b} together form a 5- or 6-membered hetercycloalkyl ring;
R₄ is C₁-C₄alkyl, acyl, —C(O)CF₃ or hydrogen;
W is —(CH₂)₁₋₄, or —CH(R_{c})(CH₂)₀₋₃, in which R_{c} is CN or C₁-C₄alkyl;

### Y is N or C;

### X is N or C;

Z is O or (CH₂)_{q}, wherein q is 0-2, when q is 0, Z represents a bond;
m is 0-3, n is 0-3;
provided that when Z is O, Y is N and X is C;
also provided that when X is C, at least one of the R₃ groups attached to X is not hydrogen. Compounds of formula (XI) can be prepared by the methods disclosed in WO2012/135113, the disclosure of which is incorporated by reference herein in its entirety.

Preferably, the compound of formula (XI) is a compound from examples 1 to 150 in WO2012/135113 or a pharmaceutically acceptable salt thereof. Still more preferably, the compound of formula (XI) is 4-((4-((((1R,2S)-2-phenylcyclopropyl)amino)methyl)piperidin-1-yl)methyl)benzoic acid or a pharmaceutically acceptable salt thereof.

In the methods and uses according to the invention, the LSD1 inhibitor can be a compound of formula (XII) or an enantiomer, a diastereomer or a mixture of stereoisomers (such as a racemic mixture or a diastereomer mixture) thereof, or a pharmaceutically acceptable salt or solvate thereof:

A compound represented by the formula (XII):

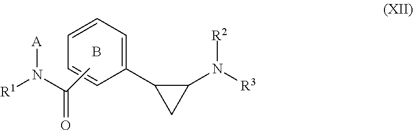

wherein A is a hydrocarbon group optionally having substituent(s), or a heterocyclic group optionally having substituent(s);
B is a benzene ring optionally having further substituent(s);
R¹, R² and R³ are each independently a hydrogen atom, a hydrocarbon group optionally having substituent(s), or a heterocyclic group optionally having substituent(s);
A and R¹ are optionally bonded to each other to form, together with the adjacent nitrogen atom, a cyclic group optionally having substituent(s); and
R² and R³ are optionally bonded to each other to form, together with the adjacent nitrogen atom, a cyclic group optionally having substituent(s).

Compounds of formula (XII) can be prepared by the methods disclosed in WO2014/058071, the disclosure of which is incorporated by reference herein in its entirety.

Preferably, the compound of formula (XII) is a compound from examples 1 to 273 in WO2014/058071 or a pharmaceutically acceptable salt thereof. More preferably, the compound of formula (XII) is 3-(trans-2-((cyclopropylmethyl)amino)cyclopropyl)-N-(5-methyl-1,2-oxazol-3-yl)benzamide, 3-(trans-2-((1-cyclopropylpiperidin-4-yl)amino)cyclopropyl)-N-(5-methyl-1,3,4-thiadiazol-2-yl)benzamide, 3-(trans-2-((cyclobutylamino)cyclopropyl)-N-(tetrahydro-2H-pyran-4-yl)benzamide, or a salt thereof.

In the methods and uses according to the invention, the LSD1 inhibitor can be a compound of formula (XIII) or an enantiomer, a diastereomer or a mixture of stereoisomers (such as a racemic mixture or a diastereomer mixture) thereof, or a pharmaceutically acceptable salt or solvate thereof:

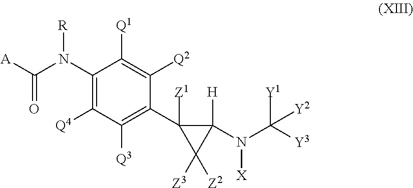

wherein A is a hydrocarbon group optionally having substituent(s), or a heterocyclic group optionally having substituent(s);
R is a hydrogen atom, a hydrocarbon group optionally having substituent(s), or a heterocyclic group optionally having substituent(s); or
A and R are optionally bonded to each other to form a ring optionally having substituent(s);
Q¹, Q², Q³ and Q⁴ are each independently a hydrogen atom or a substituent; Q¹ and Q², and Q³ and Q⁴, are
each optionally bonded to each other to form a ring optionally having substituent(s);
X is a hydrogen atom, an acyclic hydrocarbon group optionally having substituent(s), or a saturated cyclic group optionally having substituent(s);
Y¹, Y² and Y³ are each independently a hydrogen atom, a hydrocarbon group optionally having substituent(s), or a heterocyclic group optionally having substituent(s);
X and Y¹, and Y¹ and Y², are each optionally bonded to each other to form a ring optionally having substituent(s); and
Z¹, Z² and Z³ are each independently a hydrogen atom or a substituent,

Compounds of formula (XIII) can be prepared by the methods disclosed in WO2013/022047, the disclosure of which is incorporated by reference herein in its entirety.

Preferably, the compound of formula (XIII) is a compound from examples 1 to 166 in WO2013/022047, or a pharmaceutically acceptable salt thereof. More preferably, the compound of formula (XIII) is N-(4-(trans-2-[(cyclopropylmethyl)amino]cyclopropyl)phenyl)biphenyl-4-carboxamide, N-(4-(trans-2-[(1-methylpiperidin-4-yl)amino]cyclopropyl)phenyl)-3-(trifluoromethyl)benzamide, N-(4-(trans-2-[(cyclopropylmethyl)amino]cyclopropyl)phenyl)-1H-pyrazole-4-carboxamide, or a salt thereof.

Preferably, the LSD1 inhibitor to be used in the methods and therapeutic applications of the present invention is a selective LSD1 inhibitor or a dual LSD1/MAO-B inhibitor.

As used herein, a selective LSD1 inhibitor is a compound that inhibits LSD1 and has an IC50 value for LSD1 which is at least two-fold lower (i.e. more potent) than the IC50 value for MAO-A and MAO-B. More preferably, a selective LSD1 inhibitor has an IC50 value for LSD1 which is at least five-fold lower than the IC50 value for MAO-A and MAO-B. Even more preferably, selective LSD1 inhibitor have IC50 values for LSD1 which are at least ten-fold lower than the IC50 value for MAO-A and MAO-B.

As used herein, a dual LSD1/MAO-B inhibitor is a compound that inhibits LSD1 and MAO-B and has IC50 values for LSD1 and MAO-B which are at least two-fold lower (i.e. more potent) than the IC50 value for MAO-A. More preferably, dual LSD1/MAO-B inhibitors have IC50 values for LSD1 and MAO-B which are at least five-fold lower than the IC50 value for MAO-A. Even more preferably, dual LSD1/MAO-B inhibitors have IC50 values for LSD1 and MAO-B which are at least ten-fold lower than the IC50 value for MAO-A.

The ability of a compound to inhibit LSD1, MAO-A and MAO-B and its IC50 values for LSD1, MAO-A and MAO-B can be determined in accordance with the methods described in Example 1.

Preferred LSD1 inhibitors for use in the methods of the invention are the compounds of formulae (I) to (XIII), preferably the compounds of formulae (III), (VI), (VIII), (IX), (X), (XI), (XII) and (XIII), more preferably the compounds recited in the lists of examples provided above for compounds of formulae (Ill), (VI), (VIII), (IX), (X) and (XI), and still more preferably the compounds recited in the lists of examples provided above for compounds of formulae (VIII), (IX), (X) and (XI).

A particularly preferred LSD1 inhibitor for use in the methods of the invention is (−) 5-((((trans)-2-(4-(benzyloxy)phenyl)cyclopropyl)amino)methyl)-1,3,4-oxadiazol-2-amine or a pharmaceutically acceptable salt or solvate thereof.

Other preferred LSD1 inhibitors for use in the methods of the invention are:
(trans)-N1-((1R,2S)-2-phenylcyclopropyl)cyclohexane-1,4-diamine, or
4-((4-((((1R,2S)-2-phenylcyclopropyl)amino)methyl)piperidin-1-yl)methyl)benzoic acid,
   or a pharmaceutically acceptable salt or solvate thereof.

While it is possible that an active compound, i.e. the LSD1 inhibitor, may be administered for use in therapy directly as such, it is typically administered in the form of a pharmaceutical composition, which comprises said compound as active pharmaceutical ingredient together with one or more pharmaceutically acceptable excipients or carriers.

The active compounds may be administered by any means that accomplish their intended purpose. Examples include administration by the oral, parenteral, intravenous, subcutaneous or topical routes.

For oral delivery, the active compounds can be incorporated into a formulation that includes pharmaceutically acceptable carriers such as binders (e.g., gelatin, cellulose, gum tragacanth), excipients (e.g., starch, lactose), lubricants (e.g., magnesium stearate, silicon dioxide), disintegrating agents (e.g., alginate, Primogel, and corn starch), and sweetening or flavoring agents (e.g., glucose, sucrose, saccharin, methyl salicylate, and peppermint). The formulation can be orally delivered in the form of enclosed gelatin capsules or compressed tablets. Capsules and tablets can be prepared in any conventional techniques. The capsules and tablets can also be coated with various coatings known in the art to modify the flavors, tastes, colors, and shapes of the capsules and tablets. In addition, liquid carriers such as fatty oil can also be included in capsules.

Suitable oral formulations can also be in the form of suspension, syrup, chewing gum, wafer, elixir, and the like. If desired, conventional agents for modifying flavors, tastes, colors, and shapes of the special forms can also be included. In addition, for convenient administration by enteral feeding tube in patients unable to swallow, the active compounds can be dissolved in an acceptable lipophilic vegetable oil vehicle such as olive oil, corn oil and safflower oil.

The active compounds can also be administered parenterally in the form of solution or suspension, or in lyophilized form capable of conversion into a solution or suspension form before use. In such formulations, diluents or pharmaceutically acceptable carriers such as sterile water and physiological saline buffer can be used. Other conventional solvents, pH buffers, stabilizers, anti-bacteria agents, surfactants, and antioxidants can all be included. For example, useful components include sodium chloride, acetates, citrates or phosphates buffers, glycerin, dextrose, fixed oils, methyl parabens, polyethylene glycol, propylene glycol, sodium bisulfate, benzyl alcohol, ascorbic acid, and the like. The parenteral formulations can be stored in any conventional containers such as vials and ampoules.

Routes of topical administration include nasal, bucal, mucosal, rectal, or vaginal applications. For topical administration, the active compounds can be formulated into lotions, creams, ointments, gels, powders, pastes, sprays, suspensions, drops and aerosols. Thus, one or more thickening agents, humectants, and stabilizing agents can be included in the formulations. Examples of such agents include, but are not limited to, polyethylene glycol, sorbitol, xanthan gum, petrolatum, beeswax, or mineral oil, lanolin, squalene, and the like. A special form of topical administration is delivery by a transdermal patch. Methods for preparing transdermal patches are disclosed, e.g., in Brown, et al. (1988) Ann. Rev. Med. 39:221-229 which is incorporated herein by reference.

Subcutaneous implantation for sustained release of the active compounds may also be a suitable route of administration. This entails surgical procedures for implanting an active compound in any suitable formulation into a subcutaneous space, e.g., beneath the anterior abdominal wall. See, e.g., Wilson et al. (1984) J. Clin. Psych. 45:242-247. Hydrogels can be used as a carrier for the sustained release of the active compounds. Hydrogels are generally known in the art. They are typically made by crosslinking high molecular weight biocompatible polymers into a network, which swells in water to form a gel like material. Preferably, hydrogels are biodegradable or biosorbable. For purposes of this invention, hydrogels made of polyethylene glycols, collagen, or poly(glycolic-co-L-lactic acid) may be useful. See, e.g., Phillips et al. (1984) J. Pharmaceut. Sci., 73: 1718-1720.

The active compounds can also be conjugated, to a water soluble non-immunogenic non-peptidic high molecular weight polymer to form a polymer conjugate. For example, an active compound is covalently linked to polyethylene glycol to form a conjugate. Typically, such a conjugate exhibits improved solubility, stability, and reduced toxicity and immunogenicity. Thus, when administered to a patient, the active compound in the conjugate can have a longer half-life in the body, and exhibit better efficacy. See generally, Burnham (1994) Am. J. Hosp. Pharm. 15:210-218. PEGylated proteins are currently being used in protein replacement therapies and for other therapeutic uses. For example, PEGylated interferon (PEG-INTRON A®) is clinically used for treating Hepatitis B. PEGylated adenosine deaminase (ADAGEN®) is being used to treat severe combined immunodeficiency disease (SCIDS). PEGylated L-asparaginase (ONCAPSPAR®) is being used to treat acute lymphoblastic leukemia (ALL). It is preferred that the covalent linkage between the polymer and the active compound and/or the polymer itself is hydrolytically degradable under physiological conditions. Such conjugates known as “prodrugs” can readily release the active compound inside the body. Controlled release of an active compound can also be achieved by incorporating the active ingredient into microcapsules, nanocapsules, or hydrogels generally known in the art. Other pharmaceutically acceptable prodrugs of the compounds of this invention include, but are not limited to, esters, carbonates, thiocarbonates, N-acyl derivatives, N-acyloxyalkyl derivatives, quaternary derivatives of tertiary amines, N-Mannich bases, Schiff bases, amino acid conjugates, phosphate esters, metal salts and sulfonate esters.

Liposomes can also be used as carriers for the active compounds. Liposomes are micelles made of various lipids such as cholesterol, phospholipids, fatty acids, and derivatives thereof. Various modified lipids can also be used. Liposomes can reduce the toxicity of the active compounds, and increase their stability. Methods for preparing liposomal suspensions containing active ingredients therein are generally known in the art. See, e.g., U.S. Pat. No. 4,522,811; Prescott, Ed., Methods in Cell Biology, Volume XIV, Academic Press, New York, N.Y. (1976).

Unless otherwise stated, any description of a method of treatment includes use of the compounds to provide such treatment as is described herein, as well as use of the compounds to prepare a medicament to treat such disease.

In the herein disclosed therapeutic uses and methods of treatment, the LSD1i can also be administered in combination with another active agent that synergistically treats the same symptoms or is effective for another disease or symptom in the patient treated so long as the other active agent does not interfere with or adversely affect the effects of the active compounds of this invention. Such other active agents include but are not limited to anti-inflammatory agents, antibiotics, antifungal agents, antithrombotic agents, cardiovascular drugs, cholesterol lowering agents, anti-cancer drugs, hypertension drugs, and the like.

Combination therapy includes administration of a single pharmaceutical dosage formulation which contains an LSD1 inhibitor and one or more additional active agents, as well as administration of the LSD1 inhibitor and each additional active agent in its own separate pharmaceutical dosage formulation. If administered separately, the administration can be simultaneous, sequential or separate, and the LSD1i and the additional therapeutic agent(s) can be administered via the same administration route or using different administration routes, for example one compound can be administered orally and the other intravenously.

In another aspect, the invention relates to a combination comprising a LSD1 inhibitor and a S100A9 and/or S100A8 inhibitor. In a related aspect, the invention relates to a combination comprising a LSD1 inhibitor and an S100A9 and/or S100A8 inhibitor for use in the treatment of a disease characterized by S100A9 and/or S100A8 induction, such as the ones disclosed above. In a related aspect, the invention relates to a method for treating a disease characterized by S100A9 and/or S100A8 induction, such as the ones disclosed above, in a patient, comprising administering a combination comprising a LSD1 inhibitor and an S100A9 and/or S100A8 inhibitor. As used herein, a “S100A9 and/or S100A8 inhibitor” is an active agent (other than an LSD1 inhibitor) that either blocks S100A9 and/or S100A8 function or decreases S100A9 and/or S100A8 expression levels.

A non-limiting example of a S100A9 and/or S100A8 inhibitor is a corticosteroid. Corticosteroids have been described to downregulate S100A9 levels but are not recommended for long term treatment due to side effects. A combination comprising a corticosteroid and an LSD1 inhibitor may allow to reduce the dose of corticosteroid to be administered.

Another non-limiting example of a S100A9 and/or S100A8 inhibitor is paquinimod, tasquinimod, laquinimod and other related quinoline-3-carboxamides; these compounds have been reported to block S100A9 and/or S100A8 function by inhibiting the interaction between S100A9 and two types of pro-inflammatory receptors: Toll-like Receptor 4 (TLR4) and RAGE (receptor for advance glycation end products) (P Björk et al, PLoS Biol. 2009, 7(4), e1000097. doi:10.1371/journal.pbio.1000097). A combination comprising an LSD1 inhibitor, which reduces S100A9/A8 expression levels, and an agent that inhibits the interaction between S100A9 and/or S100A8 and TLR4 or RAGE such as paquinimod, tasquinimod, laquinimod and related compounds may allow to produce the desired therapeutic effects in the treatment of diseases characterized by S100A9 and/or S100A8 induction with reduced non-Si 00A9/A8-related side effects on both type of compounds.

Another non-limiting example of a S100A9 and/or S100A8 inhibitor is an S100A9 and/or S100A8 binding molecule such as an antiS100A9 or antiS100A8 antibody.

In another aspect, the invention relates to a combination comprising an LSD1 inhibitor and an antibacterial agent. In a related aspect, the invention relates to a combination comprising an LSD1 inhibitor and an antibacterial agent for use in the treatment of bacterial infections and diseases caused by bacterial infections, including the ones listed earlier. In a related aspect, the invention relates to a method for treating a bacterial infection or a disease caused by a bacterial infection, such as the ones disclosed above, in a patient, comprising administering a combination comprising a LSD1 inhibitor and an antibacterial agent. Any known antibacterial agent is suitable for use in the combinations of the invention, including: aminoglycosides such as amikacin, gentamicin, kanamycin, neomycin, netilmicin, tobramycin, paromomycin, streptomycin and spectinomycin; ansamycins such as rifaximin; carbapenems such as ertapenem, doripenem, imipenem, and meropenem; cephalosporins such as cefadroxil, cefazolin, cefalexin, cefaclor, cefprozil, cefuroxime, cefixime,cefdinir,cefditoren, cefotaxime, cefpodoxime, ceftazidime, ceftibuten, ceftriaxone, cefepime, ceftaroline fosamil, and ceftobiprole; glycopeptides such as teicoplanin, vancomycin, telavancin, dalbavancin, and oritavancin; lincosamides such as clindamycin and lincomycin; lipopeptides such as daptomycin; macrolides such as azithromycin, clarithromycin, erythromycin, roxithromycin, telithromycin and spiramycin; monobactams such as aztreonam; nitrofurans such as furazolidone and nitrofurantoin; oxazolidinones such as linezolid, posizolid, radezolid and torezolid; penicillins such as amoxicillin, azlocillin, flucloxacillin, penicillin G, amoxicillin/clavulanate, ampicillin/sulbactam, piperacillin/tazobactam and ticarcillin/clavulanate; polypeptides such as bacitracin, colistin, and polymyxin B; quinolones such as ciprofloxacin, enoxacin, gatifloxacin, gemifloxacin, levofloxacin, lomefloxacin, moxifloxacin, nalidixic acid, norfloxacin, and ofloxacin; sulfonamides such as mafenide, sulfacetamide, sulfadiazine, sulfadimethoxine, sulfamethizole, sulfamethoxazole, sulfasalazine, sulfisoxazole, trimethoprim-sufamethoxazole; tetracyclines such as demeclocycline, doxycycline, minocycline, oxytetracycline and tetracycline; and other antibacterial agents such as flofazimine, dapsone, capreomycin, cycloserine, ethambutol, ethionamide, isoniazid, pyrazinamide, rifampicin, rifabutin, rifapentine, streptomycin, chloramphenicol, fosfomycin, fusidic acid, metronidazole, mupirocin, platensimycin, quinupristin/dalfopristin, thiamphenicol, tigecycline, tinidazole and trimethoprim.

It is to be understood that the present invention specifically relates to each and every combination of features or embodiments described herein, including any combination of general and/or preferred features/embodiments. In particular, the invention specifically relates to all combinations of preferred features/embodiments (including all degrees of preference) of the methods and uses provided herein.

The following definitions apply throughout the present specification and claims, unless specifically indicated otherwise.

As used herein, the term “sample” refers to a sample obtained from a subject. The sample may be of any biological tissue, cell or fluid. Such samples include but are not limited to Cerebrospinal fluid, Blood, Plasma, Serum, Stool, Urine, Saliva, Sputum, Gingival crevicular fluid, Hair follicles and tissue biopsy (skin, liver, etc). A sample is preferably a peripheral sample.

The sample to be assessed in accordance with the present invention (i.e. samples whose level of a biomarker selected from S100A9 and S100A8 is to be determined), can be obtained from a subject or a patient as defined herein.

Non-limiting examples of peripheral samples from patients having an infection or an infectious disease include Cerebrospinal fluid (CSF), Blood, Plasma, Serum, Stool, Urine, Saliva, Sputum, and Gingival crevicular fluid. Non-limiting examples of peripheral samples from patients having an autoimmune disease include Cerebrospinal fluid (CSF), Blood, Plasma, Serum, Stool, Urine, Saliva, Sputum, Gingival crevicular fluid, skin biopsy and hair follicles. Non-limiting examples of peripheral samples from patients having cancer include Cerebrospinal fluid (CSF), Blood, Plasma, Serum, Stool, Urine, skin biopsy and hair follicles. Non-limiting examples of peripheral samples from patients having a cardiovascular disease include Blood, Plasma, and Serum. Non-limiting examples of peripheral samples from patients having a CNS disease include Cerebrospinal fluid (CSF), Blood, Plasma, and Serum.

A “patient” or “subject” for the purposes of the present invention includes both humans and other animals, particularly mammals, and other organisms. Thus, the methods are applicable to both human therapy and veterinary applications. In a preferred aspect the subject or patient is a mammal, and in the most preferred aspect the subject or patient is human.

As used herein a “subject” is an individual (preferably a human) from which samples are obtained for analysis of biomarker levels. Thus, the term “subject” encompasses both a healthy individual (like a healthy volunteer enrolled in clinical trials) and a patient.

A “patient” is a subject with a presymptomatic, prodromal, incipient, mild, severe, active, or dormant disease. As used herein “patient” also includes subjects identified to have a high risk for the development of a disease. As used herein, the term “a patient having a disease” refers to a patient suffering from a disease as defined herein, a patient suspected to suffer from a disease as defined herein or being prone to suffer from a disease as defined herein. A patient that is prone to suffer from a disease as defined herein refers to a patient that is at risk of developing a disease as defined herein.

As used in the methods for monitoring of the invention, a “decrease” in relation to the level of a biomarker means that the level of S100A9 and/or S100A8 in a test sample is lower than the level of the same biomarker in a control. Preferably, the decrease is a significant decrease. As used herein, a “significant decrease” of the biomarker level in a test sample means a decrease with a probability p<0.05 to fit the null hypothesis; i.e. that the biomarker levels measured before treatment have not varied after treatment (between and/or within subjects) but not excluding other comparisons (Fisher, 1925, Statistical Methods for Research Workers).

A non-limiting example of a “control” is a healthy control, which can be either samples obtained from healthy subjects, as well as samples obtained from biobanks and similar sources. A further non-limiting example of a “control” are data published in the scientific literature relating to such healthy subjects. As used herein, a “healthy subject” is a subject with matched age and gender as a patient and showing neither presymptomatic, prodromal, incipient, mild, severe, active, nor dormant disease nor a high risk for the development of the disease. A further non-limiting example of a “control” may be a sample obtained from a subject prior to the initiation of treatment with the LSD1 inhibitor. Prior to the initiation of treatment means that no LSD1 inhibitor has been administered to the subject at least 1 week, but preferentially 2 weeks prior to obtaining the control sample.

Preferentially, controls are of the same type as the sample to be compared with, and cover the expected range in that sample type.

As mentioned herein, the herein provided biomarkers can be used as monitoring biomarkers or as predictive biomarkers.

Thus, the biomarkers can be used to monitor the response to an LSD1 inhibitor after treatment has started (e.g. during treatment with an LSD1 inhibitor, encompassing treatment breaks).

The biomarkers can be used to predict the likeliness of response to an LSD1 inhibitor. It is known in the art that predictive factors indicate which therapy may be the most appropriate. It is therefore contemplated herein that the level of a biomarker which is S100A9 and/or S100A8 can be determined in a sample from a patient prior to treatment with the LSD1 inhibitor, i.e prior to (the start of) the treatment with the LSD1 inhibitor. In this context, the terms “predicting whether a patient is (likely) to respond to an LSD1 inhibitor comprising determining the level of a biomarker which is S100A9 and/or S100A8 in a sample from the patient” and “determining whether a patient is (likely) to respond to an LSD1 inhibitor comprising determining the level of a biomarker which is S100A9 and/or S100A8 in a sample from the patient prior to the treatment with the LSD1 inhibitor” can be used interchangeably herein. “Prior to the treatment” as used in this context can relate to a sample obtained from a patient that has never received a treatment with an LSD1 inhibitor (i.e. a “naive” patient), or from a patient that had previously been treated with an LSD1 inhibitor but is not receiving treatment with an LSD1 inhibitor at the time of taking the sample for the purpose of predicting his/her response to an LSD1 inhibitor and has not received treatment with an LSD1 inhibitor for at least 2 weeks before taking the sample.

As used herein, “response” means a variation in a relevant biological or clinical parameter; including the biomarker level in the sample vs control sample; a relevant analyte level analyzed in the subject pre and post treatment (p.e. inflammatory markers including cytokines); a relevant disease symptom; an observational test and the like. Preferably, the response is a significant variation of the biological or clinical parameter, meaning a change with a probability p<0.05 to fit the null hypothesis.

As used herein, “elevated” in relation to the level of a biomarker in a sample means that the level of the biomarker is increased above a threshold level. Threshold values or threshold levels can be determined following methods known in the art.

A non-limiting method to establish a threshold value is based on a control average biomarker level and grouped based on age, gender, ethnicity, analysis method and other variables that affect the biomarker levels. Then, a control population would be used as a reference population and the elevated threshold level can be defined as >mean biomarker level in the control subject population+X times the standard deviation of the biomarker level in the subject population. Most frequently, the control population will consist of healthy subjects. Alternatively, the control population can be a subgroup of patients with common symptoms but differential diagnosis from a second subgroup of patients characterized by an increased biomarker level.

Most pathologies show a progression usually accompanied by dynamic changes in biomarker levels, variations that can be used to differentiate subject populations in different disease stages. Therefore, in order to discriminate these populations, different threshold values could be determined using the above described method: i.e. >mean of the healthy subpopulation+2 times the standard deviation of the biomarker level in the healthy subject population. Threshold values will be adapted for age, gender, ethnicity and other variables that affect biomarker levels in a population. Threshold method levels are also adapted for variation in clinical diagnosis in follow-up analysis. Threshold levels are also adapted for technical variables in the analysis method.

Another method to determine threshold value can be established according to the distributions of control and diseased subject biomarker levels in a population diagnosed using a golden standard method. Receiver Operating Characteristic (ROC) curve analysis is performed to identify the optimal criterion for threshold value (https://www.medcalc.org/manuallroc-curves.php). Threshold values are chosen such that specificity is >85%, >90% or >95%. Threshold values are further chosen such that sensitivity is >80%, >85%, >90% or >95%. Threshold values are adapted for disease prevalence, age, gender, ethnicity and other variables that affect biomarker levels in a population. Threshold method levels are also adapted for variation in clinical diagnosis in follow-up analysis. Threshold levels are also adapted for technical variables in the analysis method.

A person skilled in the art appreciates that a positive test for the herein provided biomarkers S100A9 and/or S100A8 by the herein provided methods, does not necessarily translate 1:1 into a successful treatment. However, by these methods sub-groups of patients are identified that have a higher chance of response (=show a better response rate) to a treatment with a LSD1 inhibitor as compared to the sub-group of patients not showing these positive test results. With other words, a positive result indicates that the individual or patient has a higher chance to respond to treatment with a LSD1 inhibitor as compared to e.g. a patient having “normal” levels of S100A9 and/or S100A8 (like levels comparable to control levels). Thus, the individual or patient having an elevated level of the biomarkers S100A9 and/or S100A8 in a sample, is likely to respond to a treatment with an LSD1 inhibitor.

It is to be understood that the term “in vitro” is used in relation to methods in the sense of experiments, methods or procedures performed “outside of a living human or animal body”. Accordingly, as used herein “in vitro” encompasses ex-vivo.

The terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a patient and includes: (a) preventing a disease in a patient which may be predisposed/at risk of developing the disease; (b) inhibiting the disease, i.e. arresting its development; or (c) relieving the disease, i.e. causing regression of the disease. As used herein, the term “treating a disease” or “treatment of a disease” refers particularly to a slowing of or a reversal of the progress of the disease. Treating a disease includes treating a symptom and/or reducing the symptoms of the disease.

As used herein, the term “therapeutically effective amount”, such as the therapeutically effective amount of a compound of the present invention, refers to the amount sufficient to produce a desired biological effect (e.g., a therapeutic effect) in a subject. Accordingly, a therapeutically effective amount of a compound may be an amount which is sufficient to treat a disease, and/or delay the onset or progression of a disease, and/or alleviate one or more symptoms of the disease, when administered to a subject suffering from or susceptible to that disease.

As used herein, a “pharmaceutically acceptable salt” is intended to mean a salt that retains the biological effectiveness of the free acids and bases of the specified compound and that is not biologically or otherwise undesirable. A compound for use in the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid, such as hydrochlorides, hydrobromides, sulfates, pyrosulfates, bisulfates, sulfites, bisulfites, phosphates, monohydrophosphates, dihydrophosphates, metaphosphates, pyrophosphates, chlorides, bromides, iodides, nitrates, acetates, propionates, decanoates, caprylates, acrylates, formates, isobutyrates, caproates, heptanoates, propiolates, oxalates, malonates, succinates, suberates, sebacates, fumarates, maleates, butyne-1,4 dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, gamma-hydroxybutyrates, glycollates, tartrates, methane-sulfonates, ethane-sulfonates, propanesulfonates, benzenesulfonates, toluenesulfonates, trifluoromethansulfonates, naphthalene-1-sulfonates, naphthalene-2-sulfonates, mandelates, pyruvates, stearates, ascorbates, or salicylates. When the compounds of the invention carry an acidic moiety, suitable pharmaceutically acceptable salts thereof may include alkali metal salts, e.g. sodium or potassium salts; alkaline earth metal salts, e.g. calcium or magnesium salts; and salts formed with suitable organic ligands such as ammonia, alkylamines, hydroxyalkylamines, lysine, arginine, N-methylglucamine, procaine and the like. Pharmaceutically acceptable salts are well known in the art.

As used herein, a “pharmaceutically acceptable solvate” refers to a complex of variable stoichiometry formed by a solute (like a compound of formula I to XIII or a salt thereof) and a pharmaceutically acceptable solvent such as water, ethanol and the like. A complex with water is known as a hydrate.

As used herein, a “pharmaceutically acceptable carrier” or “pharmaceutically acceptable excipient” refers to a non-API (API refers to Active Pharmaceutical Ingredient) substances such as disintegrators, binders, fillers, and lubricants used in formulating pharmaceutical products. They are generally safe for administering to humans according to established governmental standards, including those promulgated by the United States Food and Drug Administration and the European Medical Agency. Pharmaceutically acceptable carriers or excipients are well known to those skilled in the art.

In the definitions of LSD1i provided above, particularly in the definitions of compounds of formula (I) to (XIII), the following definitions apply, when applicable:

Any definition herein may be used in combination with any other definition to describe a composite structural group. By convention, the trailing element of any such definition is that which attaches to the parent moiety. For example, the composite group cyclylC₁₋₈ alkyl would represent a cyclyl group attached to the parent molecule through a C₁₋₈ alkyl group.

As used herein, the term “acyl” refers to a carbonyl attached to an alkenyl, alkyl, aryl, cycloalkyl, heteroaryl, heterocyclyl, or any other moiety where the atom attached to the carbonyl is carbon. Preferably, the term “acyl” refers to a group of formula —C(═O)R″, wherein R″ represents alkenyl, alkyl, aryl, cycloalkyl, heteroaryl or heterocyclyl. An “acetyl” group refers to a —C(═O)CH₃ group. An “alkylcarbonyl” or “alkanoyl” group refers to an alkyl group attached to the parent molecular moiety through a carbonyl group. Examples of such groups include, but are not limited to, methylcarbonyl or ethylcarbonyl. Examples of acyl groups include, but are not limited to, formyl, alkanoyl or aroyl.

As used herein, the term “alkenyl” refers to a straight-chain or branched-chain hydrocarbon group having one or more double bonds and containing from 2 to 20 carbon atoms. A C₂₋₈ alkenyl is an alkenyl group having from 2 to 8 carbon atoms.

As used herein, the term “alkoxy” refers to an alkyl ether group (ie a group of formula alkyl-O—), wherein the term alkyl is as defined below. Examples of suitable alkyl ether groups include, but are not limited to, methoxy, ethoxy, n-propoxy, isopropoxy, n-butoxy, iso-butoxy, sec-butoxy, tert-butoxy, or n-pentoxy. The term C_{1-z} alkoxy refers to an alkoxy group wherein the alkyl moiety has from 1 to z carbon atoms; for example a C₁₋₈ alkoxy is an alkoxy group wherein the alkyl moiety is C₁₋₈ alkyl, i.e. a group of formula C₁₋₈ alkyl-O—.

As used herein, the term “alkyl” refers to a straight-chain or branched-chain alkyl group containing from 1 to 20 carbon atoms. A C_{1-z} alkyl is an alkyl from 1 to z carbon atoms; thus, a C_{1-z} alkyl has from 1 to 8 carbon atoms, a C₁₋₄ alkyl has from 1 to 4 carbon atoms and a C₁₋₂ alkyl has from 1 to 2 carbon atoms. Examples of alkyl groups include, but are not limited to, methyl, ethyl, n-propyl, isopropyl, n-butyl, isobutyl, sec-butyl, tert-butyl, pentyl, isopentyl, neo-pentyl, iso-amyl, hexyl, heptyl, octyl, or nonyl.

As used herein, the term “C₁₋₄ alkylene” refers to an C₁₋₄ alkyl group attached at two positions, i.e. an alkanediyl group. Examples include, but are not limited to, methylene (i.e. a group of formula —CH₂—), ethylene (including ethane-1,2-diyl and ethane-1,1-diyl), propylene (e.g. propane-1,3-diyl, propane-1,2-diyl and propane-1,1-diyl) and butylene (e.g. butane-1,4-diyl, butane-1,3-diyl or butane-1,1-diyl). Accordingly, the term “C₁₋₄ alkylene” may refer to a straight-chain or branched-chain alkylene group having from 1 to 4 carbon atoms. A “linear C₁₋₄ alkylene” refers to a straight chain alkylene group having from 1 to 4 carbon atoms, i.e. a —(CH₂)_{y}— group wherein y is 1, 2, 3 or 4.

As used herein, the term “alkylamino,” refers to an alkyl group attached to the parent molecular moiety through an amino group. Suitable alkylamino groups may be mono- or dialkylated, forming groups including, but not limited to N-methylamino, N-ethylamino, N,N-dimethylamino, N,N-ethylmethylamino, N,N-diethylamino, N-propylamino, and N,N-methylpropylamino.

As used herein, the term “alkynyl” refers to a straight-chain or branched-chain hydrocarbon group having one or more triple bonds and containing from 2 to 20 carbon atoms. A C₂₋₈ alkynyl has from 2 to 8 carbon atoms. Examples of alkynyl groups include, but are not limited to, ethynyl, propynyl, hydroxypropynyl, butyn-1-yl, butyn-2-yl, pentyn-1-yl, 3-methylbutyn-1-yl, or hexyn-2-yl.

As used herein, the term “amido” and “carbamoyl” refers to an amino group as described below attached to the parent molecular moiety through a carbonyl group (e.g., —C(═O)NRR′), or vice versa (—N(R)C(═O)R′). “Amido” and “carbamoyl” encompasses “C-amido” and “N-amido” as defined herein. R and R′ are as defined herein.

As used herein, the term “C-amido” refers to a —C(═O)NRR′ group with R and R′ as defined herein.

As used herein, the term “N-amido” refers to a —N(R)C(═O)R′ group with R and R′ as defined herein.

As used herein, the term “amino” refers to —NRR′, wherein R and R′ are independently selected from the group consisting of hydrogen, alkyl, heteroalkyl, aryl, carbocyclyl, and heterocyclyl. Additionally, R and R′ may be combined to form a heterocyclyl. Exemplary “amino” groups include, without being limited thereto, —NH₂, —NH(C₁₋₄ alkyl) and —N(C₁₋₄ alkyl)(C₁₋₄ alkyl).

As used herein, the term “aryl” refers to a carbocyclic aromatic system containing one ring, or two or three rings fused together where in the ring atoms are all carbon. The term “aryl” includes, but is not limited to groups such as phenyl, naphthyl, or anthracenyl. The term “monocyclic aryl” refers to phenyl.

As used herein, the term “arylalkoxy” or “aralkoxy,” refers to an aryl group attached to the parent molecular moiety through an alkoxy group. Examples of arylalkoxy groups include, but are not limited to, benzyloxy or phenethoxy.

As used herein, the term “arylalkyl” or “aralkyl,” refers to an aryl group attached to the parent molecular moiety through an alkyl group.

As used herein, the term “aryloxy” refers to an aryl group attached to the parent molecular moiety through an oxy (—O—).

As used herein, the term “carbamate” refers to an O-carbamyl or N-carbamyl group as defined herein. An N-carbamyl group refers to —NR—COOR′, wherein R and R′ are as defined herein. An O-carbamyl group refers to —OCO—NRR′, wherein R and R′ are as defined herein.

As used herein, the term “carbonyl” when alone includes formyl —C(═O)H and in combination is a —C(═O)— group.

As used herein, the term “carboxyl” or “carboxy” refers to —C(═O)OH or the corresponding “carboxylate” anion, such as is in a carboxylic acid salt.

An “O-carboxy” group refers to a RC(═O)O— group, where R is as defined herein.

A “C-carboxy” group refers to a —C(═O)OR groups where R is as defined herein.

As used herein, the term “cyano” refers to —CN.

As used herein, the term “carbocyclyl” refers to a saturated or partially saturated monocyclic or a fused bicyclic or tricyclic group wherein the ring atoms of the cyclic system are all carbon and wherein each cyclic moiety contains from 3 to 12 carbon atom ring members. “Carbocyclyl” encompasses benzo fused to a carbocyclyl ring system. One group of carbocyclyls have from 5 to 7 carbon atoms. Examples of carbocyclyl groups include, but are not limited to, cyclopropyl, cyclobutyl, cyclopentyl, cyclohexyl, cycloheptyl, tetrahydronaphthyl, indanyl, octahydronaphthyl, 2,3-dihydro-1H-indenyl, or adamantyl.

As used herein, the term “cycloalkyl”, unless otherwise specified, refers to a saturated monocyclic, bicyclic or tricyclic group wherein the ring atoms of the cyclic system are all carbon and wherein each cyclic moiety contains from 3 to 12 carbon atom ring members. A C₃₋₆ cycloalkyl is a cycloalkyl that has from 3 to 6 carbon atoms, i.e. cyclopropyl, cyclobutyl, cyclopentyl and cyclohexyl. A cycloalkyl containing from 4 to 7 C atoms includes cyclobutyl, cyclopentyl, cyclohexyl and cycloheptyl. Examples of cycloalkyl groups include, but are not limited to, cyclopropyl, cyclobutyl, cyclopentyl, cyclohexyl, cycloheptyl, or adamantyl.

As used herein, the term “cycloalkenyl” refers to a partially saturated monocyclic, bicyclic or tricyclic group wherein the ring atoms of the cyclic system are all carbon and wherein each cyclic moiety contains from 3 to 12 carbon atom ring members. One group of carboalkenyls have from 5 to 7 carbon atoms. Examples of cycloalkenyl groups include, but are not limited to, cyclobutenyl, cyclopentenyl, or cyclohexenyl.

As used herein, the term “cyclyl” refers to an aryl, heterocyclyl, or carbocyclyl group as defined herein.

As used herein, the term “cyclylC₁₋₈ alkyl” refers to a C₁₋₈ alkyl as defined above wherein one hydrogen atom in the C₁₋₈ alkyl group has been replaced with one cyclyl group as defined above.

As used herein, the term “halo” or “halogen” refers to fluorine, chlorine, bromine, or iodine.

As used herein, the term “haloalkoxy” refers to a haloalkyl group (as defined below) attached to the parent molecular moiety through an oxygen atom. A haloC₁₋₈ alkoxy group refers to a haloalkoxy group wherein the haloalkyl moiety has from 1 to 8 C atoms. Examples of haloalkoxy groups include, but are not limited to, trifluoromethoxy, 2-fluoroethoxy, pentafluoroethoxy, or 3-chloropropoxy.

As used herein, the term “haloalkyl” refers to an alkyl group having the meaning as defined above wherein one or more hydrogens are replaced with a halogen. A haloC₁₋₈ alkyl group refers to a haloalkyl group wherein the alkyl moiety has from 1 to 8 C atoms. Specifically embraced are monohaloalkyl, dihaloalkyl or polyhaloalkyl groups. A monohaloalkyl group, for one example, may have an iodo, bromo, chloro or fluoro atom within the group. Dihalo or polyhaloalkyl groups may have two or more of the same halo atoms or a combination of different halo groups. Examples of haloalkyl groups include, but are not limited to, fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl or dichloropropyl.

As used herein, the term “heteroalkyl” refers to a straight or branched alkyl chain, wherein one, two, or three carbons forming the alkyl chain are each replaced by a heteroatom independently selected from the group consisting of O, N, and S, and wherein the nitrogen and/or sulfur heteroatom(s) (if present) may optionally be oxidized and the nitrogen heteroatom(s) (if present) may optionally be quaternized. The heteroatom(s) O, N and S may, for example, be placed at the end(s) or at an interior position of the heteroalkyl group, i.e., the heteroalkyl may be bound to the remainder of the molecule via a heteroatom or a carbon atom. Up to two heteroatoms may be consecutive, such as, for example, —CH₂—NH—OCH₃. Accordingly, a further example for a “heteroalkyl” group is a straight or branched alkyl group, in which two consecutive carbon atoms are replaced by the heteroatoms S and N, respectively, and the sulfur heteroatom is furthermore oxidized, resulting in moieties such as, e.g., —S(═O)₂—NH₂, —S(═O)₂—NH(alkyl) or —S(═O)₂—N(alkyl)(alkyl).

As used herein, the term “heteroalkylene” refers to a heteroalkyl group attached at two positions. Examples include, but are not limited to, —CH₂OCH₂—, —CH₂SCH₂—, and —CH₂NHCH₂—, —CH₂S—, or —CH₂NHCH(CH₃)CH₂—. Accordingly, the term “heteroalkylene” may, e.g., refer to a straight or branched alkylene group (i.e., a straight or branched alkanediyl group) having from 1 to 6 carbon atoms, wherein 1, 2 (if present) or 3 (if present) of said carbon atoms are each replaced by a heteroatom independently selected from O, N or S. It is to be understood that the presence of hydrogen atoms will depend on the valence of the heteroatom replacing the respective carbon atom. If, for example, the carbon atom in a —CH₂— group is replaced by O or S, the resulting group will be —O— or —S—, respectively, while it will be —N(H)— when the carbon atom replaced by N. Likewise, if the central carbon atom in a group —CH₂—CH(—CH₃)—CH₂— is replaced by N, the resulting group will be —CH₂—N(—CH₃)—CH₂—. An example for a “heteroalkylene” group is a straight or branched alkylene group, in which two consecutive carbon atoms are replaced by the heteroatoms S and N, respectively, and the sulfur heteroatom is furthermore oxidized, resulting in moieties such as, e.g., —S(═O)₂—N(H)— or —S(═O)₂—N(alkyl)-. Accordingly, the groups —S(═O)₂—N(H)— and —S(═O)₂—N(alkyl)- (e.g., —S(═O)₂—N(C₁-C₆ alkyl)-) are exemplary “heteroalkylene” groups.

As used herein, the term “heteroC₁₋₄ alkylene” refers to a straight or branched C₁₋₄ alkylene group (i.e., a straight or branched C₁₋₄ alkanediyl group) linked to one heteroatom selected from O, N and S and also refers to a straight or branched C₁₋₄ alkylene group wherein one or more (e.g., 1, 2 (if present) or 3 (if present)) of the carbon atoms of said alkylene group are each replaced by a heteroatom independently selected from O, N or S. The nitrogen and/or sulfur heteroatom(s) (if present) may optionally be oxidized and the nitrogen heteroatom(s) (if present) may optionally be quaternized. The heteroatom(s) O, N and S may be placed at the end(s) and/or at an interior position of the heteroC₁₋₄ alkylene group. It is to be understood that the presence of hydrogen atoms will depend on the valence of the heteroatom replacing the respective carbon atom. If, for example, the carbon atom in a —CH₂— group is replaced by O or S, the resulting group will be —O— or —S—, respectively, while it will be —N(H)— when the carbon atom is replaced by N. Likewise, if the central carbon atom in a group —CH₂—CH(—CH₃)—CH₂— is replaced by N, the resulting group will be —CH₂—N(—CH₃)—CH₂—. An example for a “heteroC₁₋₄ alkylene” group is a straight or branched C₁₋₄ alkylene group, in which two consecutive carbon atoms are replaced by the heteroatoms S and N, respectively, and the sulfur heteroatom is furthermore oxidized, resulting in moieties such as, e.g., —S(═O)₂—N(H)— or —S(═O)₂—N(CH₃)—.

As used herein, the term “heteroaryl” refers to a 5 to 6 membered unsaturated monocyclic ring, or a fused bicyclic or tricyclic ring system in which the rings are aromatic and in which at least one ring contains at least one heteroatom selected from the group consisting of O, S, and N. Preferred heteroaryl groups are 5- to 6-membered monocyclic or 9- to 10-membered bicyclic heteroaryl groups. Examples of heteroaryl groups include, but are not limited to, pyridinyl, imidazolyl, imidazopyridinyl, pyrimidinyl, pyrazolyl, triazolyl, pyrazinyl, tetrazolyl, furyl, thienyl, isoxazolyl, thiazolyl, oxadiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, or furopyridinyl.

As used herein, the term “heterocyclyl” or “heterocycle” each refer to a saturated, partially unsaturated, or fully unsaturated monocyclic, bicyclic, or tricyclic heterocyclic group containing at least one heteroatom as a ring member, wherein each said heteroatom may be independently selected from the group consisting of nitrogen, oxygen, and sulfur wherein the nitrogen or sulfur atoms may be oxidized (e.g., —N═O, —S(═O)—, or —S(═O)₂—). Additionally, 1, 2, or 3 of the carbon atoms of the heterocyclyl may be optionally oxidized (e.g., to give an oxo group or ═O). One group of heterocyclyls has from 1 to 4 heteroatoms as ring members. Another group of heterocyclyls has from 1 to 2 heteroatoms as ring members. One group of heterocyclyls has from 3 to 8 ring members in each ring. Yet another group of heterocyclyls has from 3 to 7 ring members in each ring. Again another group of heterocyclyls has from 5 to 6 ring members in each ring. “Heterocyclyl” is intended to encompass a heterocyclyl group fused to a carbocyclyl or benzo ring systems. Examples of heterocyclyl groups include, but are not limited to, pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, homopiperazinyl, azetidinyl, oxetanyl, thietanyl, homopiperidinyl, oxepanyl, thiepanyl, oxazepinyl, diazepinyl, thiazepinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinylimidazolinyl, or imidazolidinyl. Examples of heteroaryls that are heterocyclyls include, but are not limited to, pyridinyl, imidazolyl, imidazopyridinyl, pyrimidinyl, pyrazolyl, triazolyl, pyrazinyl, tetrazolyl, furyl, thienyl, isoxazolyl, thiazolyl, oxadiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, or furopyridinyl.

As used herein, the term “heterocycloalkyl” refers to a heterocyclyl group that is not fully unsaturated e.g., one or more of the rings systems of a heterocycloalkyl is not aromatic. Examples of heterocycloalkyls include piperazinyl, morpholinyl, piperidinyl, or pyrrolidinyl.

As used herein, the term “hydroxyl” or “hydroxy” refers to —OH.

As used herein, the term “hydroxyalkyl,” as used herein, refers to a hydroxyl group attached to the parent molecular moiety through an alkyl group.

As used herein, the term “hydroxyC₁₋₈ alkyl” refers to an C₁₋₈ alkyl group, wherein one or more hydrogen atoms (preferably one or two) have been replaced by hydroxy groups.

As used herein, the term “R¹²R¹³N—C₁₋₈ alkyl” refers to an C₁₋₈ alkyl group, wherein one or more hydrogen atoms (preferably one or two, more preferably one) have been replaced by —NR¹²R¹³.

As used herein, the phrase “in the main chain,” refers to the longest contiguous or adjacent chain of carbon atoms starting at the point of attachment of a group to the compounds of any one of the formulas disclosed herein.

As used herein, the term phrase “linear chain of atoms” refers to the longest straight chain of atoms independently selected from carbon, nitrogen, oxygen and sulfur.

As used herein, the term “lower” where not otherwise specifically defined, means containing from 1 to and including 6 carbon atoms.

As used herein, the term “lower aryl,” means phenyl or naphthyl.

As used herein, the term “nitro” refers to —NO₂.

As used herein, the term “saturated” in relation to a ring means that the ring does not contain any unsaturation.

As used herein, the terms “sulfonate” “sulfonic acid” and “sulfonic” refer to the —SO₃H group and its anion as the sulfonic acid is used in salt formation.

As used herein, the term “sulfanyl,” to —S—.

As used herein, the term “sulfinyl” refers to —S(═O)(R), with R as defined herein.

As used herein, the term “sulfonyl” refers to —S(═O)₂R, with R as defined herein.

As used herein, the term “sulfonamide” refers to an N-sulfonamido or S-sulfonamido group as defined herein.

As used herein, the term “N-sulfonamido” refers to a RS(═O)₂N(R′)— group with R and R′ as defined herein.

Preferred N-sulfonamido groups are —NHSO₂R, wherein R is as defined herein, preferably R is alkyl, cycloalkyl, heteroalkyl, aryl, heteroaryl or heterocycloalkyl, more preferably R is alkyl, aryl, heteroaryl or heterocycloalkyl, wherein said alkyl, said cycloalkyl, said heteroalkyl, said aryl, said heteroaryl and said heterocycloalkyl are each optionally substituted. The optional substituents on said alkyl, said cycloalkyl, said heteroalkyl, said aryl, said heteroaryl and said heterocycloalkyl may be selected independently from lower alkyl, lower alkenyl, lower alkynyl, lower alkanoyl, lower heteroalkyl, lower heterocycloalkyl, lower haloalkyl, lower cycloalkyl, phenyl, aryl, heteroaryl, pyridyl, aryloxy, lower alkoxy, lower haloalkoxy, oxo, lower acyloxy, carbonyl, carboxyl, lower alkylcarbonyl, lower carboxyester, lower carboxamido, cyano, halogen, hydroxyl, amino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, carbamate, and urea. Preferably, the optional substituents are independently selected from hydroxyl, halo, alkyl, alkoxy, haloalkyl, haloalkoxy, —N(C₁₋₃ alkyl)₂, —NH(C₁₋₃ alkyl), —NHC(═O)(C₁₋₃ alkyl), —C(═O)OH, —C(═O)O(C₁₋₃ alkyl), —C(═O)(C₁₋₃ alkyl), —C(═O)NH₂, —C(═O)NH(C₁₋₃ alkyl), —C(═O)NH(cycloalkyl), —C(═O)N(C₁₋₃ alkyl)₂, —S(═O)₂(C₁₋₃ alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, or tetrazolyl. Particularly preferred N-sulfonamido groups are —NHSO₂R, wherein R is alkyl, cycloalkyl, heteroalkyl, aryl, heteroaryl or heterocycloalkyl, and preferably R is alkyl, aryl, heteroaryl or heterocycloalkyl, and —NHSO₂ (optionally substituted aryl). Still more preferred N-sulfonamido groups are —NHSO₂alkyl and —NHSO₂ (optionally substituted aryl). Exemplary, non-limiting N-sulfonamido groups are —NHSO₂alkyl such as —NHSO₂CH₃, —NHSO₂CH₂CH₃ or —NHSO₂ (isopropyl), and —NHSO₂ (optionally substituted aryl) such as —NHSO₂-phenyl, —NHSO₂-(2-cyanophenyl), —NHSO₂-(3-cyanophenyl), —NHSO₂-(4-cyanophenyl), —NHSO₂-(2-aminophenyl), —NHSO₂-(3-aminophenyl) or —NHSO₂-(4-aminophenyl). Other exemplary N-sulfonamido groups are —NHSO₂ (optionally substituted heterocycloalkyl) such as —NHSO₂-(piperazin-1-yl) and —NHSO₂ (optionally substituted heteroaryl) such as —NHSO₂-(optionally substituted pyridyl) like —NHSO₂-(3-pyridyl) or —NHSO₂-(6-amino-3-pyridyl).

As used herein, the term “S-sulfonamido” refers to a —S(═O)₂NRR′, group, with R and R′ as defined herein.

As used herein, the term “urea” refers to a —N(R)C(═O)N(R)(R′) group wherein R and R′ are as defined herein.

As used herein, “hydrogen bonding group” refers to a substituent group, which is capable of taking part in a non-covalent bonding between hydrogen and another atom (usually nitrogen or oxygen). Examples include, but are not limited to, —NH₂, —OH, amido, —S(O)₂NH₂, —C(═O)NH₂, —CH₂—C(═O)NH₂, — and —CH₂—NH₂. Other non-limiting examples include NHC(═O)CH₃ or —NHCH₃.

As used herein, the term “amide isostere” refers to a monocyclic or bicyclic ring system that is isosteric or bioisosteric with an amide moiety. Examples of amide isoteres include but are not limited to those disclosed in, e.g., Meanwell (2011) J. Med. Chem. PMID: 21413808,

The term R or the term R′, appearing by itself and without a number designation, unless otherwise defined, refers to a moiety selected from the group consisting of hydrogen, alkyl, cycloalkyl, heteroalkyl, aryl, heteroaryl and heterocycloalkyl. Both unsubstituted and substituted forms of the above groups are encompassed.

Whether an R group has a number designation or not, every R group, including R, R′ and R^{z} where z=(1, 2, 3, . . . z), every substituent, and every term should be understood to be independent of every other in terms of selection from a group. Should any variable, substituent, or term (e.g., aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(═O)N(R)— may be attached to the parent moiety at either the carbon or the nitrogen.

As used herein, the term “optionally substituted” means the preceding or anteceding group may be substituted or unsubstituted. When substituted and unless otherwise specified, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower cycloalkyl, phenyl, aryl, heteroaryl, pyridyl, aryloxy, lower alkoxy, lower haloalkoxy, oxo, lower acyloxy, carbonyl, carboxyl, lower alkylcarbonyl, lower carboxyester, lower carboxamido, cyano, halogen, hydroxyl, amino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, carbamate, and urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.” In one specific definition, the optional substituents are chosen from hydroxyl, halo, alkyl, alkoxy, haloalkyl, haloalkoxy, —N(C₁₋₃ alkyl)₂, —NH(C₁₋₃ alkyl), —NHC(═O)(C₁₋₃ alkyl), —C(═O)OH, —C(═O)O(C₁₋₃ alkyl), —C(═O)(C₁₋₃ alkyl), —C(═O)NH₂, —C(═O)NH(C₁₋₃ alkyl), —C(═O)NH(cycloalkyl), —C(═O)N(C₁₋₃ alkyl)₂, —S(═O)₂(C₁₋₃ alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, —NO₂, or tetrazolyl.

As used herein, the term “optional substituent” denotes that the corresponding substituent may be present or may be absent. Accordingly, a compound having 1, 2 or 3 optional substituents may be unsubstituted or may be substituted with 1, 2 or 3 substituents, which may be the same or different.

The following examples illustrate various aspects of the invention. The examples should, of course, be understood to be merely illustrative of only certain embodiments of the invention and not to constitute limitations upon the scope of the invention.

### EXAMPLES

### Example 1: LSD1 Inhibitors and In Vitro Biochemical Assays

This example describes the LSD1 inhibitors used in the subsequent examples and methods to assess the activity of test compounds against LSD1 and related enzymes MAO-A and MAO-B.

1.1 LSD1 Inhibitors Used

Compound 1 is the compound (−) 5-((((trans)-2-(4-(benzyloxy)phenyl)cyclopropyl)amino)methyl)-1,3,4-oxadiazol-2-amine, which can be obtained as disclosed in WO2012/013728.
Compound 2 is the enantiomer of compound 1 and it is the compound (+) 5-((((trans)-2-(4-(benzyloxy)phenyl)cyclopropyl)amino)methyl)-1,3,4-oxadiazol-2-amine. It can be obtained as disclosed in WO2012/013728.
Compound 3 is the compound with the following chemical name and structure, and can be obtained as disclosed in WO2011/042217:

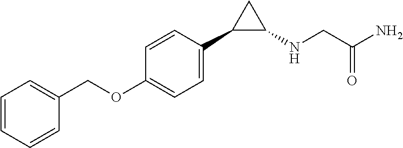 [SMILES: NC(=O)CN[C@H]1C[C@@H]1c1ccc(OCc2ccccc2)cc1]

2-(((trans)-2-(4-(benzyloxy)phenyl)cyclopropyl)amino)acetamide.
   Compound 4 is the compound with the following chemical name and structure, and can be obtained as disclosed in WO2010/043721:

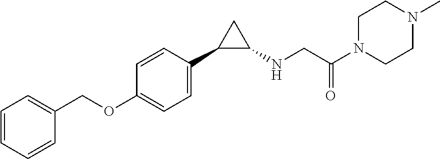 [SMILES: CN1CCN(C(=O)CN[C@H]2C[C@@H]2c2ccc(OCc3ccccc3)cc2)CC1]

2-(((trans)-2-(4-(benzyloxy)phenyl)cyclopropyl)amino)-1-(4-methylpiperazin-1-yl)ethan-1-one.
   Compound 5 is the compound with the following chemical name and structure, and can be obtained as disclosed in WO2011/035941:

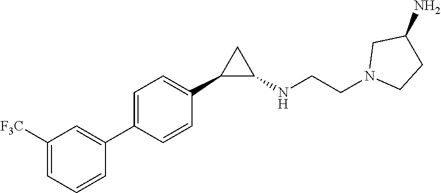 [SMILES: N[C@H]1CCN(CCN[C@H]2C[C@@H]2c2ccc(-c3cccc(C(F)(F)F)c3)cc2)C1]

(S)-1-(2-(((trans)-2-(3′-(trifluoromethyl)-[1,1′-biphenyl]-4-yl)cyclopropyl)amino)ethyl)pyrrolidin-3-amine.

Compounds 1 and 2 are optically active stereoisomers, whereas compounds 3 to 5 are “trans” racemic mixtures. The stereochemistry shown in the chemical structures depicted above for the cyclopropyl moiety in compounds 3 to 5 is thus only intended to show that the compounds have a “trans” configuration in respect to the substituents on the cyclopropyl ring, it does not indicate absolute stereochemistry for said carbon atoms.

1.2 In Vitro Biochemical Assays

1.2.1 LSD1

The inhibitory activity of a compound of interest against LSD1 can be tested using the method described below: Human recombinant LSD1 protein from BPS Bioscience Inc (catalog reference number 50100: human recombinant LSD1, GenBank accession no. NM_015013, amino acids 158-end with N-terminal GST tag, MW: 103 kDa) was used. In order to monitor LSD1 enzymatic activity and/or its inhibition rate by a test compound, di-methylated H3-K4 peptide (Anaspec) was chosen as a substrate. The demethylase activity was estimated, under aerobic conditions, by measuring the release of H₂O₂ produced during the catalytic process, using the Amplex® Red hydrogen peroxide/peroxidase assay kit (Invitrogen).

Briefly, a fixed amount of LSD1 was incubated on ice for 15 minutes, in the absence and/or in the presence of at least eight 3-fold serial dilutions of the respective inhibitor (e.g., from 0 to 75 μM, depending on the inhibitor strength). Tranylcypromine (Biomol International) was used as a control for inhibition. Within the experiment, each concentration of inhibitor was tested in duplicate. After leaving the enzyme interacting with the inhibitor, K_{M} of di-methylated H3-K4 peptide was added to each reaction and the experiment was left for 30 minutes at 37° C. in the dark. The enzymatic reactions were set up in a 50 mM sodium phosphate, pH 7.4 buffer. At the end of the incubation, Amplex® Red reagent and horseradish peroxidase (HPR) solution were added to the reaction according to the recommendations provided by the supplier (Invitrogen), and left to incubate for 5 extra minutes at room temperature in the dark. A 1 μM H₂O₂ solution was used as a control of the kit efficiency. The conversion of the Amplex® Red reagent to resorufin due to the presence of H₂O₂ in the assay, was monitored by fluorescence (excitation at 540 nm, emission at 590 nm) using a microplate reader (Infinite 200, Tecan). Arbitrary units were used to measure level of H₂O₂ produced in the absence and/or in the presence of inhibitor. The maximum demethylase activity of LSD1 was obtained in the absence of inhibitor and corrected for background fluorescence in the absence of LSD1. The IC50 value of each inhibitor was calculated with GraphPad Prism Software.

1.2.2 Monoamine Oxidase A (MAO-A) and B (MAO-B)

LSD1 has a fair degree of structural similarity and amino acid identity/homology with the flavin-dependent amine oxidases monoamine oxidase A (MAO-A) and B (MAO-B). To determine the level of selectivity of a LSD1 inhibitor versus MAO-A and MAO-B, the inhibitory activity of a compound of interest against MAO-A and MAO-B can be tested using the method described below: Human recombinant monoamine oxidase proteins MAO-A and MAO-B were purchased from Sigma Aldrich.

MAOs catalyze the oxidative deamination of primary, secondary and tertiary amines. In order to monitor MAO enzymatic activities and/or their inhibition rate by inhibitor(s) of interest, a fluorescence-based (inhibitor)-screening assay was set up. 3-(2-Aminophenyl)-3-oxopropanamine (kynuramine dihydrobromide, Sigma Aldrich), a non fluorescent compound was chosen as a substrate. Kynuramine is a non-specific substrate for both MAO-A and MAO-B activities. While undergoing oxidative deamination by MAO activities, kynuramine is converted into 4-hydroxyquinoline (4-HQ), a resulting fluorescent product.

The monoamine oxidase activity was estimated by measuring the conversion of kynuramine into 4-hydroxyquinoline. Assays were conducted in 96-well black plates with clear bottom (Corning) in a final volume of 100 μL. The assay buffer was 100 mM HEPES, pH 7.5. Each experiment was performed in duplicate within the same experiment.

Briefly, a fixed amount of MAO was incubated on ice for 15 minutes in the reaction buffer, in the absence and/or in the presence of at least eight 3-fold serial dilutions each. Clorgyline and Deprenyl (Sigma Aldrich) was used as a control for specific inhibition of MAO-A and MAO-B respectively.

After leaving the enzyme(s) interacting with the inhibitor, K_{M} of kynuramine was added to each reaction for MAO-B and MAO-A assay respectively, and the reaction was left for 1 hour at 37° C. in the dark. The oxidative deamination of the substrate was stopped by adding 50 μL of NaOH 2N. The conversion of kynuramine to 4-hydroxyquinoline, was monitored by fluorescence (excitation at 320 nm, emission at 360 nm) using a microplate reader (Infinite 200, Tecan). Arbitrary units were used to measure levels of fluorescence produced in the absence and/or in the presence of inhibitor.

The maximum of oxidative deamination activity was obtained by measuring the amount of 4-hydroxyquinoline formed from kynuramine deamination in the absence of inhibitor and corrected for background fluorescence in the absence of MAO enzymes. The IC50 values of each inhibitor were calculated with GraphPad Prism Software.

1.2.3 Results

Exemplary IC50 values against LSD1, MAO-A and MAO-B obtained using the above methods for compounds 1 to 5 are shown in the table below:

| | LSD1 | MAO B | MAO A |
|---|---|---|---|
| Compound # | (IC50-μM) | (IC50-μM) | (IC50-μM) |
| Comp1 | 0.09 | 0.06 | 5.3 |
| Comp2 | 2.3 | 0.18 | 3.8 |
| Comp3 | 0.16 | 0.07 | 2.3 |
| Comp4 | 0.1 | 4.7 | 4.6 |
| Comp5 | 0.06 | 5.1 | 3.4 |

As can be seen from the above data, Compound 1 is a potent dual LSD1/MAO-B inhibitor, wherein its enantiomer, Compound 2, is a much weaker LSD1 inhibitor while retaining potent MAO-B inhibitory activity. Compound 3 exhibits LSD1 and MAO-B inhibitory activity, and Compounds 4 and 5 are potent LSD1 inhibitors with selectivity versus MAO-A and MAO-B.

### Example 2: Gene Expression Analysis by Microarray Hybridization

This example describes the general method used in subsequent examples to perform microarray gene expression analysis.

2.1 RNA Extraction and Labeling Method

Total RNA was extracted from samples using the RNeasy extraction kit (Qiagen). The quality and concentration of the RNA was analyzed using the Agilent 2100 bioanalyzer and NanoDrop™ ND-1000 (Thermo Scientific). Samples with RNA integrity number (RIN)<6.0 were discarded. Total RNA (0.5 μg) amplification and labelling with Cy3 or Cy5 was carried out using the Eberwein mRNA amplification procedure (Van Gelder et al, Proc Natl Acad Sci USA 1990, 87:1663-1667) employing the MessageAmp™ aRNA amplification kit from Ambion (Applied Biosystems) following the manufacturer's instructions with minor modifications. As hybridization controls, plant mRNAs were transcribed from a plasmid containing the Zea mays Xet (xyloglucan endo-transglycosylase) cDNA and from a plasmid containing the Zea mays Zmmyb42 cDNA, independently prepared Cy3 and Cy5 labelled aRNA from these two RNAs using the Eberwein mRNA amplification procedure (as disclosed in Cerdà et al, Gen Comp Endocrinol 2008, 156:470-481).

2.2 Hybridization and Data Acquisition

The Cy3- and Cy5-labelled cRNAs and spikes were combined and hybridized to the microarray described in Example 2.6 for 17 h at 60° C. using Agilent's gaskets G2534-60002, G2534A hybridization chambers and DNA Hybridization Oven G2545A, according to the manufacturer's instructions. More specifically, equal amounts of Cy3 and Cy5 labeled Xet aRNA as well as equal amounts of Cy3 and Cy5 labeled Zmmyb42 aRNA were spiked into each mixture hybridized to the array. Arrays were washed and raw data were obtained using Agilent's DNA Microarray Scanner G2505B and Feature Extraction software (v10.1). The raw fluorescence intensity data were processed using applicant's proprietary software, and consists in the following operations:

1) spatial data compensation based on the hybridization pattern of the spiked-in controls, 2) global data filtering, and 3) data normalization.

Data compensation was performed based on the behavior of the plant aRNAs spiked into the array. Briefly, the labeled spike aRNAs hybridized to its corresponding spike i.e. control probes, represented in multiple copies and distributed strategically over the array, generates signals distributed over the expected dynamic range. The signal intensities derived from each specific repeated probe, form a data surface (x_{array}, y_{array}, Z_{signal intensity}). The data compensation algorithm uses these data surfaces to calculate a function that corrects all data surface to horizontality, and then applies the same operation to the total gene probe dataset. This data compensation can absorb most systematic spatial deviation generated by array synthesis, hybridization defects or scanner deviations between Cy3 and Cy5.

Global data filtering: using the specific controls and comparing all the samples in an experiment, the probes which are not expressed in any sample are systematically removed in order not to bias the posterior statistics. The decision on which probes are not expressed is based on the background probes and the negative control probes, designed to recognize maize expansin RNA (not included in the spikes) but not any mouse RNA.

2.3 Microarray Data Normalization

Data normalization was carried out by an improved version of the nonlinear Q-splines normalization method (Workman et al., Genome Biol 2002, 3(9):research0048.1-0048.16). Normalization results were presented graphically as MA plots, as first described by Dudoit et al (Dudoit et al, Statistica Sinica, 2002, 12:111-139). Each point corresponds to a probe of the DNA array. M values (vertical axis) are the log-differential expression ratios. A values (horizontal axis) are the log-intensities of the spots. M and A are calculated from the Cy5 and Cy3 intensity values as of each spot. The DNA Array data is normalized using an improved version of non-linear Q-Splines normalization described by Workman et al., Genome Biol 2002, 3(9):research0048.1-0048.16. This process is useful to correct the deviation of the M values from the statistical assumption that most of the spots have M=0 in MA plots. The method allows the adjustment of all M values to form a cloud centered at M=0 in all intensity ranges. The data used to calculate the normalization fitting curve is the totality of the raw measurement dataset, excluding the signals for the probes for the spiked-in plant RNA controls and negative controls. In order to be able to normalize properly the data among all the experiments, the probe-set must include an important group of genes that should have no change (M=0) in the experiment. The normalization function is then applied to all the data, including controls.

Normalized and log-transformed data were used to calculate log 2 (sample/control) and Fold Change (FC) values. No background subtraction was applied to the signal intensity values during data processing, permitting robust selection of differentially expressed genes with low expression levels at the cost of potential sub-estimation of change when intensity levels are close to the array background level.

2.4 Replica Analysis

The technical replicate analysis is the statistical processing of the microarray data. Replicates were calculated to measure oligo replicates on an array or between replicates of experiments on different arrays (i.e. hybridization of the same sample). The output of the Replicate Analysis is a list of selected genes with associated mean log 2 (sample/control), Fold Change values (presented as the sample/control ratio when expression is induced and control/sample ratio when expression is reduced in the sample), and their corresponding p-values.

During replica analysis, datapoints may be identified which appear to lie outside the group of datapoints being analyzed, the so called outliers. Outliers are data that differ in a statistically important manner from the rest of a group of data for a given gene oligo. Outlier exclusion was applied to technical replicates. Outlier data on technical replicates can be caused by array imperfections like dust or synthesis defects. Outlier data can also be caused simply by errors, for example mislabeling or mixing up of a sample. What is to be considered “statistically different” was defined by comparison of the observed variation for the replicas of a gene probe with the expected variation for a given experiment, which was calculated based on the variation in Fold Changes observed for the control values yielded by the spike signals. In no case more than 20% of the data in a replica group was eliminated as outliers.

2.5 Calculation of P Values for Microarray Data

The p-values were calculated based on the absolute value of the regularized t-statistic (Baldi et al, Bioinformatics 2001, 17(6):509-519), which uses a Bayesian framework to derive the algorithm, using internal replica controls to assess the minimum technical variability of the process. The inherent experimental variation was assessed by the FC of internal controls and/or self-to-self hybridizations.

2.6 Microarray Design and Relevant Probes

The microarrays employed were designed using applicant's proprietary software based on thermodynamic simulation of hybridization. The basic parameter used for the design of oligo with a length of 50-60 bases was the melting temperature (Tm), calculated using the “Nearest-Neighbours” and applying the parameters provided by Sugimoto et al (N Sugimoto et al, Biochemistry 1995, 34:11211-11216) for DNA/RNA interactions in defined salt concentrations. To calculate the folding of the oligo the minimum free energy algorithm of Zuker & Stiegler (M Zuckler and P Stiegler, Nucleic Acids Research 1981, 9(1): 133-148) was used, and for cases where the minimum free energy equaled 0, the algorithms of Wutchy et al were used (S Wutchy et al, Biopolymers 1999, 49:145-165). The secondary structures of oligonucleotides and (fragmented) nucleic acids in solution were calculated. To model the hybridization process, the candidate oligonucleotide probes were first aligned with the transcriptome using the BLAST (http://www.ncbi.nlm.nih.gov/BLAST) algorithm (Altschul et al, J Mol Biol 1990, 215:403-410). The software performed total alignment (no mismatches allowed), mismatch alignment, and partial alignment (i.e. partial overlap) and calculated the Tm of all interactions based the sequence of all nucleic acids (sample, oligos, spiked in controls) and other relevant parameters (nucleic acid and salt concentration, temperature) of the hybridization reaction.

Next, Tm range limitations were applied, aiming for a narrow Tm distribution and homogeneous behavior of the oligos that generated desired target interactions and imposing maximum values to the Tm of undesired interactions to limited cross-hybridizations and secondary structures. Next, a quality factor was calculated based on the lineal combination of the following parameters: distance of the oligo to the 3′ end of the mRNA sequence (3′ bias of Eberwein labeling), Tm of the oligo, length of the oligo, distance of the Tm of the oligo to the maximum cross-hybridization Tm, distance of the Tm of the oligo to the maximum secondary structure Tm, GC content. The quality factor was used to rank the different possible oligos for a given gene and select the best possible oligos in function of the available spot positions.

For the design of this array, parameters were set as follows:

Array type: gene expression (DNA/RNA); Oligo size min 50, max 60, Distance 3′ max 1500, Tm range 70-80, max Tm secondary structure 60, max Tm cross-hybridization 60, 1 oligo per target sequence. Salt concentration and nucleic acid concentration: as per Agilent gene expression hybridization protocol.

The final microarrays contain triplicate gene probes for each of the different mouse genes as well as thousands of replicas probes for the spike controls.

Gene probes were designed using the ENSEMBL database. For sequences where we did not find high quality probes, we complemented the design with suboptimal probes (R-probes). DNA microarrays synthesis was outsourced to Agilent. The mouse Whole Genome Gene Expression Array contains:
17386 Oryzon High Quality probes designed based on ENSEMBL Database built 53, may 2009 (based on built NCBI36).
10736 Oryzon Recovery probes designed based on ENSEMBL Database.

The total amount of mouse gene probes is 28122.

In addition, the mouse array design contains probes that recognize the spiked-in plant aRNA; as well as negative controls.

The following probes were examined in detail:

| TYPE | NAME | Microarray Probe Sequence |
|---|---|---|
| BIOMARKER | S100a8 | AAAAGTGGGTGTGGCATCTCACAAAGACAG |
| | | CCACAAGGAGTAGCAGAGCTTCTGG |
| BIOMARKER | S100a9 | GCCATGTGACAGCTGCCCAACCAAGTCTAA |
| | | AGGGAATGGCTTACTCAATGGC |
| HOUSEKEEPING | Tubb3 | ACTGGGTTGTGTTTATATTCGGGGGGAGGG |
| | | GTATACTTAATAAAGTTACTGCTGTCTGTC |
| HOUSEKEEPING | Tubb2c | GCTGTCCTGTGTCCTGACATCACTTGTACA |
| | | GATACCACCATTAAAGCAATTCATAGT |

### Example 3: S100A8 and S100A9 are Up-Requlated in SAMP-8 vs SAMR1 Mice and Down-Regulated in the Hippocampus of SAMP-8 Mice after Treatment with LSD1 Inhibitors

This example illustrates that S100A8 and mostly S100A9 are over-expressed in the SAMP-8 mice, a model for accelerated aging and Alzheimer's disease, and that the over-expression of these genes can be modulated by treatment with LSD1 inhibitors in the absence of significant effects on hematology, and with beneficial effects on memory as assessed by the Novel Object Recognition Test (NORT).

3.1 Mice Strains and Treatment

The Senescence Accelerated Mouse Prone 8 (SAMP8) strain is a non-transgenic model for neurodegeneration reminiscent of Alzheimer's disease (T Takeda, Neurobiol Aging 1999, 20(2):105-10). Memory deficits appear around 5 months of age in SAMP8 mice and can be reliably assessed using the Novel Object Recognition Test (NORT). The Senescence Accelerated Mouse Resistant 1 (SAMR1) strain shows no memory deficits and is used as a control.

SAMP8 and SAMR1 mice were maintained 5 individuals par cage under standard conditions (temperature 23±1° C., humidity 50-60%, 12:12-h light-dark cycle, lights on at 7:00 a.m.), with food (A04, Harlan, Spain) and tap water available ad libitum until the treatment started. Body weight (g) was measured weekly.

Test compound: Compound 1, as defined in Example 1 above. Compound 1 is orally available and has been shown to cross the blood-brain barrier.

Males and females were separated in two different cohorts and all treatments started at 5 months of age. In a first experiment, female mice were randomly distributed in 4 experimental groups (n=16/group): SAMR1 vehicle, SAMP8 vehicle, SAMP8 Compound 1 at 0.96 mg/kg/day and SAMP8 Compound 1 at 3.2 mg/kg/day. Based on the results obtained in the first experiment, doses were adjusted in a second experiment using male mice. Male mice were randomly distributed in 4 experimental groups (n=16/group): SAMR1 vehicle, SAMP8 vehicle, SAMP8 Compound 1 at 0.32 mg/kg/day and SAMP8 Compound 1 at 0.96 mg/kg/day.

The test compound (Compound 1) was diluted in vehicle (1.8% hydroxypropyl-beta-cyclodextrin, Sigma-Aldrich) and administered in drinking water. The dose was calculated according to the animal water consumption average par cage and adjusted weekly. The test compound (or vehicle) was available for 5 days followed by a 2 day clearance in a weekly period.

3.2 Novel Object Recognition Test (NORT)

3.2.1 Method

NORT is used to assess animal's behavior when it is exposed to a novel and a familiar object (M Antunes and G Biala, Cogn Process. 2012, 13(2): 93-110). Animals explore the novel object as their natural propensity to the novelty, and it is possible to evaluate the index of stimulus recognition (Discrimination Index or DI, see below for description). After training or habituation, the DI can be configured to measure working memory (minutes after training), midterm (hours after training) and and long term memory (24 h and beyond) when information can remain indefinitely (Taglialatela et al., 2009, Behav Brain Res 200:95-99).

The NORT test was performed after 2 (n=16/group) and repeated at 4 (n=10/group) months of treatment (between 12:30 am and 6:30 pm). Animals were placed in a 90° two-arm, 25-cm-long, 20-cm-high, 5-cm-wide black maze. The 20-cm-high walls could be lifted off for easy cleaning. The light intensity in the middle of the field was 30 lux. The objects to be discriminated were made of plastic with different shape and color clearly distinguishable. For the first 3 days, the mice were individually habituated to the apparatus for 10 min. On the 4th day, the animals were submitted to a 10-min acquisition trial (first trial) during which they were placed in the maze in the presence of two identical novel objects (A+A or B+B) placed at the end of each arm. A 10-min retention trial was performed 2 h (both in males and females) and 24 h later (males only) in order to evaluate mid- and long-term memory, respectively. During this second trial, objects A and B were placed in the maze, and the time that the animal explored the new object (tn) and the old object (to) were video-recorded. A discrimination index (DI) was defined as (tn−to)/(tn+to). In order to avoid object preference biases, objects A and B were counterbalanced so that half of the animals in each experimental group were first exposed to object A and then to object B, whereas the other half saw first object B and then object A. The maze and the objects were cleaned with 96° ethanol between different animals, so as to eliminate olfactory cues.

3.2.2 Statistical Analysis

Statistical analysis was performed using the GraphPad Prism 6.0 software package. NORT data from SAMP8 mice was analyzed by one-way ANOVA with treatment as the main factor. Post-hoc comparisons with Bonferroni test were done, if appropriate. Student t-Test was used to compare SAMR1 vehicle and SAMP8 vehicle.

3.2.3 Results

Compound 1 completely prevents memory loss in SAMP8 as assessed by NORT after 2 m and 4m of treatment both in males and females, as discussed in more detail below.

3.2.3.1 Females

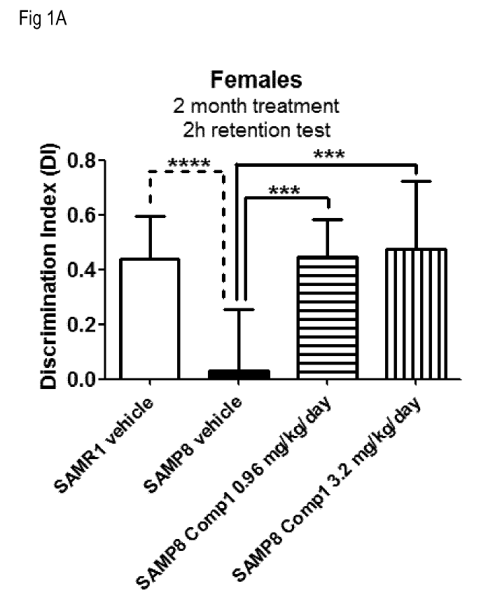

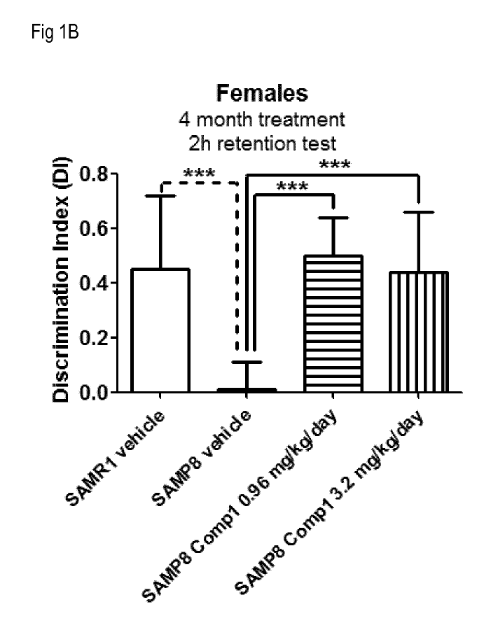

Treatment with Compound 1 administered orally in drinking water rescued the memory deficits in mid-term memory (2 h trial test) in SAMP8 females. This effect was observed at the two doses tested and both after 2 and 4 months of treatment, as shown in FIGS. 1A and 1B. The t-Student test showed differences in the discrimination index DI between vehicle-treated SAMR1 and vehicle-treated SAMP8 animals after 2 (p<0.0001) and 4 (p<0.001) months of treatment. The ANOVA showed differences due to the treatment in the discrimination index (p<0.0001) after 2 or 4 months of treatment with Compound 1. Post-hoc analysis showed higher discrimination index in the SAMP8 groups treated with Compound 1 compared to SAMP8 vehicle. **** p<0.0001; *** p<0.001

3.2.3.2 Males

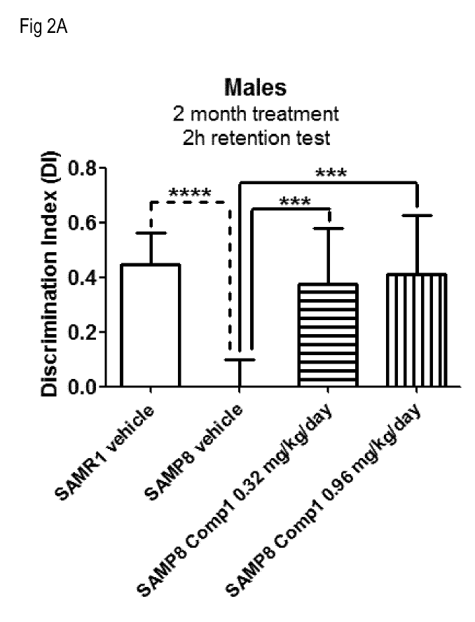

Treatment with Compound 1 administered orally in drinking water rescued the memory deficits of SAMP8 males. This effect was observed with the two doses tested and after a 2- and 4-month treatment, with positive effects both on medium- and long-term memory. The results are shown in FIG. 2A (mid-term memory, 2m treatment), 2B (mid-term memory, 4m treatment), 3A (long-term memory, 2m treatment) and 3B (long-term memory, 4m treatment).

Midterm Memory (2 h Trial Test):

As shown in FIGS. 2A and 2B, the t-Student test showed differences in the discrimination index between vehicle-treated SAMR1 and SAMP8 animals after 2 (p<0.0001) and 4 (p<0.0001) months of treatment. The ANOVA showed differences due to the treatment in the discrimination index DI after 2 (p<0.0001) and 4 (p<0.0001) months of treatment with Compound 1. Post-hoc analysis showed higher discrimination index in the SAMP8 groups treated with Compound 1 compared to SAMP8 vehicle. **** p<0.0001; *** p<0.001

Long Term Memory (24 h Trial Test):

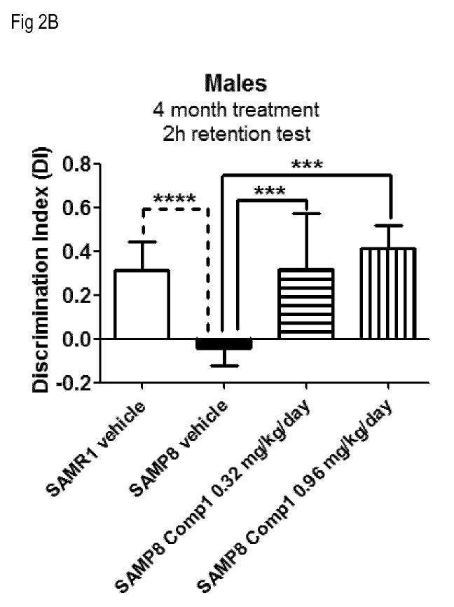

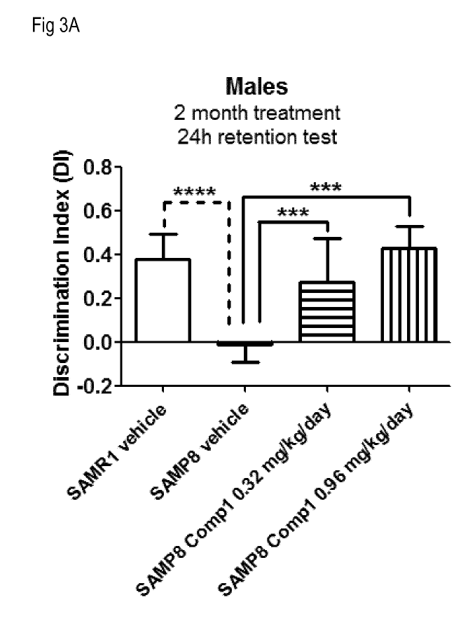

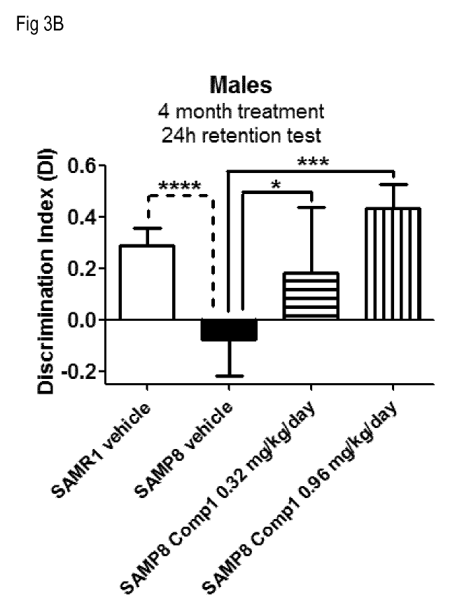

As shown in FIGS. 3A and 3B, the t-Student test showed differences in the discrimination index DI between vehicle-treated SAMR1 and SAMP8 animals after 2 (p<0.0001) and 4 (p<0.0001) months of treatment. The ANOVA showed differences due to the treatment in the discrimination index after 2 (p<0.001) and 4 (p<0.001) months of treatment with Compound 1. Post-hoc analysis showed higher discrimination index in the SAMP8 groups treated with Compound 1 compared to SAMP8 vehicle. **** p<0.0001; *** p<0.001; * p<0.05

3.3 Sampling Methods

One day after the NORT test, 7 (n=2-6/group) or 9 (n=2-4/group) month old animals were deeply anesthetized with 80 mg/kg of sodium pentobarbital. Blood samples were obtained by intracardiac puncture, collected in EDTA tubes and stored at 4° C. until analyzed. Afterwards, hippocampi were dissected and snap frozen on dry ice for further RNA extraction.

3.4 Effects on Hematopoiesis

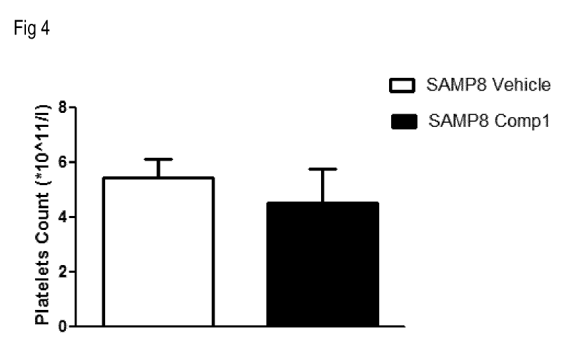

It is known that LSD1 is implicated in normal hematopoiesis (SprDssel et al, Leukemia 2012, 26(9)2039-51). To assess whether treatment with LSD1 inhibitors has an effect in hematopoiesis in SAMP8 mice at the doses administered, the effect on platelet levels in males of the higher dose tested of Compound 1 (0.96 mg/kg/day) was evaluated after 2 or 4 months of treatment. The mice were sacrificed and blood was collected in sodium citrate-containing tubes for hemogram analysis. Platelets levels were determined in a standard hematology analyzer (Abacus Junior Vet, from Diatron) following the manufacturer's instructions. The results obtained after 16 weeks of treatment are shown in FIG. 4. While a tendency towards reduction in platelet levels was observed, no statistically significant effects of Compound 1 treatment compared to vehicle-treated SAMP8 mice were observed.

3.5 Gene Expression Analysis by Microarray

3.5.1 Sample Preparation

Hippocampi samples from female mice from the above-described experiment obtained as described in section 3.3 were used for microarray GE analysis.

RNA extraction and labeling for gene expression analysis was performed using the general methods described in Example 2, to obtain the following samples:

3.5.1.1 Individual Sample List

2 month treatment

| Strain | Treatment | Sample | Female |
|---|---|---|---|
| SAMR1 | Vehicle | 1530 | R1FV7-SAMR1-F-Vehicle |
| | | 1531 | R1FV10-SAMR1-F-Vehicle |
| | | 1399 | R1FV11-SAMR1-F-Vehicle |
| | | 1532 | R1FV12-SAMR1-F-Vehicle |
| | | 1533 | R1FV15-SAMR1-F-Vehicle |
| | | 1400 | R1FV16 SAMR1-F-Vehicle |
| SAMP8 | Vehicle | 1535 | S8FV6-SAMP8-F-Vehicle |
| | | 1536 | S8FV7-SAMP8-F-Vehicle |
| | | 1537 | S8FV8-SAMP8-F-Vehicle |
| | | 1401 | S8FV9 SAMP8-F-Vehicle |
| | | 1402 | S8FV14 SAMP8 Vehicle |
| | | 1403 | S8FV15 SAMP8 Vehicle |
| SAMP8 | Comp1 | 1539 | S8FH9-SAMP8-F-3.2 mg |
| | 3.2 mg/kg/day | 1540 | S8FH11-SAMP8-F-3.2 mg |
| | | 1408 | S8FH12 SAMP8 3.2 mg |
| | | 1409 | S8FH16 SAMP8 3.2 mg |
| Strain | Treatment | Sample | Male |
| SAMR1 | Vehicle | 1516 | R1MV1-SAMR1-M-Vehicle |
| | | 1517 | R1MV2-SAMR1-M-Vehicle |
| SAMP8 | Vehicle | 1519 | S8MV3-SAMP8-M-Vehicle |
| | | 1520 | S8MV4-SAMP8-M-Vehicle |
| SAMP8 | Comp1 | 1524 | S8ML8-SAMP8-M-0.32 mg |
| | 0.32 mg/kg/day | 1545 | S8ML9-SAMP8-M-0.32 mg |
| | | 1546 | S8ML10-SAMP8-M-0.32 mg |

4 month treatment

| Strain | Treatment | Sample | Female |
|---|---|---|---|
| SAMR1 | Vehicle | 1528 | R1FV1-SAMR1-F-Vehicle |
| | | 1398 | R1FV4-SAMR1-F-Vehicle |
| | | 1529 | R1FV5-SAMR1-F-Vehicle |
| SAMP8 | Vehicle | 1512 | S8FV1-SAMP8-F-Vehicle |
| | | 1513 | S8FV4-SAMP8-F-Vehicle |
| | | 1534 | S8FV5-SAMP8-F-Vehicle |
| SAMP8 | Comp1 | 1404 | S8FL1-SAMP8-F-0.96 mg |
| | 0.96 mg/kg/day | 1405 | S8FL4-SAMP8-F-0.96 mg |
| | | 1406 | S8FL5-SAMP8-F-0.96 mg |
| SAMP8 | Comp1 | 1514 | S8FH1-SAMP8-F-3.2 mg |
| | 3.2 mg/kg/day | 1515 | S8FH3-SAMP8-F-3.2 mg |
| | | 1538 | S8FH4-SAMP8-F-3.2 mg |
| | | 1407 | S8FH5 SAMP8-F-3.2 mg |
| Strain | Treatment | Sample | Male |
| SAMR1 | Vehicle | 1518 | R1MV6-SAMR1-M-Vehicle |
| | | 1541 | R1MV7-SAMR1-M-Vehicle |
| | | 1542 | R1MV12-SAMR1-M-Vehicle |
| SAMP8 | Vehicle | 1521 | S8MV6-SAMP8-M-Vehicle |
| | | 1543 | S8MV7-SAMP8-M-Vehicle |
| | | 1544 | S8MV8-SAMP8-M-Vehicle |
| SAMP8 | Comp1 | 1522 | S8ML1-SAMP8-M-0.32 mg |
| | 0.32 mg/kg/day | 1523 | S8ML4-SAMP8-M-0.32 mg |
| SAMP8 | Comp1 | 1525 | S8MH5-SAMP8-M-0.96 mg |
| | 0.96 mg/kg/day | 1526 | S8MH6-SAMP8-M-0.96 mg |
| | | 1527 | S8MH8-SAMP8-M-0.96 mg |

The following RNA samples were pooled for labeling and microarray analysis:

Pool Samples List

| SAMPLES | POOL SAMPLE | |
|---|---|---|
| IN POOL | CODE | POOL SAMPLE DESCRIPTION |
| 1398, 1399, 1400 | 1413 Cy5 | POOL SAMR1 VEH: |
| 1401, 1402, 1403 | 1414 Cy5 | POOL SAMP8 VEH |
| 1404, 1405, 1406 | 1415 Cy5 | POOL SAMP8 Comp1 LD |
| 1407, 1408, 1409 | 1416 Cy5 | POOL SAMP8 Comp1 HD |
| 1401, 1402, 1403 | 1414 Cy3 | POOL SAMP8 VEH |

As used herein, Comp1 means Compound 1. LD means the low dose of Compound 1 administered to female mice, i.e. 0.96 mg/kg/day, and HD means the high dose of Compound 1 administered to female mice, i.e. 3.2 mg/kg/day. VEH means Vehicle.

3.5.2 Hybridization List

The following hybridizations were performed as described above

| HYB. # | CODE | CONTROL DESCRIPTION | CODE | SAMPLE DESCRIPTION |
|---|---|---|---|---|
| 1 | 1414 Cy3 | POOL SAMP8 VEHICLE | 1413 Cy5 | POOL SAMR1 VEH |
| 2 | 1414 Cy3 | POOL SAMP8 VEHICLE | 1414 Cy5 | POOL SAMP8 VEH |
| 3 | 1414 Cy3 | POOL SAMP8 VEHICLE | 1415 Cy5 | POOL SAMP8 Comp1 |
| | | | | (0.96 mg/kg/day) LD |
| 4 | 1414 Cy3 | POOL SAMP8 VEHICLE | 1416 Cy5 | POOL SAMP8 Comp1 |
| | | | | (3.2 mg/kg/day) HD |

3.5.3 Replica Analysis

Replica analysis was intra-array.

GROUP CODE

1414 Cy3 vs 1413 Cy5
1414 Cy3 vs 1415 Cy5
1414 Cy3 vs 1416 Cy5
Self-to-Self 1414

3.5.4 Gene Expression Results

The results obtained are shown in the table below:

| | | Hybridization signal | | Statistics |
|---|---|---|---|---|
| HIPPOCAMPUS | log2 (sample/control) | control_Cy3 | sample_Cy5 | p-value |
| | log2 (1414 Cy5/1414 Cy3) | | | |
| | log2 (SAMP-8 VEH/SAMP8 VEH) | | | |
| S100A8 | −0.108507 | 81.4243 | 75.5249 | 0.0676827 |
| S100A9 | −0.217568 | 105.749 | 90.9456 | 0.0117551 |
| TUBB3 | 0.0224714 | 2432.8 | 2470.99 | 0.326168 |
| TUBB2C | 0.24601 | 6856.62 | 8131.42 | 0.0011662 |
| Low | | 61 | 63 | |
| | log2 (1415 Cy5/1414 Cy3) | | | |
| | log2 (SAMP-8 LD/SAMP8 VEH) | | | |
| S100A8 | −0.225482 | 82.6741 | 70.7119 | 0.0276339 |
| S100A9 | −0.838056 | 118.85 | 66.4843 | 0.0010589 |
| TUBB3 | 0.0449797 | 2910.14 | 3002.3 | 0.101615 |
| TUBB2C | 0.119916 | 8643.34 | 9392.47 | 0.0063925 |
| Low | | 66 | 57 | |
| | log2 (1416 Cy5/1414 Cy3) | | | |
| | log2 (SAMP-8 HD/SAMP8 VEH) | | | |
| S100A8 | −0.181268 | 87.3979 | 77.0786 | 3.35E−02 |
| S100A9 | −0.764724 | 124.418 | 73.2282 | 1.11E−03 |
| TUBB3 | 0.0576913 | 3044.2 | 3168.4 | 0.0618207 |
| TUBB2C | 0.132662 | 8367.12 | 9173 | 0.0051698 |
| Low | | 71 | 64 | |
| | log2 (1413 Cy5/1414 Cy3) | | | |
| | log2 (SAMR1 VEH/SAMP8 VEH) | | | |
| S100A8 | −0.42018 | 92.615 | 69.2141 | 0.014109 |
| S100A9 | −0.858165 | 120.373 | 66.4042 | 1.33E−03 |
| TUBB3 | −0.153249 | 3119.47 | 2805.1 | 0.0027589 |
| TUBB2C | 0.296597 | 8426.99 | 10350.4 | 0.001325 |
| Low | | 67 | 43 | |

Low: Lowest above background signal on the array, rounded to the lower unit. Log 2 (sample/control) values were calculated without background subtraction as described above.

S100A8 and particularly S100A9 were up-regulated in SAMP8 versus reference strain SAMR1 and were down-regulated by treatment with Compound 1. Two housekeeping genes i.e. genes that showed a relatively constant levels of expression across the different experiments, Tubb3 and Tubb2c, were included for comparison.

Down-regulation of S100A9 and S100A8 with Compound 1 was observed at HD, at which a significant reduction of platelet levels was observed, but importantly, also at LD, where no significant reduction of platelet levels was observed. Full rescue of the memory capacity as assessed by the NORT test was also observed in SAMP-8 animals treated at HD and LD.

### Example 4: Validation of the Effects of LSD1 Inhibitors on S100A9 Expression by RNA Sequencing

Microarray hybridization results disclosed in Example 3 were confirmed using Illumina RNA sequencing as an alternative gene expression technology, using samples from female and male mice treated for 4 months with Compound 1 or vehicle in Example 3.

4.1 Illumina RNA-Seq Technology

Illumina dye sequencing begins with the attachment of cDNA molecules to primers on a slide, followed by amplification of that DNA to produce local colonies. The four types (adenine, cytosine, guanine, and thymine) of reversible terminate bases are added, each fluorescently labeled with a different color and attached with a blocking group. The four bases then compete for binding sites on the template cDNA to be sequenced and non-incorporated molecules are washed away. After each synthesis, a laser is used to excite the dyes and a photograph of the incorporated base is taken. A chemical deblocking step is then used in the removal of the 3′ terminal blocking group and the dye in a single step. The process is repeated until the full cDNA molecule is sequenced.

Illumina RNA-Seq technology records the numerical frequency of sequences in a library population. 50 bp single reads with multiples of 30M single reads are guaranteed using Illumina sequencing technology. The RNA-Seq reads are aligned to the reference genome or reference transcriptome using Bowtie generating genome/transcriptome alignments. TopHat identifies the potential exon-exon splice junctions of the initial alignment. Then Cufflinks identifies and quantifies the transcripts from the preprocessed RNA-Seq alignment assembly. After this, Cuffmerge merges the identified transcript pieces to full length transcripts and annotates the transcripts based on the given annotations. Finally, merged transcripts from two or more samples/conditions are compared using Cuffdiff to determine the differential expression levels at transcript and gene level including a measure of significance between samples/conditions.

Differential gene expression. Operating on the RNA-Seq alignments and Cuffinks processing, Cuffdiff tracks the mapped reads and determines the fragment per kilo base per million mapped reads (FPKM) for each transcript in all the samples. Primary transcripts and gene FPKMs are then computed by adding up the FPKMs of each primary transcript group or gene group. For each pair of samples (control vs. case), the differential expression values such as fold change and p-value are computed.

4.2 Sample Preparation

RNA extraction for gene expression analysis was performed as described in Example 2 above to obtain the following pool samples:

Samples List

| SAMPLE CODES | SAMPLE DESCRIPTION |
|---|---|
| 1512, 1513, 1534 | POOL SAMP8 VEH females 4 months |
| 1404, 1405, 1406 | POOL SAMP8 Comp1 (0.96 mg/kg/day) |
| | females 4 months |
| 1521, 1543, 1544 | POOL SAMP8 VEH males 4 months |
| 1522, 1523 | POOL SAMP8 Comp1 (0.32 mg/kg/day) |
| | males 4 months |

4.3 Gene Expression Results

The results obtained are shown in the table below:

| | | Male FPKM | | | Female FPKM | | |
|---|---|---|---|---|---|---|---|
| | | | Comp1 0.32 | Fold | | Comp1 0.96 | Fold |
| gene | refseq_id | VEH | mg/kg/day | Change | VEH | mg/kg/day | Change |
| S100A9 | NM_001281852 | 1.64 | 1.03 | −0.67 | 0.82 | 0.61 | −0.43 |

As used herein, Comp1 means Compound 1, VEH means vehicle

Changes in S100A9 expression in the hippocampus of Compound 1-treated relative to vehicle-treated SAMP8 mice were validated by RNA-seq analysis. Treatment with Compound 1 down-regulated S100A9 expression compared to vehicle-treated animals of the same sex.

### Example 5: Validation of Effects of LSD1 Inhibitors on S100A9 and S100A8 Expression by qRT-PCR

5.1 Quantitative RT-PCR

qRT-PCR is a variant of the PCR (Polymerase Chain Reaction) method that permits the simultaneous exponential amplification and detection of specific cDNA fragments. The Taqman gene expression assays employ the principle of doubly labeled hydrolysis probes. The probes are marked with a fluorescent moiety at their 5′ end and with a quencher moiety at the 3′ end, which prevents the generation of fluorescence according to the Förster energy transfer principle.

During the amplification process, the hydrolysis probe hybridizes to its complementary sequence in the target amplicon. During each cycle, the Taq polymerase initiates the production of a copy of the target sequence starting from the primer. When the Taq polymerase reaches the hydrolysis probe, its 5′-3′ exonuclease activity fragments the hydrolysis probe, and liberates the fluorescent group from the quencher moiety, resulting in the emission of a fluorescent signal.

In the exponential phase of the amplification reaction, the intensity of the fluorescence is directly proportional to the quantity of PCR product formed. The LightCycler® 480 Software determines the “crossing point” (Cp), i.e. the point where the reaction's fluorescence reaches the maximum of the second derivative of the amplification curve, which corresponds to the point where the acceleration of the fluorescence signal is at its maximum. Hence, this crossing point should always be located in the middle of the log-linear portion of the PCR amplification plot. The 2-Cp values are proportional to the target mRNA concentration in the original RNA sample.

qRT-PCR analysis of gene expression levels of S100A8 was performed using Taqman assay Mm00496696_g1, Life Technologies; amplicon length 131 bp, targeting exon 2-3 boundary, RefSeq NM_013650.2, assay location 191) and of S100A9 using Taqman assay Mm00656925_m1, Life Technologies; amplicon length 162 bp, targeting exon 2-3 boundary, RefSeq NM_001281852.1, assay location 212) on total RNA extracted from the hippocampus of SAMR1 mice and of SAMP8 mice treated for 2 or 4 months with vehicle or with Compound 1 obtained as described in Example 3. Samples from animals receiving treatment for 2 months and 4 months were processed and statistically analyzed together. After extraction (RNeasy Mini KIT; QIAGEN), total RNA was reverse transcribed to obtain 1st strand cDNA (High Capacity RNA-to-cDNA Master Mix; Applied Biosystems). A serial dilution of 1st strand product from hippocampus was used to perform triplicate qRT-PCR (Taqman gene expression assay, Life technologies) reactions to analyze the Cp values of S100A8 and S100A9. Cp increase was normalized relative to the expression level of an endogenous reference gene (GADPH).

5.2 Results

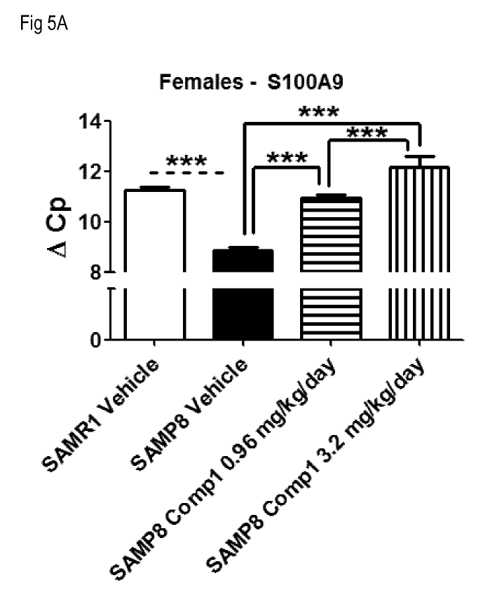

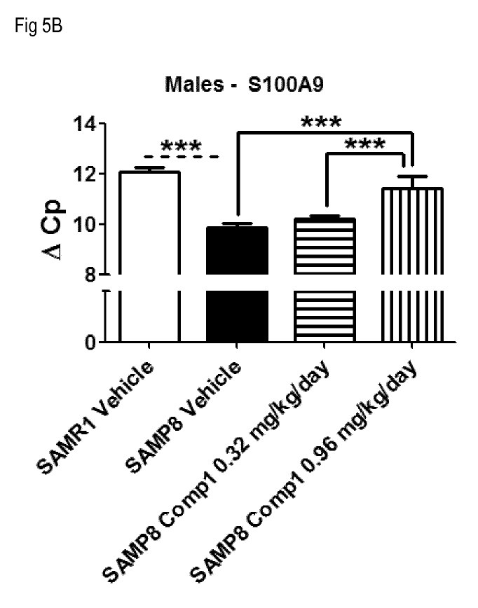

Changes in S100A9 expression in the hippocampus of Compound 1-treated relative to vehicle-treated SAMP8 mice were validated by qRT-PCR. The results obtained are shown in FIGS. 5A and 5B. S100A9 was up-regulated in SAMP8 vs SAMR1 mice and treatment with Compound 1 down-regulated S100A9 expression in a dose-dependent fashion in females at 0.96 mg/kg/day (p<0.001) and 3.2 mg/kg/day (p<0.001); and also in males at 0.96 mg/kg (p<0.001). Similarly, S100A8 was up-regulated in SAMP8 vs SAMR1 female mice and treatment with Compound 1 resulted in a down-regulated S100A8 expression tendency. *** p<0.001

### Example 6: S100A9 and S100A8 are Down-Regulated in Brain Upon Treatment with Compound 1 or Compound 2

This example illustrates that the degree of S100A9 and S100A8 down-regulation in the brain is dependent on the degree of LSD1 inhibition.

As part of Maximum Tolerated Dose (MTD) studies, LSD1 inhibitors were administered to mice at various doses and brain samples were collected and subjected to GE analysis.

6.1 Test Compounds

Compound 1, Compound 2.

These compounds are enantiomers with very similar pharmacokinetic profile and biochemical potency for MAO-B and MAO-A inhibitions, but highly distinct biochemical potency for LSD1 inhibition, as shown by the data provided in Example 1.

6.2 Preparation of Test Compound for Administration

Appropriate quantities of powered Compound 1 or Compound 2 were dissolved in vehicle (20% 2-hydroxypropyl-β-cyclodextrin; 80% H₂O), vortexed and placed in an ultrasonic bath for 10 minutes.

6.3 Mice Strain and Treatment

Male Hsd:ICR (CD1®) mice were maintained in air- and temperature-controlled cages with regular supply of water and food. A maximum of 3 mice were raised per cage. Three mice were assigned to each group. Before the first administration, each mouse was labeled and weighed. Test compounds were administered orally using 1 ml syringes using animal feeding needles proper for mice at 10 ml/kg as follows.

(Binary code; 1=dose and 0=no dose)
G1: Compound 2, 3 mg/kg, (1111100), oral (n=3) one week
G2: Compound 2, 10 mg/kg, (1111100), oral (n=3) one week
G3: Compound 2, 30 mg/kg, (1111100), oral (n=3) one week
G4: Compound 2, 100 mg/kg, (1111100), oral (n=3) one week
G5: Compound 1, 3 mg/kg, (1111100), oral (n=3) one week
G6: Compound 1, 10 mg/kg, (1111100), oral (n=3) one week
G7: Compound 1, 30 mg/kg, (1111100), oral (n=3) one week
G8: Compound 1, 100 mg/kg, (1111100), oral (n=3) one week
G9: Vehicle, (1), oral (n=3) one week

As used herein, Comp1 means Compound 1 and Comp2 means Compound 2.

6.4 Samples

After killing the animals, tissue samples of brain (left hemisphere) were extracted and placed immediately on liquid nitrogen and stored at −80° C. The left brain hemisphere samples were pre-processed for RNA extraction with 0.5 ml of RLT lysis buffer from Qiagen using an Ultraturrax.

6.5 Gene Expression Analysis

6.5.1 Sample Preparation

RNA extraction and labeling for gene expression analysis was performed as described above to obtain the following pool samples:

Samples List

| SAMPLE CODE | SAMPLE DESCRIPTION |
|---|---|
| Pool Comp2 B5 G1 Cy5 | Comp2, 5d Brain 3 mg/kg |
| Pool Comp2 B5 G2 Cy5 | Comp2, 5d Brain 10 mg/kg |
| Pool Comp2 B5 G3 Cy5 | Comp2, 5d Brain 30 mg/kg |
| Pool Comp2 B5 G4 Cy5 | Comp2, 5d Brain 100 mg/kg |
| Pool Comp1 B5 G5 Cy5 | Comp1, 5d Brain 3 mg/kg |
| Pool Comp1 B5 G6 Cy5 | Comp1, 5d Brain 10 mg/kg |
| Pool Comp1 B5 G7 Cy5 | Comp1, 5d Brain 30 mg/kg |
| Pool Comp1 B5 G8 Cy5 | Comp1, 5d Brain 100 mg/kg |
| Pool V B5 G9 Cy5 | 5d Brain vehicle |
| Pool V B5 G9 Cy3 | 5d Brain vehicle |

6.5.2 Hybridization List

The following hybridizations were performed as described above

| | | CONTROL | | |
|---|---|---|---|---|
| HYB. # | CODE | DESCRIPTION | CODE | SAMPLE DESCRIPTION |
| 1 | Pool V B5 G9 Cy3 | 5d Brain vehicle | Pool Comp2 B5 G1 Cy5 | Comp2 5d Brain 3 mg/kg |
| 2 | Pool V B5 G9 Cy3 | 5d Brain vehicle | Pool Comp2 B5 G2 Cy5 | Comp2 5d Brain 10 mg/kg |
| 3 | Pool V B5 G9 Cy3 | 5d Brain vehicle | Pool Comp2 B5 G3 Cy5 | Comp2 5d Brain 30 mg/kg |
| 4 | Pool V B5 G9 Cy3 | 5d Brain vehicle | Pool Comp2 B5 G4 Cy5 | Comp2 5d Brain 100 mg/kg |
| 5 | Pool V B5 G9 Cy3 | 5d Brain vehicle | Pool Comp1 B5 G5 Cy5 | Comp1 5d Brain 3 mg/kg |
| 6 | Pool V B5 G9 Cy3 | 5d Brain vehicle | Pool Comp1 B5 G6 Cy5 | Comp1 5d Brain 10 mg/kg |
| 7 | Pool V B5 G9 Cy3 | 5d Brain vehicle | Pool Comp1 B5 G7 Cy5 | Comp1 5d Brain 30 mg/kg |
| 8 | Pool V B5 G9 Cy3 | 5d Brain vehicle | Pool Comp1 B5 G8 Cy5 | Comp1 5d Brain 100 mg/kg |
| 9 | Pool V B5 G9 Cy3 | 5d Brain vehicle | Pool V B5 G9 Cy5 | 5d Brain vehicle |
| 10 | Pool V B5 G9 Cy3 | 5d Brain vehicle | Pool V B5 G9 Cy5 | 5d Brain vehicle |
| 11 | Pool V B5 G9 Cy3 | 5d Brain vehicle | Pool V B5 G9 Cy5 | 5d Brain vehicle |

6.5.3 Replica Analysis

Three replica hybridizations were included for the vehicle vs vehicle comparison (SELF TO SELF); which were grouped for replica analysis. No biological replicas or repeat hybridizations were included for the other comparisons, therefore replica analysis was intra-array.

GROUP CODE

3x Pool V B5 G9 Cy3 vs Pool V B5 G9 Cy5
Pool V B5 G9 Cy3 vs Pool Comp2 B5 G1 Cy5
Pool V B5 G9 Cy3 vs Pool Comp2 B5 G2 Cy5
Pool V B5 G9 Cy3 vs Pool Comp2 B5 G3 Cy5
Pool V B5 G9 Cy3 vs Pool Comp2 B5 G4 Cy5
Pool V B5 G9 Cy3 vs Pool Comp1 B5 G5 Cy5
Pool V B5 G9 Cy3 vs Pool Comp1 B5 G6 Cy5
Pool V B5 G9 Cy3 vs Pool Comp1 B5 G7 Cy5
Pool V B5 G9 Cy3 vs Pool Comp1 B5 G8 Cy5

6.5.4 Gene Expression Results

The results obtained are shown in the table below:

| | | Hybridization signal | | Statistics |
|---|---|---|---|---|
| RAIN DAY 5 | log2 (sample/control) | control_Cy3 | sample_Cy5 | p-value |
| | Comp2 - 3 mg/kg/vehicle | | | |
| S100A8 | −0.101464 | 135.57 | 126.363 | 0.0655865 |
| S100A9 | −0.251184 | 347.361 | 291.855 | 0.0140746 |
| TUBB3 | 0.094582 | 3419.03 | 3650.69 | 0.0120556 |
| TUBB2C | 0.190822 | 5034.64 | 5746.61 | 0.0014985 |
| Low | | 38 | 38 | |
| | Comp2 - 10 mg/kg/vehicle | | | |
| S100A8 | −1.15568 | 155.933 | 69.9912 | 0.0000057 |
| S100A9 | −1.51472 | 334.82 | 117.175 | 0.0004347 |
| TUBB3 | −0.123943 | 4039.16 | 3706.64 | 0.0079356 |
| TUBB2C | 0.117903 | 5291.46 | 5742.06 | 0.0111321 |
| Low | | 37 | 37 | |
| | Comp2 - 30 mg/kg/vehicle | | | |
| S100A8 | −1.02864 | 163.592 | 80.188 | 2.34E−05 |
| S100A9 | −1.285 | 364.406 | 149.542 | 5.95E−05 |
| TUBB3 | −0.00203348 | 4415.82 | 4409.6 | 0.899688 |
| TUBB2C | 0.179776 | 5584.88 | 6326.04 | 0.0018191 |
| Low | | 36 | 36 | |
| | Comp2 - 100 mg/kg/vehicle | | | |
| S100A8 | −1.92058 | 149.004 | 39.3591 | 0.0000023 |
| S100A9 | −2.87556 | 332.776 | 45.3442 | 3.58E−05 |
| TUBB3 | −0.433804 | 3044.33 | 2253.74 | 0.0001474 |
| TUBB2C | −0.398341 | 4908.57 | 3724.28 | 0.0012381 |
| Low | | 37 | 35 | |
| | Comp1 - 3 mg/kg/vehicle | | | |
| S100A8 | −1.45228 | 151.321 | 55.2994 | 2.32E−04 |
| S100A9 | −1.93493 | 374.416 | 97.9225 | 2.06E−05 |
| TUBB3 | −0.185238 | 4039.96 | 3553.16 | 0.0011209 |
| TUBB2C | −0.0321599 | 5120.92 | 5008.03 | 0.162332 |
| Low | | 35 | 35 | |
| | Comp1 - 10 mg/kg/vehicle | | | |
| S100A8 | −1.80982 | 138.972 | 39.6385 | 3.44E−05 |
| S100A9 | −2.96162 | 360.46 | 46.2723 | 9.00E−06 |
| TUBB3 | −0.596407 | 3665.88 | 2424.61 | 0.0000767 |
| TUBB2C | −0.308977 | 4912.07 | 3965.09 | 0.0013168 |
| Low | | 36 | 36 | |
| | Comp1 - 30 mg/kg/vehicle | | | |
| S100A8 | −1.85879 | 147.249 | 40.5977 | 4.00E−06 |
| S100A9 | −2.9408 | 348.843 | 45.4319 | 9.50E−06 |
| TUBB3 | −0.11023 | 3142.63 | 2911.46 | 0.0066891 |
| TUBB2C | 0.035435 | 4937.3 | 5060.07 | 0.158613 |
| Low | | 38 | 39 | |
| | Comp1 - 100 mg/kg/vehicle | | | |
| S100A8 | −1.78625 | 139.552 | 40.4596 | 2.52E−04 |
| S100A9 | −2.79213 | 392.777 | 56.7063 | 8.00E−07 |
| TUBB3 | −0.0257939 | 3733.62 | 3667.46 | 0.211211 |
| TUBB2C | 0.0423985 | 5417.68 | 5579.26 | 0.0819403 |
| Low | | 35 | 35 | |
| | vehicle/vehicle | | | |
| S100A8 | 0.0608739 | 145.671 | 158.755 | 6.90E−03 |
| S100A9 | 0.0608739 | 344.235 | 342.735 | 0.772812 |
| TUBB3 | −0.0512777 | 3170.5 | 3059.79 | 0.121021 |
| TUBB2C | 0.0289686 | 4789.85 | 4887 | 0.398542 |
| Low | | 36 | 36 | |

Low: Lowest above background signal on the array, rounded to the lower unit. Log2 (sample/control) values were calculated without background subtraction as described above.
S100A8 and S100A9 were down-regulated by treatment with LSD1 inhibitors, Compound 1 and Compound 2. Two HOUSEKEEPING genes, Tubb3 and Tubb2c, were included for comparison. The potency of the effect was correlated to the biochemical LSD1 inhibitory potency in vitro, i.e Compound 2 << Compound 1. These data further confirm that the down-regulation of S100A8 and S100A9 observed by treatment with Compound 1 in Example 3 is due to the LSD1-inhibitory component of Compound 1.

### Example 7: S100A8 and S100A9 are Down-Regulated Upon Treatment with LSD1 Inhibitors in Spleen, Liver and Brain Tissue

This example further illustrates that LSD1 inhibitors downregulate S100A8 and S100A9 gene expression in a variety of tissues and that the degree of downregulation is related to the degree of LSD1 inhibition. Tissue samples of animals treated with LSD1 inhibitors obtained from MTD studies were analyzed for gene expression of S100A9 and S100A8.

7.1 Test Compounds

Compounds 3, 4 and 5, as described in Example 1.

Compound 3 is a LSD1/MAO-B inhibitor, whereas Compounds 2 and 3 are more selective LSD1 inhibitors with biochemical potency for LSD1, MAO-B and MAO-A as disclosed in Example 1.

7.2 Preparation of Test Compounds for Administration

Powered Compound 3, 4 or 5 was dissolved in a 20% solution of 2-hydroxypropyl-R-cyclodextrin in water at the appropriate concentrations, vortexed and placed in an ultrasonic bath for 5 minutes.

### 7.3 MICE STRAIN AND TREATMENT

Hsd:Athymic Nude-Foxn1nu mice were maintained in air- and temperature-controlled cages with regular supply of water and food. A maximum of 6 mice/cage were raised. Before the first administration, the mice were labeled and weighed.

Intraperitoneal injection was done with 1 ml syringes using 27G needles at 15 ml/kg.

Animals were administered compound by i.p. injection on 5 consecutive days. Treatment schemes were as follows (1: administration; 0: no administration)

### OG 044/23:

G1: Comp3, 5 mg/kg, (1111100), i.p. (n=6) one week
G2: Comp3, 10 mg/kg, (1111100)), i.p. (n=6), one week
G3: Comp3, 20 mg/kg, (1111100), i.p. (n=6), one week
G4: Comp3, 40 mg/kg, (1111100), i.p. (n=6), one week
G8: Vehicle, (1111100), i.p. (n=6) one week

### OG 044/24

G1: Comp4, 1 mg/kg, (1111100), i.p. (n=6) one week
G2: Comp4, 3 mg/kg, (1111100)), i.p. (n=6), one week
G3: Comp4, 10 mg/kg, (1111100), i.p. (n=6), one week
G4: Comp5, 3.3 mg/kg, (1111100), i.p. (n=6), one week
G5: Comp5, 11 mg/kg, (1111100), i.p. (n=6), one week
G6: Comp5, 33 mg/kg, (1111100), i.p. (n=6), one week
G7: Vehicle, (1111100), i.p. (n=6) one week

### OG 044/25

G5: Vehicle, (1111100), i.p. (n=6) one week

7.4 Sampling Procedures:

Immediately after killing each animal, samples of spleen, liver (caudate lobule) and brain were extracted. These tissues were rinsed in physiological solution and frozen on liquid nitrogen. The samples were thereafter homogenized with 10×RLT buffer (1 ml/sample) (Qiagen #79216) with Ultraturrax and stored at −80° C. for further processing and RNA extraction.

7.5 Gene Expression Analysis

7.5.1 Sample Preparation

RNA extraction and labeling for gene expression analysis was performed as described above to obtain the following pool samples:

Pool 23-B5-G1 Cy5 means Cy5 Labeled aRNA derived from Pool from RNA from brain from mice from treatment group G1 of OG 044/23 sacrificed on day 5 of treatment

“OG 044/23 G1-1,2,3-Brain-day 5th” means RNA derived from the brain of mouse No. 1, 2, 3 from treatment group G1 of OG 044/23 sacrificed on day 5 of treatment

| SAMPLE CODE | SAMPLE DESCRIPTION | MOUSE TREATMENT |
|---|---|---|
| Pool 23-B5-G1 Cy5 | OG 044/23 cr (G1-1,2,3) - Brain - day 5th | Comp3 5 mg/kg |
| Pool 23-B5-G2 Cy5 | OG 044/23 cr (G2-1,2,3) - Brain - day 5th | Comp3 10 mg/kg |
| Pool 23-B5-G3 Cy5 | OG 044/23 cr (G3-1,2,3) - Brain - day 5th | Comp3 20 mg/kg |
| Pool 23-B5-G4 Cy5 | OG 044/23 cr (G4-1,2,3) - Brain - day 5th | Comp3 40 mg/kg |
| Pool 23-S5-G1 Cy5 | OG 044/23 cr (G1-1,2,3) - Spleen - day 5th | Comp3 5 mg/kg |
| Pool 23-S5-G2 Cy5 | OG 044/23 cr (G2-1,2,3) - Spleen - day 5th | Comp3 10 mg/kg |
| Pool 23-S5-G3 Cy5 | OG 044/23 cr (G3-1,2,3) - Spleen - day 5th | Comp3 20 mg/kg |
| Pool 23-S5-G4 Cy5 | OG 044/23 cr (G4-1,2,3) - Spleen - day 5th | Comp3 40 mg/kg |
| Pool 23-L5-G1 Cy5 | OG 044/23 cr (G1-1,2,3) - Liver - day 5th | Comp3 5 mg/kg |
| Pool 23-L5-G2 Cy5 | OG 044/23 cr (G2-1,2,3) - Liver - day 5th | Comp3 10 mg/kg |
| Pool 23-L5-G3 Cy5 | OG 044/23 cr (G3-1,2,3) - Liver - day 5th | Comp3 20 mg/kg |
| Pool 23-L5-G4 Cy5 | OG 044/23 cr (G4-1,2,3) - Liver - day 5th | Comp3 40 mg/kg |
| Pool 24-B5-G1 Cy5 | OG 044/24 cr (G1-1,2,3) - Brain - day 5th | Comp4 1 mg/kg |
| Pool 24-B5-G2 Cy5 | OG 044/24 cr (G2-1,2,3) - Brain - day 5th | Comp4 3 mg/kg |
| Pool 24-B5-G3 Cy5 | OG 044/24 cr (G3-1,2,3) - Brain - day 5th | Comp4 10 mg/kg |
| Pool 24-B5-G4 Cy5 | OG 044/24 cr (G4-1,2,3) - Brain - day 5th | Comp5 3.3 mg/kg |
| Pool 24-B5-G5 Cy5 | OG 044/24 cr (G5-1,2,3) - Brain - day 5th | Comp5 11 mg/kg |
| Pool 24-B5-G6 Cy5 | OG 044/24 cr (G6-1,2,3) - Brain - day 5th | Comp5 33 mg/kg |
| Pool 24-S5-G1 Cy5 | OG 044/24 cr (G1-1,2,3) - Spleen - day 5th | Comp4 1 mg/kg |
| Pool 24-S5-G2 Cy5 | OG 044/24 cr (G2-1,2,3) - Spleen - day 5th | Comp4 3 mg/kg |
| Pool 24-S5-G3 Cy5 | OG 044/24 cr (G3-1,2,3) - Spleen - day 5th | Comp4 10 mg/kg |
| Pool 24-S5-G4 Cy5 | OG 044/24 cr (G4-1,2,3) - Spleen - day 5th | Comp5 3.3 mg/kg |
| Pool 24-S5-G5 Cy5 | OG 044/24 cr (G5-1,2,3) - Spleen - day 5th | Comp5 11 mg/kg |
| Pool 24-S5-G6 Cy5 | OG 044/24 cr (G6-1,2,3) - Spleen - day 5th | Comp5 33 mg/kg |
| Pool 24-L5-G1 Cy5 | OG 044/24 cr (G1-1,2,3) - Liver - day 5th | Comp4 1 mg/kg |
| Pool 24-L5-G2 Cy5 | OG 044/24 cr (G2-1,2,3) - Liver - day 5th | Comp4 3 mg/kg |
| Pool 24-L5-G3 Cy5 | OG 044/24 cr (G3-1,2,3) - Liver - day 5th | Comp4 10 mg/kg |
| Pool 24-L5-G4 Cy5 | OG 044/24 cr (G4-1,2,3) - Liver - day 5th | Comp5 3.3 mg/kg |
| Pool 24-L5-G5 Cy5 | OG 044/24 cr (G5-1,2,3) - Liver - day 5th | Comp5 11 mg/kg |
| Pool 24-L5-G6 Cy5 | OG 044/24 cr (G6-1,2,3) - Liver - day 5th | Comp5 33 mg/kg |
| Pool 25-B5-G5 Cy5 | OG 044/25 cr (G5-1,2,3) - Brain - day 5th | VEH |
| Pool 25-S5-G5 Cy5 | OG 044/25 cr (G5-1,2,3) - Spleen - day 5th | VEH |
| Pool 25-L5-G5 Cy5 | OG 044/25 cr (G5-1,2,3) - Liver - day 5th | VEH |
| Pool 25-B5-G5 Cy3 | OG 044/25 cr (G5-1,2,3) - Brain - day 5th | VEH |
| Pool 25-S5-G5 Cy3 | OG 044/25 cr (G5-1,2,3) - Spleen - day 5th | VEH |
| Pool 25-L5-G5 Cy3 | OG 044/25 cr (G5-1,2,3) - Liver - day 5th | VEH |
| Pool 23-B5-G8 Cy3 | OG 044/23 cr (G8-1,2,3) - Brain - day 5th | VEH |
| Pool 23-S5-G8 Cy3 | OG 044/23 cr (G8-1,2,3) - Spleen - day 5th | VEH |
| Pool 23-L5-G8 Cy3 | OG 044/23 cr (G8-1,2,3) - Liver - day 5th | VEH |

7.5.2 Hybridizations

The following labeled samples were co-hybridized on applicant's mouse WGA arrays and analyzed as described above in Example 2:

| HYB. # | CODE | CONTROL DESCRIPTION | CODE | SAMPLE DESCRIPTION |
|---|---|---|---|---|
| 1 | Pool 23-B5-G8 Cy3 | OG 044/23 cr (G8-1,2,3) - | Pool 23-B5-G1 | OG 044/23 cr (G1-1,2,3) - |
| | | Brain - day 5th | Cy5 | Brain - day 5th |
| 2 | Pool 23-B5-G8 Cy3 | OG 044/23 cr (G8-1,2,3) - | Pool 23-B5-G2 | OG 044/23 cr (G2-1,2,3) - |
| | | Brain - day 5th | Cy5 | Brain - day 5th |
| 3 | Pool 23-B5-G8 Cy3 | OG 044/23 cr (G8-1,2,3) - | Pool 23-B5-G3 | OG 044/23 cr (G3-1,2,3) - |
| | | Brain - day 5th | Cy5 | Brain - day 5th |
| 4 | Pool 23-B5-G8 Cy3 | OG 044/23 cr (G8-1,2,3) - | Pool 23-B5-G4 | OG 044/23 cr (G4-1,2,3) - |
| | | Brain - day 5th | Cy5 | Brain - day 5th |
| 7 | Pool 23-S5-G8 Cy3 | OG 044/23 cr (G8-1,2,3) - | Pool 23-S5-G1 | OG 044/23 cr (G1-1,2,3) - |
| | | Spleen - day 5th | Cy5 | Spleen - day 5th |
| 8 | Pool 23-S5-G8 Cy3 | OG 044/23 cr (G8-1,2,3) - | Pool 23-S5-G2 | OG 044/23 cr (G2-1,2,3) - |
| | | Spleen - day 5th | Cy5 | Spleen - day 5th |
| 9 | Pool 23-S5-G8 Cy3 | OG 044/23 cr (G8-1,2,3) - | Pool 23-S5-G3 | OG 044/23 cr (G3-1,2,3) - |
| | | Spleen - day 5th | Cy5 | Spleen - day 5th |
| 10 | Pool 23-S5-G8 Cy3 | OG 044/23 cr (G8-1,2,3) - | Pool 23-S5-G4 | OG 044/23 cr (G4-1,2,3) - |
| | | Spleen - day 5th | Cy5 | Spleen - day 5th |
| 13 | Pool 23-L5-G8 Cy3 | OG 044/23 cr (G8-1,2,3) - | Pool 23-L5-G1 | OG 044/23 cr (G1-1,2,3) - |
| | | Liver - day 5th | Cy5 | Liver - day 5th |
| 14 | Pool 23-L5-G8 Cy3 | OG 044/23 cr (G8-1,2,3) - | Pool 23-L5-G2 | OG 044/23 cr (G2-1,2,3) - |
| | | Liver - day 5th | Cy5 | Liver - day 5th |
| 15 | Pool 23-L5-G8 Cy3 | OG 044/23 cr (G8-1,2,3) - | Pool 23-L5-G3 | OG 044/23 cr (G3-1,2,3) - |
| | | Liver - day 5th | Cy5 | Liver - day 5th |
| 16 | Pool 23-L5-G8 Cy3 | OG 044/23 cr (G8-1,2,3) - | Pool 23-L5-G4 | OG 044/23 cr (G4-1,2,3) - |
| | | Liver - day 5th | Cy5 | Liver - day 5th |
| 19 | Pool 25-B5-G5 Cy3 | OG 044/25 cr (G5-1,2,3) - | Pool 24-B5-G1 | OG 044/24 cr (G1-1,2,3) - |
| | | Brain - day 5th | Cy5 | Brain - day 5th |
| 20 | Pool 25-B5-G5 Cy3 | OG 044/25 cr (G5-1,2,3) - | Pool 24-B5-G2 | OG 044/24 cr (G2-1,2,3) - |
| | | Brain - day 5th | Cy5 | Brain - day 5th |
| 21 | Pool 25-B5-G5 Cy3 | OG 044/25 cr (G5-1,2,3) - | Pool 24-B5-G3 | OG 044/24 cr (G3-1,2,3) - |
| | | Brain - day 5th | Cy5 | Brain - day 5th |
| 22 | Pool 25-B5-G5 Cy3 | OG 044/25 cr (G5-1,2,3) - | Pool 24-B5-G4 | OG 044/24 cr (G4-1,2,3) - |
| | | Brain - day 5th | Cy5 | Brain - day 5th |
| 23 | Pool 25-B5-G5 Cy3 | OG 044/25 cr (G5-1,2,3) - | Pool 24-B5-G5 | OG 044/24 cr (G5-1,2,3) - |
| | | Brain - day 5th | Cy5 | Brain - day 5th |
| 24 | Pool 25-B5-G5 Cy3 | OG 044/25 cr (G5-1,2,3) - | Pool 24-B5-G6 | OG 044/24 cr (G6-1,2,3) - |
| | | Brain - day 5th | Cy5 | Brain - day 5th |
| 25 | Pool 25-S5-G5 Cy3 | OG 044/25 cr (G5-1,2,3) - | Pool 24-S5-G1 | OG 044/24 cr (G1-1,2,3) - |
| | | Spleen - day 5th | Cy5 | Spleen - day 5th |
| 26 | Pool 25-S5-G5 Cy3 | OG 044/25 cr (G5-1,2,3) - | Pool 24-S5-G2 | OG 044/24 cr (G2-1,2,3) - |
| | | Spleen - day 5th | Cy5 | Spleen - day 5th |
| 27 | Pool 25-S5-G5 Cy3 | OG 044/25 cr (G5-1,2,3) - | Pool 24-S5-G3 | OG 044/24 cr (G3-1,2,3) - |
| | | Spleen - day 5th | Cy5 | Spleen - day 5th |
| 28 | Pool 25-S5-G5 Cy3 | OG 044/25 cr (G5-1,2,3) - | Pool 24-S5-G4 | OG 044/24 cr (G4-1,2,3) - |
| | | Spleen - day 5th | Cy5 | Spleen - day 5th |
| 29 | Pool 25-S5-G5 Cy3 | OG 044/25 cr (G5-1,2,3) - | Pool 24-S5-G5 | OG 044/24 cr (G5-1,2,3) - |
| | | Spleen - day 5th | Cy5 | Spleen - day 5th |
| 30 | Pool 25-S5-G5 Cy3 | OG 044/25 cr (G5-1,2,3) - | Pool 24-S5-G6 | OG 044/24 cr (G6-1,2,3) - |
| | | Spleen - day 5th | Cy5 | Spleen - day 5th |
| 31 | Pool 25-L5-G5 Cy3 | OG 044/25 cr (G5-1,2,3) - | Pool 24-L5-G1 | OG 044/24 cr (G1-1,2,3) - |
| | | Liver - day 5th | Cy5 | Liver - day 5th |
| 32 | Pool 25-L5-G5 Cy3 | OG 044/25 cr (G5-1,2,3) - | Pool 24-L5-G2 | OG 044/24 cr (G2-1,2,3) - |
| | | Liver - day 5th | Cy5 | Liver - day 5th |
| 33 | Pool 25-L5-G5 Cy3 | OG 044/25 cr (G5-1,2,3) - | Pool 24-L5-G3 | OG 044/24 cr (G3-1,2,3) - |
| | | Liver - day 5th | Cy5 | Liver - day 5th |
| 34 | Pool 25-L5-G5 Cy3 | OG 044/25 cr (G5-1,2,3) - | Pool 24-L5-G4 | OG 044/24 cr (G4-1,2,3) - |
| | | Liver - day 5th | Cy5 | Liver - day 5th |
| 35 | Pool 25-L5-G5 Cy3 | OG 044/25 cr (G5-1,2,3) - | Pool 24-L5-G5 | OG 044/24 cr (G5-1,2,3) - |
| | | Liver - day 5th | Cy5 | Liver - day 5th |
| 36 | Pool 25-L5-G5 Cy3 | OG 044/25 cr (G5-1,2,3) - | Pool 24-L5-G6 | OG 044/24 cr (G6-1,2,3) - |
| | | Liver - day 5th | Cy5 | Liver - day 5th |
| 40 | Pool 25-B5-G5 Cy3 | OG 044/25 cr (G5-1,2,3) - | Pool 25-B5-G5 | OG 044/25 cr (G5-1,2,3) - |
| | | Brain - day 5th | Cy5 | Brain - day 5th |
| 41 | Pool 25-S5-G5 Cy3 | OG 044/25 cr (G5-1,2,3) - | Pool 25-S5-G5 | OG 044/25 cr (G5-1,2,3) - |
| | | Spleen - day 5th | Cy5 | Spleen - day 5th |
| 42 | Pool 25-L5-G5 Cy3 | OG 044/25 cr (G5-1,2,3) - | Pool 25-L5-G5 | OG 044/25 cr (G5-1,2,3) - |
| | | Liver - day 5th | Cy5 | Liver - day 5th |

7.5.3 Replica Analysis

Replica analysis was intra-array.

| | COMPARISON |
|---|---|
| GROUP CODE | (CONTROL-sample) |
| Pool 23-B5-G8 Cy3 vs Pool 23-B5-G1 Cy5 | Brain VEH-Comp3 |
| | 5 mg/kg |
| Pool 23-B5-G8 Cy3 vs Pool 23-B5-G2 Cy5 | Brain VEH-Comp3 |
| | 10 mg/kg |
| Pool 23-B5-G8 Cy3 vs Pool 23-B5-G3 Cy5 | Brain VEH-Comp3 |
| | 20 mg/kg |
| Pool 23-B5-G8 Cy3 vs Pool 23-B5-G4 Cy5 | Brain VEH-Comp3 |
| | 40 mg/kg |
| Pool 23-L5-G8 Cy3 vs Pool 23-L5-G1 Cy5 | Liver VEH-Comp3 |
| | 5 mg/kg |
| Pool 23-L5-G8 Cy3 vs Pool 23-L5-G2 Cy5 | Liver VEH-Comp3 |
| | 10 mg/kg |
| Pool 23-L5-G8 Cy3 vs Pool 23-L5-G3 Cy5 | Liver VEH-Comp3 |
| | 20 mg/kg |
| Pool 23-L5-G8 Cy3 vs Pool 23-L5-G4 Cy5 | Liver VEH-Comp3 |
| | 40 mg/kg |
| Pool 23-S5-G8 Cy3 vs Pool 23-S5-G1 Cy5 | Spleen VEH-Comp3 |
| | 5 mg/kg |
| Pool 23-S5-G8 Cy3 vs Pool 23-S5-G2 Cy5 | Spleen VEH-Comp3 |
| | 10 mg/kg |
| Pool 23-S5-G8 Cy3 vs Pool 23-S5-G3 Cy5 | Spleen VEH-Comp3 |
| | 20 mg/kg |
| Pool 23-S5-G8 Cy3 vs Pool 23-S5-G4 Cy5 | Spleen VEH-Comp3 |
| | 40 mg/kg |
| Pool 25-B5-G5 Cy3 vs Pool 24-B5-G1 Cy5 | Brain VEH-Comp4 |
| | 1 mg/kg |
| Pool 25-B5-G5 Cy3 vs Pool 24-B5-G2 Cy5 | Brain VEH-Comp4 |
| | 3 mg/kg |
| Pool 25-B5-G5 Cy3 vs Pool 24-B5-G3 Cy5 | Brain VEH-Comp4 |
| | 10 mg/kg |
| Pool 25-B5-G5 Cy3 vs Pool 24-B5-G4 Cy5 | Brain VEH-Comp5 |
| | 3.3 mg/kg |
| Pool 25-B5-G5 Cy3 vs Pool 24-B5-G5 Cy5 | Brain VEH-Comp5 |
| | 11 mg/kg |
| Pool 25-B5-G5 Cy3 vs Pool 24-B5-G6 Cy5 | Brain VEH-Comp5 |
| | 33 mg/kg |
| Pool 25-B5-G5 Cy3 vs Pool 25-B5-G5 Cy5 | Brain VEH-veh |
| Pool 25-L5-G5 Cy3 vs Pool 24-L5-G1 Cy5 | Liver VEH-Comp4 1 mg/kg |
| Pool 25-L5-G5 Cy3 vs Pool 24-L5-G2 Cy5 | Liver VEH-Comp4 3 mg/kg |
| Pool 25-L5-G5 Cy3 vs Pool 24-L5-G3 Cy5 | Liver VEH-Comp4 |
| | 10 mg/kg |
| Pool 25-L5-G5 Cy3 vs Pool 24-L5-G4 Cy5 | Liver VEH-Comp5 |
| | 3.3 mg/kg |
| Pool 25-L5-G5 Cy3 vs Pool 24-L5-G5 Cy5 | Liver VEH-Comp5 |
| | 11 mg/kg |
| Pool 25-L5-G5 Cy3 vs Pool 24-L5-G6 Cy5 | Liver VEH-Comp5 |
| | 33 mg/kg |
| Pool 25-L5-G5 Cy3 vs Pool 25-L5-G5 Cy5 | Liver VEH-veh |
| Pool 25-S5-G5 Cy3 vs Pool 24-S5-G1 Cy5 | Spleen VEH-Comp4 |
| | 1 mg/kg |
| Pool 25-S5-G5 Cy3 vs Pool 24-S5-G2 Cy5 | Spleen VEH-Comp4 |
| | 3 mg/kg |
| Pool 25-S5-G5 Cy3 vs Pool 24-S5-G3 Cy5 | Spleen VEH-Comp4 |
| | 10 mg/kg |
| Pool 25-S5-G5 Cy3 vs Pool 24-S5-G4 Cy5 | Spleen VEH-Comp5 |
| | 3.3 mg/kg |
| Pool 25-S5-G5 Cy3 vs Pool 24-S5-G5 Cy5 | Spleen VEH-Comp5 |
| | 11 mg/kg |
| Pool 25-S5-G5 Cy3 vs Pool 24-S5-G6 Cy5 | Spleen VEH-Comp5 |
| | 33 mg/kg |
| Pool 25-S5-G5 Cy3 vs Pool 25-S5-G5 Cy5 | Spleen VEH-Veh |

7.5.4 Gene Expression Results

The results obtained for S100A9 and S100A8 gene expression in brain, liver and spleen are shown in the following tables:

| | log2 | Hybridization signal | | Statistics |
|---|---|---|---|---|
| BRAIN | (sample/control) | control_Cy3 | sample_Cy5 | p-value |
| | Comp3 - | | | |
| | 5 mg/kg/VEH | | | |
| S100A8 | −0.116017 | 248.95 | 229.714 | 0.0528036 |
| S100A9 | 0.110561 | 701.012 | 756.846 | 0.0067591 |
| TUBB3 | −0.223014 | 3004.34 | 2574.04 | 0.0006934 |
| TUBB2C | 0.091149 | 5541.7 | 5903.12 | 0.019801 |
| | Comp3 - | | | |
| | 10 mg/kg/VEH | | | |
| S100A8 | −0.330416 | 232.91 | 185.235 | 0.0006585 |
| S100A9 | −0.55889 | 685.045 | 465.025 | 0.0001163 |
| TUBB3 | −0.0628319 | 3393.48 | 3248.86 | 0.0342762 |
| TUBB2C | −0.00895453 | 6267.43 | 6228.65 | 0.704312 |
| | Comp3 - | | | |
| | 20 mg/kg/VEH | | | |
| S100A8 | −1.21795 | 253.211 | 108.854 | 1.17E−05 |
| S100A9 | −1.38783 | 703.749 | 268.93 | 5.90E−06 |
| TUBB3 | −0.0238012 | 3129.74 | 3078.53 | 0.202855 |
| TUBB2C | 0.0728923 | 6593.57 | 6935.27 | 0.0201539 |
| | Comp3 - | | | |
| | 40 mg/kg/VEH | | | |
| S100A8 | −1.59783 | 271.032 | 89.5415 | 0.0004946 |
| S100A9 | −1.79791 | 724.946 | 208.488 | 5.20E−06 |
| TUBB3 | 0.0205359 | 3523.25 | 3573.76 | 0.316423 |
| TUBB2C | 0.063281 | 7087.54 | 7405.34 | 0.0378478 |
| | Comp5 - | | | |
| | 3.3 mg/kg/VEH | | | |
| S100A8 | −0.646209 | 419.687 | 268.162 | 3.46E−05 |
| S100A9 | −0.963497 | 1455.77 | 746.537 | 2.11E−05 |
| TUBB3 | −0.0512589 | 4912.3 | 4740.83 | 0.0376315 |
| TUBB2C | −0.0139633 | 10745.5 | 10642 | 0.636057 |
| | Comp5 - | | | |
| | 11 mg/kg/VEH | | | |
| S100A8 | −3.26296 | 369.474 | 38.4889 | 5.91E−05 |
| S100A9 | −4.54044 | 1408.99 | 60.5479 | 2.00E−07 |
| TUBB3 | −0.0283428 | 6340.6 | 6217.25 | 0.151714 |
| TUBB2C | −0.0612686 | 11645.3 | 11161.1 | 0.0314123 |
| | Comp5 - | | | |
| | 33 mg/kg/VEH | | | |
| S100A8 | −3.56994 | 435.767 | 36.6941 | 3.59E−05 |
| S100A9 | −4.69667 | 1514.28 | 58.3941 | 2.88E−05 |
| TUBB3 | 0.107328 | 5311.82 | 5722.06 | 0.0077311 |
| TUBB2C | −0.0514525 | 13041 | 12584.1 | 0.105786 |
| | Comp4 - | | | |
| | 1 mg/kg/VEH | | | |
| S100A8 | −1.76098 | 361.771 | 106.74 | 2.30E−06 |
| S100A9 | −2.14315 | 1352.76 | 306.245 | 3.20E−06 |
| TUBB3 | −0.038249 | 5126.16 | 4992.04 | 0.0853952 |
| TUBB2C | −0.0491179 | 10363.6 | 10016.7 | 0.0518424 |
| | Comp4 - | | | |
| | 3 mg/kg/VEH | | | |
| S100A8 | −2.35046 | 335.347 | 65.756 | 4.00E−06 |
| S100A9 | −3.28645 | 1294.92 | 132.716 | 0.0001198 |
| TUBB3 | 0.0785165 | 4857.36 | 5129.04 | 0.0113096 |
| TUBB2C | −0.0658487 | 10338.7 | 9877.42 | 0.0465475 |
| | Comp4 - | | | |
| | 10 mg/kg/VEH | | | |
| S100A8 | −3.04151 | 385.173 | 46.781 | 4.53E−05 |
| S100A9 | −4.22221 | 1469.58 | 78.7372 | 2.75E−05 |
| TUBB3 | −0.177847 | 6721.42 | 5941.88 | 0.0009661 |
| TUBB2C | −0.042907 | 12353.7 | 11991.7 | 0.0850895 |
| | Vehicle/VEH | | | |
| S100A8 | −0.418623 | 427.068 | 319.506 | 0.0056197 |
| S100A9 | −0.425301 | 1399.83 | 1042.43 | 0.0002849 |
| TUBB3 | 0.12973 | 5781.97 | 6325.99 | 0.0035088 |
| TUBB2C | 0.0473502 | 12132.4 | 12537.2 | 0.058029 |
| | log2 | Hybridization signal | | Statistics |
| LIVER | (sample/control) | control_Cy3 | sample_Cy5 | p-value |
| | Comp3 - | | | |
| | 5 mg/kg/VEH | | | |
| S100A8 | −1.37141 | 647.329 | 250.202 | 0.0000055 |
| S100A9 | −1.469 | 2270.08 | 820.026 | 0.0000061 |
| TUBB3 | −0.0974207 | 69.2938 | 64.7691 | 0.0591413 |
| TUBB2C | −0.119352 | 2906.83 | 2676.03 | 0.0188672 |
| | Comp3 - | | | |
| | 10 mg/kg/VEH | | | |
| S100A8 | −1.1185 | 608.779 | 280.387 | 0.0000137 |
| S100A9 | −1.41023 | 2235.37 | 841.06 | 0.0000077 |
| TUBB3 | 0.0234532 | 67.5989 | 68.7068 | 0.428766 |
| TUBB2C | −0.268688 | 2772.56 | 2301.43 | 0.002107 |
| | Comp3 - | | | |
| | 20 mg/kg/VEH | | | |
| S100A8 | −1.31838 | 663.39 | 266.01 | 3.50E−04 |
| S100A9 | −1.44015 | 2341.46 | 862.896 | 2.90E−06 |
| TUBB3 | −0.0422453 | 49.8966 | 48.4567 | 0.166853 |
| TUBB2C | −0.577977 | 3073.85 | 2059.18 | 0.0000399 |
| | Comp3 - | | | |
| | 40 mg/kg/VEH | | | |
| S100A8 | −2.17684 | 729.021 | 161.23 | 0.0001201 |
| S100A9 | −2.57138 | 2450.08 | 412.209 | 7.00E−07 |
| TUBB3 | 0.0192666 | 46.2878 | 46.9101 | 0.277263 |
| TUBB2C | −0.972821 | 3044.5 | 1551.2 | 0.0000148 |
| | Comp5 - | | | |
| | 3.3 mg/kg/VEH | | | |
| S100A8 | −0.47611 | 438.772 | 315.439 | 2.34E−03 |
| S100A9 | −0.572778 | 1468.58 | 987.357 | 6.64E−05 |
| TUBB3 | −0.448273 | 66.9185 | 49.0459 | 0.0002499 |
| TUBB2C | 0.369152 | 2841.97 | 3670.67 | 0.000257 |
| | Comp5 - | | | |
| | 11 mg/kg/VEH | | | |
| S100A8 | −2.35673 | 375.405 | 73.2915 | 9.81E−05 |
| S100A9 | −3.05631 | 1421.86 | 170.929 | 1.04E−04 |
| TUBB3 | −0.641122 | 67.3675 | 43.197 | 0.0035129 |
| TUBB2C | 0.457003 | 2903.16 | 3985.13 | 0.000075 |
| | Comp5 - | | | |
| | 33 mg/kg/VEH | | | |
| S100A8 | −2.90929 | 432.068 | 57.5133 | 2.20E−06 |
| S100A9 | −3.81082 | 1599.43 | 113.971 | 1.60E−06 |
| TUBB3 | −0.421099 | 69.6654 | 52.03 | 0.0005435 |
| TUBB2C | 0.320464 | 3471.85 | 4335.41 | 0.0006955 |
| | Comp4 - | | | |
| | 1 mg/kg/VEH | | | |
| S100A8 | −0.871751 | 404.016 | 220.788 | 8.02E−05 |
| S100A9 | −1.19204 | 1522.62 | 666.426 | 1.10E−05 |
| TUBB3 | −0.40896 | 121.463 | 91.4818 | 0.0029989 |
| TUBB2C | 0.297059 | 3521.82 | 4327.04 | 0.0010995 |
| | Comp4 - | | | |
| | 3 mg/kg/VEH | | | |
| S100A8 | −1.18111 | 478.702 | 211.114 | 6.78E−04 |
| S100A9 | −1.63169 | 1519.07 | 490.218 | 0.0000036 |
| TUBB3 | −0.243158 | 78.9793 | 66.7291 | 0.0010767 |
| TUBB2C | 0.106902 | 2972.69 | 3201.33 | 0.0064836 |
| | Comp4 - | | | |
| | 10 mg/kg/VEH | | | |
| S100A8 | −1.33533 | 435.176 | 172.461 | 4.02E−04 |
| S100A9 | −1.92803 | 1471.75 | 386.758 | 3.25E−04 |
| TUBB3 | 0.681142 | 64.8556 | 103.99 | 0.0000383 |
| TUBB2C | 0.329165 | 2502.36 | 3143.68 | 0.0006093 |
| | Vehicle/VEH | | | |
| S100A8 | 0.762602 | 303.336 | 514.624 | 0.0000682 |
| S100A9 | 0.387227 | 1092.61 | 1429 | 0.0008392 |
| TUBB3 | 0.299751 | 87.1803 | 107.313 | 0.0068201 |
| TUBB2C | 0.523662 | 2346.8 | 3373.76 | 0.0000781 |
| | log2 | Hybridization signal | | Statistics |
| SPLEEN | (sample/control) | control_Cy3 | sample_Cy5 | p-value |
| | Comp3 - | | | |
| | 5 mg/kg/VEH | | | |
| S100A8 | −1.18074 | 10277.2 | 4533.52 | 0.0004379 |
| S100A9 | −1.21321 | 25045.6 | 10802.4 | 0.0000119 |
| TUBB3 | −0.0294416 | 59.8944 | 58.6845 | 0.21592 |
| TUBB2C | −0.0527213 | 6478.39 | 6245.92 | 0.0762385 |
| | Comp3 - | | | |
| | 10 mg/kg/VEH | | | |
| S100A8 | −0.470554 | 8788.86 | 6342.81 | 0.0001133 |
| S100A9 | −0.581246 | 22715.6 | 15182.8 | 0.000045 |
| TUBB3 | −0.0225851 | 53.4995 | 52.6685 | 0.586351 |
| TUBB2C | −0.486263 | 5631.11 | 4019.89 | 0.0001504 |
| | Comp3 - | | | |
| | 20 mg/kg/VEH | | | |
| S100A8 | −0.529633 | 10126.5 | 7014.94 | 8.21E−05 |
| S100A9 | −0.517902 | 22505.3 | 15717.4 | 1.45E−04 |
| TUBB3 | −0.118253 | 67.8779 | 62.5361 | 0.0281949 |
| TUBB2C | −0.409736 | 6107.86 | 4597.76 | 0.0000958 |
| | Comp3 - | | | |
| | 40 mg/kg/VEH | | | |
| S100A8 | −1.65026 | 15074 | 4802.3 | 0.0000088 |
| S100A9 | −1.71866 | 44306.9 | 13461.8 | 1.30E−06 |
| TUBB3 | 0.168976 | 78.5777 | 88.3418 | 0.0016755 |
| TUBB2C | −0.947642 | 17333 | 8986.8 | 0.0001762 |
| | Comp5 - | | | |
| | 3.3 mg/kg/VEH | | | |
| S100A8 | −1.65026 | 15074 | 4802.3 | 8.80E−06 |
| S100A9 | −1.71866 | 44306.9 | 13461.8 | 1.30E−06 |
| TUBB3 | 0.168976 | 78.5777 | 88.3418 | 0.0016755 |
| TUBB2C | −0.947642 | 17333 | 8986.8 | 0.0001762 |
| | Comp5 - | | | |
| | 11 mg/kg/VEH | | | |
| S100A8 | −5.38002 | 15948.8 | 382.984 | 2.00E−06 |
| S100A9 | −5.62508 | 47530.9 | 963.073 | 1.20E−06 |
| TUBB3 | 1.52085 | 80.0591 | 229.738 | 0.0000132 |
| TUBB2C | −0.873906 | 16901.7 | 9222.71 | 0.0019696 |
| | Comp5 - | | | |
| | 33 mg/kg/VEH | | | |
| S100A8 | −5.24323 | 15486.8 | 408.877 | 3.00E−07 |
| S100A9 | −5.68502 | 48567.9 | 944.034 | 9.00E−07 |
| TUBB3 | 1.39838 | 63.6658 | 167.826 | 0.0000182 |
| TUBB2C | −1.41024 | 17370.5 | 6535.64 | 0.0000229 |
| | Comp4 - | | | |
| | 1 mg/kg/VEH | | | |
| S100A8 | −1.31235 | 14941 | 6016.2 | 2.81E−05 |
| S100A9 | −1.57948 | 41089.3 | 13748.6 | 4.40E−06 |
| TUBB3 | 0.23143 | 64.757 | 76.0246 | 0.0097577 |
| TUBB2C | −0.312444 | 16522.9 | 13305.5 | 0.0004177 |
| | Comp4 - | | | |
| | 3 mg/kg/VEH | | | |
| S100A8 | −3.14813 | 16439.6 | 1854.43 | 8.00E−07 |
| S100A9 | −3.54115 | 44522.6 | 3824.62 | 0.0000002 |
| TUBB3 | 0.335203 | 90.5137 | 114.188 | 0.0002296 |
| TUBB2C | −0.433932 | 15791.6 | 11689.6 | 0.000515 |
| | Comp4 - | | | |
| | 10 mg/kg/VEH | | | |
| S100A8 | −3.58663 | 14588.6 | 1214.31 | 8.77E−05 |
| S100A9 | −4.16055 | 44755.7 | 2502.64 | 1.70E−06 |
| TUBB3 | 0.916853 | 81.7996 | 154.437 | 0.0000333 |
| TUBB2C | −1.31564 | 16281.9 | 6541.2 | 0.0000418 |
| | Vehicle/VEH | | | |
| S100A8 | −0.230196 | 16016.3 | 13654.2 | 0.0027948 |
| S100A9 | −0.249258 | 41150.6 | 34621.2 | 0.0005574 |
| TUBB3 | −0.141257 | 64.3274 | 58.3275 | 0.0393814 |
| TUBB2C | 0.0808866 | 16317.6 | 17258.6 | 0.0759403 |

As shown in the three tables above, S100A8 and S100A9 were down-regulated by treatment with different LSD1 inhibitors in brain, spleen and liver. Two HOUSEKEEPING genes, Tubb3 and Tubb2c, were included for comparison. The potency of the effect on S100A9 and S100A8 expression was correlated to the biochemical LSD1 inhibitory potency in vitro, i.e Compound 3<Compound 4, Compound 5.

### Example 8: Quantification of S100A9 Expression by qRT-PCR in Cerebrospinal Fluid from Human Alzheimer's Disease Donors

8.1 Quantitative RT-PCR

Human S100A9 gene expression levels were analyzed by qRT-PCR using Taqman assay probe Hs00610058_m1, (Life Technologies; amplicon length 83 bp, targeting exon 2-3 boundary, RefSeq NM_002965.2, assay location 188) on total RNA extracted from the cell pellet obtained after centrifugation of 10 mL of human cerebrospinal fluid (CSF) from five different Alzheimer's Disease (AD) patient donors obtained from a biobank (PrecisionMed). After extraction (RNeasy Mini KIT; QIAGEN), all the RNA obtained was reverse transcribed to obtain 1st strand cDNA (using the kit iScript Reverse Transcription Supermix, Bio-Rad Ref. 170-8841)) in 20 μL of final volume. 1 μl of 1st strand product was used to perform in triplicate qRT-PCR reactions to analyze the Cp values of S100A9 as described in example 5 with an additional previous 10 cycle pre-amplification. Cp was normalized relative to the expression level of an endogenous reference gene (GADPH, Glyceraldehyde 3-phosphate dehydrogenase, also known as GAPDH) using Taqman assay probe Hs02758991_g1 (Life Technologies; amplicon length 93 bp, targeting exon 7-8 boundary, RefSeq NM_002046.4, assay location 704) and the results are expressed as Δ Cp (S100A9-GADPH).

8.2 Results

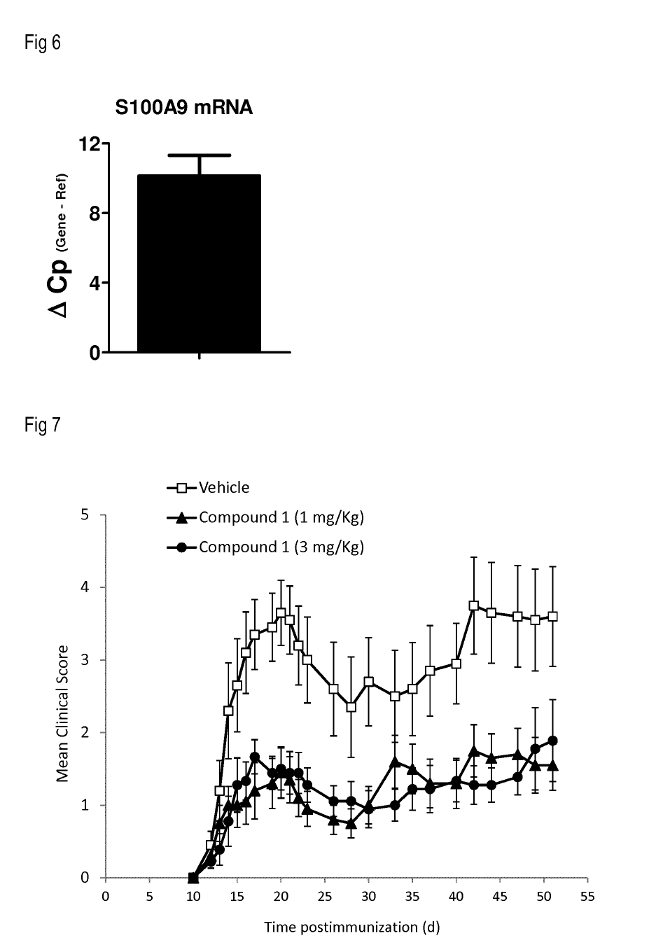

S100A9 expression in the CSF of human AD patient donors was quantified by qRT-PCR. The results obtained are shown in FIG. 6 as a mean+SEM value of the five different donors. These data show that S100A9 mRNA levels were detectable and quantifiable in human CSF samples.

Quantification of S100A9 expression in CSF from human healthy donors can be performed by qRT-PCR following an analogous method to the one described in Example 8.1.

### Example 9: Evaluation of the Efficacy of Compound 1 on Experimental Autoimmune Encephalomyelitis in Mice

The Experimental Autoimmune Encephalomyelitis (EAE) model shows pathologic and clinical similarities to human multiple sclerosis (MS) and is widely used as a model for MS. In particular, the murine EAE model as described herein, using MOG₃₅₋₅₅ and C57BL/6 mice strain, is considered a validated preclinical model of the chronic progressive form of MS.

9.1 Method

To induce chronic EAE by active immunization, C57BL/6 mice were immunized s.c. with 100 μg of myelin oligodendrocyte glycoprotein MOG₃₅₋₅₅ emulsified in complete Freund's adjuvant (CFA) containing 4 mg/ml Mycobacterium tuberculosis H37 RA. Mice also received i.p. injections of 200 ng of pertussis toxin on days 0 and 2. Treatment consisted in the oral administration of Compound 1 (at 1 mg/kg or 3 mg/kg) after the onset of the disease (day 12 postimmunization), once a day, for five consecutive days from day 12 to day 16 postimmunization and from day day 19 to to day 23 postimmunization. Control mice were orally treated with vehicle [2% v/v Tween-80+98% HPβCD (13% w/v)] following the same regime of administration as Compound 1. n=10 mice/group, with the exception of group treated with Compound 1 at 3 mg/kg where n=9.

Mice were scored daily for signs of EAE according to the following clinical scoring system: 0, no clinical signs; 0.5, partial loss of tail tonicity; 1, complete loss of tail tonicity; 2, flaccid tail and abnormal gait; 3, hind leg paralysis; 4, hind leg paralysis with hind body paresis; 5, hind and fore leg paralysis; and 6, death.

9.2 Results

Untreated control mice developed moderate (30% of animals reached a maximal clinical score of 1.5-3) to severe (70% of animals reached a maximal clinical score of 3.5-6) signs of EAE, and showed a mortality rate of 40% due to severe paralysis. Treatment with Compound 1 greatly inhibited the development of EAE and reduced disease incidence and severity measured by daily clinical score, as shown in FIG. 7. In the group treated with Compound 1, 40-70% of the mice displayed mild symptoms, and 30% almost completely recovered 40 days after disease onset. The protective effect of Compound 1 was maintained for a long-period of time after cessation of the treatment.

Based on the results obtained in this assay, Compound 1 is expected to be useful for the treatment of multiple sclerosis, including the chronic progressive form of multiple sclerosis.

### SEQUENCE LISTING

SEQ ID No. 1: Nucleotide Sequence Encoding Homo sapiens S100 Calcium Binding Protein A9 (S100A9), mRNA

NCBI Reference Sequence: NM_002965.3 The coding region ranges from nucleotide 44 to nucleotide 385 (highlighted in bold). It is understood that the mRNA corresponds to the sequence below (i.e. is identical to that sequence) with the exception that the “t” (thymidine) residue is replaced by a “uracil” (u) residue.

| ORIGIN | | |
|---|---|---|
| | 1 | aaacactctg tgtggctcct cggctttgac agagtgcaag acgatgactt gcaaaatgtc |
| | 61 | gcagctggaa cgcaacatag agaccatcat caacaccttc caccaatact ctgtgaagct |
| | 121 | ggggcaccca gacaccctga accaggggga attcaaagag ctggtgcgaa aagatctgca |
| | 181 | aaattttctc aagaaggaga ataagaatga aaaggtcata gaacacatca tggaggacct |
| | 241 | ggacacaaat gcagacaagc agctgagctt cgaggagttc atcatgctga tggcgaggct |
| | 301 | aacctgggcc tcccacgaga agatgcacga gggtgacgag ggccctggcc accaccataa |
| | 361 | gccaggcctc ggggagggca ccccctaaga ccacagtggc caagatcaca gtggccacgg |
| | 421 | ccacggccac agtcatggtg gccacggcca cagccactaa tcaggaggcc aggccaccct |
| | 481 | gcctctaccc aaccagggcc ccggggcctg ttatgtcaaa ctgtcttggc tgtggggcta |
| | 541 | ggggctgggg ccaaataaag tctcttcctc caagtcaaaa aaaaaa |
| // | | |

SEQ ID No. 2: Amino Acid Sequence of Homo sapiens S100 Calcium Binding Protein A9 (S100A9), Protein

UniProtKB/Swiss-Prot: S10A9_HUMAN, P06702

MTCKMSQLER NIETIINTFH QYSVKLGHPD TLNQGEFKEL VRKDLQNFLK KENKNEKVIE HIMEDLDTNA DKQLSFEEFI MLMARLTWAS HEKMHEGDEG PGHHHKPGLG EGTP

SEQ ID No. 3: Nucleotide Sequence Encoding Mus musculus S100 Calcium Binding Protein A9 (Calgranulin B) (S100a9), Transcript Variant 1, mRNA

NCBI Reference Sequence: NM_001281852.1 The coding region ranges from nucleotide 67 to nucleotide 405 (highlighted in bold). It is understood that the mRNA corresponds to the sequence below (i.e. is identical to that sequence) with the exception that the “t” (thymidine) residue is replaced by a “uracil” (u) residue.

ORIGIN

| | | |
|---|---|---|
| | 1 | tataaatact gggcttacac tgctcttacc aacatctgtg actctttagc cttgaagagc |
| | 61 | aagaagatgg ccaacaaagc accttctcag atggagcgca gcataaccac catcatcgac |
| | 121 | accttccatc aatactctag gaaggaagga caccctgaca ccctgagcaa gaaggaattc |
| | 181 | agacaaatgg tggaagcaca gttggcaacc tttatgaaga aagagaagag aaatgaagcc |
| | 241 | ctcataaatg acatcatgga ggacctggac acaaaccagg acaatcagct gagctttgag |
| | 301 | gagtgtatga tgctgatggc aaagttgatc tttgcctgtc atgagaagct gcatgagaac |
| | 361 | aacccacgtg ggcatggcca cagtcatggc aaaggctgtg ggaagtaatt aagaggtcag |
| | 421 | ccatgtgaca gctgcccaac caagtctaaa gggaatggct tactcaatgg cctttgttct |
| | 481 | gggaaatgat aagataaata ataaataagt ctttatccat tccaaaaaaa aaaaaaa |
| // | | |

SEQ ID No. 4: Amino Acid Sequence of Mus musculus S100 Calcium Binding Protein A9 (Calgranulin B) (S100a9), Protein

UniProtKB/Swiss-Prot: P31725-S10A9_MOUSE
MANKAPSQME RSITTIIDTF HQYSRKEGHP DTLSKKEFRQ
MVEAQLATFM KKEKRNEALI NDIMEDLDTN QDNQLSFEEC
MMLMAKLIFA CHEKLHENNP RGHGHSHGKG CGK

SEQ ID No. 5: Nucleotide Sequence Encoding Homo sapiens S100 Calcium Binding Protein A8 (S100A8), mRNA

NCBI Reference Sequence: NM_002964.4 The coding region ranges from nucleotide 71 to nucleotide 449 (highlighted in bold). It is understood that the mRNA corresponds to the sequence below (i.e. is identical to that sequence) with the exception that the “t” (thymidine) residue is replaced by a “uracil” (u) residue.

| ORIGIN | | |
|---|---|---|
| | 1 | gagaaaccag agactgtagc aactctggca gggagaagct gtctctgatg gcctgaagct |
| | 61 | gtgggcagct ggccaagcct aaccgctata aaaaggagct gcctctcagc cctgcatgtc |
| | 121 | tcttgtcagc tgtctttcag aagacctggt ggggcaagtc cgtgggcatc atgttgaccg |
| | 181 | agctggagaa agccttgaac tctatcatcg acgtctacca caagtactcc ctgataaagg |
| | 241 | ggaatttcca tgccgtctac agggatgacc tgaagaaatt gctagagacc gagtgtcctc |
| | 301 | agtatatcag gaaaaagggt gcagacgtct ggttcaaaga gttggatatc aacactgatg |
| | 361 | gtgcagttaa cttccaggag ttcctcattc tggtgataaa gatgggcgtg gcagcccaca |
| | 421 | aaaaaagcca tgaagaaagc cacaaagagt agctgagtta ctgggcccag aggctgggcc |
| | 481 | cctggacatg tacctgcaga ataataaagt catcaatacc tcaaaaaaaa aa |
| // | | |

SEQ ID No. 6: Amino Acid Sequence of Homo sapiens S100 Calcium Binding Protein A8 (S100A8), Protein

UniProtKB/Swiss-Prot: S10A8_HUMAN, P05109
MLTELEKALN SIIDVYHKYS LIKGNFHAVY RDDLKKLLET
ECPQYIRKKG ADVWFKELDI NTDGAVNFQE FLILVIKMGV
AAHKKSHEES HKE

SEQ ID No. 7: Nucleotide Sequence Encoding Mus musculus S100 Calcium Binding Protein A8 (Calgranulin A) (S100a8), mRNA

NCBI Reference Sequence: NM_013650.2 The coding region ranges from nucleotide 56 to nucleotide 322 (highlighted in bold). It is understood that the mRNA corresponds to the sequence below (i.e. is identical to that sequence) with the exception that the “t” (thymidine) residue is replaced by a “uracil” (u) residue.

| ORIGIN | | |
|---|---|---|
| | 1 | atcctttgtc agctccgtct tcaagacatc gtttgaaagg aaatctttcg tgacaatgcc |
| | 61 | gtctgaactg gagaaggcct tgagcaacct cattgatgtc taccacaatt attccaatat |
| | 121 | acaaggaaat caccatgccc tctacaagaa tgacttcaag aaaatggtca ctactgagtg |
| | 181 | tcctcagttt gtgcagaata taaatatcga aaacttgttc agagaattgg acatcaatag |
| | 241 | tgacaatgca attaacttcg aggagttcct tgcgatggtg ataaaagtgg gtgtggcatc |
| | 301 | tcacaaagac agccacaagg agtagcagag cttctggcct agggctgggt ccctggatat |
| | 361 | gtctacagaa taaagtcatc atatctcagg tc |
| // | | |

SEQ ID No. 8: Amino Acid Sequence of Mus musculus S100 Calcium Binding Protein A8 (Calgranulin A) (S100a8), Protein

UniProtKB/Swiss-Prot: P27005-S10A8_MOUSE
MPSELEKALS NLIDVYHNYS NIQGNHHALY KNDFKKMVTT
ECPQFVQNIN IENLFRELDI NSDNAINFEE FLAMVIKVGV
ASHKDSHKE

Unless defined otherwise, all technical and scientific terms used herein have the same meaning as commonly understood by one of ordinary skill in the art to which this invention pertains.

All publications, patents and patent applications cited herein are hereby incorporated herein by reference in their entireties.

The publications, patents and patent applications mentioned in the specification are provided solely for their disclosure prior to the filing date of the present application. Nothing herein is to be construed as an admission that they are prior art to the instant application.

While the invention has been described in connection with specific embodiments thereof, it will be understood that it is capable of further modifications and this application is intended to cover any variations, uses or adaptations of the invention following, in general, the principles of the invention and including such departures from the present disclosure as come within known or customary practice within the art to which the invention pertains and as may be applied to the essential features hereinbefore set forth and as follows in the appended claims.

1. A method for monitoring the response of a subject to treatment with an LSD1 inhibitor, the method comprising determining the level of a biomarker which is S100A9 and/or S100A8 in a sample obtained from the subject, wherein a decrease in the level of the biomarker in the sample as compared to the level of the biomarker in a control indicates a response to the treatment with the LSD1 inhibitor.

2. The method of claim 1, wherein the subject has a CNS disease.

3. The method of claim 1, wherein the subject has Alzheimer's disease.

4. The method of claim 1, wherein the subject has multiple sclerosis.

5. The method of claim 1, wherein the biomarker is S100A9.

6. The method of claim 1, wherein the LSD1 inhibitor is a 2-(hetero)arylcyclopropylamino compound.

7. (canceled)

8. (canceled)

9. A method for determining whether a subject is likely to respond to treatment with an LSD1 inhibitor, the method comprising determining the level of a biomarker which is S100A9 and/or S100A8 in a sample obtained from the subject prior to treatment with the LSD1 inhibitor, wherein if the level of the biomarker in the sample is elevated as compared to a control, the subject is likely to respond to the treatment with an LSD1 inhibitor.

10. The method of claim 9, wherein the subject has a CNS disease.

11. The method of claim 9, wherein the subject has Alzheimer's disease.

12. The method of claim 9, wherein the subject has multiple sclerosis.

13. The method of claim 9, wherein the biomarker is S100A9.

14. The method of claim 9, wherein the LSD1 inhibitor is a 2-(hetero)arylcyclopropylamino compound.

15-22. (canceled)

23. The method of claim 1, wherein the LSD1 inhibitor is a compound of formula (VIII) or an enantiomer, a diastereomer or a mixture of stereoisomers thereof, or a pharmaceutically acceptable salt or solvate thereof: 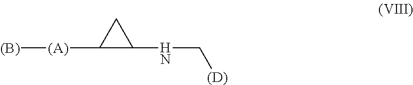
wherein:
(A) is a cyclyl group having n substituents (R3);
(B) is chosen from a cyclyl group and an -(L1)-cyclyl group, wherein said cyclyl group or the cyclyl moiety comprised in said -(L1)-cyclyl group has n substituents (R2);
(L1) is chosen from —O—, —NH—, —N(alkyl)-, alkylene and heteroalkylene;
(D) is chosen from heteroaryl group and an -(L2)-heteroaryl group, wherein the heteroaryl group or the heteroaryl moiety comprised in the -(L2)-heteroaryl group has one substituent (R1), and further wherein the heteroaryl group is covalently bonded to the remainder of the molecule through a ring carbon atom or the heteroaryl moiety comprised in the -(L2)-heteroaryl group is covalently bonded to the (L2) moiety through a ring carbon atom;
(L2) is chosen from —O—, —NH—, —N(alkyl)-, alkylene and heteroalkylene;
(R1) is a hydrogen bonding group chosen from —OH, —NH₂, amido, —S(O)₂NH₂, —C(═O)NH₂, —CH₂—C(═O)NH₂, —NH—C(═O)CH₃, —NHCH₃, —N(CH₃)₂ and —CH₂—NH₂;
(R2) is independently chosen from alkyl, alkenyl, alkynyl, cyclyl, amino, amido, C-amido, alkylamino, hydroxyl, nitro, halo, haloalkyl, haloalkoxy, cyano, sulfinyl, sulfonyl, sulfonamide, alkoxy, acyl, carboxyl, carbamate and urea;
(R3) is independently chosen from alkyl, alkenyl, alkynyl, cyclyl, amino, amido, C-amido, alkylamino, hydroxyl, nitro, halo, haloalkyl, haloalkoxy, cyano, sulfinyl, sulfonyl, sulfonamide, alkoxy, acyl, carboxyl, carbamate, and urea; and
n is independently chosen from 0, 1, 2, 3 and 4.

24. The method of claim 23, wherein the LSD1 inhibitor is a compound chosen from:
5-(((trans)-2-(4-(benzyloxy)phenyl)cyclopropylamino)methyl)pyrimidin-2-amine;
5-(((trans)-2-(4-(benzyloxy)phenyl)cyclopropylamino)methyl)thiazol-2-amine;
5-(((trans)-2-(6-(3-(trifluoromethyl)phenyl)pyridin-3-yl)cyclopropylamino)methyl) pyrimidin-2-amine;
5-(((trans)-2-(6-(3-(trifluoromethyl)phenyl)pyridin-3-yl)cyclopropylamino)methyl) thiazol-2-amine;
3-(5-((trans)-2-((2-aminopyrimidin-5-yl)methylamino)cyclopropyl)pyridin-2-yl)phenol;
3-(5-((trans)-2-((2-aminothiazol-5-yl)methylamino)cyclopropyl)pyridin-2-yl)phenol;
4′-((trans)-2-((2-aminopyrimidin-5-yl)methylamino)cyclopropyl)biphenyl-3-ol;
4′-((trans)-2-((2-aminothiazol-5-yl)methylamino)cyclopropyl)biphenyl-3-ol;
5-(((trans)-2-(4-(benzyloxy)phenyl)cyclopropylamino)methyl)-1,2,4-oxadiazol-3-amine;
5-(((trans)-2-(4-(benzyloxy)phenyl)cyclopropylamino)methyl)-1,3,4-oxadiazol-2-amine;
5-((((trans)-2-(4-((4-fluorobenzyl)oxy)phenyl)cyclopropyl)amino)methyl)-1,3,4-oxadiazol-2-amine;
5-((((trans)-2-(4-((3-fluorobenzyl)oxy)phenyl)cyclopropyl)amino)methyl)-1,3,4-oxadiazol-2-amine;
5-((((trans)-2-(4-((3,5-difluorobenzyl)oxy)phenyl)cyclopropyl)amino)methyl)-1,3,4-oxadiazol-2-amine;
5-((((trans)-2-(4-((4-chlorobenzyl)oxy)phenyl)cyclopropyl)amino)methyl)-1,3,4-oxadiazol-2-amine;
5-((((trans)-2-(4-((3-chlorobenzyl)oxy)phenyl)cyclopropyl)amino)methyl)-1,3,4-oxadiazol-2-amine;
5-((((trans)-2-(4-((2-fluorobenzyl)oxy)phenyl)cyclopropyl)amino)methyl)-1,3,4-oxadiazol-2-amine;
5-((((trans)-2-(4-(benzyloxy)phenyl)cyclopropyl)amino)methyl)-N-methyl-1,3,4-oxadiazol-2-amine;
N-(5-((((trans)-2-(4-(benzyloxy)phenyl)cyclopropyl)amino)methyl)-1,3,4-oxadiazol-2-yl)acetamide;
4′-((trans)-2-(((5-amino-1,3,4-oxadiazol-2-yl)methyl)amino)cyclopropyl)-[1,1′-biphenyl]-3-ol;
5-((((trans)-2-(6-(3-(trifluoromethyl)phenyl)pyridin-3-yl)cyclopropyl)amino)methyl)-1,3,4-oxadiazol-2-amine;
5-((((trans)-2-(4-(benzyloxy)phenyl)cyclopropyl)amino)methyl)-1,3,4-thiadiazol-2-amine;
2-((((trans)-2-(4-(benzyloxy)phenyl)cyclopropyl)amino)methyl)thiazol-5-amine;
4-((((trans)-2-(3′-(trifluoromethyl)-[1,1′-biphenyl]-4-yl)cyclopropyl)amino)methyl)thiazol-2-amine;
2-((((trans)-2-(4-(benzyloxy)phenyl)cyclopropyl)amino)methyl)oxazol-5-amine;
3-((((trans)-2-(4-(benzyloxy)phenyl)cyclopropyl)amino)methyl)isoxazol-5-amine;
5-((((trans)-2-(4-(benzyloxy)phenyl)cyclopropyl)amino)methyl)-N,N-dimethyl-1,3,4-oxadiazol-3-amine;
3-((((trans)-2-(4-(benzyloxy)phenyl)cyclopropyl)amino)methyl)-1,2,4-oxadiazol-5-amine;
5-((((trans)-2-(4-(benzyloxy)phenyl)cyclopropyl)amino)methyl)-1,2,4-thiadiazol-3-amine;
5-((((trans)-2-(4-(benzyloxy)phenyl)cyclopropyl)amino)methyl)pyridin-2-amine;
6-((((trans)-2-(4-(benzyloxy)phenyl)cyclopropyl)amino)methyl)pyridazin-3-amine;
5-((((trans)-2-(4-(benzyloxy)phenyl)cyclopropyl)amino)methyl)pyrazin-2-amine;
2-((((trans)-2-(4-(benzyloxy)phenyl)cyclopropyl)amino)methyl)pyrimidin-5-amine;
6-((((trans)-2-(4-(benzyloxy)phenyl)cyclopropyl)amino)methyl)-1,2,4-triazin-3-amine;
3-((((trans)-2-(4-(benzyloxy)phenyl)cyclopropyl)amino)methyl)-1,2,4-triazin-6-amine;
(−) 5-((((trans)-2-(4-(benzyloxy)phenyl)cyclopropyl)amino)methyl)-1,3,4-oxadiazol-2-amine;
(−) 5-((((trans)-2-(4-((3-fluorobenzyl)oxy)phenyl)cyclopropyl)amino)methyl)-1,3,4-oxadiazol-2-amine;
(−) 5-((((trans)-2-(4-(benzyloxy)phenyl)cyclopropyl)amino)methyl)-N-methyl-1,3,4-oxadiazol-2-amine;
(−)N-(5-((((trans)-2-(4-(benzyloxy)phenyl)cyclopropyl)amino)methyl)-1,3,4-oxadiazol-2-yl)acetamide;
(−) 5-((((trans)-2-(4-(benzyloxy)phenyl)cyclopropyl)amino)methyl)pyrimidin-2-amine;
(−) 5-((((trans)-2-(4-(benzyloxy)phenyl)cyclopropyl)amino)methyl)-1,3,4-thiadiazol-2-amine;
(−) 5-((((trans)-2-(4-((2-fluorobenzyl)oxy)phenyl)cyclopropyl)amino)methyl)-1,3,4-oxadiazol-2-amine;
or a pharmaceutically acceptable salt or solvate thereof.

25. The method of claim 1, wherein the LSD1 inhibitor is a compound of formula (IV) or an enantiomer, a diastereomer or a mixture of stereoisomers thereof, or a pharmaceutically acceptable salt or solvate thereof:
(A′)_{X}-(A)-(B)-(Z)-(L)-C(═O)NH₂ (IV)
wherein:
(A) is chosen from heteroaryl and aryl;
each (A′), when present, is independently chosen from aryl, arylalkoxy, arylalkyl, heterocyclyl, aryloxy, halo, alkoxy, haloalkyl, cycloalkyl, haloalkoxy, and cyano, wherein each (A′) is substituted with 0, 1, 2 or 3 substituents independently chosen from halo, haloalkyl, aryl, arylalkoxy, alkyl, alkoxy, cyano, sulfonyl, sulfinyl, and carboxamide;
X is chosen from 0, 1, 2, and 3;
(B) is a cyclopropyl ring, wherein (A) and (Z) are covalently bonded to different carbon atoms of (B);
(Z) is —NH—; and
(L) is —(CH₂)ₘCR₁R₂—, wherein m is chosen from 0, 1, 2, 3, 4, 5, and 6, and wherein R₁ and R₂ are each independently chosen from hydrogen and C₁-C₆ alkyl;
provided that, if (L) is —CH₂— or —CH(CH₃)—, then X is not 0.

26. The method of claim 25, wherein the LSD1 inhibitor is a compound chosen from:
2-((trans)-2-(4-(4-cyanobenzyloxy)phenyl)cyclopropylamino)acetamide,
2-((trans)-2-(4-(3-cyanobenzyloxy)phenyl)cyclopropylamino)acetamide,
2-((trans)-2-(4-(benzyloxy)phenyl)cyclopropylamino)acetamide,
2-((trans)-2-(4-(4-fluorobenzyloxy)phenyl)cyclopropylamino)acetamide,
2-((trans)-2-(4-(3-fluorobenzyloxy)phenyl)cyclopropylamino)acetamide,
2-((trans)-2-(4-(3-chlorobenzyloxy)phenyl)cyclopropylamino)acetamide,
2-((trans)-2-(4-(4-chlorobenzyloxy)phenyl)cyclopropylamino)acetamide,
2-((trans)-2-(4-(3-bromobenzyloxy)phenyl)cyclopropylamino)acetamide,
2-((trans)-2-(4-(3,5-difluorobenzyloxy)phenyl)cyclopropylamino)acetamide,
2-((trans)-2-(4-phenethoxyphenyl)cyclopropylamino)acetamide,
2-((trans)-2-(3′-(trifluoromethyl)biphenyl-4-yl)cyclopropylamino)acetamide,
2-((trans)-2-(3′-chlorobiphenyl-4-yl)cyclopropylamino)acetamide,
2-((trans)-2-(6-(4-chlorophenyl)pyridin-3-yl)cyclopropylamino)acetamide,
(R)-2-((trans)-2-(4-(3-fluorobenzyloxy)phenyl)cyclopropylamino)propanamide,
(S)-2-((trans)-2-(4-(4-fluorobenzyloxy)phenyl)cyclopropylamino)propanamide,
(R)-2-((trans)-2-(4-(4-fluorobenzyloxy)phenyl)cyclopropylamino)propanamide,
(S)-2-((trans)-2-(4-(4-fluorobenzyloxy)phenyl)cyclopropylamino)propanamide,
(R)-2-((trans)-2-(4-(benzyloxy)phenyl)cyclopropylamino)propanamide,
(S)-2-((trans)-2-(4-(benzyloxy)phenyl)cyclopropylamino)propanamide,
2-(2-[1,1′;4′,1″]Terphenyl-4″-yl-cyclopropylamino)acetamide,
5′-((trans)-2-(2-amino-2-oxoethylamino)cyclopropyl)-2′-(benzyloxy)biphenyl-3-carboxamide,
5-((trans)-2-(4′-chlorobiphenyl-4-yl)cyclopropylamino)pentanamide,
3-((trans)-2-(4-(3-bromobenzyloxy)phenyl)cyclopropylamino)propanamide,
4-((trans)-2-phenylcyclopropylamino)butanamide,
5-((trans)-2-phenylcyclopropylamino)pentanamide,
5-((trans)-2-(4′-chlorobiphenyl-4-yl)cyclopropylamino)-2-methylpentanamide,
4-((trans)-2-(4′-chlorobiphenyl-4-yl)cyclopropylamino)-2-methylbutanamide,
3-((trans)-2-(4-(3-fluorobenzyloxy)phenyl)cyclopropylamino)-2,2-dimethylpropanamide,
3-((trans)-2-(4′-chlorobiphenyl-4-yl)cyclopropylamino)propanamide,
4-((trans)-2-(4′-chlorobiphenyl-4-yl)cyclopropylamino)butanamide,
4-((trans)-2-(4-(3-bromobenzyloxy)phenyl)cyclopropylamino)butanamide,
5-((trans)-2-(4-(3-bromobenzyloxy)phenyl)cyclopropylamino)pentanamide,
5-((trans)-2-(6-(benzyloxy)biphenyl-3-yl)cyclopropylamino)pentanamide,
4-((trans)-2-(6-(benzyloxy)biphenyl-3-yl)cyclopropylamino)butanamide,
or a pharmaceutically acceptable salt or solvate thereof.

27. The method of claim 1, wherein the LSD1 inhibitor is a compound of formula (I) or an enantiomer, a diastereomer or a mixture of stereoisomers thereof, or a pharmaceutically acceptable salt or solvate thereof: 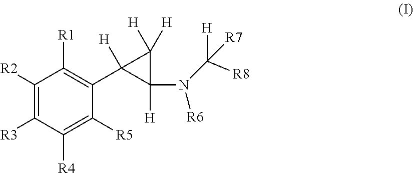
wherein:
each of R1-R5 is optionally substituted and independently chosen from —H, halo, alkyl, alkoxy, cycloalkoxy, haloalkyl, haloalkoxy, -L-aryl, -L-heteroaryl, -L-heterocyclyl, -L-carbocycle, acylamino, acyloxy, alkylthio, cycloalkylthio, alkynyl, amino, aryl, arylalkyl, arylalkenyl, arylalkynyl, arylalkoxy, aryloxy, arylthio, heteroarylthio, cyano, cyanato, haloaryl, hydroxyl, heteroaryloxy, heteroarylalkoxy, isocyanato, isothiocyanato, nitro, sulfinyl, sulfonyl, sulfonamide, thiocarbonyl, thiocyanato, trihalomethanesulfonamido, O-carbamyl, N-carbamyl, O-thiocarbamyl, N-thiocarbamyl, and C-amido;
R6 is chosen from —H and alkyl;
R7 is chosen from —H, alkyl, and cycloalkyl;
R8 is chosen from —C(═O)NRₓR_{y} and —C(═O)R_{z};
Rₓ when present is chosen from —H, alkyl, alkynyl, alkenyl, -L-carbocycle, -L-aryl, -L-heterocyclyl, all of which are optionally substituted;
R_{y} when present is chosen from —H, alkyl, alkynyl, alkenyl, -L-carbocycle, -L-aryl, -L-heterocyclyl, all of which are optionally substituted;
R_{z} when present is chosen from —H, alkoxy, -L-carbocyclic, -L-heterocyclic, -L-aryl, wherein the aryl, heterocyclyl, or carbocycle is optionally substituted;
each L is saturated, partially saturated, or unsaturated, and is independently chosen from —(CH₂)ₙ—(CH₂)ₙ—, —(CH₂)ₙC(═O)(CH₂)ₙ—, —(CH₂)ₙC(═O)NH(CH₂)ₙ—, —(CH₂)ₙNHC(═O)O(CH₂)ₙ—, —(CH₂)ₙNHC(═O)NH(CH₂)ₙ—, —(CH₂)ₙNHC(═S)S(CH₂)ₙ—, —(CH₂)ₙOC(═O)S(CH₂)ₙ—, —(CH₂)ₙNH(CH₂)ₙ—, —(CH₂)ₙO(CH₂)ₙ—, —(CH₂)ₙS(CH₂)ₙ—, and —(CH₂)ₙNHC(═S)NH(CH₂)ₙ—, where each n is independently chosen from 0, 1, 2, 3, 4, 5, 6, 7, and 8;
wherein optionally substituted refers to zero or 1 to 4 optional substituents independently chosen from acylamino, acyloxy, alkenyl, alkoxy, cycloalkoxy, alkyl, alkylthio, cycloalkylthio, alkynyl, amino, aryl, arylalkyl, arylalkenyl, arylalkynyl, arylalkoxy, aryloxy, arylthio, heteroarylthio, carbocyclyl, cyano, cyanato, halo, haloalkyl, haloaryl, hydroxyl, heteroaryl, heteroaryloxy, heterocyclyl, heteroarylalkoxy, isocyanato, isothiocyanato, nitro, sulfinyl, sulfonyl, sulfonamide, thiocarbonyl, thiocyanato, trihalomethanesulfonamido, O-carbamyl, N-carbamyl, O-thiocarbamyl, N-thiocarbamyl, and C-amido.

28. The method of claim 27, wherein the LSD1 inhibitor is a compound chosen from:
N-cyclopropyl-2-{[(trans)-2-phenylcyclopropyl]amino}acetamide;
2-{[(trans)-2-phenylcyclopropyl]amino}acetamide;
N-cyclopropyl-2-{[(trans)-2-phenylcyclopropyl]amino}propanamide;
2-{[(trans)-2-phenylcyclopropyl]amino}-N-prop-2-ynylacetamide;
N-isopropyl-2-{[(trans)-2-phenylcyclopropyl]amino}acetamide;
N-(tert-butyl)-2-{[(trans)-2-phenylcyclopropyl]amino}acetamide;
N-(2-morpholin-4-yl-2-oxoethyl)-N-[(trans)-2-phenylcyclopropyl]amine;
2-{[(trans)-2-phenylcyclopropyl]amino}propanamide;
Methyl 2-{[(trans)-2-phenylcyclopropyl]amino}propanoate;
N-cyclopropyl-2-{methyl[(trans)-2-phenylcyclopropyl]amino}acetamide;
2-{methyl[(trans)-2-phenylcyclopropyl]amino}acetamide;
N-methyl-trans-2-(Phenylcyclopropylamino)propanamide;
1-(4-methylpiperazin-1-yl)-2-((trans)-2-phenylcyclopropylamino)ethanone;
1-(4-ethylpiperazin-1-yl)-2-((trans)-2-phenylcyclopropylamino)ethanone;
1-(4-benzylpiperazin-1-yl)-2-((trans)-2-phenylcyclopropylamino)ethanone;
2-((trans)-2-phenylcyclopropylamino)-1-(4-phenylpiperazin-1-yl)ethanone;
2-((trans)-2-(4-(benzyloxy)phenyl)cyclopropylamino)-1-(4-methylpiperazin-1-yl)ethanone;
2-((trans)-2-(4-(benzyloxy)phenyl)cyclopropylamino)-N-cyclopropylacetamide;
2-((trans)-2-(4-(3-fluorobenzyloxy)phenyl)cyclopropylamino)-1-(4-methylpiperazin-1-yl)ethanone;
2-((trans)-2-(4-(3-chlorobenzyloxy)phenyl)cyclopropylamino)-1-(4-methylpiperazin-1-yl)ethanone;
2-((trans)-2-(biphenyl-4-yl)cyclopropylamino)-1-(4-methylpiperazin-1-yl)ethanone;
1-(4-methylpiperazin-1-yl)-2-((trans)-2-(4-phenethoxyphenyl)cyclopropylamino) ethanone;
2-((trans)-2-(4-(4-fluorobenzyloxy)phenyl)cyclopropylamino)-1-(4-methylpiperazin-1-yl)ethanone;
2-((trans)-2-(4-(biphenyl-4-ylmethoxy)phenyl)cyclopropylamino)-1-(4-methylpiperazin-1-yl)ethanone;
2-({(trans)-2-[4-(benzyloxy)phenyl]cyclopropyl}amino)-N-cyclopropylacetamide,
N-[(trans)-2-(4-benzyloxyphenyl)cyclopropyl]}-N-[2-(4-methylpiperazin-1-yl)-2-oxoethyl]amine,
N-[2-oxo-2-(4-phenylpiperazin-1-yl)ethyl]-N-[(trans)-2-phenylcyclopropyl]amine,
N-[2-(4-benzylpiperazin-1-yl)-2-oxoethyl]-N-[(trans)-2-phenylcyclopropyl]amine,
N-[2-(4-ethylpiperazin-1-yl)-2-oxoethyl]-N-[(trans)-2-phenylcyclopropyl]amine,
N-[2-(4-methylpiperazin-1-yl)-2-oxoethyl]-N-[(trans)-2-phenylcyclopropyl]amine,
2-((trans)-2-(4-pyridin-3-ylphenyl)cyclopropylamino)-1-(4-methylpiperazin-1-yl)ethanone,
2-((trans)-2-(3′-methoxy-1,1′-biphenyl-4-yl)cyclopropylamino)-1-(4-methylpiperazin-1-yl)ethanone,
or a pharmaceutically acceptable salt or solvate thereof.

29. The method of claim 1, wherein the LSD1 inhibitor is a compound of formula (III) or an enantiomer, a diastereomer or a mixture of stereoisomers thereof, or a pharmaceutically acceptable salt or solvate thereof:
(A′)_{X}-(A)-(B)-(Z)-(L)-(D) (III)
(A) is heteroaryl or aryl;
each (A′), if present, is independently chosen from aryl, arylalkoxy, arylalkyl, heterocyclyl, aryloxy, halo, alkoxy, haloalkyl, cycloalkyl, haloalkoxy, and cyano, wherein each (A′) is substituted with 0, 1, 2, or 3 substituents independently chosen from halo, haloalkyl, aryl, arylalkoxy, alkyl, alkoxy, cyano, sulfonyl, amido, and sulfinyl;
X is chosen from 0, 1, 2, and 3;
(B) is a cyclopropyl ring, wherein (A) and (Z) are covalently bonded to different carbon atoms of (B);
(Z) is —NH—;
(L) is chosen from —CH₂CH₂—, —CH₂CH₂CH₂—, and —CH₂CH₂CH₂CH₂—; and
(D) is chosen from —N(—R1)-R2, —O—R3, and —S—R3, wherein:
R1 and R2 are mutually linked to form a heterocyclic ring together with the nitrogen atom that R1 and R2 are attached to, wherein the heterocyclic ring has 0, 1, 2, or 3 substituents independently chosen from —NH₂, —NH(C₁-C₆ alkyl), —N(C₁-C₆ alkyl)(C₁-C₆ alkyl), alkyl, halo, cyano, alkoxy, haloalkyl, and haloalkoxy, or
R1 and R2 are independently chosen from —H, alkyl, cycloalkyl, haloalkyl, and heterocyclyl, wherein the sum of substituents on R1 and R2 together is 0, 1, 2, or 3, and the substituents are independently chosen from —NH₂, —NH(C₁-C₆ alkyl), —N(C₁-C₆ alkyl)(C₁-C₆ alkyl), and fluoro; and
R3 is chosen from —H, alkyl, cycloalkyl, haloalkyl, and heterocyclyl, wherein R3 has 0, 1, 2, or 3 substituents independently chosen from —NH₂, —NH(C₁-C₆ alkyl), —N(C₁-C₆ alkyl)(C₁-C₆ alkyl), and fluoro.

30. The method of claim 29, wherein the LSD1 inhibitor is a compound chosen from:
N-[2-(4-methylpiperazin-1-yl)ethyl]-N-[(trans)-2-phenylcyclopropyl]amine;
N-cyclopropyl-N′-[(trans)-2-phenylcyclopropyl]ethane-1,2-diamine;
N,N-dimethyl-N′-(2-{[(trans)-2-phenylcyclopropyl]amino}ethyl)ethane-1,2-diamine;
(3R)-1-(2-{[(trans)-2-phenylcyclopropyl]amino}ethyl)pyrrolidin-3-amine;
(3S)—N,N-dimethyl-1-(2-{[(trans)-2-phenylcyclopropyl]amino}ethyl) pyrrolidin-3-amine;
(3R)—N,N-dimethyl-1-(2-{[(trans)-2-phenylcyclopropyl]amino}ethyl)pyrrolidin-3-amine;
N-[(trans)-2-phenylcyclopropyl]-N-(2-piperazin-1-ylethyl)amine;
N1,N1-diethyl-N2-((trans)-2-phenylcyclopropyl)ethane-1,2-diamine;
N-[(trans)-2-phenylcyclopropyl]-N-(2-piperidin-1-ylethyl)amine;
(trans)-2-(4-(benzyloxy)phenyl)-N-(2-(4-methylpiperazin-1-yl)ethyl)cyclopropanamine;
(trans)-N-(2-(4-methylpiperazin-1-yl)ethyl)-2-(3′-(trifluoromethyl) biphenyl-4-yl)cyclopropanamine;
(trans)-2-(3′-chlorobiphenyl-4-yl)-N-(2-(4-methylpiperazin-1-yl)ethyl)cyclopropanamine;
(R)-1-(2-((trans)-2-(3′-(trifluoromethyl)biphenyl-4-yl)cyclopropylamino)ethyl) pyrrolidin-3-amine; and
N¹-cyclopropyl-N²-((trans)-2-(3′-(trifluoromethyl)biphenyl-4-yl)cyclopropyl)ethane-1,2-diamine;
N1-((trans)-2-(4-(3-bromobenzyloxy)phenyl)cyclopropyl)-N2-cyclopropylethane-1,2-diamine;
N1-((trans)-2-(3′-chlorobiphenyl-4-yl)cyclopropyl)-N2-cyclopropylethane-1,2-diamine;
N1-cyclopropyl-N2-((trans)-2-(4-phenethoxyphenyl)cyclopropyl)ethane-1,2-diamine;
N1,N1-diethyl-N2-((trans)-2-(4-(3-fluorobenzyloxy)phenyl)cyclopropyl)ethane-1,2-diamine;
(trans)-2-(4-bromophenyl)-N-(2-(4-methylpiperazin-1-yl)ethyl)cyclopropanamine;
N1-((trans)-2-(terphenyl-4-yl)cyclopropyl)-N2-cyclopropylethane-1,2-diamine;
(trans)-N-(2-(piperidin-1-yl)ethyl)-2-(3′-(trifluoromethyl)biphenyl-4-yl)cyclopropanamine;
N1,N1-diethyl-N2-((trans)-2-(3′-(trifluoromethyl)biphenyl-4-yl)cyclopropyl)ethane-1,2-diamine;
(trans)-N-(2-(piperazin-1-yl)ethyl)-2-(3′-(trifluoromethyl)biphenyl-4-yl)cyclopropanamine;
(S)-1-(2-((trans)-2-(3′-(trifluoromethyl)biphenyl-4-yl)cyclopropylamino)ethyl) pyrrolidin-3-amine;
(R)-1-(2-((trans)-2-(3′-chlorobiphenyl-4-yl)cyclopropylamino)ethyl)pyrrolidin-3-amine;
(R)-1-(2-((trans)-2-(4′-chlorobiphenyl-4-yl)cyclopropylamino)ethyl)pyrrolidin-3-amine;
(R)-1-(2-((trans)-2-(3′-methoxybiphenyl-4-yl)cyclopropylamino)ethyl)pyrrolidin-3-amine;
(R)-1-(2-((trans)-2-(4-(3-bromobenzyloxy)phenyl)cyclopropylamino)ethyl) pyrrolidin-3-amine; and
(R)-1-(2-((trans)-2-(6-(3-(trifluoromethyl)phenyl)pyridin-3-yl)cyclopropylamino)ethyl)pyrrolidin-3-amine;
N-(trans)-2-(isobutylthio)-ethyl-2-phenylcyclopropanamine,
N-trans-(2-ethoxyethyl)-2-phenylcyclopropanamine,
N-trans-(2-methoxyethyl)-2-phenylcyclopropanamine,
(R)-1-(2-((trans)-2-(4-(4-bromobenzyloxy)phenyl)cyclopropylamino)ethyl)pyrrolidin-3-amine;
(R)-1-(2-((trans)-2-(4-(4-chlorobenzyloxy)phenyl)cyclopropylamino)ethyl) pyrrolidin-3-amine;
(R)-1-(2-((trans)-2-(4-(biphenyl-4-ylmethoxy)phenyl)cyclopropylamino) ethyl)pyrrolidin-3-amine;
(R)-1-(2-((trans)-2-(3′,5′-dichlorobiphenyl-4-yl)cyclopropylamino)ethyl)pyrrolidin-3-amine;
N1-((trans)-2-(2-[1,1′;4′,1″]terphenyl-4″-yl-cyclopropyl)-N2-cyclopropylethane-1,2-diamine;
(R)-1-(2-((trans)-2-(6-(benzyloxy)-4′-(trifluoromethyl)biphenyl-3-yl)cyclopropylamino)ethyl)pyrrolidin-3-amine; and
(R)-1-(2-((trans)-2-(6-(benzyloxy)biphenyl-3-yl)cyclopropylamino)ethyl)pyrrolidin-3-amine;
(R)-1-(2-((trans)-2-(4-phenethoxyphenyl)cyclopropylamino)ethyl)pyrrolidin-3-amine;
(R)-1-(2-((trans)-2-(6-(3-methoxyphenyl)pyridin-3-yl)cyclopropylamino)ethyl) pyrrolidin-3-amine;
(R)-1-(2-((trans)-2-(6-(4-chlorophenyl)pyridin-3-yl)cyclopropylamino)ethyl) pyrrolidin-3-amine; and
4-((4-((trans)-2-(2-((R)-3-aminopyrrolidin-1-yl)ethylamino)cyclopropyl)phenoxy) methyl)benzonitrile;
or a pharmaceutically acceptable salt or solvate thereof.
